(19) United States
(12) Patent Application Publication
(10) Pub. No.: US 2023/0203004 A1
(43) Pub. Date: Jun. 29, 2023

(54) RSV INHIBITING 3-SUBSTITUTED QUINOLINE AND CINNOLINE DERIVATIVES

(71) Applicant: Janssen Sciences Ireland Unlimited Company, Co Cork (IE)

(72) Inventors: Sandrine Céline GROSSE, Beerse (BE); Jérôme Émile Georges GUILLEMONT, Issy-les-Moulineaux Cedex (FR); Pierre Jean-Marie Bernard RABOISSON, Beerse (BE); David Craig MC GOWAN, Beerse (BE); Werner Constant J. EMBRECHTS, Beerse (BE); Ludwig Paul COOYMANS, Beerse (BE); Tim Hugo Maria JONCKERS, Beerse (BE)

(21) Appl. No.: 17/995,756

(22) PCT Filed: Apr. 21, 2021
(86) PCT No.: PCT/EP2021/060384

§ 371 Date: Oct. 7, 2022

(30) Foreign Application Priority Data
Apr. 21, 2020 (EP) 20170578.7

(51) Int. Cl. C07D 401/12; A61K 45/06; A61P 31/14

(52) U.S. Cl. CPC C07D 401/12; A61K 45/06; A61P 31/14

(57) ABSTRACT

The invention concerns compounds of formula (I) having antiviral activity, in particular, having an inhibitory activity on the replication of the respiratory syncytial virus (RSV). The invention further concerns pharmaceutical compositions comprising these compounds and the compounds for use in the treatment of respiratory syncytial virus infection.

### FIELD OF THE INVENTION

The invention concerns compounds having antiviral activity, in particular having an inhibitory activity on the replication of the respiratory syncytial virus (RSV). The invention further concerns pharmaceutical compositions comprising these compounds and the compounds for use in the treatment of respiratory syncytial virus infection.

### BACKGROUND

Human RSV or Respiratory Syncytial Virus is a large RNA virus, member of the family of Pneumoviridae, genus Orthopneumovirus together with bovine RSV virus. Human RSV is responsible for a spectrum of respiratory tract diseases in people of all ages throughout the world. It is the major cause of lower respiratory tract illness during infancy and childhood. Over half of all infants encounter RSV in their first year of life, and almost all within their first two years. The infection in young children can cause lung damage that persists for years and may contribute to chronic lung disease in later life (chronic wheezing, asthma). Older children and adults often suffer from a (bad) common cold upon RSV infection. In old age, susceptibility again increases, and RSV has been implicated in a number of outbreaks of pneumonia in the aged resulting in significant mortality.

RSV has been classified in two antigenic subtypes: A and B, with subtype A typically associated with more severe symptoms. Infection with a virus from a given subgroup does not protect against a subsequent infection with an RSV isolate from the same subgroup in the following winter season. Re-infection with RSV is thus common, despite the existence of only two subtypes, A and B.

Today only two drugs have been approved for use against RSV infection. A first one is ribavirin, a nucleoside analogue that provides an aerosol treatment for serious RSV infection in hospitalized children. The aerosol route of administration, the toxicity (risk of teratogenicity), the cost and the highly variable efficacy limit its use. Synagis® (palivizumab a monoclonal antibody, is used for passive immunoprophylaxis. Although the benefit of Synagis® has been demonstrated, the treatment is expensive, requires parenteral administration and is restricted to children at risk for developing severe pathology.

Clearly there is a need for an efficacious non-toxic and easy to administer drug against RSV replication.

Compounds that exhibit anti-RSV activity are disclosed in WO-2015/026792.

### DETAILED DESCRIPTION OF THE INVENTION

The present invention relates to compounds of formula (I)

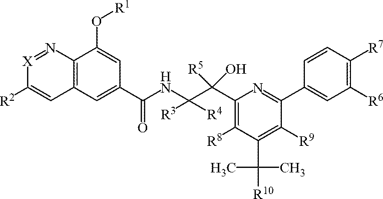

including any stereochemically isomeric form thereof, wherein
X is CH, CF or N;
R¹ is C₁₋₃alkyl, cyclopropyl, CHF₂ or CF₃;
R² is CH₃, CD₃, C₃₋₄cycloalkyl, CH₂F, CHF₂, or CF₃;
R³ and R⁴ are each individually selected from hydrogen and deuterium;
R⁵ is CF₃, CHF₂, CH₃, ethyl, isopropyl or cyclopropyl, wherein isopropyl or cyclopropyl are unsubstituted or substituted with one or two substituents each individually selected from halo, hydroxy, CH₃, or CH₃O;
R⁶ is hydrogen, CH₃ or halo;
R⁷ is hydrogen, halo, CF₃ or cyclopropyl;
R⁸ is hydrogen, CH₃, F, or Cl;
R⁹ is hydrogen, F, or Cl; and
R¹⁰ is hydroxy, C₁₋₄alkyl—SO₂—NH— or C₁-₄alkyl-CO-NH-;
or a pharmaceutically acceptable acid addition salt thereof.

The compounds of the present invention differ structurally over the exemplified compounds in WO-2015/026792 due to the mandatory presence of the R² substituent as a non-hydrogen substituent. As demonstrated in Example 5.3 the compounds of the present invention have unexpectedly improved antiviral properties against the respiratory syncytial virus (RSV).

As used in the foregoing definitions :
halo is generic to fluoro, chloro, bromo and iodo;
C₁₋₄alkyl defines straight and branched chain saturated hydrocarbon radicals having from 1 to 4 carbon atoms such as, for example, methyl, ethyl, propyl, butyl, 1-methylethyl, 2-methylpropyl and the like; and
C₃₋₄cycloalkyl is generic to cyclopropyl and cyclobutyl.

The term “compounds of the invention” as used herein, is meant to include the compounds of formula (I), and the salts and solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the terms “compound of formula (1)” and “intermediates of synthesis of formula (1)” are meant to include the stereoisomers thereof and the tautomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers. Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. Substituents on bivalent cyclic (partially) saturated radicals may have either the cis- or trans-configuration; for example, if a compound contains a disubstituted cycloalkyl group, the substituents may be in the cis or trans configuration.

The term “stereoisomers” also includes any rotamers, also called conformational isomers, the compounds of formula (I) may form.

Therefore, the invention includes enantiomers, diastereomers, racemates, E isomers, Z isomers, cis isomers, trans isomers, rotamers, and mixtures thereof, whenever chemically possible.

The meaning of all those terms, i.e. enantiomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (-) depending on the direction in which they rotate plane polarized light. For instance, resolved enantiomers whose absolute configuration is not known can be designated by (+) or (-) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

Some of the compounds according to formula (I) may also exist in their tautomeric form. Such forms in so far as they may exist, although not explicitly indicated in the above formula (I) are intended to be included within the scope of the present invention.

It follows that a single compound may exist in both stereoisomeric and tautomeric form.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

Conversely said salt forms can be converted by treatment with an appropriate base into the free base form.

The compounds of formula (I) may exist in both unsolvated and solvated forms. The term ‘solvate’ is used herein to describe a molecular association comprising a compound of the invention and one or more pharmaceutically acceptable solvent molecules, e.g. water or ethanol. The term ‘hydrate’ is used when said solvent is water.

For the avoidance of doubt, compounds of formula (I) may contain the stated atoms in any of their natural or non-natural isotopic forms. In this respect, embodiments of the invention that may be mentioned include those in which (a) the compound of formula (I) is not isotopically enriched or labelled with respect to any atoms of the compound; and (b) the compound of formula (I) is isotopically enriched or labelled with respect to one or more atoms of the compound. Compounds of formula (I) that are isotopically enriched or labelled (with respect to one or more atoms of the compound) with one or more stable isotopes include, for example, compounds of formula (I) that are isotopically enriched or labelled with one or more atoms such as deuterium, ¹³C, ¹⁴C, ¹⁴N, ¹⁵O or the like.

The compounds of formula (I) of the present invention all have at least one chiral carbon atom as indicated in the figure below by the carbon atom labelled with * :

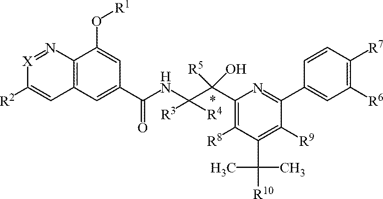

Due to the presence of said chiral carbon atom, a “compound of formula (1)” can be the racemic form, the (R)-enantiomer, the (S)-enantiomer, or any possible combination of the two individual enantiomers in any ratio. When the absolute (R)- or (S)-configuration of an enantiomer is not known, this enantiomer can also be identified by indicating whether the enantiomer is dextrorotatory (+)- or levorotatory (-)- after measuring the specific optical rotation of said particular enantiomer.

In an aspect the present invention relates to a first group of compounds of formula (I) wherein the compounds of formula (I) have the (+) specific rotation.

In a further aspect the present invention relates to a second ground of compounds of formula (I) wherein the compounds of formula (I) have the (-) specific rotation.

In another aspect, the present invention relates to compounds of formula (I)

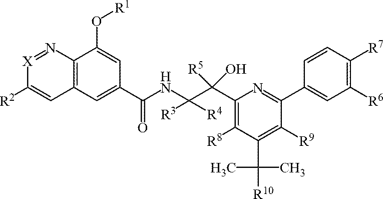

including any stereochemically isomeric form thereof, wherein
X is CH, CF or N;
R¹ is C₁₋₃alkyl, cyclopropyl, CHF₂ or CF₃;
R² is CH₃, CD₃, C₃₋₄cycloalkyl, CH₂F, CHF₂, or CF₃;
R³ and R⁴ are each individually selected from hydrogen and deuterium;
R⁵ is CF₃, CHF₂, CH₃, isopropyl or cyclopropyl, wherein isopropyl or cyclopropyl are unsubstituted or substituted with one or two substituents each individually selected from halo, hydroxy, CH₃, or CH₃O;
R⁶ is hydrogen, CH₃ or halo;
R⁷ is hydrogen, halo, CF₃ or cyclopropyl;
R⁸ is hydrogen, CH₃, F, or Cl;
R⁹ is hydrogen, F, or Cl; and
with the proviso that when R⁸ is F or Cl then R⁹ is other than hydrogen;
R¹⁰ is hydroxy, C₁₋₄alkyl—SO₂—NH— or C₁₋₄alkyl-CO-NH-;
or a pharmaceutically acceptable acid addition salt thereof.

A first group of compounds are compounds of formula (I) wherein X is CH or CF, in particular X is CH.

A second group of compounds are compounds of formula (I) wherein X is N.

A third group of compounds are compounds of formula (I) wherein R¹ is C₁₋₃alkyl, in particular R¹ is CH₃.

A fourth group of compounds are compounds of formula (I) wherein R¹ is cyclopropyl.

A fifth group of compounds are compounds of formula (I) wherein R² is CH₃.

A sixth group of compounds are compounds of formula (I) wherein R² is cyclopropyl.

A seventh group of compounds are compounds of formula (I) wherein R² is CHF₂.

An eight group of compounds are compounds of formula (I) wherein R¹⁰ is hydroxy.

Interesting compounds of formula (I) are those compounds of formula (I) wherein one or more of the following restrictions apply :
a) X is CH or CF; or
b) X is N; or
c) R¹ is CH₃ or cyclopropyl; or
d) R² is CH₃, CHF₂ or cyclopropyl; or
e) R² is CH₃; or
f) R³ and R⁴ are hydrogen; or
g) R⁵ is CF₃ or cyclopropyl; or
h) R⁶ is hydrogen or F; or
i) R⁷ is F; or
j) R⁸ is hydrogen and R⁹ is halo; or
k) R⁸ is F and R⁹ is F; and
l) R¹⁰ is hydroxy.

A particular group of compounds are compounds of formula (I) wherein X is N; R¹ is CH₃ or cyclopropyl; and R¹⁰ is hydroxy

Another particular group of compounds are compounds of formula (I) wherein X is CH; R¹ is CH₃ or cyclopropyl; R² is CH₃, CHF₂ or cyclopropyl; R³ and R⁴ are hydrogen; R⁵ is CF₃ or cyclopropyl; R⁶ is hydrogen or F; R⁷ is F; R⁸ is hydrogen or F and R⁹ is halo; and R¹⁰ is hydroxy.

Yet another particular group of compounds are compounds of formula (I) wherein X is N; R¹ is CH₃ or cyclopropyl; R² is CH₃, CHF₂ or cyclopropyl; R³ and R⁴ are hydrogen; R⁵ is CF₃ or cyclopropyl; R⁶ is hydrogen or F; R⁷ is F; R⁸ is hydrogen or F and R⁹ is halo; and R¹⁰ is hydroxy.

Specific examples of compounds of formula (I) are :

| | |
|---|---|
| 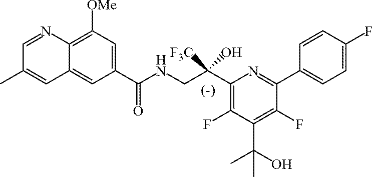 | 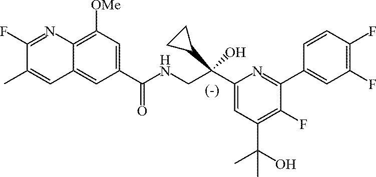 |
| Co. No. 201 | Co. No. 218 |
| 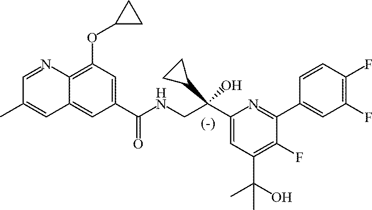 | 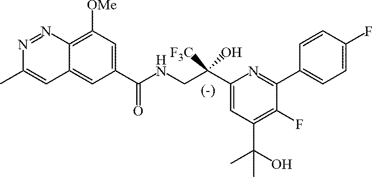 |
| Co. No. 225 | Co. No. 234 |
| 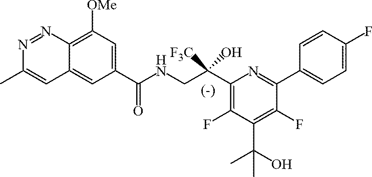 | 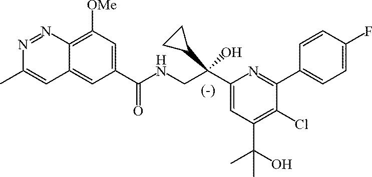 |
| Co. No. 240 | Co. No. 251 |
| 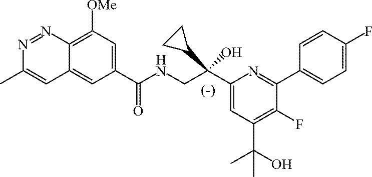 | 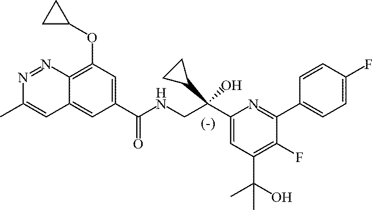 |
| Co. No. 252 | Co. No. 265 |
| 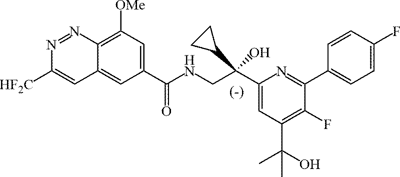 | 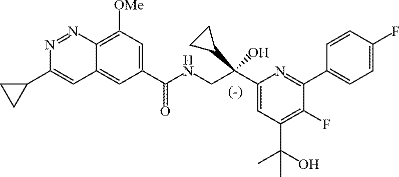 |
| Co. No. 269 | Co. No. 271 |

In general compounds of formula (I) can be prepared by an art-known amide bond formation reaction between a carboxylic acid compound of formula (II) and an amine of formula (III) wherein said amide-bond formation may be performed by stirring the intermediate compounds of formula (II) and (III) in an appropriate solvent, such as e.g. acetonitrile, dimethyl acetamide, dichloromethane, tetrahydrofuran, or DMF, optionally in the presence of a base, such as triethylamine, DIPEA (diisopropylamine) DMAP (dimethylaminopyridine), or N-methylmorpholine. The carboxylic acid compound of formula (II) can be used as such or can be converted first into a reactive functional derivative thereof, such as, e.g carbonyl imidazole derivatives, acyl halides or mixed anhydrides. Conveniently a coupling agent such as HATU (1-[bis(dimethylamino)methylene]-1H-1,2,3-triazolo[4,5-b]pyridinium 3-oxid hexafluorophosphate), DEPC (diethyl cyanophosphonate), EDC (1-ethyl-3-(3-dimethylaminopropyl)-carbodiimide), BOP, PYBOP, HBTU is used. Stirring may enhance the rate of the reaction. The reaction may conveniently be carried out at a temperature ranging between room temperature and the reflux temperature of the reaction mixture.

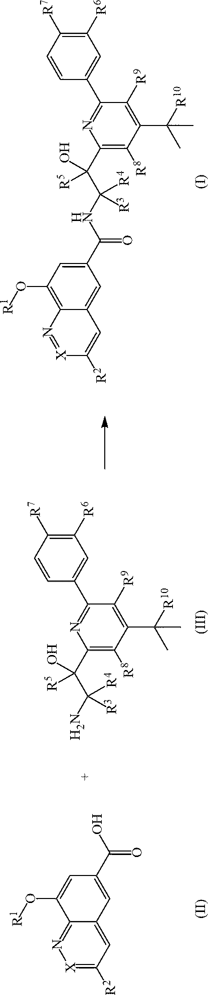

Other synthetic pathways for preparing compounds of formula (I) have been described in the experimental party as general methods of preparation and specific working examples.

The compounds of formula (I) may further be prepared by converting compounds of formula (I) into each other according to art-known group transformation reactions.

The starting materials and some of the intermediates are known compounds and are commercially available or may be prepared according to conventional reaction procedures generally known in the art.

The compounds of formula (I) as prepared in the hereinabove described processes may be synthesized in the form of racemic mixtures of enantiomers which can be separated from one another following art-known resolution procedures. Those compounds of formula (I) that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

The in vitro antiviral activity against RSV of the present compounds was demonstrated in an antiviral assay as described in the experimental part 5.1 of the description and may also be demonstrated in a virus yield reduction assay. The in vivo antiviral activity against RSV of the present compounds may also be demonstrated in a test model using cotton rats as described in Wyde et al. in Antiviral Research, 38, p. 31 – 42 (1998).

The compounds of formula (I) show antiviral properties. Viral infections preventable or treatable using the compounds and methods of the present invention include those infections brought on by Pneumoviridae and in particular by human and bovine respiratory syncytial virus (RSV).

Therefore the present compounds of formula (I), or a pharmaceutically acceptable acid addition salt thereof, may be used as a medicine, in particular may be used as a medicine for the treatment or prevention of infections brought on by Pneumoviridae and in particular by human and bovine respiratory syncytial virus (RSV).

The present invention also provides the use of a compound of formula (I) or a pharmaceutically acceptable salt thereof for the manufacture of a medicament for the treatment or prevention of infections brought on by Pneumoviridae and in particular by human and bovine respiratory syncytial virus (RSV).

In other aspects, provided are methods of treating a respiratory syncytial virus (RSV) infection in an individual in need thereof comprising administering to the individual a therapeutically effective amount of a compound of formula (I) provided herein, or a pharmaceutically acceptable salt thereof, or a pharmaceutical composition comprising a therapeutically effective amount of a compound of formula (I) provided herein, or a pharmaceutically acceptable salt thereof. In some embodiments, the individual has one or more symptoms of an RSV infection. In some embodiments, the RSV is RSV Type A. In some embodiments, the RSV is RSV Type B.

Also provided are methods of ameliorating one or more symptoms of an RSV infection in an individual in need thereof comprising administering to the individual a therapeutically effective amount of a compound of formula (I) provided herein, or a pharmaceutically acceptable salt thereof, or a pharmaceutical composition comprising a therapeutically effective amount of a compound of formula (I) provided herein, or a pharmaceutically acceptable salt thereof. In some embodiments, the symptom is one or more of: coughing, sneezing, runny nose, sore throat, fever, decrease of appetite, irritability, decreased activity, apnea, and wheezing. In some embodiments, the individual has a lower respiratory tract infection. In some embodiments, the individual has bronchiolitis, pneumonia, or croup. In some embodiments, the individual has been diagnosed with an RSV infection. In some embodiments, the RSV is RSV Type A. In some embodiments, the RSV is RSV Type B. In some embodiments, the RSV infection has been confirmed by a laboratory test. In some embodiments, the method further comprises obtaining the results of an RSV detecting laboratory test. In some embodiments, the laboratory test comprises detecting RSV in a nasal sample.

Also provided are methods of preventing an RSV infection in an individual at risk of developing an RSV infection comprising administering to the individual a prophylactically effective amount of a compound of formula (I) provided herein, or a pharmaceutically acceptable salt thereof, or a pharmaceutical composition comprising a prophylactically effective amount of a compound of formula (I) provided herein, or a pharmaceutically acceptable salt thereof. In some embodiments, the individual is between 0 and about 2 years of age. In some embodiments, the individual was born prematurely. In other embodiments, the individual is greater than 65 years of age. In some embodiments, the individual is immunocompromised.

As used herein, “treatment” or “treating” is an approach for obtaining beneficial or desired results including clinical results. For example, beneficial or desired results in treating a viral infection include, but are not limited to, one or more of the following: eliminating or lessening the severity of one or more symptoms resulting from the viral infection (such as but not limited to coughing, sneezing, runny nose, sore throat, fever, decrease of appetite, irritability, decreased activity, apnea, and wheezing), increasing the quality of life of those suffering from the viral infection, decreasing the dose of other medications required to treat the viral infection, delaying the progression of the viral infection, and/or prolonging survival of an individual.

As used herein, “preventing” a viral infection is an approach for eliminating or reducing the risk of developing a viral infection or delaying the onset of a viral infection, including biochemical, histological and/or behavioral symptoms of a viral infection. Prevention may be in the context of an individual at risk of developing the viral infection, such as where the “at risk” individual does not develop the viral infection over a period of time, such as during a viral season or during a period of exposure to the virus, which may be days to weeks to months. An individual “at risk” of developing a viral infection is an individual with one or more risk factors for developing the viral infection but who has not been diagnosed with and does not display symptoms consistent with a viral infection. Risk factors for developing an RSV infection include but are not limited to an individual’s age (young children under age 5 such as children between about 0 and about 2 years of age, including infants, and individuals greater than 65 years of age), premature birth, co-morbidities associated with RSV and individuals who are immunocompromised.

As used herein, a “therapeutically effective dosage” or “therapeutically effective amount” of compound or salt thereof or pharmaceutical composition is an amount sufficient to produce a desired therapeutic outcome. A therapeutically effective amount or a therapeutically effective dosage can be administered in one or more administrations. A therapeutically effective amount or dosage may be considered in the context of administering one or more therapeutic agents (e.g., a compound, or pharmaceutically acceptable salt thereof), and a single agent may be considered to be given in a therapeutically effective amount if, in conjunction with one or more other agents, a desired therapeutic outcome is achieved. Suitable doses of any of the coadministered compounds may optionally be lowered due to the combined action (e.g., additive or synergistic effects) of the compounds.

As used herein, a “prophylactically effective dosage” or “prophylactically effective amount” is an amount sufficient to effect the preventative result of eliminating or reducing the risk of developing a viral infection or delaying the onset of a viral infection, including biochemical, histological and/or behavioral symptoms of a viral infection. A prophylactically effective amount or a prophylactically effective dosage can be administered in one or more administrations and over a period of time in which such prevention is desired. Additionally, the present invention provides pharmaceutical compositions comprising at least one pharmaceutically acceptable carrier and a therapeutically effective amount of a compound of formula (I).

Also provided are pharmaceutical compositions comprising a pharmaceutically acceptable carrier, a therapeutically active amount of a compound of formula (I), and another antiviral agent, in particular an RSV inhibiting compound.

Also, the combination of another antiviral agent and a compound of formula (I) can be used as a medicine. Thus, the present invention also relates to a product containing (a) a compound of formula (I), and (b) another antiviral compound, as a combined preparation for simultaneous, separate or sequential use in antiviral treatment. The different drugs may be combined in a single preparation together with pharmaceutically acceptable carriers. Other antiviral compounds (b) to be combined with a compound of formula (I) for use in the treatment of RSV are RSV fusion inhibitors or RSV polymerase inhibitors.

In order to prepare the pharmaceutical compositions of this invention, an effective amount of the particular compound, in free base form or acid addition salt form, as the active ingredient is combined in intimate admixture with at least one pharmaceutically acceptable carrier, which carrier may take a wide variety of forms depending on the form of preparation desired for administration. These pharmaceutical compositions are desirably in unitary dosage form suitable, preferably, for oral administration, rectal administration, percutaneous administration, parenteral or intramuscular injection.

For example in preparing the compositions in oral dosage form, any of the usual liquid pharmaceutical carriers may be employed, such as for instance water, glycols, oils, alcohols and the like in the case of oral liquid preparations such as suspensions, syrups, elixirs and solutions; or solid pharmaceutical carriers such as starches, sugars, kaolin, lubricants, binders, disintegrating agents and the like in the case of powders, pills, capsules and tablets. Because of their easy administration, tablets and capsules represent the most advantageous oral dosage unit form, in which case solid pharmaceutical carriers are obviously employed. For parenteral injection compositions, the pharmaceutical carrier will mainly comprise sterile water, although other ingredients may be included in order to improve solubility of the active ingredient. Injectable solutions may be prepared for instance by using a pharmaceutical carrier comprising a saline solution, a glucose solution or a mixture of both. Injectable suspensions may also be prepared by using appropriate liquid carriers, suspending agents and the like. In compositions suitable for percutaneous administration, the pharmaceutical carrier may optionally comprise a penetration enhancing agent and/or a suitable wetting agent, optionally combined with minor proportions of suitable additives which do not cause a significant deleterious effect to the skin. Said additives may be selected in order to facilitate administration of the active ingredient to the skin and/or be helpful for preparing the desired compositions. These topical compositions may be administered in various ways, e.g., as a transdermal patch, a spot-on or an ointment. Addition salts of the compounds of formula (I), due to their increased water solubility over the corresponding base form, are obviously more suitable in the preparation of aqueous compositions.

It is especially advantageous to formulate the pharmaceutical compositions of the invention in dosage unit form for ease of administration and uniformity of dosage. “Dosage unit form” as used herein refers to physically discrete units suitable as unitary dosages, each unit containing a predetermined amount of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

For oral administration, the pharmaceutical compositions of the present invention may take the form of solid dose forms, for example, tablets (both swallowable and chewable forms), capsules or gelcaps, prepared by conventional means with pharmaceutically acceptable excipients and carriers such as binding agents, fillers, lubricants, disintegrating agents, wetting agents and the like. Such tablets may also be coated by methods well known in the art.

Liquid preparations for oral administration may take the form of e.g. solutions, syrups or suspensions, or they may be formulated as a dry product for admixture with water and/or another suitable liquid carrier before use. Such liquid preparations may be prepared by conventional means, optionally with other pharmaceutically acceptable additives such as suspending agents, emulsifying agents, non-aqueous carriers, sweeteners, flavours, masking agents and preservatives.

The compounds of formula (I) may be formulated for parenteral administration by injection, conveniently intravenous, intramuscular or subcutaneous injection, for example by bolus injection or continuous intravenous infusion. Formulations for injection may be presented in unit dosage form, e.g. in ampoules or multi-dose containers, including an added preservative. They may take such forms as suspensions, solutions or emulsions in oily or aqueous vehicles, and may contain formulating agents such as isotonizing, suspending, stabilizing and/or dispersing agents. Alternatively, the active ingredient may be present in powder form for mixing with a suitable vehicle, e.g. sterile pyrogen free water, before use.

The compounds of formula (I) may also be formulated in rectal compositions such as suppositories or retention enemas, e.g. containing conventional suppository bases such as cocoa butter and/or other glycerides.

In general, it is contemplated that an antivirally effective daily amount would be from 0.01 mg/kg to 500 mg/kg body weight, more preferably from 0.1 mg/kg to 50 mg/kg body weight. It may be appropriate to administer the required dose as two, three, four or more sub-doses at appropriate intervals throughout the day. Said sub-doses may be formulated as unit dosage forms, for example, containing 1 to 1000 mg, and in particular 5 to 200 mg of active ingredient per unit dosage form.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention. The effective daily amount ranges mentioned hereinabove are therefore only guidelines.

### Experimental Part

### 1. General Information

### 1.1. NMR Analysis

¹H NMR spectra were recorded on 1) a Bruker Avance DRX 400 spectrometer or Bruker Advance III 400 spectrometer or 2) a Bruker Avance 500 MHz spectrometer and c) Bruker Advance III 400 spectrometer.

NMR spectra were recorded at ambient temperature unless otherwise stated. Data are reported as follow: chemical shift in parts per million (ppm) relative to TMS (δ = 0 ppm) on the scale, integration, multiplicity (s = singlet, d = doublet, t = triplet, q = quartet, quin = quintuplet, sex = sextuplet, m = multiplet, b = broad, or a combination of these), coupling constant(s) J in Hertz (Hz).

### 1.2. HPLC and LC-MS

The High-Performance Liquid Chromatography (HPLC) measurement was performed using a LC pump, a diode-array (DAD) or a UV detector and a column as specified in the respective methods. If necessary, additional detectors were included (see table of methods below).

Flow (expressed in mL/min; column temperature (T) in °C; Run time in minutes) from the column was brought to the Mass Spectrometer (MS) which was configured with an atmospheric pressure ion source. It is within the knowledge of the skilled person to set the tune parameters (e.g. scanning range, dwell time... ) in order to obtain ions allowing the identification of the compound’s nominal monoisotopic molecular weight (MW). Data acquisition was performed with appropriate software.

Compounds are described by their experimental retention times (RT) and ions.

All results were obtained with experimental uncertainties that are commonly associated with the method used.

Hereinafter, “SQD” means Single Quadrupole Detector, “MSD” Mass Selective Detector, “BEH” bridged ethylsiloxane/silica hybrid, “DAD” Diode Array Detector, “HSS” High Strength silica., “Q-Tof” Quadrupole Time-of-flight mass spectrometers, “CLND”, ChemiLuminescent Nitrogen Detector, “ELSD” Evaporative Light Scanning Detector.

| Method code | Instrument | Column | Mobile phase | Gradient | Flow-------Col T | Run time |
|---|---|---|---|---|---|---|
| A | Waters: Acquity® UPLC®- DAD and SQD | Waters: HSS T3 (1.8 µm, 2.1 x 100 mm) | A: 10 mM CH₃COONH₄ in 95% H₂O + 5% CH₃CN B: CH₃CN | From 100% A to 5% A in 2.10 min, to 0% A in 0.90 min, to 5% A in 0.5 min | 0.7 ---------55 | 3.5 |
| B | Waters: Acquity® UPLC®- DAD and SQD | Waters: BEH C18 (1.7 µm, 2.1 x 50 mm) | A: 10 mM CH₃COONH₄ in 95% H₂O + 5% CH₃CN B: CH₃CN | From 95% A to 5% A in 1.3 min, held for 0.7 min. | 0.8 ---------55 | 2 |
| C | Waters: Acquity® UPLC®- DAD and SQD | Waters: BEH (1.8 µm, 2.1 x 100 mm) | A: 10 mM CH₃COONH₄ in 95% H₂O + 5% CH₃CN B: CH₃CN | From 100% A to 5% A in 2.10 min, to 0% A in 0.90 min, to 5% A in 0.5 min | 0.6 ---------55 | 3.5 |
| D | Waters: Acquity® UPLC®- DAD and SQD | Waters: BEH C18 (1.8 µm, 2.1* 100 mm) | A: 10 mM CH₃COONH₄ in 95% H₂O + 5% CH₃CN B: CH₃OH | From 100% A to 5% A in 2.10 min, to 0% A in 0.9 min, to 5% A in 0.5 min | 0.6 ---------55 | 3.5 |
| E | Waters: Acquity® UPLC®- DAD, SQD | Waters: BEH (1.8 µm, 2.1 x 100 mm) | A: 0.1% NH₄HCO₃ in H₂O B: CH₃CN | From 100% A to 5% A in 2.10 min, to 0% A in 0.90 min, to 5% A in 0.5 min | 0.6 ---------55 | 3.5 |
| F | Waters: Acquity® UPLC®- DAD and SQD | Waters: BEH C18 (1.8 µm, 2.1* 100 mm) | A: 0.1% NH₄HCO₃ in 95% H₂O + 5% CH₃CN B: CH₃OH | From 100% A to 5% A in 2.10 min, to 0% A in 0.9 min, to | 0.6 ---------55 | 3.5 |
| | | | | 5% A in 0.5 min | | |
| G | Waters: Acquity® UPLC®- DAD, SQD and ELSD | Waters: HSS T3 (1.8 µm, 2.1x100 mm) | A: 10 mM CH₃COONH₄ in 95% H₂O + 5% CH₃CN B: CH₃CN | From 100% A to 5% A in 2.10 min, to 0% A in 0.90 min, to 5% A in 0.5 min | 0.6 ---------55 | 3.5 |
| H | Waters: Acquity® UPLC®- DAD and SQD | Waters: BEH C18 (1.8 µm, 2.1* 100 mm) | A: 10 mM CH₃COONH₄ in 95% H₂O + 5% CH₃CN B: CH₃CN | From 100% A to 5% A in 2.10 min, to 0% A in 0.9 min, to 5% A in 0.5 min | 0.6 ---------55 | 3.5 |
| I | Waters: Acquity UPLC®-DAD and Quattro Micro™ | Waters: BEH C18 (1.7 µm, 2.1x100 mm) | A: 95% CH₃COONH₄ 7 mM / 5% CH₃CN B: CH₃CN | 84.2% A for 0.49 min, to 10.5% A in 2.18 min, held for 1.94 min, back to 84.2% A in 0.73 min, held for 0.73 min. | 0.34 ---------40 | 6.2 |
| J | Waters: Acquity UPLC® H- Class -DAD and SQD 2 | Waters BEH®C18 (1.7 µm, 2.1x50 mm) | A: 95% CH₃COONH₄ 7 mM / 5% CH₃CN B: CH₃CN | From 95% A to 5% A in 1 min, held for 1.6 min, back to 95% A in 0.2 min, held for 0.5 min. | 0.5 ---------40 | 3.3 |
| K | Waters: Acquity UPLC® H-Class -DAD and QDa | BEH®-C18 (1.7 µm, 2.1x100 mm) | A: 95% CH₃COONH₄ 7 mM / 5% CH₃CN B: CH₃CN | 95% A to 5% A in 1 min, held for 1.6 min, back to 95% A in 0.2 min, held for 0.5 min. | 0.5 -------40 | 3.3 |
| L | Waters: Acquity® UPLC®- DAD and SQD | Waters : BEH C18 (1.7 µm, 2.1 *50mm) | A: 10 mM CH₃COONH₄ in 95% H₂O + 5% CH₃CN B: CH₃CN | From 95% A to 5% A in 1.3 min, held for 0.7 min. | 0.8 -------55 | 2 |
| M | Waters: Alliance®-DAD -ZMD and CLND 8060 Antek | Atlantis T3 column (5 µm, 4.6 x 100 mm) | A: 70%CH3OH, 30% H₂O B: 0.1 formic acid in H2O/methanol 95/5 | 100%B to 5%B in 9 min, hold 3.0 min to 100%B in 1 min and hold 0.5 min | 1.5 -------45 | 13.5 |
| O | Waters: Acquity® UPLC® -DAD and SQD | Waters :BEH (1.8 µm, 2.1* 100 mm) | A: 10 mM CH₃COONH₄ in 95% H₂O + 5% CH₃CN B: CH₃CN | From 100% A to 5% A in 2.10 min, to 0% A in 0.90 min, to 5% A in 0.5 min | 0.7 -------55 | 3.5 |
| P | Waters: Acquity® UPLC®- DAD and SQD | Waters :BEH (1.8 µm, 2.1* 100 mm) | A: 0.1% NH₄HCO3 in 95% H₂O + 5% CH₃CN B: CH3CN | From 100% A to 5% A in 2.10 min, to 0% A in 0.9 min, to 5% A in 0.5 min | 0.6 -------55 | 3.5 |
| Q | Waters: Acquity® UPLC®- DAD, SQD and ELSD | Waters : HSS T3 (1.8 µm, 2.1* 100 mm) | A: 10 mM CH₃COONH₄ in 95% H₂O + 5% CH₃CN B: CH₃CN | From 100% A to 5% A in 2.10 min, to 0% A in 0.90 min, to 5% A in 0.5 min | | 0.6 -------55 |
| R | Waters: Acquity® UPLC®- DAD and SQD | Waters : BEH (1.8 µm, 2.1* 100 mm) | A: 10 mM CH₃COONH₄ in 95% H₂O + 5% CH₃CN B: CH₃CN | From 100% A to 5% A in 2.10 min, to 0% A in 0.90 min, to 5% A in 0.5 min | 0.6 -------55 | 3.5 |
| S | Waters: Acquity® UPLC®- DAD and SQD2 | Waters :BEH (1.7 µm, 2.1* 100 mm) | A: 0.1% NH4HCO3 in 95% H2O + 5% CH₃CN B: CH3CN | From 100% A to 5% A in 2.10 min, to 0% A in 0.9 min, to 5% A in 0.5 min | 0.6 -------55 | 3.5 |
| T | Waters: Acquity® UPLC®- DAD and SQD2 | Waters :BEH (1.7 µm, 2.1* 100 mm) | A: 0.1% NH₄HCO3 in 95% H2O + 5% CH₃CN B: CH3CN | From 100% A to 5% A in 2.10 min, to 0% A in 1.4 min | 0.6 -------55 | 3.5 |
| U | Waters: Acquity® UPLC®- DAD and SQD | Waters :BEH (1.7 µm, 2.1* 100 mm) | A: 10 mM CH₃COONH₄ in 95% H2O + 5% CH₃CN B: MeOH | From 100% A to 5% A in 2.10 min, to 0% A in 0.9 min, to 5% A in 0.5 min | 0.6 -------55 | 3.5 |

Description of SFC Method

| Method code | column | mobile phase | gradient | Flow ------------Col T | Run time ------------BPR |
|---|---|---|---|---|---|
| Method SFC | Daicel Chiralpak® IH3 column (3.0 µm, 150 x 4.6 mm) | A:CO₂ B: iPrOH +0.2% iPrNH₂ | 10%-50% B in 6 min, hold 3.5 min | 2.5 -------40 | 9.5 -------130 |

### 1.3. Optical Rotation

Optical rotations were measured on a Perkin Elmer 341 polarimeter and reported as follow [α]_{λ}^{T}. λ is the wavelength of light used in nm (if the wavelength of light used is 589 nm, the sodium D line, then the symbol D is used) and T is the temperature in degree Celsius. The sign (+ or -) of the rotation is given. The concentration and the solvent of the sample are provided in brackets after the rotation. The rotation is reported in degrees and no units of concentration are given (it is assumed to be g/100 mL).

2. Abbreviations

| | |
|---|---|
| 2-MeTHF | 2-Methyltetrahydrofuran |
| AcOH | Acetic acid |
| aq. | Aqueous |
| Boc₂O | Di-tert-butyl dicarbonate |
| DAST | (Diethylamino)sulfur trifluoride |
| DCE | 1,2-Dichloroethane |
| DDQ | 2,3-Dichloro-5,6-dicyano-p-benzoquinone |
| DEPC | Diethyl cyanophosphonate CAS [2942-58-7] |
| DIPEA | N,N-Diisopropylethylamine |
| DMAP | 4-(Dimethylamino)pyridine |
| DME | 1,2-Dimethoxyethane |
| DMF | N,N-Dimethylformamide |
| DMP | Dess-Martin periodinane, CAS [87413-09-0] |
| DMSO | Dimethyl sulfoxide |
| dppf | 1,1′-Ferrocenediyl-bis(diphenylphosphine) |
| EDC | N-(3-Dimethylaminopropyl)-N′-ethylcarbodiimide hydrochloride |
| Et₂O | Diethyl ether |
| Et₃N | Triethylamine |
| EtI | Ethyl iodide |
| EtOAc | Ethyl acetate |
| EtOH | Ethanol |
| h | Hour |
| HATU | 1-[Bis(dimethylamino)methylene]-1H-1,2,3-triazolo[4,5-b]pyridinium 3-oxid hexafluorophosphate, CAS [148893-10-1] |
| HOBt.H₂O | 1-Hydroxybenzotriazole hydrate |
| HPLC | High Performance Liquid Chromatography |
| i-PrMgBr | Isopropylmagnesium bromide |
| i-PrNH₂ | Isopropylamine |
| i-PrOH | Isopropyl alcohol |
| LC-MS | Liquid Chromatography Mass Spectrometry |
| LDA | Lithium diisopropylamide |
| m-CPBA | 3-Chloroperbenzoic acid |
| min | Minute |
| MsCl | Methanesulfonyl chloride |
| NBS | N-Bromosuccinimide |
| n-BuLi | n-Butyllithium |
| NMP | Methylpyrrolidone |
| Pd(dppf)Cl₂ | [1,1′-Bis(diphenylphosphino)ferrocene]dichloropalladium(II) CAS [72287-26-4] |
| Pd(dppf)Cl₂.CH₂Cl₂ | [1,1′-Bis(diphenylphosphino)ferrocene]dichloropalladium(II), complex with dichloromethane CAS [95464-05-4] |
| Pd(dtpbf)Cl₂ | [1,1′-Bis(di-tert-butylphosphino)ferrocene]dichloropalladium(II) CAS [95408-45-0] |
| Pd(OAc)₂ | Palladium(II) acetate |
| Pd(PPh₃)₄ | Tetrakis(triphenylphosphine)palladium(0), CAS [14221-01-3] |
| Pd₂(dba)₃ | Tris(dibenzylideneacetone)dipalladium(0), CAS [51364-51-3] |
| rt | Room temperature |
| RT | Retention time |
| SFC | Supercritical Fluid Chromatography |
| TBAF | Tetrabutylammonium fluoride |
| t-BuOH | tert-Butyl alcohol |
| t-BuOK | Potassium tert-butoxide |
| TFA | Trifluoroacetic acid |
| TFAA | Trifluoroacetic anhydride |
| THF | Tetrahydrofuran |
| XPhos | 2-Dicyclohexylphosphino-2′,4′,6′-triisopropylbiphenyl CAS [564483-18-7] |
| XPhos Pd G2 | Chloro(2-dicyclohexylphosphino-2’,4’,6’-triisopropyl-1,1’-biphenyl)[2-(2’-amino-1,1’-biphenyl)]palladium(II) CAS [1310584-14-5] |

The stereochemical configuration for some compounds has been designated as R* or S* (or *R or *S) when the absolute stereochemistry is undetermined (even if the bonds are drawn stereospecifically) although the compound itself has been isolated as a single stereoisomer and is enantiomerically pure. This means that the absolute stereoconfiguration of the stereocentre indicated by * is undetermined (even if the bonds are drawn stereospecifically) although the compound is enantiomerically pure at the indicated centre.

### 3. Synthesis of Intermediates

### 3.1. Synthesis of the Quinoline Intermediates

### 3.1.1 Synthesis of 1

### 8-Methoxy-3-Methylquinoline-6-Carboxylic Acid 1

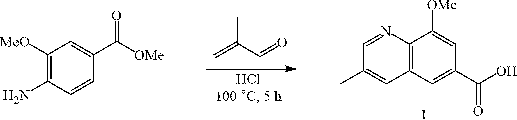

A mixture of 4-amino-3-methoxybenzoate (3.00 g, 18.0 mmol), methacrylaldehyde (3.00 g, 43.0 mmol) and HC1 (12 M aq., 12 mL, 12.0 mmol) was stirred at 100° C. for 5 h. The reaction mixture was cooled in an ice/water bath. The suspension was filtered off. The solid was purified by trituration with EtOAc and petroleum ether (1:30, 10 mL) (twice), to afford 1 (600 mg, 15%) as a gray solid. ¹H NMR (400 MHz, DMSO-d6) δ 13.26 (s, 1H), 8.81 (d, J=2.20 Hz, 1H), 8.25 (d, J=0.88 Hz, 1H), 8.12 (d, J=1.54 Hz, 1H), 7.50 (d, J=1.76 Hz, 1H), 4.01 (s, 3H), 2.50 (s, 3H).

### 3.1.2. Synthesis of 6

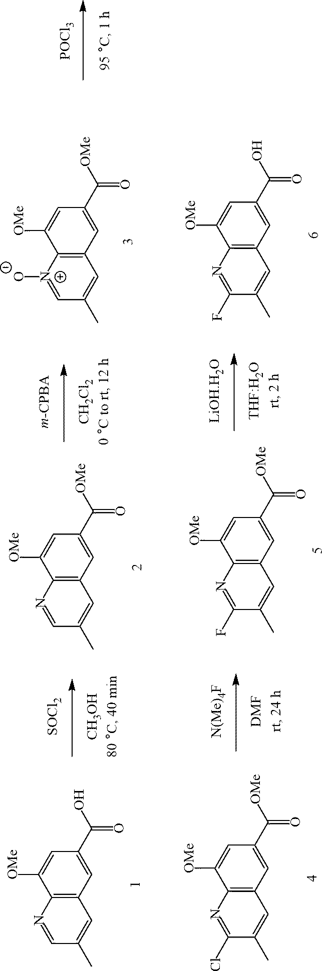

### Methyl 8-methoxy-3-methylquinoline-6-carboxylate 2

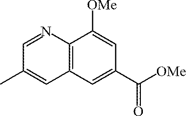 [SMILES: COC(=O)c1cc(OC)c2ncc(C)cc2c1]

SOCl₂ (33.6 mL, 460 mmol) was added to a solution of 1 (25.0 g, crude) in CH₃OH (300 mL) at 0° C. The reaction mixture was stirred at 80° C. for 40 min. The reaction mixture was concentrated to dryness under reduced pressure. The residue was dissolved in water (100 mL) and the pH of the solution was adjusted to pH 7-8 with NaHCO₃ (sat., aq.). The aqueous layer was extracted with EtOAc (3 x 200 mL). The combined organic extracts were washed with brine (200 mL), and dried (Na₂SO₄). The solids were removed by filtration and the filtrate was concentrated to dryness under reduced pressure. The crude mixture was purified by silica column chromatography (petroleum ether/EtOAc, gradient from 5:1 to 1:2) to afford 2 (10 g, 37 %) as a brown solid.

### 8-Methoxy-6-(methoxycarbonyl)-3-methylquinoline 1-oxide 3

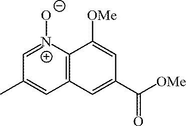 [SMILES: COC(=O)c1cc(OC)c2c(cc(C)c[n+]2[O-])c1]

The reaction was performed on two batches of 5.00 g of 2. m-CPBA (14.0 g, 64.9 mmol, 80% pure) was added to a solution of 2 (5.00 g, 22.0 mmol) in CH₂Cl₂ (50 mL) at 0° C. The reaction mixture was stirred at rt for 12 h and concentrated to dryness under reduced pressure. The two batches were combined. The crude mixture was purified by silica column chromatography (petroleum ether/EtOAc, gradient from 10:1 to 1:1) to afford 3 (5.1 g, 85%) as a brown solid.

### Methyl 2-chloro-8-methoxy-3-methylquinoline-6-carboxylate 4

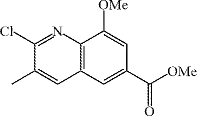 [SMILES: COC(=O)c1cc(OC)c2nc(Cl)c(C)cc2c1]

A mixture of 3 (3.80 g, 15.4 mmol) and POCl₃ (30.0 g, 196 mmol) was stirred at 95° C. for 1 h. The reaction mixture was concentrated to dryness under reduced pressure. The residue was dissolved in water (100 mL) and the aqueous phase was extracted with EtOAc (3 x 30 mL). The combined organic extracts were dried (Na₂SO₄). The solids were removed by filtration and the filtrate was concentrated to dryness under reduced pressure. The crude mixture was purified by silica column chromatography (petroleum ether/EtOAc, gradient from 1:0 to 1:1) to afford 4 (2.5 g, 61%) as a white solid.

### Methyl 2-fluoro-8-methoxy-3-methylquinoline-6-carboxylate 5

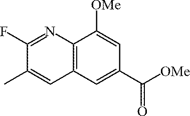 [SMILES: COC(=O)c1cc(OC)c2nc(F)c(C)cc2c1]

Tetramethylammonium fluoride (1.80 g, 19.3 mmol) was added to a solution of 4 (2.50 g, 9.41 mmol) in anhydrous DMF (20 mL). The reaction mixture was stirred at rt for 24 h. The reaction mixture was concentrated to dryness under reduced pressure. The crude mixture was purified by silica column chromatography (petroleum ether/EtOAc, gradient from 1:0 to 1:1) to afford 5 (1.8 g, 68%) as a yellow solid.

### 2-Fluoro-8-Methoxy-3-Methylquinoline-6-Carboxylic Acid 6

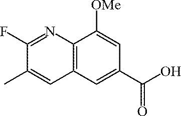 [SMILES: COc1cc(C(=O)O)cc2cc(C)c(F)nc12]

LiOH.H₂O (612 mg, 14.6 mmol) was added to a solution of 5 (1.8 g, 7.22 mmol) in THF (12 mL) and H₂O (6 mL) at 0° C. The resulting reaction mixture was stirred at rt for 2 h. The mixture was combined with another batch (1.30 g, 5.22 mmol), acidified to pH 5 with acetic acid and was concentrated to dryness under reduced pressure. The crude mixture was purified by preparative HPLC (Phenomenex Synergi Max-RP 250 x 50 mm x 10 µm, mobile phase gradient: 5% to 45% (v/v) water (0.225%FA)-CH₃CN). The product was suspended in water (50 mL). The mixture was frozen and lyophilized to dryness to give 6 (2.15 g, 72%, 97% pure) as a white solid. ¹H NMR (400 MHz, DMSO-d6) δ ppm 13.28 (br s, 1H), 8.51 (d, J=10.1 Hz, 1H), 8.17 (d, J=1.3 Hz, 1H), 7.57 (d, J=1.3 Hz, 1H), 4.00 (s, 3H), 2.41 (s, 3H).

### 3.1.3. Synthesis of 9

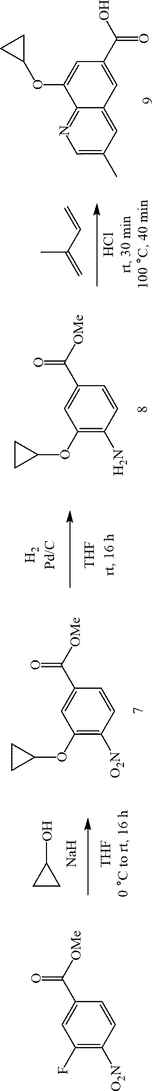

### Methyl 3-(cyclopropyloxy)-4-nitrobenzoate 7

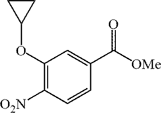 [SMILES: COC(=O)c1ccc([N+](=O)[O-])c(OC2CC2)c1]

NaH (2.00 g, 50.0 mmol, 60% in mineral oil) was added to a solution of cyclopropanol (1.75 g, 30.1 mmol) in THF (15 mL) under N₂ atmosphere at 0° C. The mixture was stirred for 1 h and a solution of methyl 3-fluoro-4-nitrobenzoate (5.00 g, 25.1 mmol) in THF (50 mL) was added at 0° C. The reaction mixture was stirred with gradual warming to rt for 16 h. The reaction was quenched with NH₄Cl (sat., aq., 30 mL). The layers were separated, and the aqueous phase was extracted with EtOAc (3 x 4 mL). The aqueous solution was acidified to pH 5.5 with HCl (1 M) and stirred for 1 h. The solid was collected by filtration, washed with H₂O (10 mL) and dried under reduced pressure to afford 7 (4.1 g, 73%) as a yellow solid that was used in the next step without further purification.

### Methyl 4-Amino-3-(Cyclopropyloxy) Benzoate 8

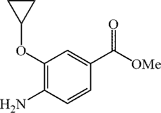 [SMILES: COC(=O)c1ccc(N)c(OC2CC2)c1]

A mixture of 7 (2.00 g, 8.96 mmol) and 10% Pd/C (200 mg) in THF (20 mL) was stirred under H₂ atmosphere (15 psi) at rt for 16 h. The suspension was filtered through a pad of Celite® and washed with THF (3 x 20 mL). The filtrate was concentrated to dryness under reduced pressure to afford 8 (1.8 g, 97% pure) as a yellow solid.

### 8-(Cyclopropyloxy)-3-Methylquinoline-6-Carboxylic Acid 9

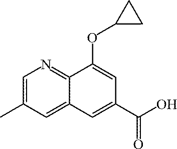 [SMILES: Cc1cnc2c(OC3CC3)cc(C(=O)O)cc2c1]

Methacrylaldehyde (2.70 g, 38.5 mmol) was added to a solution of 8 (2.50 g, 12.9 mmol) in HCl (6 M aq., 30 mL). The reaction mixture was stirred at rt for 30 min, then stirred at 100° C. for 40 min. The reaction mixture was concentrated to dryness under reduced pressure. The crude mixture was purified by preparative HPLC (Phenomenex Synergi Max-RP 250 x 50 mm x 10 µm, mobile phase gradient: 2% to 30% (v/v) CH₃CN and H₂O with 0.225% HCOOH). The product was suspended in water (30 mL). The mixture was frozen and then lyophilized to dryness to afford 9 (758 mg, 23%, 95% pure) as a yellow solid. ¹H NMR (400 MHz, DMSO-d6) δ ppm 8.78 (d, J=2.2 Hz, 1H), 8.23 (d, J=0.9 Hz, 1H), 8.13 (d, J=1.5 Hz, 1H), 7.85 (d, J=1.8 Hz, 1H), 4.07 (tt, J=2.9, 6.0 Hz, 1H), 2.47 (s, 3H), 0.93 - 0.87 (m, 2H), 0.82 - 0.76 (m, 2H).

### 3.1.4. Synthesis of 15

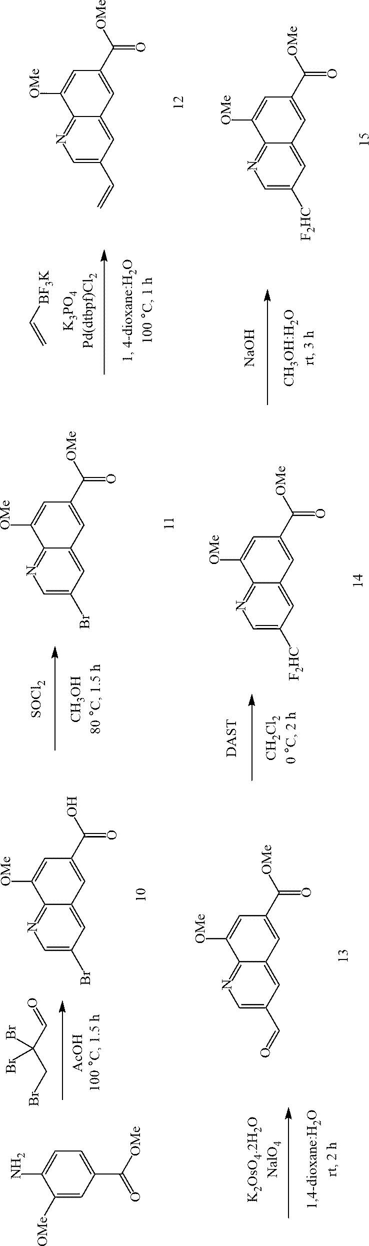

### 3-BromoMethoxyquinoline-6-Carboxylic Acid 10

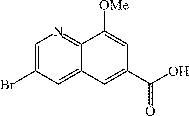 [SMILES: COc1cc(C(=O)O)cc2cc(Br)cnc12]

2,2,3-Tribromopropanal (18.0 g, 61.1 mmol) was added to a solution of methyl 4-amino-3-methoxybenzoate (10.0 g, 55.2 mmol) in AcOH (120 mL). The reaction mixture was stirred at 100° C. for 1.5 h. The reaction mixture was concentrated to dryness under reduced pressure to afford 10 (18.0 g), which was used in the next step without further purification.

### Methyl 3-Bromo-8-Methoxyquinoline-6-Carboxylate 11

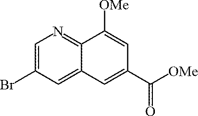 [SMILES: COC(=O)c1cc(OC)c2ncc(Br)cc2c1]

SOCl2 (8.0 mL, 110 mmol) was added to a solution of 10 in CH₃OH (150 mL). The reaction mixture was stirred at 80° C. for 1.5 h. The reaction mixture was concentrated to dryness under reduced pressure. The residue was triturated in EtOAc and CH₃OH (8:1, 100 mL) and the suspension was isolated via filtration. The filter cake was washed with EtOAc and CH₃OH (8:1, 2 x 50 mL) and dried under vacuum to afford 11.

### Methyl 3-ethenyl-8-methoxyquinoline-6-carboxylate 12

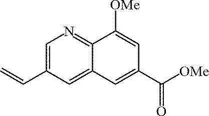 [SMILES: C=Cc1cnc2c(OC)cc(C(=O)OC)cc2c1]

A mixture of 11, potassium trifluoro(prop-1-en-2-yl)borate (7.60 g, 56.7 mmol), and K₃PO₄ (21.6 g, 102 mmol) in 1,4-dioxane (125 mL) and H₂O (25 mL) was purged with N₂ for 5 min. Pd(dtbpf)Cl₂ (3.30 mg, 5.06 mmol) was added and the mixture was purged with N₂ for another 5 min. The reaction mixture was stirred at 100° C. for 1 h. The mixture was cooled to rt and the reaction was quenched with water (80 mL). The layers were separated, and the aqueous phase was extracted with EtOAc (3 x 120 mL). The combined organic extracts were dried (Na₂SO₄). The solids were removed by filtration and the filtrate was concentrated to dryness under reduced pressure. The crude mixture was purified by column chromatography (petroleum ether/EtOAc, gradient from 12:1 to 1:2) to afford 12 (5.0 g, 33% over 3 steps) as a yellow solid.

### Methyl 3-formyl-8-methoxyquinoline-6-carboxylate 13

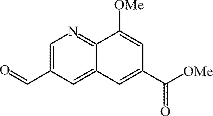 [SMILES: COC(=O)c1cc(OC)c2ncc(C=O)cc2c1]

K₂O_{S}O₄•2H₂O (310 mg, 0.84 mmol) was added to a solution of 12 (5.0 g, 21 mmol) in 1,4-dioxane (50 mL) and H₂O (50 mL). NaIO₄ (14.0 g, 65.4 mmol) was added. The reaction mixture was stirred at rt for 2 h. The reaction was poured into water (50 mL), and the aqueous phase was extracted with EtOAc (3 x 80 mL). The combined organic extracts were dried (Na₂SO₄). The solids were removed by filtration and the filtrate was concentrated to dryness under reduced pressure. The crude mixture was purified by column chromatography (petroleum ether/EtOAc, gradient from 10:1 to 1:2) to afford 13 (4.0 g, 79%) as a light yellow solid.

### Methyl 3-(difluoromethyl)-8-methoxyquinoline-6-carboxylate 14

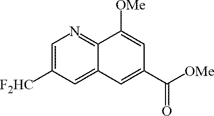 [SMILES: COC(=O)c1cc(OC)c2ncc(C(F)F)cc2c1]

DAST (10.0 mL, 75.7 mmol) was added dropwise to a solution of 13 (4.00 g, 16.3 mmol) in CH₂Cl₂ (50 mL) at 0° C. under N₂ atmosphere. The reaction mixture was stirred at 0° C. for 2 h. The reaction mixture was diluted with CH₂Cl₂ (20 mL) and poured into NaHC03 (sat., aq., 50 mL). The layers were separated, and the aqueous phase was extracted with CH₂Cl₂ (2 x 50 mL). The combined organic extracts were dried (Na₂SO₄). The solids were removed by filtration and the filtrate was concentrated to dryness under reduced pressure. The crude mixture was purified by silica column chromatography (petroleum ether/EtOAc, gradient from 10:1 to 2:3) to afford 14 (2.1 g, 46%, 95% pure) as a yellow solid.

### 3-(Difluoromethyl)-8-Methoxyquinoline-6-Carboxylic Acid 15

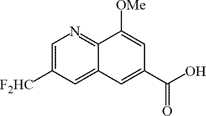 [SMILES: COc1cc(C(=O)O)cc2cc(C(F)F)cnc12]

NaOH (630 mg, 15.7 mmol) was added to a solution of 14 (2.10 g, 7.86 mmol) in CH₃OH (20 mL) and H₂O (4 mL). The reaction mixture was stirred at rt for 3 h and diluted with H₂O (25 mL) and CH₂Cl₂ (30 mL). The layers were separated, and the aqueous phase was diluted with HCl (1N) until pH 6. The suspension was isolated via filtration and washed with H₂O (3 x 15 mL) and dried under vacuum. The residue was suspended in H₂O (20 mL) and the mixture was frozen and lyophilized to dryness to afford 15 (1.71 g, 85%) as a light yellow solid. ¹H NMR (400 MHz, DMSO-d6) δ ppm 9.10 (s, 1H), 8.80 (s, 1H), 8.35 (s, 1H), 7.66 (s, 1H), 7.52 - 7.19 (m, 1H), 4.04 (s, 3H).

### 3.1.5. Synthesis of 17

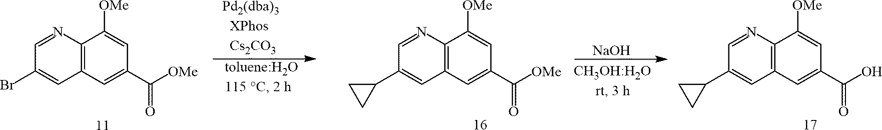

### Methyl 3-cyclopropyl-8-methoxyquinoline-6-carboxylate 16

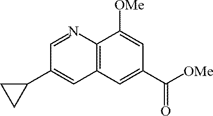 [SMILES: COC(=O)c1cc(OC)c2ncc(C3CC3)cc2c1]

11 (3.00 g, 10.1 mmol), cyclopropylboronic acid (990 mg, 11.5 mmol) and C_{S2}CO₃ (4.95 g, 15.2 mmol) were dissolved in toluene (40 mL) and H₂O (2 mL). The mixture was purged with Ar for 5 min and Pd₂(dba)₃ (4.62 g, 5.05 mmol) and XPhos (480 mg, 1.01 mmol) were added. The reaction mixture was purged with Ar for another 5 min and stirred at 115° C. for 2 h. The mixture was cooled to rt and the reaction was quenched with water (40 mL). The layers were separated, and the aqueous phase was extracted with EtOAc (3 x 60 mL). The combined organic extracts were dried (Na₂SO₄). The solids were removed by filtration and the filtrate was concentrated to dryness under reduced pressure. The residue was purified by silica column chromatography (petroleum ether/EtOAc, gradient from 12:1 to 4:1) to afford 16 (2.5 g, 71%) as a yellow solid.

### 3-Cyclopropyl-8-Methoxyquinoline-6-Carboxylic Acid 17

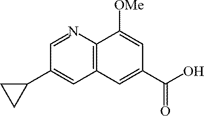 [SMILES: COc1cc(C(=O)O)cc2cc(C3CC3)cnc12]

NaOH (665 mg, 16.6 mmol) was added to a solution of 16 (2.00 g, 7.77 mmol) in CH₃OH (20 mL) and H₂O (4 mL). The reaction mixture was stirred at rt for 3 h. The mixture was diluted with water (30 mL) and CH2Cl₂ (20 mL). The layers were separated, and the pH of the aqueous layer was adjusted to 6 with HCl (1N, aq.). The suspension was isolated by filtration and washed with water (3 x 20 mL) and dried under reduced pressure. The residue was suspended in water (30 mL) and the mixture was frozen then lyophilized to dryness to afford 17 (1.3 g, 67%) as a yellow solid. ¹H NMR (400 MHz, DMSO-d6) δ ppm 8.79 (d, J=2.2 Hz, 1H), 8.11 (d, J=1.3 Hz, 1H), 8.07 (d, J=2.0 Hz, 1H), 7.47 (d, J=1.1 Hz, 1H), 3.99 (s, 3H), 2.19 - 2.11 (m, 1H), 1.13 -1.06 (m, 2H), 0.92 - 0.86 (m, 2H).

### 3.2. Synthesis of Cinnoline Intermediates

### 3.2.1. Synthesis of 23

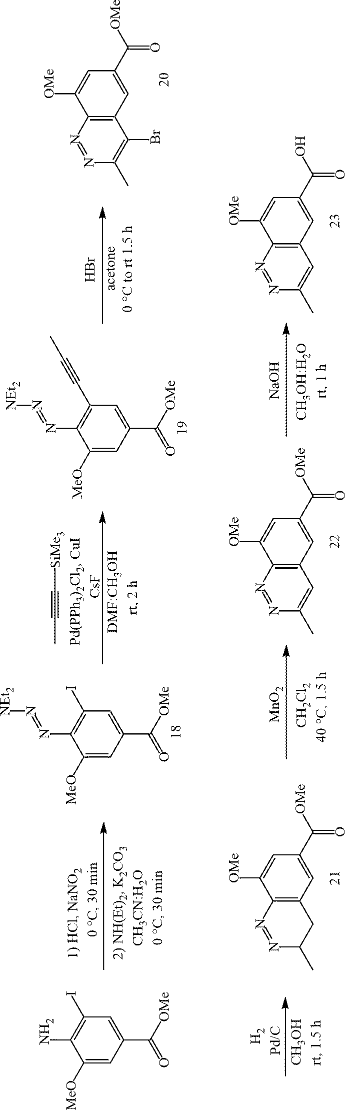

### Methyl 4-[3,3-diethyltriaz-1-en-1-yl]-3-iodo-5-methoxybenzoate 18

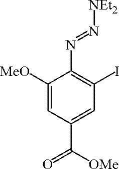

A solution of methyl 4-amino-3-iodo-5-methoxybenzoate (45.0 g, 146 mmol) and HCl (6 M in H₂O, 180 mL, 1.08 mol) was cooled in an ice bath (0° C.) while a solution of NaNO₂ (12.5 g, 181 mmol) in cold water (50 mL) was added dropwise. The resulting solution of diazonium salt was stirred at 0° C. for 30 min and was added to a solution of diethylamine (39 mL, 377 mmol) and K₂CO₃ (102 g, 734 mmol) in CH₃CN and H₂O (1:2, 390 mL) dropwise. The reaction mixture was stirred at 0° C. for 30 min. The reaction mixture was extracted with CH₂Cl₂ (2 x 500 mL). The combined organic extracts were dried (Na₂SO₄). The solids were removed by filtration and the filtrate was concentrated to dryness under reduced pressure. The crude mixture was purified by silica column chromatography (petroleum ether/EtOAc, gradient from 10:1 to 8:1) to afford 18 (53 g, 84%, 91% pure) as a yellow oil.

### Methyl 4-[3,3-diethyltriaz-1-en-1-yl]-3-methoxy-5-(prop-1-yn-1-yl)benzoate 19

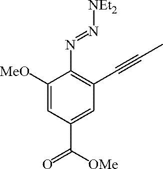

The reaction was performed on two batches of 25 g of 18. 18 (25 g, 63.9 mmol), trimethyl(prop-1-yn-1-yl)silane (57.5 g, 512 mmol), CuI (2.5 g, 13.1 mmol) and CsF (49.0 g, 323 mmol) were dissolved in DMF (250 mL) and CH₃OH (50 mL). The mixture was purged with Ar for 5 min and Pd(PPh₃)₂Cl₂ (2.25 g, 3.21 mmol) was added. The mixture was purged with Ar for another 5 minutes and the reaction mixture was stirred at rt for 2 h. The two batches were combined and poured into a brine (500 mL). The layers were separated, and the aqueous phase was extracted with EtOAc (3 x 500 mL). The combined organic extracts were washed with brine (50 mL), and dried (Na₂SO₄). The solids were removed by filtration and the filtrate was concentrated to dryness under reduced pressure. The crude mixture was purified by silica column chromatography (petroleum ether/EtOAc, 15:1) to afford 19 (36 g, 88%, 95% pure) as a yellow oil.

### Methyl 4-bromo-8-methoxy-3-methylcinnoline-6-carboxylate 20

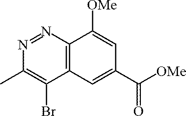 [SMILES: COC(=O)c1cc(OC)c2nnc(C)c(Br)c2c1]

HBr (48% aq., 54 mL, 477 mmol) was added to a solution of 19 (18.0 g, 59.3 mmol) in acetone (200 mL) at 0° C. The reaction mixture was stirred for 1.5 h with gradual warming to rt. Acetone was evaporated under reduced pressure and the residue was dissolved in CH₂Cl₂ (250 mL). The solution was washed with NaHCO₃ (sat., aq., 150 mL). The aqueous layer was extracted with CH₂Cl₂ (2 x 250 mL). The combined organic extracts were dried (Na₂SO₄). The solids were removed by filtration and the filtrate was concentrated to dryness under reduced pressure. The crude product 20 (18 g, 88%, 90% pure) was used in the next step without further purification.

### Methyl 8-methoxy-3-methyl-3,4-dihydrocinnoline-6-carboxylate 21

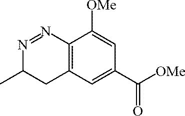 [SMILES: COC(=O)c1cc2c(c(OC)c1)N=NC(C)C2]

10% Pd/C (5.0 g) was added to a solution of 20 (20.0 g, 64.3 mmol) in CH₃OH (300 mL). The reaction mixture was stirred under H₂ atmosphere (15 psi) at rt for 1.5 h. The reaction mixture was filtered through a pad of Celite® and washed with CH₃OH (300 mL). The combined organic extracts were dried (Na₂SO₄). The solids were removed by filtration and the filtrate was concentrated to dryness under reduced pressure. The crude 21 (13 g) was used in the next step without further purification.

### Methyl 8-methoxy-3-methylcinnoline-6-carboxylate 22

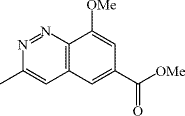 [SMILES: COC(=O)c1cc(OC)c2nnc(C)cc2c1]

MnO₂ (14.5 g, 167 mmol) was added to a solution of 21 (13 g, crude) in CH₂Cl₂ (130 mL). The reaction mixture was stirred at 40° C. for 1.5 h. The reaction mixture was filtered through a pad of Celite® and washed with CH2Cl₂ (300 mL). The combined organic extracts were dried (Na₂SO₄). The solids were removed by filtration and the filtrate was concentrated to dryness under reduced pressure. The crude mixture was purified by silica column chromatography (petroleum ether/EtOAc, gradient from 10:1 to 0:1) to afford 22 (4.2 g, 25% over 2 steps, 91% pure) as a yellow solid.

### 8-Methoxy-3-Methylcinnoline-6-Carboxylic Acid 23

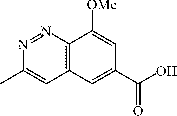 [SMILES: COc1cc(C(=O)O)cc2cc(C)nnc12]

NaOH (1.05 g, 26.3 mmol) was added to a solution of 22 (3.80 g, 16.4 mmol) in CH₃OH (30 mL) and H₂O (6 mL). The reaction mixture was stirred at rt for 1 h. CH₃OH was evaporated under reduced pressure and the aqueous phase was washed with CH₂Cl₂ (30 mL). The pH of the solution was adjusted to pH 2 with HCl (2 M aq.). The suspension was isolated by filtration, the solid was washed with water (20 mL) and the solvent removed under reduced pressure. The product was triturated in EtOAc (10 mL), isolated by filtration and washed with EtOAc (10 mL) before being dried under reduced pressure to afford 23 (3.02 g, 85%) as a brown solid. ¹H NMR (400 MHz, DMSO-d6) δ ppm 13.59 (br. s., 1H), 8.17 - 8.11 (m, 2H), 7.57 - 7.53 (m, 1H), 4.12 (s, 3H), 2.88 (s, 3H).

### 3.2.2. Synthesis of 29

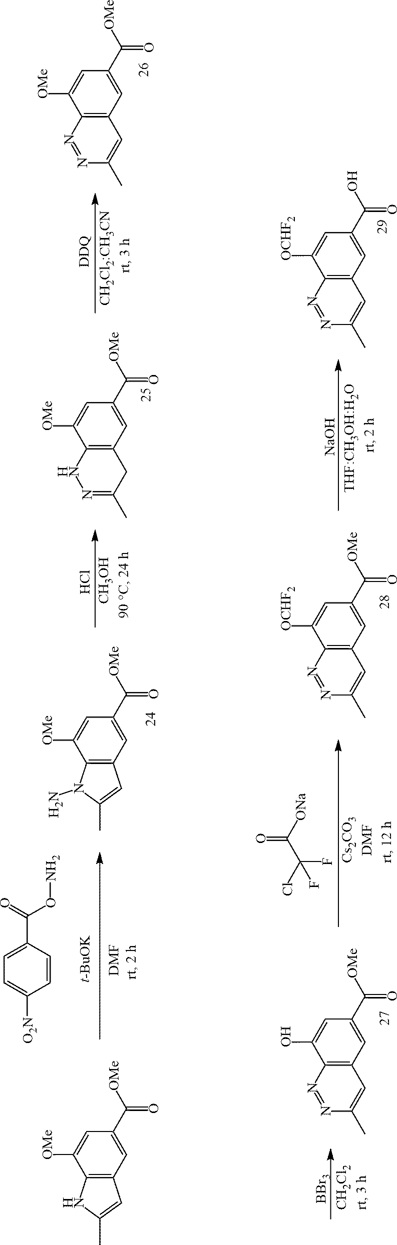

### Methyl 1-Amino-7-Methoxy-2-Methyl-1H-Indole-5-Carboxylate 24

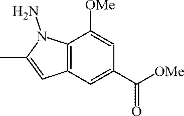 [SMILES: COC(=O)c1cc(OC)c2c(c1)cc(C)n2N]

To a mixture of methyl 7-methoxy-2-methyl-1H-indole-5-carboxylate (10.0 g, 45.6 mmol) and t-BuOK (7.68 g, 68.4 mmol) in DMF (150 mL) was added O-(4-nitrobenzoyl)hydroxylamine (12.5 g, 68.4 mmol). The reaction mixture was stirred at rt for 2 h. The reaction was quenched with NH₄Cl (sat., aq.) and diluted with EtOAc (300 mL). The layers were separated and the organic phase was washed with water (5 x 300 mL) and brine (3 times). The organic layer was dried (MgSO₄). The solids were removed by filtration and the filtrate was concentrated to dryness under reduced pressure. The residue was purified by silica column chromatography (petroleum ether/EtOAc, gradient from 100:0 to 80:20) to afford 24 (6.6 g, 62%).

### Methyl 8-Methoxy-3-Methyl-1,4-Dihydrocinnoline-6-Carboxylate 25

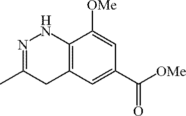 [SMILES: COC(=O)c1cc2c(c(OC)c1)NN=C(C)C2]

To a solution of 24 (700 mg, 3.00 mmol) in CH₃OH (50 mL) was added HCl (0.90 mmol). The reaction mixture was stirred at 90° C. for 24 h. The mixture was cooled to rt, filtered and concentrated under reduced pressure. The crude was purified by silica column chromatography (petroleum ether/EtOAc, gradient from 100:0 to 50:50) to afford 25 (0.3 g, 43%) as a yellow solid.

### Methyl 8-methoxy-3-methylcinnoline-6-carboxylate 26

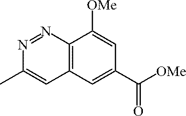 [SMILES: COC(=O)c1cc(OC)c2nnc(C)cc2c1]

To a solution of 25 (1.90 g, 5.04 mmol) in CH₂Cl₂ (10 mL) and CH₃CN (10 mL) was added 2,3-dichloro-5,6-dicyano-1,4-benzoquinone (5.94 g, 26.2 mmol). The reaction mixture was stirred at rt for 3 h. The mixture was filtered and concentrated under reduced pressure. The residue was purified by silica column chromatography (petroleum ether/EtOAc, gradient from 100:0 to 50:50) to afford 26 (2.9 g, 73%) as a yellow solid.

### Methyl 8-hydroxy-3-methylcinnoline-6-carboxylate 27

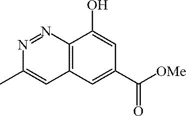 [SMILES: COC(=O)c1cc(O)c2nnc(C)cc2c1]

To a solution of 26 (900 mg, 3.88 mmol) in CH₂Cl₂ (50 mL) was added BBr3 (50 mL, 50 mmol). The reaction mixture was stirred at rt for 3 h, filtered and concentrated under reduced pressure. The residue was purified by silica column chromatography (petroleum ether/EtOAc, gradient from 100:0 to 50:50) to afford 27 (2.9 g, 73%) as a yellow solid.

### Methyl 8-(difluoromethoxy)-3-methylcinnoline-6-carboxylate 28

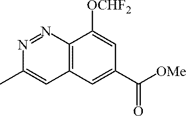 [SMILES: COC(=O)c1cc(OC(F)F)c2nnc(C)cc2c1]

To a solution of 27 (800 mg, 3.67 mmol) in DMF (50 mL) were added sodium chlorodifluoroacetate (1.12 g, 7.33 mmol) and C_{S2}CO₃ (3.58 g. 11.0 mmol). The reaction mixture was stirred at rt for 12 h. The mixture was filtered and concentrated under reduced pressure. The residue was purified by silica column chromatography (petroleum ether/EtOAc, gradient from 100:0 to 50:50) to afford 28 (250 mg, 25%) as a yellow solid.

### 8-(Difluoromethoxy)-3-Methylcinnoline-6-Carboxylic Acid 29

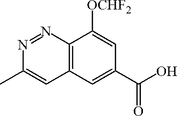 [SMILES: Cc1cc2cc(C(=O)O)cc(OC(F)F)c2nn1]

To a solution of 28 (170 mg, 0.63 mmol) in CH₃OH (5 mL), THF (5 mL) and H₂O (1 mL) was added NaOH (101 mg, 2.54 mmol). The reaction mixture was stirred at rt for 2 h. The reaction was neutralized with HCl (1N, aq., 4 mL) and the aqueous phase was extracted with EtOAc (3 x 20 mL). The combined organic extracts were washed with brine, dried (Na₂SO₄). The solids were removed by filtration and the filtrate was concentrated under reduced pressure. The crude mixture was purified by C-18 column chromatography (5-60% CH₃CN/H₂O (0.05% HCl) to deliver 29 (109 mg, 66%) as a yellow solid. ¹H NMR (400 MHz, DMSO-d6) δ ppm 8.52 (d, J=1.2 Hz, 1H), 8.32 (s, 1H), 7.90 - 7.51 (m, 1H), 2.93 (s, 3H).

### 3.2.3. Synthesis of 36

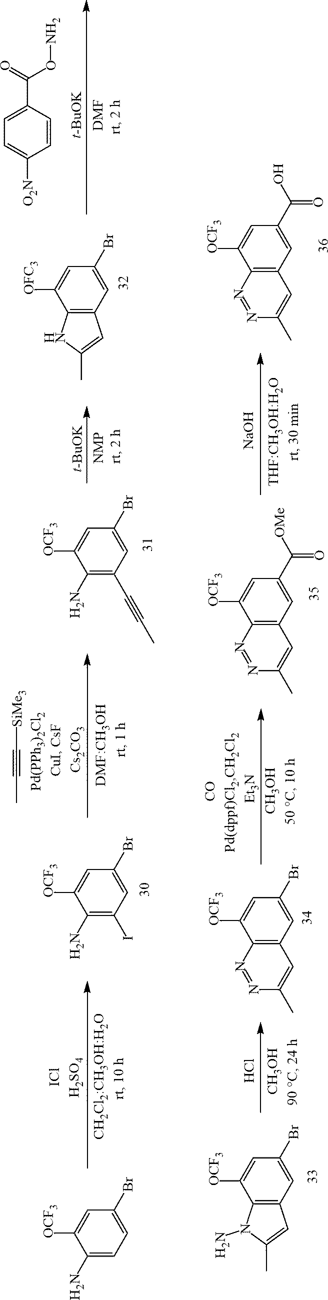

### 4-Bromoiodo-6-(trifluoromethoxy)aniline 30

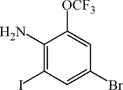 [SMILES: Nc1c(I)cc(Br)cc1OC(F)(F)F]

To a mixture of CH₃OH (75 mL) and water (8 mL) was added 4-bromo-2-(trifluoromethoxy)-aniline 5.00 g, 19.5 mmol). The mixture was cooled in an ice bath and concentrated H₂SO₄ (3 mL) was added followed by ICl (3.17 g, 19.5 mmol) in CH₂Cl₂ (19.5 mL). The reaction mixture was stirred at rt for 10 h. The reaction was quenched Na₂S₂O₃ (sat., aq.). The layers were separated, and the aqueous phase was extracted with EtOAc (3 x 100 mL). The combined organic extracts were dried (Na₂SO₄). The solids were removed by filtration and the filtrate was concentrated under reduced pressure. The crude mixture was purified by silica column chromatography (petroleum ether/EtOAc, gradient from 100:0 to 90:10) to afford 30 (3.75 g, 50%) as a yellow solid.

### 4-Bromo(prop-1-yn-1-yl)-6-(trifluoromethoxy)aniline 31

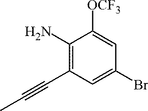 [SMILES: CC#Cc1cc(Br)cc(OC(F)(F)F)c1N]

30 (3.70 g, 9.69 mmol), CuI (369 mg, 1.94 mmol) and CsF (4.42 g, 29.1 mmol) were dissolved in DMF (120 mL) and CH₃OH (30 mL). The mixture was cooled to 0° C. and trimethyl(prop-1-yn-1-yl)silane (2.18 g, 19.4 mmol) was added. The mixture was purged with N₂ and PdCl2(PPh₃)₂ (0.34 g, 0.48 mmol) was added. The reaction mixture was purged again with N₂ and stirred at rt for 1 h. The reaction was quenched with NH₄Cl (sat., aq.). The layers were separated, and the aqueous phase was extracted with EtOAc (3 x 200 mL). The combined organic extracts were dried (Na₂SO₄). The solids were removed by filtration and the filtrate was concentrated under reduced pressure. The residue was purified by silica column chromatography (petroleum ether/EtOAc, gradient from 100:0 to 90:10) to afford 31 (2.5 g, 88%) as a yellow solid.

### 5-BromoMethyl-7-(Trifluoromethoxy)-1H-Indole 32

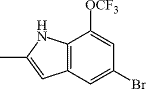 [SMILES: Cc1cc2cc(Br)cc(OC(F)(F)F)c2[nH]1]

31 (2.30 g, 7.82 mmol) was dissolved in NMP (50 mL). The mixture was purged with N₂ and t-BuOK (2.19 g, 19.5 mmol) was added. The mixture was purged again with N₂ and stirred at rt for 2 h. The reaction was quenched with NH₄Cl (sat., aq.). The layers were separated, and the aqueous phase was extracted with EtOAc (3 x 200 mL). The combined organic extracts were dried (Na₂SO₄). The solids were removed by filtration and the filtrate was concentrated under reduced pressure. The residue was purified by silica column chromatography (petroleum ether/EtOAc, gradient from 100:0 to 90:10) to afford 32 (1.95 g, 85%) as a yellow solid.

### 5-BromoMethyl-7-(Trifluoromethoxy)-1H-Indol-1-Amine 33

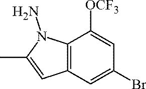 [SMILES: Cc1cc2cc(Br)cc(OC(F)(F)F)c2n1N]

32 (1.90 g, 6.46 mmol) was dissolved in DMF (50 mL). The mixture was purged with N₂ and t-BuOK (1.09 g, 9.69 mmol) was added followed by O-(4-nitrobenzoyl)hydroxylamine (1.76 g, 9.69 mmol). The reaction mixture was purged again with N₂ and stirred at rt for 2 h. The reaction was quenched with NH₄Cl (sat., aq.). The layers were separated, and the aqueous phase was extracted with EtOAc (3 x 200 mL). The combined organic extracts were dried (Na₂SO₄). The solids were removed by filtration and the filtrate was concentrated under reduced pressure. The residue was purified by silica column chromatography (petroleum ether/EtOAc, gradient from 100:0 to 80:20) to afford 33 (1.74 g, 87%) as a yellow solid.

### 6-Bromomethyl-8-(trifluoromethoxy)cinnoline 34

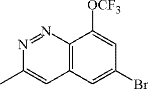 [SMILES: Cc1cc2cc(Br)cc(OC(F)(F)F)c2nn1]

To a solution of 33 (400 mg, 1.29 mmol) in CH₃OH (30 mL) was added HCl (14.2 mg, 0.39 mmol). The reaction mixture was stirred at 90° C. for 24 h. The mixture was cooled to rt. The solids were removed by filtration and the filtrate was concentrated under reduced pressure. The residue was purified by silica column chromatography (petroleum ether/EtOAc, gradient from 100:0 to 90:10) to afford 34 (90 mg, 23%) as a yellow solid.

### Methyl 3-methyl-8-(trifluoromethoxy)cinnoline-6-carboxylate 35

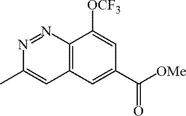 [SMILES: COC(=O)c1cc(OC(F)(F)F)c2nnc(C)cc2c1]

To a solution of 34 (2.00 g, 6.51 mmol) in CH₃OH (50 mL) were added Pd(dppf)Cl₂.CH₂Cl₂ (0.55 g, 0.65 mmol) and Et₃N (1.32 g, 13.0 mmol). The reaction mixture was purged with CO (1 atm) and stirred at 50° C. for 10 h. The mixture was cooled to rt, filtered and the filtrate was concentrated under reduced pressure. The residue was purified by silica column chromatography (CH2Cl2/EtOAc, gradient from 100:0 to 95:5) to afford 35 (853 mg, 46%) as a yellow solid.

### 3-Methyl-8-(Trifluoromethoxy)Cinnoline-6-Carboxylic Acid 36

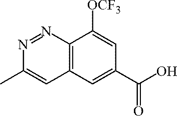 [SMILES: Cc1cc2cc(C(=O)O)cc(OC(F)(F)F)c2nn1]

To a solution of 35 (923 mg, 3.23 mmol) in CH₃OH (15 mL) and THF (15 mL) was added NaOH (387 mg, 9.68 mmol) and H₂O (1 mL). The reaction mixture was stirred at rt for 30 min. The mixture was filtered, and the filtrate was concentrated under reduced pressure. The residue was purified by reverse phase chromatography to afford 36 (562 mg, 64%) as a yellow solid. ¹H NMR (300 MHz, DMSO-d6) δ ppm 8.69 (d, J=1.6 Hz, 1H), 8.37 (s, 1H), 8.14 - 8.07 (m, 1H), 2.94 (s, 3H).

### 3.2.4. Synthesis of 48

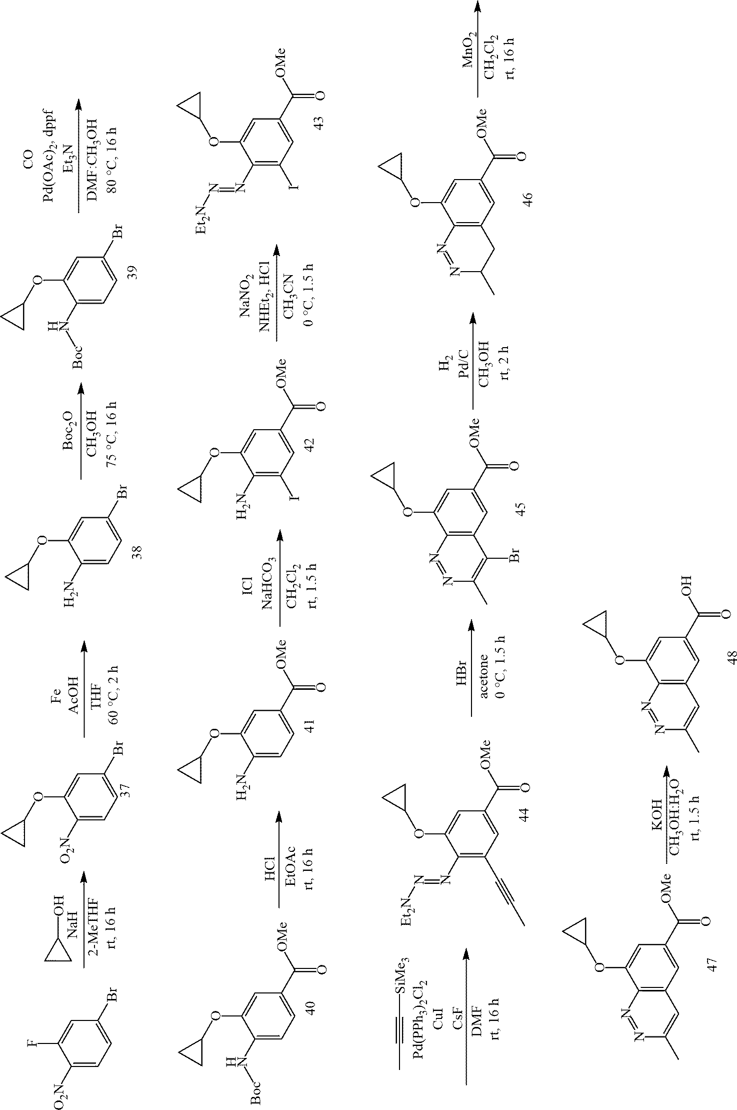

### 4-Bromo(cyclopropyloxy)-l -nitrobenzene 37

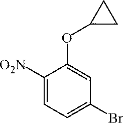 [SMILES: O=[N+]([O-])c1ccc(Br)cc1OC1CC1]

To a mixture of 4-bromo-2-cyclopropoxy-1-nitrobenzene (350 g, 1.59 mol) and cyclopropanol (166 g, 2.86 mol) in 2-MeTHF (3.5 L) was added NaH (60% pure, 114 g, 2.86 mol) at 0° C. The reaction mixture was stirred at rt for 16 h under N₂ atmosphere. The reaction was quenched with NH₄Cl (sat., aq., 2.5 L). The layers were separated, and the aqueous phase was extracted with EtOAc (3 x 1 L). The combined organic extracts were washed with brine (2 L), and dried (Na₂SO₄). The solids were removed by filtration and the filtrate was concentrated under reduced pressure to afford 37 that was used without further purfication in the next step.

### 4-BromoCyclopropoxyaniline 38

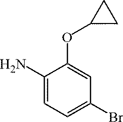 [SMILES: Nc1ccc(Br)cc1OC1CC1]

To a solution of 37 (410 g, 1.59 mol) in AcOH (909 mL, 15.9 mol) in THF (2.5 L) was added Fe (444 g, 7.94 mol). The reaction mixture was stirred at 60° C. for 2 h. The solids were removed by filtration and the filtrate was concentrated under reduced pressure. The residue was purified by silica column chromatography (petroleum ether/EtOAc, gradient from 90:10 to 85:15) to afford 38 (320.5 g, 84% over 2 steps, 96% pure) as yellow oil.

### Tert-Butyl [4-Bromo-2-(Cyclopropyloxy)Phenyl]Carbamate 39

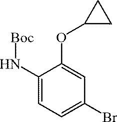 [SMILES: CC(C)(C)OC(=O)Nc1ccc(Br)cc1OC1CC1]

A mixture of 38 (320 g, 1.41 mol) and Boc₂O (368 g, 1.69 mol) in CH₃OH (3 L) was stirred at 75° C. for 16 h. The reaction mixture was concentrated under reduced pressure. The residue was purified by silica column chromatography (petroleum ether/EtOAc, 20:1) to afford 39 (420.5 g, 91% yield) as light yellow solid.

### Methyl 4-[(tert-butoxycarbonyl)amino]-3-(cyclopropyloxy)benzoate 40

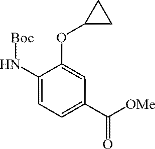 [SMILES: COC(=O)c1ccc(NC(=O)OC(C)(C)C)c(OC2CC2)c1]

Three reactions were carried out in parallel. To a mixture of 39 (110 g, 335 mmol), Et₃N (136 g, 1.34 mol), and dppf (18.6 g, 33.5 mmol) in CH₃OH (600 mL) and DMF (300 mL) was added Pd(OAc)₂ (3.76 g, 16.7 mmol). The reaction mixture was stirred at 80° C. for 16 h under CO atmosphere (50 psi). The solids were removed by filtration and the filtrate was concentrated under reduced pressure. The residue was diluted with H₂O (4 L) and extracted with EtOAc (3 x 1.5 L). The combined organic extracts were washed with brine (2 L) and dried (Na₂SO₄). The solids were removed by filtration and the filtrate was concentrated under reduced pressure. The crude mixture was purified by silica column chromatography (petroleum ether/EtOAc, gradient from 100:0 to 90:10) to afford 40 (210.5 g, 65%, 96% pure) as light yellow oil.

### Methyl 4-amino-3-cvclopropoxybenzoate 41

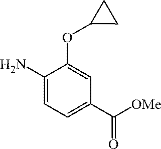 [SMILES: COC(=O)c1ccc(N)c(OC2CC2)c1]

To a solution of 40 (211 g, 685 mmol) in CH₂Cl₂ (1.6 L) was added TFA (588.1 g, 5.16 mol) and the reaction mixture was stirred at rt for 16 h. The reaction mixture was concentrated under reduced pressure. The residue was dissolved in EtOAc (1 L) and NaHCOs (aq., 1 L) was added. The layers were separated, and the aqueous phase was extracted with EtOAc (2 x 500 mL). The combined organic extracts were washed with brine (1 L) and dried (Na₂SO₄). The solids were removed by filtration and the filtrate was concentrated under reduced pressure to afford 41 (140.5 g, 97%, 98% pure) as yellow oil.

### Methyl 4-amino-3-(cyclopropyloxy)-5-iodobenzoate 42

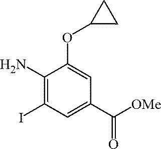 [SMILES: COC(=O)c1cc(I)c(N)c(OC2CC2)c1]

To a solution of 41 (140 g, 676 mmol) and NaHCO₃ (116 g, 1.38 mol) in CH₂Cl₂ (1 L) was added ICl (121 g, 743 mmol) in CH₂Cl₂ (200 mL). The reaction mixture was stirred at rt for 1.5 h. The reaction was quenched with Na₂SO₃ (sat., aq., 1 L). The layers were separated, and the aqueous phase was extracted with CH₂Cl₂ (2 x 500 mL). The combined organic extracts were washed with brine (500 mL) and dried (Na₂SO₄). The solids were removed by filtration and the filtrate was concentrated under reduced pressure. The residue was purified by silica column chromatography (petroleum ether/EtOAc, gradient from 100:0 to 90:10) to afford 42 (103.5 g, 46%) as yellow solid.

### Methyl 3-(cyclopropyloxy)-4-[3,3-diethyltriaz-1-en-1-yl]-5-iodobenzoate 43

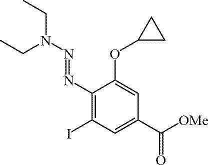 [SMILES: CCN(CC)/N=N/c1c(I)cc(C(=O)OC)cc1OC1CC1]

To a solution of 42 (103 g, 310 mmol) in HCl (6 M, 259 mL) in CH₃CN (130 mL) was added a solution of sodium nitrite (32.1 g, 466 mmol) in H₂O (65 mL) at 0° C. The reaction mixture was stirred at 0° C. for 30 min to afford the corresponding diazonium salt. To a suspension of N-ethylethanamine (45. 5 g, 621 mmol) and K₂CO₃ (215 g, 1.55 mol) in CH₃CN (250 mL) and H₂O (500 mL) was added the freshly prepared diazonium salt at 0 - 5° C. The reaction mixture was stirred at 0° C. for 1 h. The reaction mixture was diluted with H₂O (500 mL) and extracted with EtOAc (3 x 500 mL). The combined organic extracts were washed with brine (500 mL) and dried (Na₂SO₄). The solids were removed by filtration and the filtrate was concentrated under reduced pressure. The residue was purified by silica column chromatography (petroleum ether/EtOAc, gradient from 100:0 to 90:10) to afford 43 (114.5 g, 83%) as yellow oil.

### Methyl 3-cyclopropoxy-4-(3,3-diethyltriaz-1-en-1-yl)-5-(prop-1-yn-1-yl)benzoate 44

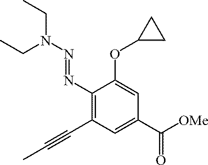 [SMILES: CC#Cc1cc(C(=O)OC)cc(OC2CC2)c1/N=N/N(CC)CC]

To a suspension of 43 (114 g, 273 mmol), CsF (207 g, 1.37 mol), CuI (10.4 g, 54.6 mmol) and Pd(PPh₃)₂Cl₂ (9.59 g, 13.7 mmol) in DMF (1 L) and CH₃OH (200 mL) was added trimethyl(prop-1-ynyl)silane (61.3 g, 546 mmol) under N₂ atmosphere. The reaction mixture was stirred at rt for 16 h under N₂. The reaction mixture was filtered, and the filtrate was diluted with H₂O (4 L). The aqueous phase was extracted with EtOAc (3 x 1.5 L). The combined organic layers were washed with brine (2 L) and dried (Na₂SO₄). The solids were removed by filtration and the filtrate was concentrated under reduced pressure. The residue was purified by silica column chromatography (petroleum ether/EtOAc, gradient from 90:10 to 85:15) to afford 44 (89.3 g, 99%) as a yellow oil.

### Methyl 4-bromo-8-(cyclopropyloxy)-3-methylcinnoline-6-carboxylate 45

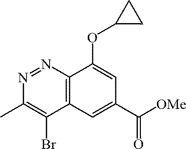 [SMILES: COC(=O)c1cc(OC2CC2)c2nnc(C)c(Br)c2c1]

Three reactions were carried out in parallel. To a solution of 44 (89.2 g, 271 mmol) in acetone (550 mL) was added HBr (137 g, 812 mmol, 48% pure) at 0 - 10° C. The reaction mixture was stirred at 0° C. for 1.5 h. The solids were isolated by filtration. The filter cake was dissolved in CH₂Cl₂ (1.5 L) and Et₃N (75 mL), washed with H₂O (500 mL), brine (500 mL), and dried (Na₂SO₄). The solids were removed by filtration and the filtrate was concentrated under reduced pressure to afford 45 (83.5 g, 246 mmol, 91%) as a yellow solid.

### Methyl 8-(cyclopropyloxy)-3-methyl-3,4-dihydrocinnoline-6-carboxylate 46

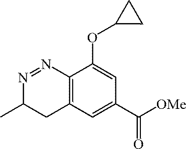 [SMILES: COC(=O)c1cc2c(c(OC3CC3)c1)N=NC(C)C2]

Two reactions were carried out in parallel. A suspension of 45 (41.5 g, 123 mmol) and wet 10% Pd/C (6.10 g) in CH₃OH (600 mL) was stirred at rt for 2.5 h under H₂ atmosphere. The solids were removed by filtration and the filtrate was concentrated under reduced pressure. The residue was purified by silica column chromatography (CH₂Cl₂/Et₂O) to afford 46 (24.5 g, 36%, 95% pure) as a yellow solid.

### Methyl 8-(cyclopropyloxy)-3-methylcinnoline-6-carboxylate 47

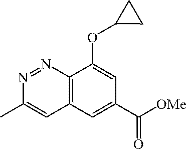 [SMILES: COC(=O)c1cc(OC2CC2)c2nnc(C)cc2c1]

A suspension of 46 (24.5 g, 94.1 mmol) and MnO₂ (40.9 g, 471 mmol) in CH₂Cl₂ (300 mL) was stirred at rt for 16 h. The reaction mixture was filtered, and the filtrate was concentrated under reduced pressure to afford 47 (23.5 g, 97%) as a yellow solid, which was used directly in the next step without purification.

### 8-(Cyclopropyloxy)-3-Methylcinnoline-6-Carboxylic Acid 48

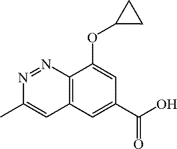 [SMILES: Cc1cc2cc(C(=O)O)cc(OC3CC3)c2nn1]

To a solution of 47 (23.5 g, 90.9 mmol) in CH₃OH (200 mL) and H₂O (100 mL) was added KOH (6.13 g, 109 mmol). The reaction mixture was stirred at rt for 1.5 h. To the reaction mixture was added TFA until pH ≈ 2. Precipitation occurred. The solids were isolated by filtration to afford 48 (16.5 g, 74%). ¹H NMR (400 MHz, DMSO-d6) δ ppm 8.16 (d, J=1.54 Hz, 1H), 8.14 (s, 1H), 7.90 (d, J=1.54 Hz, 1H), 4.17-4.26 (m, 1H), 2.88 (s, 3H), 0.92-1.01 (m, 2H), 0.84-0.92 (m, 2H); LC-MS (method D): Rt = 1.07 min; mass calcd. for C₁₃H₁₂N₂O₃ 244.0, m/z found 245.0 [M+H]⁺.

### 3.2.5. Synthesis of 56

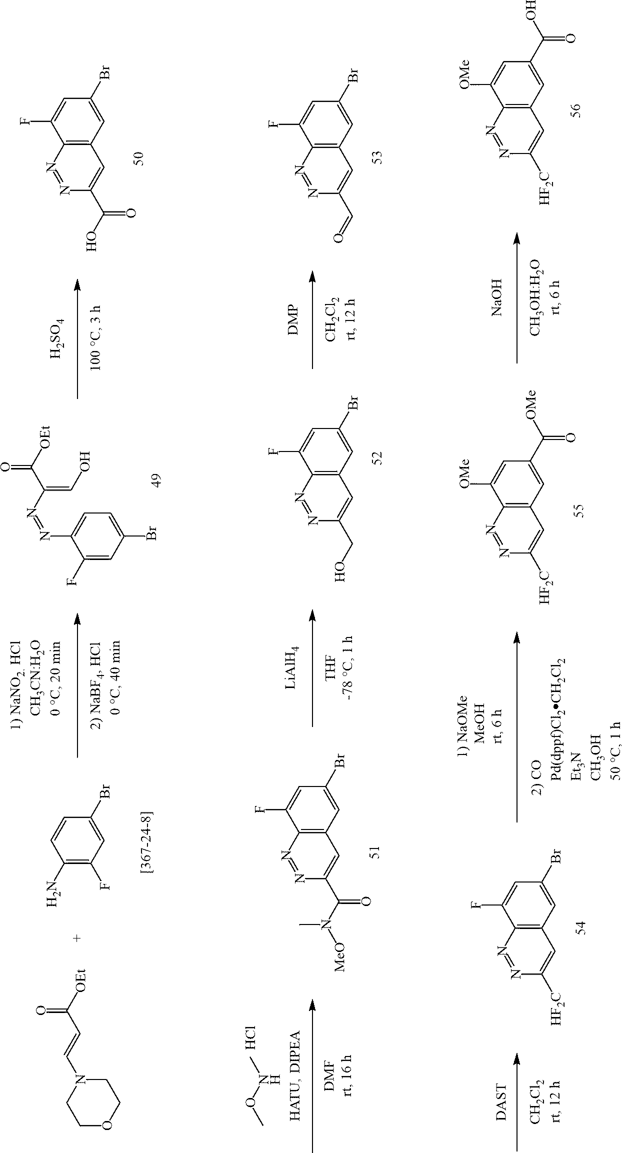

### Ethyl 2-((4-Bromo-2-Fluorophenyl)Diazenyl)-3-Hydroxy Acrylate 49

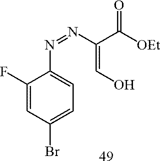

To a cooled solution of 4-bromo-2-fluoroaniline (7.60 g, 40 mmol) in H₂O (60 mL) was added –HCl (conc., 10 mL) and NaNO₂ (3.31 g, 48.0 mmol). After 20 min at 0° C., conc. HCl (13 mL) and NaBF₄ (17.6 g, 160 mmol) were added. The mixture was stirred for 40 min and the diazonium salt was isolated by filtration, washed with H₂O and Et₂O. A solution of diazonium salt in CH₃CN (60 mL) was treated with ethyl 3-morpholinoacrylate (3.26 g, 17.6 mmol). The reaction mixture was stirred at rt for 16 h. The solvent was removed under reduced pressure and the residue was purified by silica column chromatography (petroleum ether/EtOAc, gradient from 100:0 to 80:20) to afford 49 (4.5 g, 35%) as a yellow solid.

### 6-BromoFluorocinnoline-3-Carboxylic Acid 50

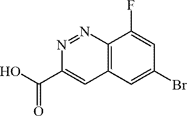 [SMILES: O=C(O)c1cc2cc(Br)cc(F)c2nn1]

A mixture of 49 (7.50 g, 23.7 mmol) in concentrated sulfuric acid (100 mL) was heated at 100° C. for 3 h. The mixture was cooled to 0° C., and the mixture was diluted with H₂O (200 mL). The brown solid was removed by filtration, and the filtrate was extracted with CH₂Cl₂ (4 x 600 mL). The combined organic extracts were dried (Na₂SO₄). The solids were removed by filtration and the filtrate was concentrated to dryness under reduced pressure to afford 50 (2.5 g, 39%) as a dark solid.

### 6-BromoFluoro-A-Methoxy-A-Methylcinnoline-3-Carboxamide 51

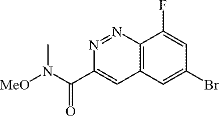 [SMILES: CON(C)C(=O)c1cc2cc(Br)cc(F)c2nn1]

To a solution of 50 (13.0 g, 48.0 mmol) in DMF (150 mL) were added N,O-dimethylhydroxylamine hydrochloride (6.09 g, 62.4 mmol), HATU (21.9 g, 57.6 mmol) and DIPEA (18.6 g, 144 mmol). The reaction mixture was stirred at rt for 6 h. The reaction was quenched with H₂O (100 mL) and extracted with EtOAc (3 x 200 mL). The combined organic layers were washed with H₂O (2 x 300 mL) and dried (Na₂SO₄). The solids were removed by filtration and the filtrate was concentrated to dryness under reduced pressure. The crude mixture was purified by silica column chromatography (petroleum ether/EtOAc, gradient from 100:0 to 50:50) to afford 51 (7.0 g, 46%) as a yellow solid.

### Bromo-8-fluorocinnolin-3-yl)methanol 52

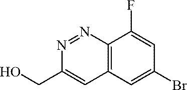 [SMILES: OCc1cc2cc(Br)cc(F)c2nn1]

To a solution of 51 (7.00 g, 22.3 mmol) in THF (6 mL) at -78° C. was added LiAlH₄ (3.38 g, 89.1 mmol). The mixture was stirred at -78° C. for 1 h. The mixture was warmed to rt and the reaction was quenched with Na₂SO₄.10 H₂O (10 g). The solids were removed by filtration and the filtrate was concentrated to dryness under reduced pressure to afford 52 (5.4 g, 94%) as a yellow solid.

### 6-Bromofluorocinnoline-3-carbaldehyde 53

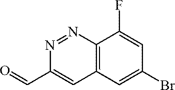 [SMILES: O=Cc1cc2cc(Br)cc(F)c2nn1]

To a solution of 52 (5.40 g, 21.0 mmol) at 0° C. in CH₂Cl₂ (30 mL) was added DMP (13.4 g, 31.5 mmol). The reaction mixture was stirred at rt for 12 h. The solvent was removed under reduced pressure and the crude mixture was purified by silica column chromatography (CH₂Cl₂/EtOAc, gradient from 100:0 to 95:5) to afford 53 (2.5 g, 47%) as a yellow solid.

### 6-Bromo(difluoromethyl)-8-fluorocinnoline 54

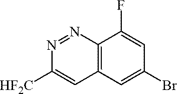 [SMILES: Fc1cc(Br)cc2cc(C(F)F)nnc12]

To a solution of 53 (408.1 mg, 1.6 mmol) at -20° C. in CH₂Cl₂ (10 mL) was added DAST (1.03 g, 6.40 mmol). The reaction mixture was stirred at rt for 12 h. The reaction was quenched with H₂O (10 mL). The layers were separated, and the aqueous phase was extracted with CH₂Cl₂ (3 x 20 mL). The combined organic extracts were dried (Na₂SO₄). The solids were removed by filtration and the filtrate was concentrated to dryness under reduced pressure. The crude mixture was purified by silica column chromatography (petroleum ether/EtOAc, gradient from 100:0 to 70:30) to afford 54 (400 mg, 90%) as a yellow solid.

### Methyl 3-(difluoromethyl)-8-methoxycinnoline-6-carboxylate 55

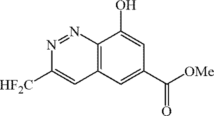 [SMILES: COC(=O)c1cc(O)c2nnc(C(F)F)cc2c1]

To a solution of 54 (2.32 g, 8.37 mmol) in CH₃OH (60 mL) was added NaOMe (1.81 g, 33.5 mmol). The reaction mixture was stirred at rt for 6 h. The reaction was quenched with H₂O (15 mL) and extracted with EtOAc (3 x 80 mL). The combined organic extracts were dried (Na₂SO₄). The solids were removed by filtration and the filtrate was concentrated under reduced pressure to afford a yellow solid (2 g).

To a solution of the residue in CH₃OH (100 mL) were added Pd(dppf)Cl₂•CH₂Cl₂ (565 mg, 0.69 mmol) and Et₃N (1.40 g, 13.8 mmol). The reaction mixture was stirred under CO atmosphere (1 atm) at 50° C. for 1 h. The reaction mixture was cooled to rt and the solvent was removed under reduced pressure. The crude mixture was purified by silica column chromatography (petroleum ether/EtOAc, gradient from 100:0 to 70:30) to afford 55 (1.8 g, 80%) as a yellow solid.

### 3-(Difluoromethyl)-8-Methoxycinnoline-6-Carboxylic Acid 56

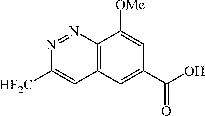 [SMILES: COc1cc(C(=O)O)cc2cc(C(F)F)nnc12]

To a solution of 55 (1.20 g, 4.47 mmol) in CH₃OH (40 mL) were added NaOH (536 mg, 13.4 mmol) and H₂O (4 mL). The reaction mixture was stirred at rt for 6 h. The reaction was neutralized with IN HCl (20 mL). The layers were separated, and the aqueous phase was extracted with EtOAc (3 x 60 mL). The combined organic extracts were washed with brine and dried (Na₂SO₄). The solids were removed by filtration and the filtrate was concentrated to dryness under reduced pressure. The crude mixture was purified by C-18 column chromatography (CH₃CN/H₂O (0.05% TFA), gradient from 85:15 to 60:40) to afford 56 (1.02 g, 89%) as a yellow solid. ¹H NMR (300 MHz, DMSO-d6) δ ppm 13.77 (brs, 1H), 8.77 (s, 1H), 8.40 (d, J=1.5 Hz, 1H), 7.75 (d, J=1.5 Hz, 1H), 7.59 (t, J=54.0 Hz, 1H), 4.18 (s, 3H).

### 3.2.6. Synthesis of 61

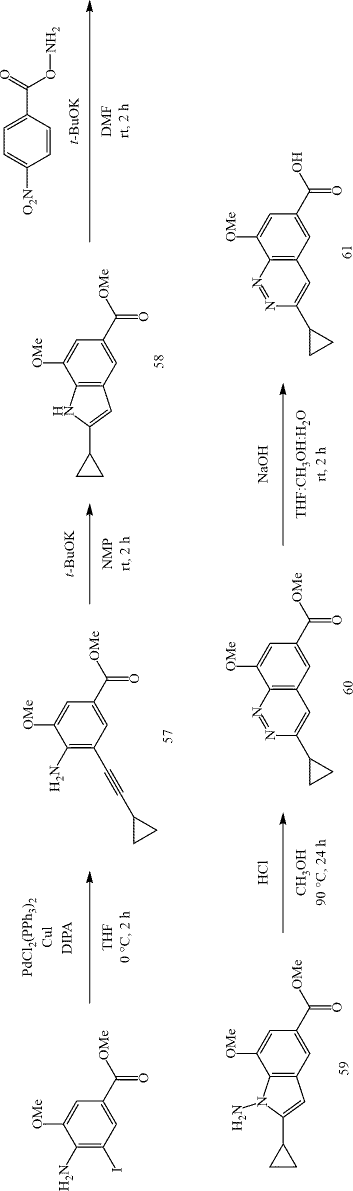

### Methyl 4-ammo-3-(cyclopropylethynyl)-5-methoxybenzoate 57

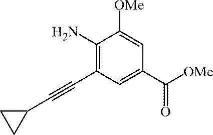 [SMILES: COC(=O)c1cc(C#CC2CC2)c(N)c(OC)c1]

Methyl 4-amino-3-iodo-5-methoxybenzoate (3.07 g, 10.0 mmol), Pd(PPh₃)₂Cl₂ (702 mg, 1.00 mmol) and CuI (381 mg, 2.00 mmol) were dissolved in THF (50 mL). The reaction mixture was purged with N₂. Then ethynylcyclopropane (1.69 mL, 20.0 mmol) was added at 0° C., followed by diisopropylamine (4.20 mL, 30.0 mmol). The mixture was purged again with N₂ and the reaction mixture was stirred at 0° C. for 2 h. NH₄Cl (sat., aq.) was added. The layers were separated, and the aqueous phase was extracted with EtOAc (3 x 100 mL). The combined organic extracts were dried (Na₂SO₄). The solids were removed by filtration and the filtrate was concentrated to dryness under reduced pressure. The residue was purified by silica column chromatography (petroleum ether/EtOAc, gradient from 100:0 to 90:10) to afford 57 (2.27 g, 94%) as a yellow solid.

### Methyl 2-cyclopropyl-7-methoxy-17/-indole-5-carboxylate 58

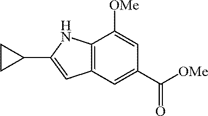 [SMILES: COC(=O)c1cc(OC)c2[nH]c(C3CC3)cc2c1]

57 (2.60 g, 10.6 mmol) was dissolved in NMP (50 mL). The mixture was purged with N₂ and t-BuOK (2.97 g, 26.5 mmol) was added. The mixture was purged again with N₂ and the reaction mixture was stirred at rt for 2 h. NH₄Cl (sat., aq.) was added. The layers were separated, and the aqueous phase was extracted with EtOAc (3 x 100 mL). The combined organic extracts were dried (Na₂SO₄). The solids were removed by filtration and the filtrate was concentrated to dryness under reduced pressure. The residue was purified by silica column chromatography (petroleum ether/EtOAc, gradient from 100:0 to 80:20) to afford 58 (1.75 g, 67%) as a yellow solid.

### Methyl 1-Amino-2-Cyclopropyl-7-Methoxy-1H-indole-5-Carboxylate 59

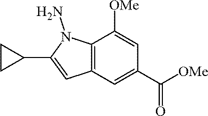 [SMILES: COC(=O)c1cc(OC)c2c(c1)cc(C1CC1)n2N]

58 (1.75 g, 7.14 mmol) was dissolved in DMF (40 mL). The mixture was purged with N₂ and t-BuOK (1.20 g, 10.7 mmol) was added followed by O-(4-nitrobenzoyl)hydroxylamine (1.95 g, 10.7 mmol). The mixture was purged again with N₂ and the reaction mixture was stirred at rt for 2 h. NH₄Cl (sat., aq.) was added. The layers were separated, and the aqueous phase was extracted with EtOAc (3 x 100 mL). The combined organic extracts were dried (Na₂SO₄), the solids were removed by filtration and the filtrate was concentrated to dryness under reduced pressure. The crude was purified by silica column chromatography (petroleum ether/EtOAc, gradient from 100:0 to 80:20) to afford 59 (0.8 g, 43%) as a yellow solid.

### Methyl 3-cyclopropyl-8-methoxycinnoline-6-carboxylate 60

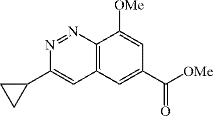 [SMILES: COC(=O)c1cc(OC)c2nnc(C3CC3)cc2c1]

To a solution of 59 (650 mg, 2.50 mmol) in CH₃OH (40 mL) was added HCl (0.75 mmol). The reaction mixture was stirred at 90° C. for 24 h. The mixture was cooled to rt, filtered and concentrated under reduced pressure. The crude mixture was purified by silica column chromatography (petroleum ether/EtOAc, gradient from 100:0 to 50:50) to afford 60 (430 mg, 62%) as a yellow solid.

### 3-Cyclopropyl-8-Methoxycinnoline-6-Carboxylic Acid 61

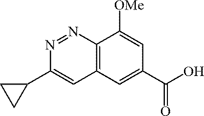 [SMILES: COc1cc(C(=O)O)cc2cc(C3CC3)nnc12]

To a solution of 60 (430 mg, 1.67 mmol) in CH₃OH (5 mL), THF (5 mL) and H₂O (0.5 mL) was added NaOH (266 mg, 6.66 mmol) and the reaction mixture was stirred at rt for 2 h. The reaction was neutralized with HCl (1N, aq., 4 mL). The layers were separated, and the aqueous phase was extracted with EtOAc (3 x 20 mL). The combined organic layers were washed with brine and dried (Na₂SO₄). The solids were removed by filtration and the filtrate was concentrated to dryness under reduced pressure. The crude mixture was purified by C-18 column chromatography (CH₃CN/H₂O (0.05% HCl), gradient from 95:5 to 60:40) to give 61 (138.5 mg, 33%) as a yellow solid. ¹H NMR (300 MHz, DMSO-d6) δ ppm 8.13 - 8.12 (m, 2H), 7.53 -7.52 (d, J= 1.2 Hz, 1H), 4.11 (s, 3H), 2.50 (s, 1H), 1.26 - 1.16 (m, 4H).

### 3.3.1. Synthesis of 67 and 68

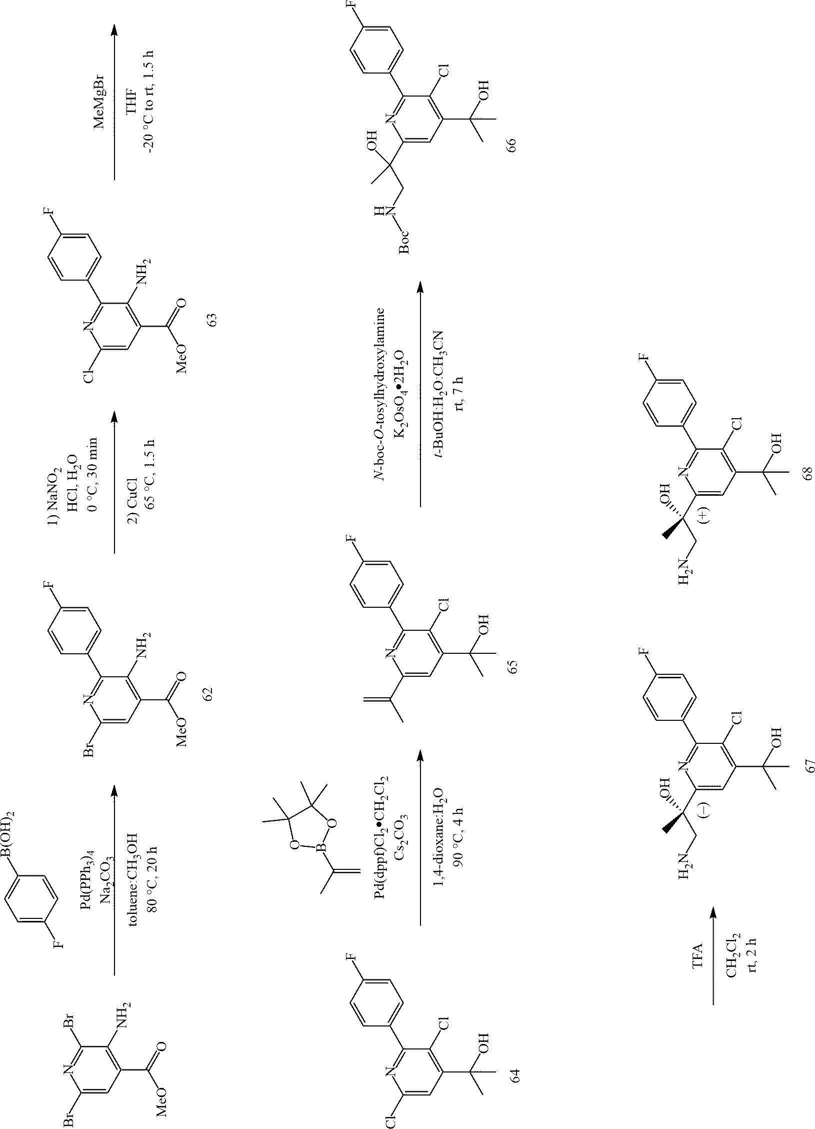

### Methyl 3-amino-6-bromo-2-(4-fluorophenyl)pyridine-4-carboxylate 62

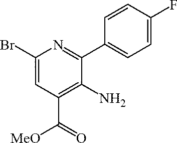 [SMILES: COC(=O)c1cc(Br)nc(-c2ccc(F)cc2)c1N]

To a solution of methyl 3-amino-2,6-dibromopyridine-4-carboxylate (30.1 g, 96.9 mmol) in toluene (250 mL) and CH₃OH (80 mL) under N₂ atmosphere were added 4-fluorophenylboronic acid (12.9 g, 92.3 mmol), Na₂CO₃ (23.7 g, 223 mmol) and Pd(PPh₃)₄ (5.60 g, 4.85 mmol). The reaction mixture was stirred at 80° C. for 20 h in a closed reactor. The catalyst was removed by filtration over Celite®. The filtrate was extracted with EtOAc, washed with water (twice) and brine, and dried (MgSO₄). The solids were removed by filtration and the filtrate was concentrated to dryness under reduced pressure. The crude mixture was combined with other fractions (96.9 mmol and 96.7 mmol). The crude mixture was purified by silica column chromatography (heptane/CH₂Cl₂, 50:50) to afford 62 (58.5 g, 65%).

### Methyl 3,6-dichloro-2-(4-fluorophenyl)pyridine-4-carboxylate 63

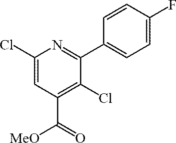 [SMILES: COC(=O)c1cc(Cl)nc(-c2ccc(F)cc2)c1Cl]

To a solution of 62 (14.2 g, 43.7 mmol) in HCl (conc., 60 mL) was added a solution of sodium nitrite (7.54 g, 109 mmol) in H₂O (15 mL) dropwise at 0° C. The mixture was stirred for 30 min and a suspension of CuCl (15.1 g, 153 mmol) in HCl (conc., 30 mL) was added slowly. Following the addition, HCl (6 M aq., 90 mL) was added in portions and the suspension was stirred at 65° C. for 1.5 h. The reaction mixture was cooled to rt and diluted with EtOAc and water. NH₃ in H₂O was added until pH 7-8. The layers were separated, and the aqueous phase was extracted with EtOAc (twice), and dried (MgSOi). The solids were removed by filtration and the filtrate was concentrated to dryness under reduced pressure. The crude mixture was purified by silica column chromatography (heptane/CH₂Cl₂, 1:1) to afford 63 (11.2 g, 86%) as a white solid.

### 2-[3,6-Dichloro-2-(4-fluorophenyl)pyridin-4-yl]propan-2-ol 64

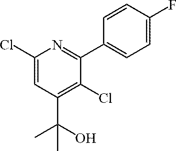 [SMILES: CC(C)(O)c1cc(Cl)nc(-c2ccc(F)cc2)c1Cl]

To a solution of 63 (11.0 g, 36.7 mmol) in THF (230 mL) was added CH₃MgBr (3.4 M in 2-MeTHF, 24.8 mL, 84.4 mmol) dropwise at -20° C. The reaction mixture was warmed to rt and stirred for 1.5 h. Additional amount of CH3MgBr (3.4 M in 2-MeTHF, 11.8 mL, 40.4 mmol) was added at -20° C. The reaction was quenched with cold NH₄Cl (sat., aq.). The layers were separated, and the aqueous phase was extracted with EtOAc, washed with brine, and dried (MgSO₄). The solids were removed by filtration and the filtrate was concentrated to dryness under reduced pressure. The residue was dissolved in a solution of heptane and EtOAc (80:20) and purified by silica column chromatography (heptane/EtOAC, gradient from 100:0 to 80:20) to afford 64 (5.18 g, 47%) as a white solid.

### 2-Chloro-2-(4-fluorophenyl)-6-(prop-1-en-2-yl)pyridin-4-yl)propan-2-ol 65

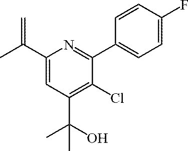 [SMILES: C=C(C)c1cc(C(C)(C)O)c(Cl)c(-c2ccc(F)cc2)n1]

To a solution of 64 (7.97 g, 26.5 mmol) in 1,4-dioxane (120 mL) were added a solution of C_{S2}CO₃ (24.3 g, 74.7 mmol) in water (12 mL), 4,4,5,5-tetramethyl-2-(prop-1-en-2-yl)-1,3,2-dioxaborolane (7.33 g, 43.6 mmol) and PdCl₂(dppf).CH₂Cl₂ (2.26 g, 2.77 mmol) under N₂ atmosphere. The reaction mixture was stirred at 90° C. for 4 h in a closed reactor. The reaction mixture was cooled to rt and mixed with another fraction (1.65 mmol). The mixture was diluted with EtOAc and filtered over Celite®. The filtrate was extracted with EtOAc, washed with water and brine, and dried (MgSO₄). The solids were removed by filtration and the filtrate was concentrated to dryness under reduced pressure. The crude mixture was purified by silica column chromatography (CH₂Cl₂) to afford 65 (7.05 g, 82%) as a yellowish solid.

### Tert-Butyl (2-[5-Chloro-6-(4-Fluorophenyl)-4-(2-Hydroxypropan-2-yl)Pyridin-2-yl]-2-Hydroxypropyl}Carbamate 66

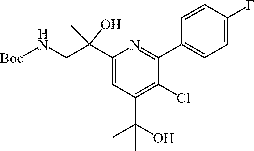 [SMILES: CC(C)(C)OC(=O)NCC(C)(O)c1cc(C(C)(C)O)c(Cl)c(-c2ccc(F)cc2)n1]

To a solution of 65 (4.70 g, 15.4 mmol) and N-boc-O-tosylhydroxylamine (6.63 g, 23.1 mmol) in t-BuOH (65 mL), CH₃CN (28 mL) and water (28.2 mL) was added K₂O_{S}O₄•2H₂O (1.13 g, 3.07 mmol). The reaction mixture was stirred at rt for 7 h. The mixture was combined with another fraction (8.17 mmol) and the mixture was extracted with EtOAc, washed with water (twice) and brine, and dried (MgSO₄). The solids were removed by filtration and the filtrate was concentrated to dryness under reduced pressure. The crude mixture was purified by silica column chromatography (CH₂Cl₂/CH₃OH, gradient from 100:0 to 90:10) to afford 66 (5.87 g, 57%) as an orange oil.

### (-)Amino-2-[5-Chloro-6-(4-Fluorophenyl)-4-(2-Hydroxypropan-2-yl)Pyridin-2-yl]Propan-2-ol 67 and (+)Amino-2-[5-Chloro-6-(4-Fluorophenyl)-4-(2-Hydroxypropan-2-yl)Pyridin-2-yl]propan-2-ol 68

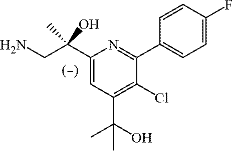

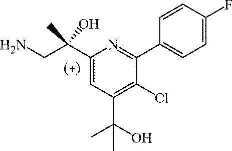

To a solution of 66 (5.80 g, 13.2 mmol) in CH₂Cl₂ (80 mL) was added TFA (10.0 mL, 131 mmol). The reaction mixture was stirred at rt for 2 h and concentrated under reduced pressure. The residue was diluted with CH₂Cl₂ and the solution was washed with NaHCO₃. The product precipitated and was collected by filtration to afford a mixture of enantiomers that were purified by silica column chromatography (CH₂Cl₂/7M NH₃ in CH₃OH) (4.13 g, 92%); ¹H NMR (400 MHz, DMSO-d6) δ ppm 8.13 (s, 1H), 7.58 - 7.68 (m, 2H), 7.27 (m, 2H), 5.56 (m, 1H), 5.56 (s, 1H), 5.20 (s, 1H), 3.09 - 3.27 (m, 2H), 1.63 (s, 6 H), 1.62, 1.36 (s, 3H); LC-MS (method D): Rt = 2.08 min, mass calcd. for C₁₇H₂₀ClN₂O₂ m/z 338.1, found 339.2 [M+H]⁺. The enantiomers were separated by SFC (stationary phase: Daicel Chiralcel-IC 5 mu 300 g, mobile phase: 80% CO₂, 20% CH₃OH + 1% i-PrNH₂) to afford 67 (1.6 g, 41%) [α]_{D}²⁰ -23.47 (c 0.375, DMF); and 68 (1.6 g, 41%) [α]_{D}²⁰ +25.51 (c 0.345, DMF).

### 3_{.}3_{.}2_{.} Synthesis of 77 and 78

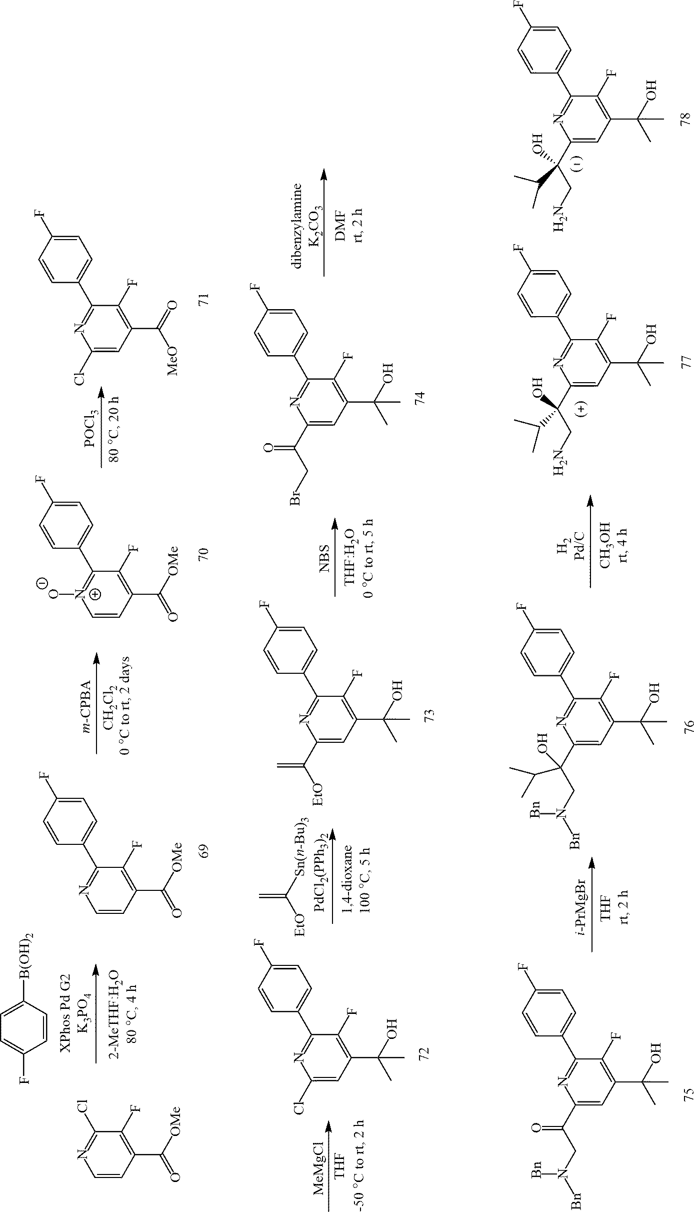

### Methyl 3-fluoro-2-(4-fluorophenyl)isonicotinate 69

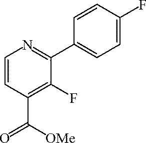 [SMILES: COC(=O)c1ccnc(-c2ccc(F)cc2)c1F]

A mixture of methyl 2-chloro-3-fluoro-4-pyidine carboxylate (23.6 g, 124 mmol), 4-fluoro-phenylboronic acid (34.8 g, 249 mmol) and K₃PO₄ (79.3 g, 373 mmol) in 2-MeTHF (1.4 L) and H₂O (292 mL) was purged with N₂. XPhos Pd G2 (7.70 g, 9.79 mmol) was added and the mixture was purged again with N₂. The reaction mixture was stirred at 80° C. for 4 h. The reaction mixture was diluted with EtOAc and water. The layers were separated, and the organic phase was dried (MgSO₄). The solids were removed by filtration and the solvent of the filtrate was removed under reduced pressure. The crude mixture was purified by silica column chromatography (heptane/EtOAc, gradient from 9:1 to 1:1) to afford 69 (28.5 g, 92%) as an off-white solid.

### 3-Fluoro-2-(4-fluorophenyl)-4-(methoxycarbonyl)pyridine1-oxide 70

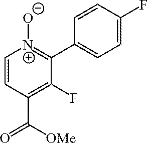 [SMILES: COC(=O)c1cc[n+]([O-])c(-c2ccc(F)cc2)c1F]

To a solution of 69 (4.00 g, 16.1 mmol) in anhydrous CH₂Cl₂ (160 mL) at 0° C. was added m-CPBA (14.8 g, 64.2 mmol, 75% pure). The mixture was warmed to rt and stirred for 2 days. The reaction was quenched with NaOH (1N, aq.). The organic phase was successively washed with NH₄Cl (sat., aq.), Na₂S₂O₃ (10%, aq.), water and brine, and dried (MgSO₄). The solids were removed by filtration and the filtrate was concentrated to dryness under reduced pressure. The crude mixture was purified by silica column chromatography (CH₂Cl₂/CH₃OH, gradient from 100:0 to 90:10) to afford 70 (3.21 g, 75%) as a pale-yellow solid.

### Methyl 6-chloro-3-fluoro-2-(4-fluorophenyl)pyridine-4-carboxylate 71

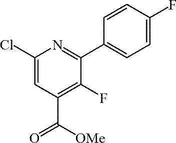 [SMILES: COC(=O)c1cc(Cl)nc(-c2ccc(F)cc2)c1F]

In a sealed tube, a solution of 70 (3.21 g, 12.1 mmol) in POCl₃ (51 mL) was stirred at 80° C. for 20 h. The reaction mixture was concentrated under reduced pressure. The brown residue was taken up in water and EtOAc and the mixture was basified with K₂CO₃ powder. The layers were separated and the aqueous phase was extracted with EtOAc (twice). The combined organic layers were dried (MgSO₄). The solids were removed by filtration and the filtrate was concentrated to dryness under reduced pressure to afford 71 (3.27 g, 95%) as a brown solid.

### 2-Chloro-3-fluoro-2-(4-fluorophenyl)pyridin-4-yl]propan-2-ol 72

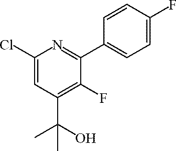 [SMILES: CC(C)(O)c1cc(Cl)nc(-c2ccc(F)cc2)c1F]

The reaction was performed on 3 batches of 38.5 g. To a solution of 71 (38.5 g, 136 mmol) in THF (550 mL) was added CH₃MgBr (3.4 M in 2-MeTHF, 100 mL, 340 mmol) dropwise at -50° C. The mixture was stirred at rt for 2 hours, then cooled to -50° C. and treated with a NH₄Cl solution. The mixture was diluted with water and extracted with EtOAc (twice). The combined organic layers were dried (MgSO₄). The solids were removed by filtration and the filtrate was concentrated to dryness under reduced pressure. The crude mixture was triturated in water. The solid was collected by filtration and dried at 50-60° C. to afford 72 (113.3 g, 99%) as a white solid.

### 2-(1-Ethoxyethenyl)-3-fluoro-2-(4-fluorophenyl)pyridin-4₋yl]propan-2-ol 73

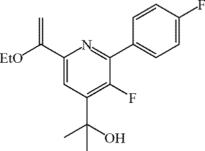 [SMILES: C=C(OCC)c1cc(C(C)(C)O)c(F)c(-c2ccc(F)cc2)n1]

A microwave vial equipped with a magnetic stir bar was charged with tributyl(1-ethoxyvinyl)tin (6.75 mL, 19.4 mmol) and 72 (5.00 g, 17.6 mmol) in 1,4-dioxane (15 mL). PdCl₂(PPh₃)₂ (1.24 g, 1.76 mmol) was added and the reaction mixture was stirred at 100° C. for 6 h. The reaction mixture was filtered through Celite® and washed with 1,4-dioxane (3 times). The filtrate was concentrated under reduced pressure. The residue was purified by silica column chromatography (heptane/EtOAc, gradient from 100:0 to 0:100) to afford 73 (1.68 g, 30%).

### 2-Bromo[5-fluoro-6-(4-fluorophenyl)-4-(2-hydroxypropan-2-yl)pyridin-2-yl]ethanone 74

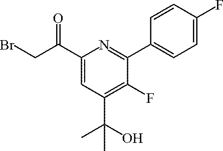 [SMILES: CC(C)(O)c1cc(C(=O)CBr)nc(-c2ccc(F)cc2)c1F]

To a solution of 73 (1.68 g, 5.26 mmol) in THF (14 mL) and water (4 mL) at 0° C. was added NBS (0.94 g, 5.26 mmol). The reaction mixture was warmed to rt and stirred for 5 h. The mixture was diluted with water and the aqueous phase was extracted with EtOAc (3 x 200 mL). The combined organic extracts were washed with NaHCO₃ (sat., aq.) and dried (MgSO₄). The solids were removed by filtration and the filtrate was concentrated to dryness under reduced pressure to afford 74 that was used in the next step without further purification.

### 2-(Dibenzylamino)-1-[5-fluoro-6-(4-fluorophenyl)-4-(2-hydroxypropan-2-yl)pyridin-2-y1]ethan-1-one 75

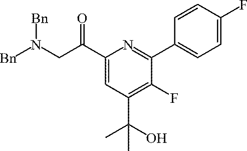 [SMILES: CC(C)(O)c1cc(C(=O)CN(Cc2ccccc2)Cc2ccccc2)nc(-c2ccc(F)cc2)c1F]

74 (11.0 g, 29.7 mmol) was added to a suspension of K₂CO₃ (4.52 g, 32.7 mmol) and dibenzylamine (6 mL, 31 mmol) in DMF (40 mL). The reaction mixture was stirred at rt for 2 h. The mixture was diluted with water (600 mL) and the aqueous phase was extracted with EtOAc (4 x 200 mL). The combined organic extracts were dried (MgSO₄). The solids were removed by filtration and the filtrate was passed through packed alumina. The solvent of the filtrate was removed under reduced pressure. The crude mixture was purified by silica column chromatography (heptane/EtOAc) to afford 75 (13.8 g, 95%).

### 1-(Dibenzylamino)-2-[5-fluoro-6-(4-fluorophenyl)-4-(2-hydroxypropan-2-yl)pyridin-2-yl]-3-methylbutan-2-ol 76

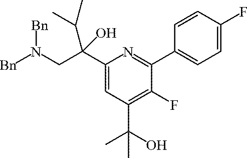 [SMILES: CC(C)C(O)(CN(Cc1ccccc1)Cc1ccccc1)c1cc(C(C)(C)O)c(F)c(-c2ccc(F)cc2)n1]

To a solution of 75 (6.68 g, 13.7 mmol) in anhydrous THF (125 mL) at 0° C. was added i-PrMgBr (2.9 M in 2-MeTHF, 23.7 mL, 68.6 mmol). The reaction mixture was stirred at rt for 2 h. CH₃OH (40 mL) and HCl (4 M, aq., 6 mL) were added and the mixture was stirred for 30 min. The solvent was removed under reduced pressure. NaHCO₃ (sat., aq.) was added and the aqueous phase was extracted with EtOAc (3 times). The combined organic extracts were dried (MgSO₄). The solids were removed by filtration and the filtrate was concentrated to dryness under reduced pressure. The crude mixture was purified by silica column chromatography (heptane/EtOAc, gradient from 100:0 to 0:100). Heptane was added to the residue and a precipitated was formed. The precipitate was collected by filtration to give a first crop of 76 (390 mg, 5%). The filtrate was purified by silica column chromatography (heptane/EtOAc, gradient from 100:0 to 40:60) to afford a second crop of 76 (3.41 g, 43%, 91% pure).

### (+)Ammo-2-[5-Fluoro-6-(4-Fluorophenyl)-4-(2-Hydroxypropan-2-yl)Pyridin-2-yl]-3-Methylbutan-2-ol 77 and (-)Amino-2-[5-Fluoro-6-(4-Fluorophenyl)-4-(2-Hydroxypropan-2-yl)Pyridin-2-yl]-3-Methylbutan-2-ol 78

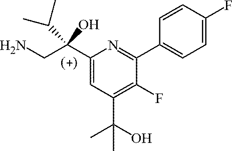

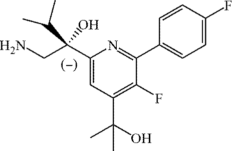

A solution of 76 (4.2 g, 7.92 mmol) in CH₃OH (150 mL) was purged with N₂ and Pd/C (10%, 842 mg) was added. The flask was sealed and exposed to H₂ atmosphere. The reaction mixture was stirred at rt for 4 h. The mixture was filtered through packed Celite® and the solvent was removed under reduced pressure. ¹H NMR (400 MHz, DMSO-d6) δ ppm 7.90 - 7.97 (m, 2 H), 7.87 (d, J=5.7 Hz, 1 H), 7.29 - 7.37 (m, 2 H), 5.54 (s, 1 H), 4.72 - 5.14 (m, 1 H), 3.16 (d, J=12.8 Hz, 2 H), 2.83 (d, J=12.8 Hz, 1 H), 2.15 (spt, J=6.8 Hz, 1 H), 1.54 (s, 6 H), 0.87 (d, J=6.8 Hz, 3 H), 0.67 (d, J=6.8 Hz, 3 H). The enantiomers were separated by SFC (stationary phase: Chiralpak Diacel AD 20 x 250 mm, mobile phase: CO₂, i-PrOH + 0.4% i-PrNH₂) to give 77 (1.0 g, 36%); LC-MS (method C): Rt = 1.77 min; mass calcd. for C₁₉H₂₄F₂N₂O₂ 350.2, m/z found 373.2 [M+CH3CO2]⁻; and 78 (1.0 g, 36%); LC-MS (method C): Rt = 1.77 min; mass calcd. for C₁₉H₂₄F₂N₂O₂ 350.2, m/z found 351.3 [M+H]⁺; [α]_{D}²⁰ -13.86 (c 0.38, DMF).

### 3.3.3. Synthesis of 81 and 82

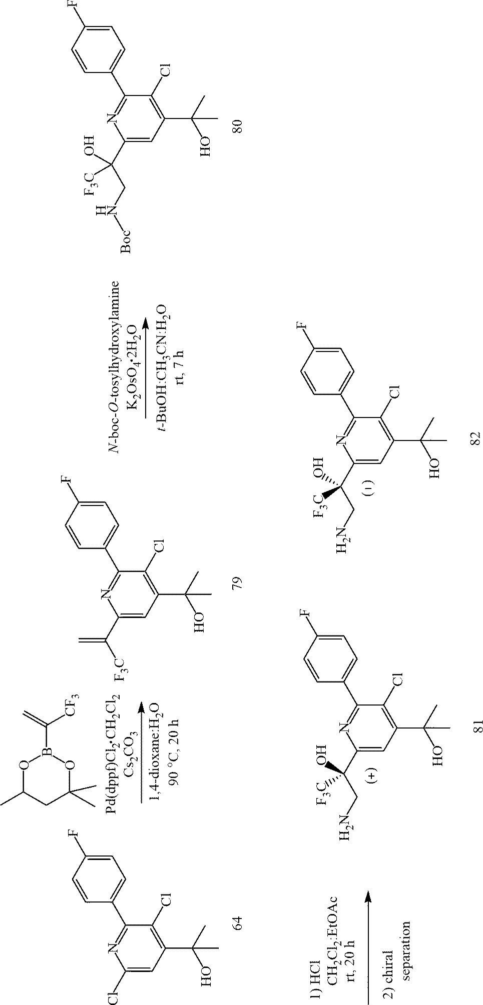

### 2-Chloro-2-(4-Fluorophenyl)-6-(3,3,3-Trifluoroprop-1-en-2-yl)Pyridin-4-yl]Propan-2-ol 79

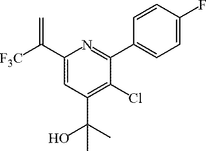 [SMILES: C=C(c1cc(C(C)(C)O)c(Cl)c(-c2ccc(F)cc2)n1)C(F)(F)F]

To a solution of 64 (80.0 g, 237 mmol) in 1,4-dioxane (1.2 L) and H₂O (120 mL) were added 4,4,6-trimethyl-2-(3,3,3-trifluoroprop-1-en-2-yl)-1,3,2-dioxaborinane (158 g, 712 mmol), C_{S2}CO₃ (216 g, 664 mmol) and Pd(dppf)Cl₂.CH₂Cl₂ (17.8 g, 21.8 mmol). The reaction mixture was degassed 3 times and stirred at 90° C. for 20 h. The reaction mixture was concentrated under reduced pressure about ⅓ of volume. The mixture was diluted with EtOAc (500 mL) and filtered over Celite®. The filtrate was extracted with EtOAc (1 L). The organic layer was washed with water (1.5 L) and brine (300 mL), and dried (Na₂SO₄). The solids were removed by filtration and the filtrate was concentrated to dryness under reduced pressure. The residue was purified by silica column chromatography (petroleum ether/EtOAc, gradient from 20:1 to 15:1) to afford 79 (55.3 g, 64%).

### Tert-Butyl {2-[5-Chloro-6-(4-Fluorophenyl)-4-(2-Hydroxypropan-2-yl)Pyridin-2-yl]-3,3,3-Trifluoro-2-Hydroxypropyl}Carbamate 80

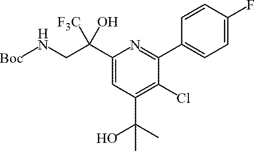 [SMILES: CC(C)(C)OC(=O)NCC(O)(c1cc(C(C)(C)O)c(Cl)c(-c2ccc(F)cc2)n1)C(F)(F)F]

To a mixture of 79 (55.3 g, 154 mmol) and A-boc-O-tosylhydroxylamine (66.2 g, 231 mmol) in t-BuOH (750 mL), CH₃CN (250 mL) and H₂O (250 mL) was added K₂O_{S}O₄•2H₂O (11.3 g, 30.7 mmol). The reaction mixture was stirred at rt for 7 h. The layers were separated, and the aqueous phase was extracted with EtOAc (1.5 L). The combined organic extracts were washed with water (1 L) and brine (300 mL), and dried (Na₂SO₄). The solids were removed by filtration and the filtrate was concentrated to dryness under reduced pressure. The residue was purified by silica column chromatography (petroleum ether/CH₂Cl₂, gradient from 1:1 to 0:1, then petroleum ether/EtOAc 5:1) to afford 80 (49.5 g, 64%).

### (+)Amino-2-[5-chloro-6-(4-fluorophenyl)-4-(2-hydroxypropan-2-yl)pyridin-2-yl]-1,1,1-trifluoropropan-2-ol 81 and (-)amino-2-[5-chloro-6-(4-fluorophenyl)-4-(2-hydroxypropan-2-yl)pyridin-2-yl]-1,1,1-trifluoropropan-2-ol 82

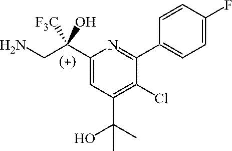

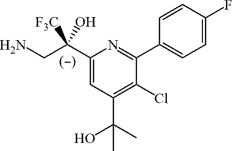

To a solution of 80 (29.0 g, 58.8 mmol) in CH₂Cl₂ (100 mL) was added HCl (4 M in EtOAc, 176.5 mL). The white suspension was stirred at rt for 20 h. The reaction mixture was diluted with petroleum ether (200 mL). The white solid was collected by filtration to give a mixture of enantiomers as a HCl salt (23 g, 91%) as a white solid. ¹H NMR (400 MHz, DMSO-d6) δ ppm 8.25 (s, 1H), 7.62 - 7.70 (m, 2H), 7.27 - 7.35 (m, 2H), 6.25 - 7.12 (m, 1H), 5.66 (br s, 1H), 3.41 (br d, J=13.4 Hz, 1H), 3.36 - 3.49 (m, 1H), 3.09 (d, J=13.4 Hz, 1H), 1.65 (d, J=5.3 Hz, 6H), 1.14 - 1.58 (m, 1H); LC-MS (method D): Rt = 2.31 min; mass calcd. for C₁₇H₁₇ClF₄N₂O₂ 392.0, m/z found 393.0 [M+H]⁺. The enantiomers were separated by SFC (stationary phase: Daicel chiralpak IB, 20 µm, 1000 gr, mobile phase: heptane/i-PrOH (+1% i-PrNH₂), 85:15) to afford 81 and 82 (25 g, 40%); [a]_{D}²² -40.96 (c 0.415, DMF).

### 3.3.4. Synthesis of 90 and 91

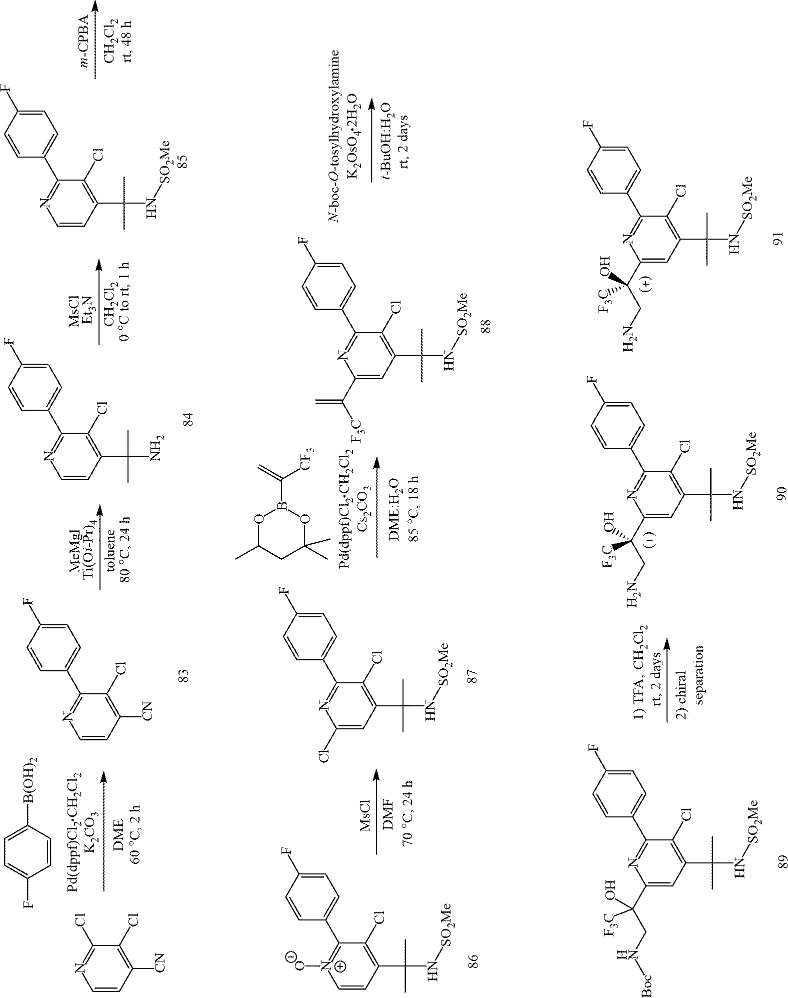

### 3-Chloro-2-(4-fluorophenyl)pyridine-4-carbonitrile 83

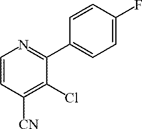 [SMILES: N#Cc1ccnc(-c2ccc(F)cc2)c1Cl]

A mixture of 2,3-dichloropyridine-4-carbonitrile (1.33 g, 7.69 mmol), 4-fluorophenylboronic acid (1.08 g, 7.69 mmol) and K₂CO₃ (2 M in H₂O, 7.69 mL, 15.4 mmol) in DME (25 mL) was purged with N₂. Pd(dppf)Cl₂.CH₂Cl₂ (0.63 g, 0.77 mmol) was added and the mixture was purged again with N₂. The reaction mixture was stirred at 60° C. for 2 h. The reaction mixture was cooled to rt and filtered over Celite® and washed with EtOAc. The filtrate was diluted with EtOAc and brine. The layers were separated, and the aqueous phase was extracted with EtOAc (twice). The combined organic extracts were dried (MgSO₄). The solids were removed by filtration and the filtrate was concentrated under reduced pressure. The crude mixture was purified by silica column chromatography (heptane/EtOAc, gradient from 90: 10 to 70:30) to afford 83 (1.43 g, 72%, 90% pure) as a white solid.

### 2-Chloro-2-(4-fluorophenyl)pyridine-4-yl]propan-2-amine 84

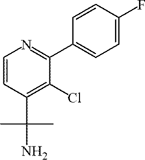 [SMILES: CC(C)(N)c1ccnc(-c2ccc(F)cc2)c1Cl]

CH₃MgBr (3 M in Et₂O, 5.53 mL, 16.6 mmol) was added to a solution of 83 (1.43 g, 5.53 mmol, 90% pure) in toluene (45 mL). After stirring for 5 min, Ti(Oi-Pr)₄ (1.65 mL, 5.59 mmol) was added and the reaction mixture was stirred at 80° C. for 2 h and at rt for 18 h. The reaction was quenched with Na₂CO₃ (sat., aq.). The mixture was stirred for 30 min, filtered over Celite® and washed with EtOAc. The layers were separated, and the aqueous phase was extracted with EtOAc. The combined organic extracts were dried (MgSO₄). The solids were removed by filtration and the filtrate was concentrated under reduced pressure. The crude mixture was purified by silica column chromatography (CH₂Cl₂/CH₃OH, gradient from 100:0 to 98:2) to afford 84 (820 mg, 56%).

### N-{2-chloro-2-(4-fluorophenyl)pyridin-4-yl]propan-2-yl}methanesulfonamide 85

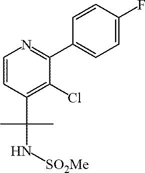 [SMILES: CC(C)(NS(C)(=O)=O)c1ccnc(-c2ccc(F)cc2)c1Cl]

To a solution of 84 (0.82 g, 310 mmol) and Et₃N (0.86 mL, 6.20 mmol) in CH₂Cl₂ (55 mL) at 0° C. was added methanesulfonyl chloride (0.31 mL, 4.03 mmol) dropwise (the internal temperature of the reaction mixture was maintained between 0 and 3° C.). The reaction mixture was warmed to rt and stirred for 1 h. The reaction mixture was diluted with water and CH₂Cl₂. The layers were separated, and the organic phase was dried (MgSO₄). The solids were removed by filtration and the filtrate was concentrated under reduced pressure. The crude mixture was purified by silica column chromatography (heptane/EtOAc, gradient from 90: 10 to 40:60) to afford 85 (895 mg, 84%).

### N-{2-chloro-2-(4-fluorophenyl)-1-oxo-1λ⁵-pyridin-4-yl]propan-2-yl}methanesulfonamide 86

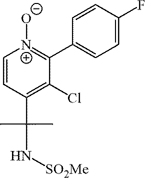 [SMILES: CC(C)(NS(C)(=O)=O)c1cc[n+]([O-])c(-c2ccc(F)cc2)c1Cl]

At 0° C., to a solution of 85 (13.00 g, 36.1 mmol, 95% pure) in CH₂Cl₂ (250 mL) was added m-CPBA (24.9 g, 108 mmol, 75% pure) portionwise. The reaction mixture was stirred at rt for 2 days. The reaction was quenched by the addition of NaHCO₃ (sat., aq.). The layers were separated, and the aqueous phase was extracted with CH₂Cl₂ (4 times). The combined organic extracts were dried (MgSO₄). The solids were removed by filtration and the filtrate was concentrated under reduced pressure to afford 86 (18.5 g, 86%, 60% pure).

### N- {2-[3.6-Dichloro-2-(4-Fluorophenyl)Pyridin-4-yl]Propan-2-yl }Methanesulfonamide 87

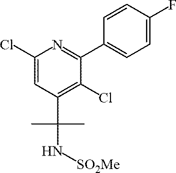 [SMILES: CC(C)(NS(C)(=O)=O)c1cc(Cl)nc(-c2ccc(F)cc2)c1Cl]

Methanesulfonyl chloride (23.9 mL, 309 mmol) was added to a solution of 86 (18.5 g, 30.9 mmol, 60% pure) in CH₂Cl₂ (110 mL) at rt then heated to 70° C. for 1 day. The reaction mixture was cooled to rt and poured into NaHC03 (sat., aq.). The mixture was diluted with EtOAc. The layers were separated, and the aqueous phase was extracted with EtOAc (twice). The combined organic extracts were dried (MgSO₄). The solids were removed by filtration and the filtrate was concentrated under reduced pressure. The crude mixture was purified by silica column chromatography (CH₂Cl₂/CH₃OH, gradient from 100:0 to 99:1) to afford 87 (11.3 g, 68%, 70% pure).

### N-{2-Chloro-2-(4-Fluorophenyl)-6-(3,3,3-Trifluoroprop-1-en-2-yl)Pyridin-4-yl]Propan-2-yl}Methanesulfonamide 88

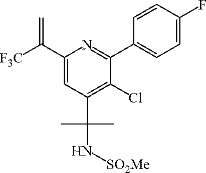 [SMILES: C=C(c1cc(C(C)(C)NS(C)(=O)=O)c(Cl)c(-c2ccc(F)cc2)n1)C(F)(F)F]

In a Schlenk tube, a mixture of 87 (6.30 g, 11.7 mmol, 70% pure), 4,4,6-trimethyl-2-(3,3,3-trifluoroprop-1-en-2-yl)-1,3,2-dioxaborinane (4.85 mL, 23.4 mmol) and C_{S2}CO₃ (11.5 g, 35.2 mmol) in H₂O (15 mL) and DME (95 mL) was purged with N₂. Pd(dppf)Cl₂.CH₂Cl₂ (1.91 g, 2.34 mmol) was added and the mixture was purged again with N₂. The reaction mixture was stirred at 85° C. for 18 h. The reaction mixture was cooled to rt and combined with another fraction (9.28 mmol). The mixture was filtered over a pad of Celite® and washed with EtOAc and the filtrate was diluted with brine. The layers were separated, and the aqueous phase was extracted with EtOAc (twice). The combined organic extracts were dried (MgSO₄). The solids were removed by filtration and the filtrate was concentrated under reduced pressure. The crude mixture was purified by silica column chromatography (heptane/EtOAc, 90:10) to afford 88 (14 g, quant., 65% pure).

### Tert-Butyl12-[5-Chloro-6-(4-Fluorophenyl)-4-{2-[(Methanesulfonyl)Amino1propan-2-yl}Pyridine-2-y 11-3,3,3-Trifluoro-2-Hydroxypropyl}Carbamate 89

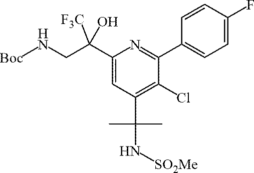 [SMILES: CC(C)(C)OC(=O)NCC(O)(c1cc(C(C)(C)NS(C)(=O)=O)c(Cl)c(-c2ccc(F)cc2)n1)C(F)(F)F]

K₂O_{S}O₄.2H₂O (0.88 g, 2.38 mmol) and A-boc-O-tosylhydroxylamine (5.47 g, 19.0 mmol) were added to a solution of 88 (8.00 g, 11.9 mmol, 65% pure) in t-BuOH (120 mL) and H₂O (8 mL). The reaction mixture was stirred at rt for 2 days. The reaction mixture was combined with another fraction (7.44 mmol) and diluted with water, brine and EtOAc. The layers were separated and the aqueous phase was extracted with EtOAc (3 times). The combined organic extracts were dried (MgSO₄). The solids were removed by filtration and the filtrate was concentrated under reduced pressure to afford 89 (21 g, 76%, 40% pure).

### N-{6-[(-)-3-Amino-1,1,1-Trifluoro-2-Hydroxypropan-2-y1]-3-Chloro-2-(4-Fluorophenyl)Pyridin-4-yl}Propan-2-yl)Methanesulfonamide90 and N-{6-r(+)-3-Amino-1,1,1-Trifluoro-2-Hydroxypropan-2-yl}-3-Chloro-2-(4-Fluorophenyl)Pyridin-4-yl}Propan-2-yl)Methanesulfonamide91

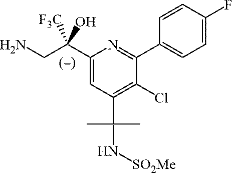

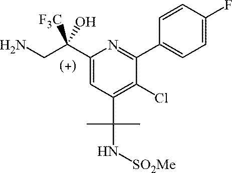

TFA (19.1 mL, 249 mmol) was added to a solution of 89 (22.8 g, 16.0 mmol, 40% pure) in CH₂C1₂ (239 mL). The reaction mixture was stirred at rt for 2 days. The reaction mixture was poured into NaHCO₃ (sat., aq.). The layers were separated, and the aqueous phase was extracted with CH₂C1₂ (3 times). The combined organic extracts were dried (MgSO₄). The solids were removed by filtration and the filtrate was concentrated under reduced pressure. The crude mixture was purified by silica column chromatography (CHZC12/CH30H, gradient from 100:0 to 95:5) to afford a mixture of enantiomers (2.4 g, 32%). The enantiomers were separated by SFC (stationary phase: CHIRALPAK AD-H 5 µm 250 x 30 mm, mobile phase: 72% CO₂, 28% i-PrOH (0.3% i-PrNH₂)) to give 90 (936 mg, 14%) and 91 (990 mg, 14%). The enantiomers were independently co-evaporated with toluene (3 times) to give 90 (920 mg, 13%); ¹H NMR (400 MHz, DMSO-d₆) δ ppm 1.81 (d, J=4.6 Hz, 6H), 2.89 (s, 3H), 3.09 (d, J=13.4 Hz, 1H), 3.37 (br d, J=13.6 Hz, 1H), 7.11 - 7.28 (m, 3H), 7.32 (t, J=8.9 Hz, 2H), 7.62 - 7.69 (m, 2H), 7.76 (s, 1H), 7.90 (s, 1H); LC-MS (method J): Rt = 1.29 min; mass calcd. for C₁₈H₂₀C1F₄N₃O₃S 469.1, m/z found 470.5 [M+H]⁺; and 91 (980 mg, 14%); ¹H NMR (400 MHz, DMSO-d₆) δ ppm 1.81 (d, J=4.6 Hz, 6H), 2.89 (s, 3H), 3.09 (d, J=13.4 Hz, 1H), 3.38 (br d, J=13.6 Hz, 1H), 7.12 - 7.28 (m, 1H), 7.29 - 7.36 (m, 2H), 7.61 - 7.68 (m, 2H), 7.76 (s, 1H), 7.90 (s, 1H);LC-MS (method J): Rt = 1.8 min; mass calcd. for C₁₈H₂₀C1F₄N₃O₃S 469.1, m/z found 470.5 [M+H]⁺.

### 3.3.5. Synthesis of 94 and 95

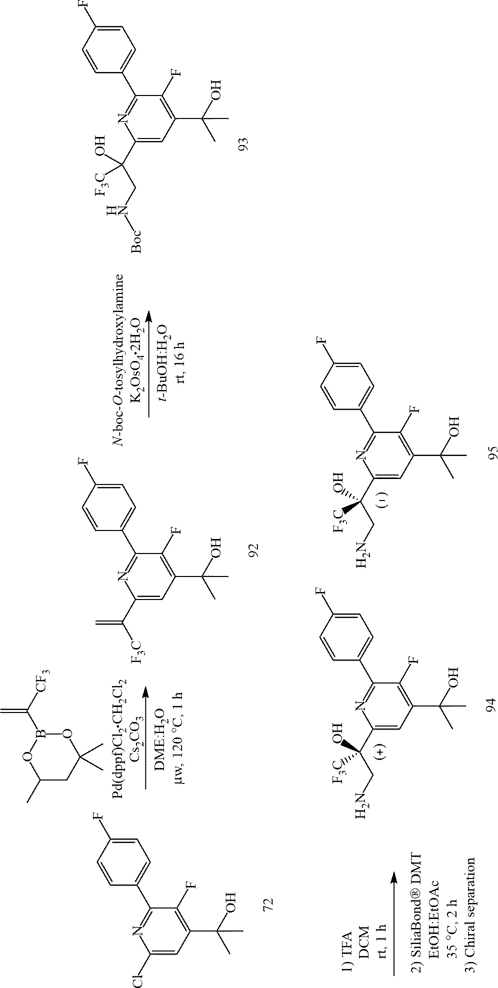

### 2-f3-Fluoro-2-(4-Fluorophenyl)-6-(3,3,3-Trifluoroprop-1-en-2-yl)Pyridin-4-yl]Propan-2-ol 92

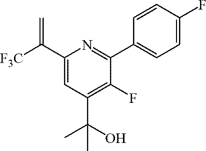 [SMILES: C=C(c1cc(C(C)(C)O)c(F)c(-c2ccc(F)cc2)n1)C(F)(F)F]

The reaction was performed on 2 batches of 1.36 g of intermediate 72. In a sealed tube, a mixture of 72 (1.36 g, 4.79 mmol), 4,4,6-trimethyl-2-(3,3,3-trifluoroprop-1-en-2-yl)-1,3,2-dioxaborinane (1.50 mL, 7.19 mmol) and C_{S2}CO₃ (4.70 mg, 14.4 mmol) in H₂O (2 mL) and DME (11 mL) was purged with N₂. Pd(dppf)Cl₂.CH₂Cl₂ (786 mg, 963 _{µ}mol) was added and the mixture was purged again with N₂. The reaction mixture was heated at 120° C. in the microwave with a power output ranging from 0 to 400 W for 1 h. The two batches were combined. The reaction mixture was diluted with EtOAc and water and the layers were separated. The aqueous phase was extracted with EtOAc (twice). The combined organic extracts were washed with brine, and dried (MgSO₄). The solids were removed by filtration and the filtrate was concentrated under reduced pressure. The crude mixture was purified by silica column chromatography (heptane/EtOAc, gradient from 1:0 to 7:3) to give 92 (2.15 g, 65%) as a yellow oil.

### T-Butyl {3,3,3-Trifluoro-2-f5-Fluoro-6-(4-Fluorophenyl)-4-(2-Hydroxypropan-2-yl)Pyridin-2-y11-2-Hydroxypropyl}Carbamate 93

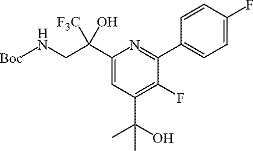 [SMILES: CC(C)(C)OC(=O)NCC(O)(c1cc(C(C)(C)O)c(F)c(-c2ccc(F)cc2)n1)C(F)(F)F]

K₂O_{S}O₄.2H₂O (8.94 g, 24.3 mmol) and A-boc-O-tosylhydroxylamine (76.7 g, 267 mmol) were added to a solution of 92 (41.6 g, 121 mmol) in t-BuOH (1.37 L) and H₂O (88 mL). The reaction mixture was stirred at rt for 16 h. The reaction mixture was diluted with water and EtOAc. The layers were separated and the aqueous phase was extracted with EtOAc (3 times). The combined organic layers were washed with NaHCO₃ (sat., aq.), and dried (MgSO₄). The solids were removed by filtration and the filtrate was concentrated under reduced pressure. The residue was purified by silica column chromatography (heptane/EtOAc, gradient from 95:5 to 7:3) to give 93 (41.7 g, 72%) as a pale yellow solid.

### (+)Amino-1,1,1,-trifluoro-2-[5-fluoro-6-(4-fluorophenyl)-4-(2-hydroxypropan-2-yl)pyridin-2-yl]propan-2-ol 94 and (-)amino-1,1,1,-trifluoro-2-[5-fluoro-6-(4-fluorophenyl)-4-(2-hydroxypropan-2-yl)pyridin-2-yl1propan-2-ol 95

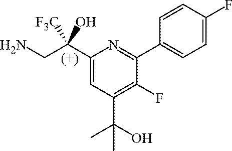

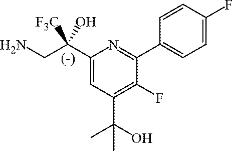

TFA (400 mL) was added to a solution of 93 (41.7 g, 87.5 mmol) in CH₂C1₂ (650 mL) and the reaction mixture was stirred at rt for 1 h. The solvent was removed under reduced pressure. The residue was diluted with EtOAc and poured into NaHCO₃ (sat., aq.). The layers were separated, and the aqueous layer was extracted with EtOAc (twice). The combined organic extracts were washed with brine, and dried (MgSOr). The solids were removed by filtration and the filtrate was concentrated under reduced pressure. The crude mixture was purified by silica column chromatography (CH₂Cl₂/(CH₃OH/aq.NH₃, 95:5), gradient from 98:2 to 95:5) to afford a mixture of enantiomers (22.9 g) as a white solid. The enantiomers were separated by chiral SFC (stationary phase: CHIRALPAK IC 5 µm 250 x 30 mm, mobile phase: 84% CO₂, 16% i-PrOH (0.3% i-PrNH₂)) to give 94 (11.05 g) and 95 (11.09 g) both as white solids. Intermediates 94 and 95 were separately re-purified by silica column chromatography (CH2Cl2/(7 M NH₃ in CH₃OH, 95:5), gradient from 100:0 to 95:5) to give 94 (9.79 g, 30%); ¹H NMR (400 MHz, DMSO-d₆) δ ppm 8.03 (d, J=5.6 Hz, 1H), 7.94 (dd, J=7.6, 5.6 Hz, 2H), 7.36 (t, J=8.8 Hz, 2H), 6.70 (br s, 1H), 5.66 (br s, 1H), 3.47 (d, J=13.1 Hz, 1H), 3.13 (d, J=13.6 Hz, 1H), 1.55 (s, 3H) 1.54 (s, 3H), 1.25 - 1.49 (m, 2H); LC-MS (method J): Rt = 1.35 min; mass calcd. for C₁₇H₁₇F₅N₂O₂ 376.1, m/z found 377.3 [M+H]⁺; and 95 (10.3 g, 31%); ¹H NMR (400 MHz, DMSO-d₆) δ ppm 8.03 (d, J=5.6 Hz, 1H), 7.94 (dd, J=7.1, 6.1 Hz, 2 H), 7.36 (t, J=8.8 Hz, 2H), 6.71 (br s, 1H), 5.66 (br s, 1H), 3.47 (br d, J=13.1 Hz, 1H), 3.13 (d, J=13.6 Hz, 1H), 1.55 (s, 3H), 1.54 (s, 3H), 1.25 - 1.50 (m, 2H); LC-MS (method K): Rt = 1.27 min; mass calcd. for C₁₇H₁₇F₅N₂O₂ 376.1, m/z found 377.2 [M+H]⁺ as white solids.

### 3.3.6. Synthesis of 99

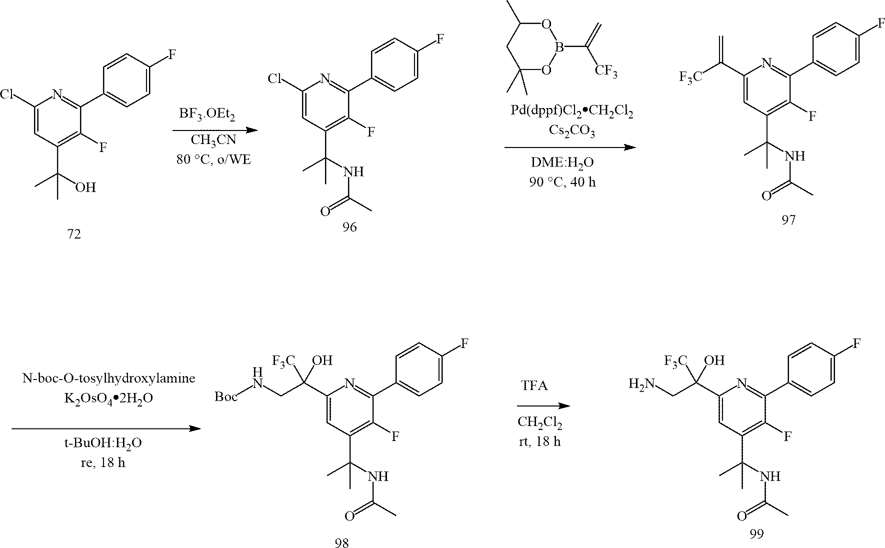

### N-{2-chloro-3-fluoro-2-(4-fluorophenyl)pyridin-4-yl]propan-2-yl}acetamide 96

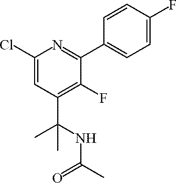 [SMILES: CC(=O)NC(C)(C)c1cc(Cl)nc(-c2ccc(F)cc2)c1F]

To a mixture of 72 (2.00 g, 6.27 mmol, 89% pure) in CH₃CN (66 mL) was slowly added BF₃•OEt₂ (2.45 mL, 19.4 mmol) while keeping the internal temperature at 20° C. The reaction mixture was stirred at 80° C. over the weekend. The reaction mixture was diluted with EtOAc and washed with NaHC03 (sat., aq., twice). The combined aqueous layers were extracted with EtOAc. The combined organic extracts were dried (MgSO₄). The solids were removed by filtration and the filtrate was concentrated under reduced pressure. The crude mixture was purified by silica column chromatography (heptane/EtOAc, gradient from 95:5 to 50:50) to afford 96 (949 mg, 47%) as a an off-white solid.

### N-{2-fluoro-2-(4-fluorophenyl)-6-(3,3,3-trifluoroprop-4-yl]propan-2-yl}acetamide 97

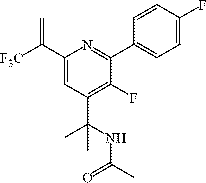 [SMILES: C=C(c1cc(C(C)(C)NC(C)=O)c(F)c(-c2ccc(F)cc2)n1)C(F)(F)F]

In a sealed tube, a mixture of 96 (949 mg, 2.92 mmol), 4,4,6-trimethyl-2-(3,3,3-trifluoroprop-1-en-2-yl)-1,3,2-dioxaborinane (1.2 mL, 5.84 mmol) and C_{S2}CO₃ (2.86 g, 8.77 mmol) in water (1.2 mL) and DME (31 mL) was purged with N₂. Pd(dppf)Cl₂.CH₂Cl₂ (477 mg, 584 µmol) was added and the mixture was purged again with N₂. The reaction mixture was stirred at 90° C. for 20 h. Additional amount of Pd(dppf)Cl₂.CH₂Cl₂ (239 mg, 292 µmol), 4,4,6-trimethyl-2-(3,3,3-trifluoroprop-1-en-2-yl)-1,3,2-dioxaborinane (606 µL, 2.92 mmol) and C_{S2}CO₃ (952 mg, 2.92 mmol) were added. The mixture was purged with N₂ and the reaction mixture was stirred at 90° C. for another 20 h. The reaction mixture was cooled to rt and diluted with EtOAc and water. The layers were separated, and the aqueous phase was extracted with EtOAc. The combined organic extracts were dried (MgSO₄). The solids were removed by filtration and the filtrate was concentrated under reduced pressure. The crude mixture was combined with another fraction (1.59 mmol). The crude mixture was purified by silica column chromatography (heptane/(EtOAc / CH₃OH, 9:1), gradient from 70:30 to 50:50) to afford 97 (1.67 g, 45%).

### Tert-Butyl-{2-[4-(2-Acetamidopropan-2-yl)-5-Fluoro-6-(4-Fluorophenyl)Pyridin-2-yl]-3,3,3-Trifluoro-2-Hydroxypropyl}Carbamate 98

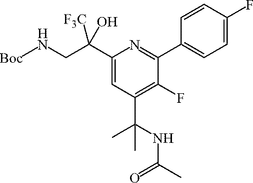 [SMILES: CC(=O)NC(C)(C)c1cc(C(O)(CNC(=O)OC(C)(C)C)C(F)(F)F)nc(-c2ccc(F)cc2)c1F]

K₂O_{S}O₄•2H₂O (320 mg, 0.87 mmol) then N-boc-0-tosylhydroxylamine (1.62 g, 5.65 mmol) were added to a solution of 97 (1.67 g, 4.35 mmol) in t-BuOH (47 mL) and water (3 mL). The reaction mixture was stirred at rt for 18 h. NaHCO₃ (10% aq.) and EtOAc were added. The aqueous layer was separated and extracted with EtOAc. The combined organic extracts were dried (MgSO₄). The solids were removed by filtration and the filtrate was concentrated under reduced pressure. The crude mixture was purified by silica column chromatography (CH₂Cl₂/CH₃OH, gradient from 100:0 to 98:2) to afford 98 (1.10 g, 49%) as a pale brown solid.

### N-{6-(3-Amino-1,1,1-Trifluoro-2-Hydroxypropan-2-yl)-3-Fluoro-2-(4-Fluorophenyl)Pyridin-4-yl}Propan-2-yl)Acetamide99

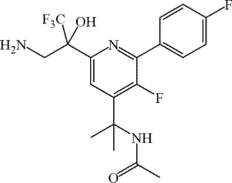 [SMILES: CC(=O)NC(C)(C)c1cc(C(O)(CN)C(F)(F)F)nc(-c2ccc(F)cc2)c1F]

TFA (1.3 mL, 17.0 mmol) was added to a solution of 98 (1.10 g, 2.13 mmol) in CH₂Cl₂ (21 mL) and the reaction mixture was stirred at rt overnight. NaHC03 (10%, aq.) was carefully added until pH was 7-8. The layers were separated, and the organic phase was dried (MgSO₄). The solids were removed by filtration and the filtrate was concentrated under reduced pressure. The crude mixture was combined with another fraction (77.3 µmol). The mixture was purified by silica column chromatography (CH₂Cl₂/(7 M NH₃ in CH₃OH, 9:1), gradient from 100:0 to 95:5) to afford a mixture of enantiomers 99 (675 mg, 74%) as a pale brown solid. ¹H NMR (400 MHz, DMSO-d6, 23° C.) δ ppm 1.31 - 1.57 (m, 1H), 1.65 (s, 6H), 1.83 (s, 3H), 3.13 (d, J=13.4 Hz, 1H), 3.17 (s, 1H), 3.43 (br d, J=13.4 Hz, 1H), 6.10 - 7.19 (m, 1H), 7.32 - 7.41 (m, 2H), 7.65 (d, J=5.7 Hz, 1H), 7.92 (dd, J=7.5, 5.7 Hz, 2H), 8.38 (s, 1H); LC-MS (method J): Rt = 1.28 min; mass calcd. for C₁₉H₂₀F₅N₃O₂ 417.1, m/z found 418.4 [M+H]⁺.

### 3.3.7. Synthesis of 107 and 108

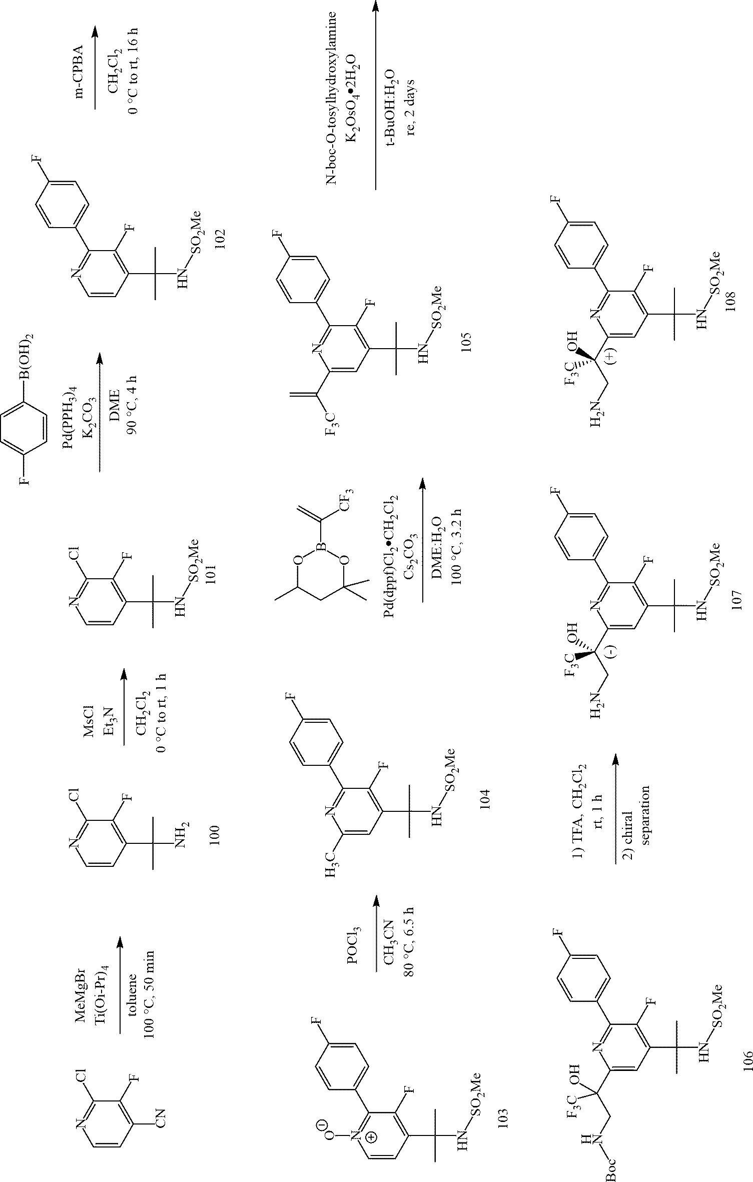

### 2-Chloro-3-fluoropyridin-4-yl)propan-2-amine 100

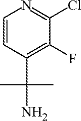

CH₃MgBr (3 M in Et₂O, 64 mL, 192 mmol) was added to a solution of 2-chloro-3-fluoropyridine-4-carbonitrile (10.0 g, 63.9 mmol) in toluene (500 mL). The mixture was stirred for 5 min and Ti(Oi-Pr)₄ (19.1 mL, 64.5 mmol) was added. The reaction mixture was stirred at 100° C. for 50 min. The reaction mixture was cooled to rt and the reaction was quenched with Na₂CO₃ (sat., aq.). The mixture was stirred overnight, filtered and the filter cake was washed with EtOAc. The layers were separated, and the aqueous phase was extracted with EtOAc. The combined organic extracts were dried (MgSO₄). The solids were removed by filtration and the filtrate was concentrated under reduced pressure to afford crude 100 (11.9 g) that was used as such in the next step.

### N-f2-Chloro-3-Fluoropvridin-4-yl)Propan-2-yl]Methanesulfonamide101

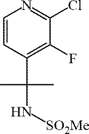 [SMILES: CC(C)(NS(C)(=O)=O)c1ccnc(Cl)c1F]

To a mixture of crude 100 (11.9 g) and Et₃N (17.6 mL, 126 mmol) in CH₂Cl₂ (1 L) at 0° C. was added methanesulfonyl chloride (6.4 mL, 82.1 mmol) dropwise (the internal temperature was maintained below 0° C. during the addition). The reaction mixture was stirred for 1 h and the reaction was quenched with water (200 mL). The layers were separated and the aqueous phase was extracted. The combined organic extracts were dried (MgSO₄). The solids were removed by filtration and the filtrate was concentrated under reduced pressure. The crude mixture was purified by silica column chromatography (CH₂Cl₂/CH₃OH, gradient from 100:0 to 96:4) to afford 101 (13.7 g, 80% over 2 steps).

### N-{2-Fluoro-2-(4-Fluorophenyl)Pyridin-4-yl]Propan-2-yl}Methanesulfonamide 102

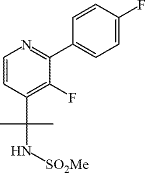 [SMILES: CC(C)(NS(C)(=O)=O)c1ccnc(-c2ccc(F)cc2)c1F]

A mixture of 101 (13.7 g, 51.4 mmol), 4-fluorophenylboronic acid (10.8 g, 77.1 mmol) and K₂CO₃ (2 M in H₂O, 51.4 mL, 103 mmol) in DME (171 mL) was purged with N₂. Pd(PPh₃)₄ (5.94 g, 5.14 mmol) was added and the mixture was purged again with N₂. The reaction mixture was stirred at 90° C. for 4 h. The reaction mixture was cooled to rt and diluted with CH₂Cl₂ and water. The layers were separated and the aqueous phase was extracted with CH2Cl₂. The organic phase was dried (MgSO₄). The solids were removed by filtration and the filtrate was concentrated under reduced pressure. The crude mixture was purified via silica column chromatography (heptane/EtOAc, gradient from 90:10 to 50:50) to afford 102 (15.8 g, 92%).

### 3-Fluoro-2-(4-fluorophenyl)-4-(2-(methylsulfonamido)propan-2-yl)pyridine 1-oxide 103

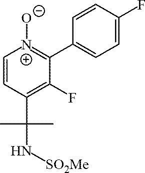 [SMILES: CC(C)(NS(C)(=O)=O)c1cc[n+]([O-])c(-c2ccc(F)cc2)c1F]

To a solution of 102 (15.8 g, 47.4 mmol) in anhydrous CH₂Cl₂ (591 mL) at 0° C. was added m-CPBA (16.4 g, 71.2 mmol, 75% pure). The reaction mixture was stirred at rt for 16 h. The mixture was successively washed with Na₂S₂O₃ (sat., aq.), and NaHC03 (sat., aq., twice). The organic phase was dried (MgSO₄). The solids were removed by filtration and the filtrate was concentrated under reduced pressure to afford 103 (14.3 g, 70%, 79% pure).

### N-{2-chloro-3-fluoro-2-(4-fluorophenyl)pyridin-4-yl]propan-2-yl}methanesulfonamide 104

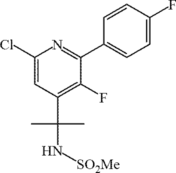 [SMILES: CC(C)(NS(C)(=O)=O)c1cc(Cl)nc(-c2ccc(F)cc2)c1F]

To a solution of 103 (14.3 g, 33 mmol, 79% pure) in CH₃CN (102 mL) was slowly added POC1₃ (12.3 mL, 132 mmol) and the reaction mixture was stirred at 80° C. overnight. The reaction mixture was cooled to rt and additional amount of POC1₃ (6.1 mL, 66 mmol) was added. The reaction mixture was stirred at 80° C. for another 2.5 h. The reaction mixture was cooled to rt and poured into ice bath (dropwise addition). The mixture was stirred for 10 min, diluted with EtOAc and basified with NaOH powder until pH was 7. The layers were separated, and the aqueous phase was extracted. The combined organic extracts were dried (MgSO₄). The solids were removed by filtration and the filtrate was concentrated under reduced pressure to afford crude 104 (13.9 g, 75% pure).

### N-{2-Fluoro-2-(4-Fluorophenyl)-6-(3,3,3-Trifluoroprop-1-en-2-yl)Pyridin-4-yl]Propan-2-yl}Methanesulfonamide 105

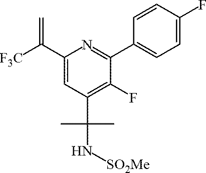 [SMILES: C=C(c1cc(C(C)(C)NS(C)(=O)=O)c(F)c(-c2ccc(F)cc2)n1)C(F)(F)F]

A mixture of 104 (12.9 g, 26.8 mmol, 75% pure), 4,4,6-trimethyl-2-(3,3,3-trifluoroprop-1-en-2-yl)-1,3,2-dioxaborinane (8.35 mL, 40.3 mmol) and C_{S2}CO₃ (26.2 g, 80.5 mmol) in water (11.1 mL) and DME (61.3 mL) was purged with N₂. Pd(dppf)Cl₂.CH₂Cl₂ (4.38 g, 5.37 mmol) was added and the mixture was purged again with N₂. The reaction mixture was stirred at 100° C. for 3.2 h. The reaction mixture was diluted with EtOAc and water. The layers were separated, and the aqueous phase was extracted with EtOAc (twice). The combined organic extracts were washed with brine, and dried (MgSO₄). The solids were removed by filtration and the filtrate was concentrated under reduced pressure. The crude mixture was combined with another fraction (2.08 mmol) and purified by silica column chromatography (CH₂Cl₂/CH₃OH, gradient from 100:0 to 90:10) to afford 105 (6.90 g, 57%) as an off-white solid.

### Tert-Butyl {3,3.3-Trifluoro-2-[5-Fluoro-6-(4-Fluorophenyl)-4-{2-[(Methanesulfonyl)Amino1-Propan-2-yl}Pyridin-2-yl]-2-Hydroxypropyl}Carbamate 106

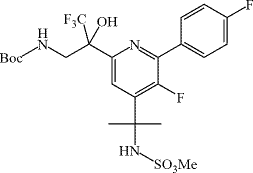

K₂O_{S}O₄.2H₂O (1.21 g, 3.28 mmol) and A-boc-O-tosylhydroxylamine (5.19 g, 18.1 mmol) were successively added to a solution of 105 (6.90 g, 16.4 mmol) in t-BuOH (186 mL) and water (11.9 mL). The reaction mixture was stirred at rt for 20 h. Additional amount of K₂O_{S}O₄•2H₂O (1.21 g, 3.28 mmol) was added and the reaction mixture was stirred at rt for another 1.5 day. Additional amount of K₂O_{S}O₄.2H₂O (1.21 g, 3.28 mmol) was added again and the reaction mixture was stirred at rt for another day. The reaction mixture was diluted with CH₂Cl₂ and NaHCO₃ (sat., aq.) was added. The layers were separated, and the organic phase was dried (MgSO₄). The solids were removed by filtration and the filtrate was concentrated under reduced pressure to afford crude 106.

### N-{6-[(-)-3-Amino-1,1,1-Trifluoro-2-Hydroxypropan-2-yl]-3-Fluoro-2-(4-FluoroPhenyl)Pyridine-4-yl}Propan-2-yl)Methanesulfonamide 107 andN-{6-r(+)-3-Amino-1,1,1-Trifluoro-2-Hvdroxvpropan-2-vl1-3-Fluoro-2-(4-Fluorophenyl)Pvridine-4-vl}Propan-2-vl)MethaneSulfonamide 108

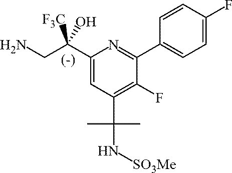

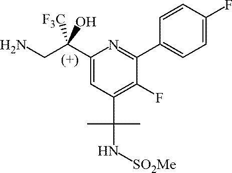

TFA (74.5 mL, 974 mmol) was added to a solution of crude 106 in CH₂Cl₂ (121 mL). The reaction mixture was stirred at rt for 1.5 h. The reaction mixture was diluted with CH₂Cl₂ and poured into NaHC03 (sat., aq.). NaHC03 powder was added until pH was 7. The layers were separated and the aqueous phase was extracted with CH2Cl₂. The combined organic extracts were dried (MgSO₄). The solids were removed by filtration and the filtrate was concentrated under reduced pressure. The crude mixture was purified by silica column chromatography (CH₂C₂/CH₃OH, gradient from 100:0 to 96:4) to afford a mixture of enantiomers (2.89 g). The residue was purified a second time by silica column chromatography (heptane/(EtOAc/CH₃OH, 9:1), gradient from 50:50 to 20:80) to afford a racemic mixture (1.90 g). The enantiomers were separated by SFC (CHIRALPAK AD-H 5 _{µm} 250 x 30 mm, mobile phase: 90% CO₂, 10% CH₃OH (1.5% i-PrNH₂)) to afford 107 (814 mg, 11%); ¹H NMR (500 MHz, DMSO-d₆) δ ppm 1.25 - 1.59 (m, 2H), 1.74 (s, 6H), 2.90 (s, 3H), 3.14 (d, J=13.6 Hz, 1H), 3.42 (d, J=13.6 Hz, 1H), 7.34 - 7.40 (m, 2H), 7.76 - 7.84 (m, 1H), 7.87 (d, J=5.4 Hz, 1H), 7.95 (dd, J=7.9, 5.7 Hz, 2H); LC-MS (method K): Rt = 1.19 min; mass calcd. for C₁₈H₂₀F₅N₃O₃S 453.1, m/z found 454.3 [M+H]⁺; and 108 (1.0 g, 13%); ¹H NMR (400 MHz, DMSO-d₆) δ ppm 1.74 (br s, 6H), 2.88 (d, J=16.0 Hz, 3H), 2.99 - 3.18 (m, 2H), 3.45 - 3.76 (m, 3H), 7.31 - 7.43 (m, 2H), 7.84 - 8.00 (m, 3H); LC-MS (method K): Rt = 1.19 min; mass calcd. for C₁₈H₂₀F₅N₃O₃S 453.1, m/z found 454.3 [M+H]⁺.

### 3.3.8. Synthesis of 114

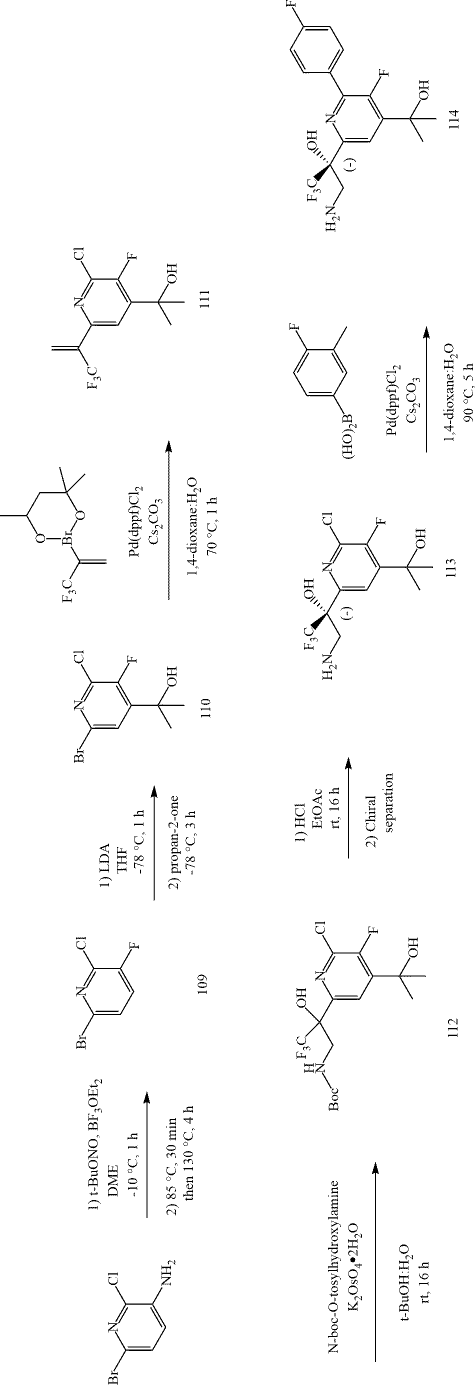

### 6-Bromochloro-3-fluoropyridine 109

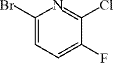

t-Butyl nitrite (3.4 mL, 29.0 mmol) was added dropwise to a mixture of 6-bromo-2-chloropyridin-3-amine (5.00 g, 24.1 mmol), (diethyloxonio)trifluoroborate (4.5 mL, 36.5 mmol) and 1,2-dimethoxyethane (30 mL) at -10° C. The reaction mixture was stirred at -10° C. for 1 h. The suspension was filtered, and the filter cake was washed with hexane (3 x 10 mL) and dried under reduced pressure to afford intermediate 6-bromo-2-chloropyridine-3-diazonium tetrafluoroborate (6.3 g), which was used in the next step without further purification. The latter was heated at 85° C. for 30 min and at 130° C. for 4 h. The mixture was purified by silica column chromatography (petroleum ether/EtOAc, 100:0 to 0:100) to afford 109 (2.53 g, 58 %) as a pink solid.

### 2-Bromo-2-chloro-3-fluoropyridin-4-yl)propan-2-ol 110

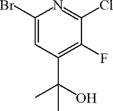

A solution of 109 (16.0 g, 76.0 mmol) in THF (50 mL) was added to a solution of LDA (2 M in THF, 57.6 mL, 115 mmol) and THF (50 mL) at -78° C. The reaction mixture was stirred at -78° C. for 1 h. Propan-2-one (24.0 mL, 322 mmol) was added dropwise at -78° C. and the reaction mixture was stirred for 3 h. The reaction was quenched with NH₄Cl (sat., aq., 30 mL). The mixture was combined with another fraction (71.3 mmol) and the aqueous phase was extracted with EtOAc (3 x 50 mL). The combined organic extracts were washed with brine (20 mL), and dried (Na₂SO₄). The solids were removed by filtration and the filtrate was concentrated to dryness under reduced pressure. The crude mixture was purified by silica column chromatography (petroleum ether/EtOAc, 100:0 to 50:50). A second purification was performed by HPLC (PREPL-X Phenomenex luna C18 250 x 50 x 10 µm column, mobile phase: 35 to 60% (v/v) CH₃CN/H₂O with 0.225% FA) to afford 110 (25 g, 63%).

### 2-f2-Chloro-3-Fluoro-6-(3,3,3-Trifluoroprop-1-en-2-yl)Pyridin-4-yl]Propan-2-ol 111

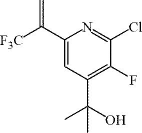 [SMILES: C=C(c1cc(C(C)(C)O)c(F)c(Cl)n1)C(F)(F)F]

110 (24.0 g, 89.4 mmol), 4,4,6-trimethyl-2-(3,3,3-trifluoroprop-1-en-2-yl)-1,3,2-dioxaborinane (19.9 g, 89.6 mmol), and C_{S2}CO₃ (88.8 g, 272 mmol) were added to a solution of 1,4-dioxane (120 mL) and H₂O (24 mL). The mixture was purged with Ar for 5 min and Pd(dppf)Cl2 (6.72 g, 9.18 mmol) was added. The mixture was purged with Ar for another 5 min and the reaction mixture was stirred at 70° C. for 1 h. The solid were removed by filtration and the filtrate was concentrated to dryness under reduced. The crude mixture was purified by silica column chromatography (petroleum ether/EtOAc, gradient from 1:0 to 4:1) to afford 111 (29.5 g, 68%, 58% pure) as a yellow oil.

### Tert-Butyl {2-f6-Chloro-5-Fluoro-4-(2-Hydroxypropan-2-yl)Pyridin-2-yl]-3,3,3-Trifluoro-2-Hydroxypropyl}Carbamate 112

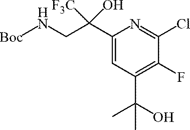 [SMILES: CC(C)(C)OC(=O)NCC(O)(c1cc(C(C)(C)O)c(F)c(Cl)n1)C(F)(F)F]

K₂O_{S}O₄•2H₂O (4.1 g, 11.1 mmol) was added to a mixture of 111 (27.0 g, 55.5 mmol, 58% pure) and N-boc-O-tosylhydroxylamine (17.6 g, 61.3 mmol) in t-BuOH (100 mL) and H₂O (15 mL). The reaction mixture was stirred at rt for 16 h. The mixture was concentrated to dryness under reduced pressure. The residue was poured into water (50 mL) and the aqueous phase was extracted with CH₂Cl₂ (3 x 50 mL). The combined organic extracts were washed with brine (50 mL), and dried (Na₂SO₄). The solids were removed by filtration and the filtrate was concentrated to dryness under reduced pressure. The crude mixture was purified by silica column chromatography (petroleum ether/EtOAc, gradient from 1:0 to 5:1) to afford 112 (15 g, 44%, 68% pure) as a yellow oil.

### (-)-3 -Amino-2- [6-Chl Oro-5-Fluoro-4-(2-Hydroxypropan-2-yl)Pyridin-2-yl]-1.1.1-Trifluoropropan-2-ol 113

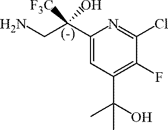

4 M HCl (85.0 mL, 340 mmol) was added dropwise to a solution of 112 (14.0 g, 33.6 mmol) in EtOAc (50 mL). The reaction mixture was stirred at rt for 16 h. The mixture was basified with NaOH (5 M, aq.) to pH 8, and poured into water (50 mL). The layers were separated, and the aqueous phase was extracted with CH₂Cl₂ (3 x 100 mL). The combined organic extracts were washed with brine (20 mL), and dried (Na₂SO₄). The solids were removed by filtration and the filtrate was concentrated to dryness under reduced pressure to afford a mixture of enantiomers (10.17 g, 92%, 96% pure) as a white solid. The enantiomers were separated by SFC (stationary phase: Daicel Chiralpak OD H, mobile phase: heptane/i-PrOH, gradient 85:15) to afford 113.

### (-)Amino-1,1,1-trifluoro-2-[5-fluoro-6-(4-fluoromethylphenyl)-4-(2-hydroxypropan-2-yl)pyridin-2-yl]propan-2-ol 114

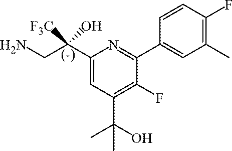

In a vial were added 113 (0.50 g, 1.58 mmol), 3-methyl-4-fluorophenylboronic acid (486 mg, 3.16 mmol) and C_{S2}CO₃ (1.24 mg, 3.79 mmol) in 1,4-dioxane (18 mL) and water (2 mL). The vial was sealed, degassed and purged with N₂ while vigorously stirred. Pd(dppf)Cl2 (57.8 mg, 78.9 µmol) was added. The vial was sealed and stirred at 90° C. for 5 h. The mixture was cooled to rt and concentrated under reduced pressure. The crude mixture was partitioned between CH₂Cl₂ and NaHCOs (sat., aq.). The organic layer was dried (MgSO₄). The solids were removed by filtration and the filtrate was concentrated under reduced pressure. The crude mixture was purified by silica column chromatography (CH₂CI₂/7M NH₃ in CH₃OH, gradient from 100:0 to 90:10). Anhydrous DMF (6 mL) was added to the residue to afford a solution (118 mg/mL) of 114 (708 mg, 86%, 75% pure) in DMF. LC-MS (method G): Rt = 1.98 min; mass calcd. for C₁₈H₁₉F₅N₂O₂ 390.1, m/z found 391.1 [M+H]⁺.

### 3.3.9. Synthesis of 116 and 117

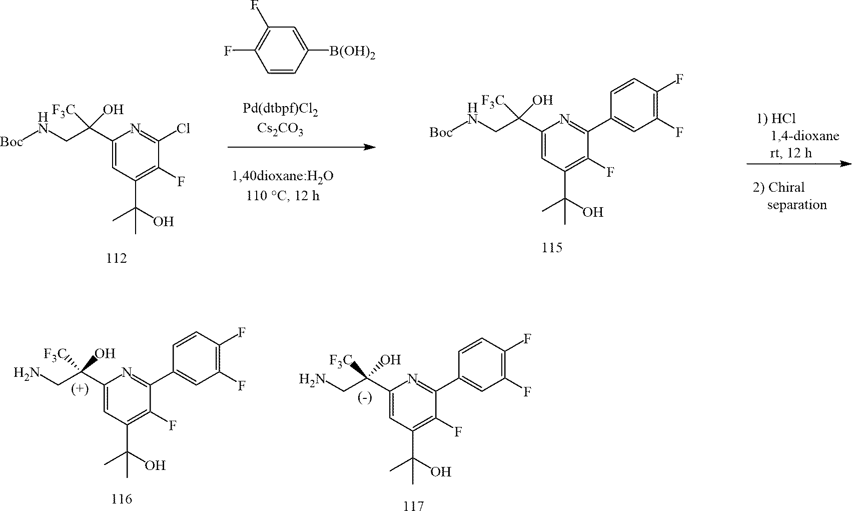

### Tert-Butyl {2-[6-(3,4-Difluorophenyl)-5-Fluoro-4-(2-Hydroxypropan-2-yl)Pyridin-2-yl]-3,3,3-Trifluoro-2-Hydroxypropyl}Carbamate 115

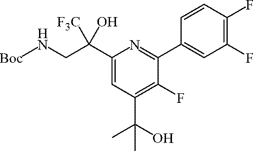 [SMILES: CC(C)(C)OC(=O)NCC(O)(c1cc(C(C)(C)O)c(F)c(-c2ccc(F)c(F)c2)n1)C(F)(F)F]

A mixture of 112 (300 mg, 0.72 mmol), (3,4-difluorophenyl)boronic acid (120 mg, 0.76 mmol), K₃PO₄ (300 mg, 1.41 mmol) in 1,4-dioxane (5 mL) and H₂O (1 mL) was purged with Ar for 5 min. Pd(dtbpf)Cl₂ (20.0 mg, 30.7 µmol) was added and the mixture was purged with Ar for another 5 min. The reaction mixture was stirred at 110° C. for 12 h. The reaction mixture was cooled to rt and quenched with water (20 mL). The layers were separated, and the aqueous phase was extracted with EtOAc (3 x 20 mL). The combined organic extracts were dried (Na₂SO₄). The solids were removed by filtration and the filtrate was concentrated to dryness under reduced pressure. The crude mixture was purified by silica column chromatography (petroleum ether/EtOAc, 12:1) to afford 115 (250 mg, 67%, 95% pure) as a yellow oil.

### (+)Amino-2-[6-(3,4-Difluorophenyl)-5-Fluoro-4-(2-Hydroxypropan-2-yl)Pyridin-2-yl]-1,1,1-Trifluoropropan-2-ol 116 and (-)Amino-2-[6-(3,4-Difluorophenyl)-5-Fluoro-4-(2-Hydroxypropan-2-yl)Pyridin-2-yl]-1,1,1-Trifluoropropan-2-ol 117

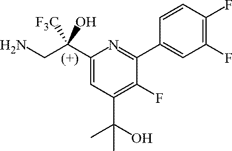

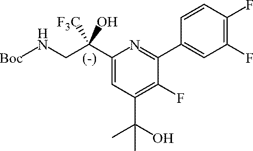

A mixture of 115 (250 mg, 506 µmol) in HCl (4 M in 1,4-dioxane, 5 mL) was stirred at rt for 12 h. The reaction mixture was concentrated to dryness under reduced pressure. The residue was dissolved in H₂O (10 mL) and the solution was basified with solid NaHC03 to pH 8. The aqueous phase was extracted with EtOAc (2 x 20 mL). The combined organic extracts were dried (Na₂SO₄). The solids were removed by filtration and the filtrate was concentrated to dryness under reduced pressure. The residue was suspended in water (20 mL). The mixture was frozen and lyophilized to dryness to afford a mixture of enantiomers (170 mg, 82%), as a white solid. The enantiomers (700 mg) were separated via SFC (Chiralpak Diacel AD 20 x 250 mm, mobile phase: CO₂, i-PrOH + 0.4% i-PrNH₂) to afford 116 (346 mg, 49%); LC-MS (method C): Rt = 1.88 min; mass calcd. for C₁₇H₁₆F₆N₂O₂ 394.1, m/z found 395.1 [M+H]⁺; [α]_{D}²⁰ +47.79 (c 0.272, DMF) and 117 (354 mg, 51%); LC-MS (method C): Rt = 1.88 min; mass calcd. for C₁₇H₁₆F₆N₂O₂ 394.1, m/z found 395.1 [M+H]⁺; [α]D²⁰ -62.65 (c 0.212, DMF).

### 3.3.10. Synthesis of 127 and 128

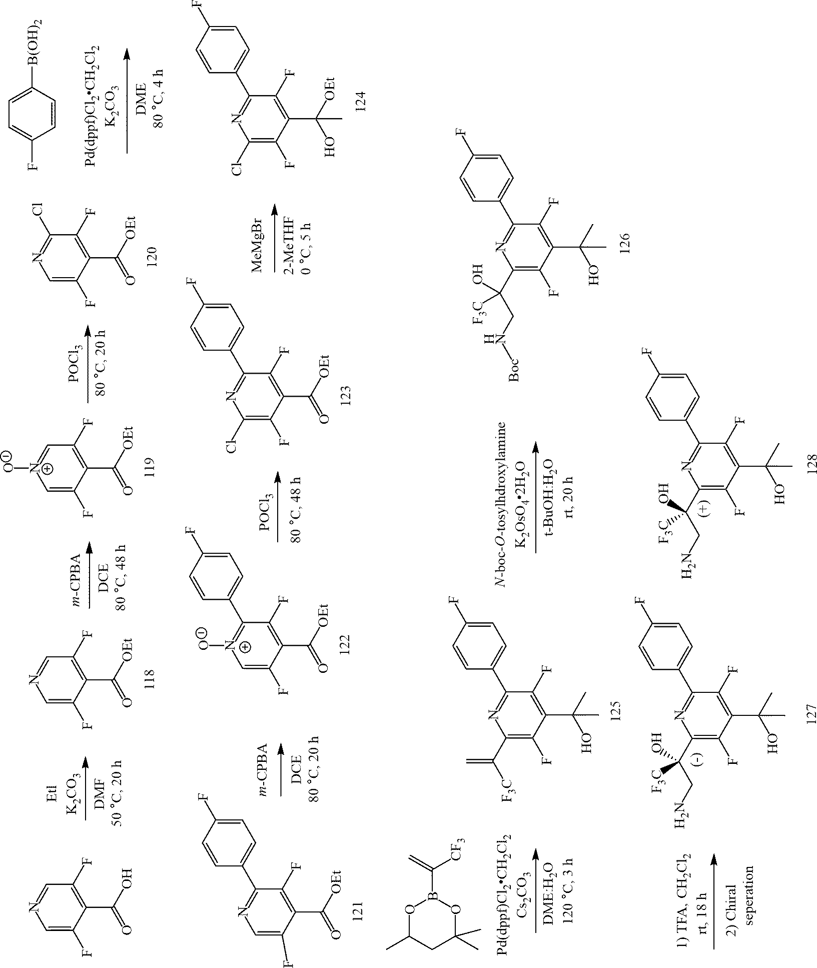

### Ethyl 3,5-difluoropyridine-4-carboxylate 118

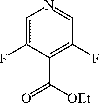 [SMILES: CCOC(=O)c1c(F)cncc1F]

In a Schlenk flask, a mixture of 3,5-difluoroisonicotinic acid (25.0 g, 157 mmol), iodoethane (15.0 mL, 185 mmol) and K₂CO₃ (23.6 g, 171 mmol) in DMF (250 mL) was stirred at 50° C. for 20 h. The reaction mixture was diluted with Et₂O and water. The layers were separated, and the aqueous phase was extracted with Et₂O (3 times). The combined organic layers were washed with brine (3 times), and dried (MgSO₄). The solids were removed by filtration and the filtrate was concentrated under reduced pressure to afford 118 (26 g, 57%, 64% pure) as a yellow oil.

### 4-(Ethoxycarbonyl)-3,5-difluoro-1-oxo-1λ⁵-pyridin-2-ylium 119

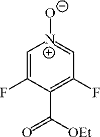 [SMILES: CCOC(=O)c1c(F)c[n+]([O-])cc1F]

In a Schlenk flask, to a solution of 118 (12.0 g, 41.0 mmol, 64% pure) in DCE (410 mL) was added m-CPBA (37.8 g, 164 mol, 75% pure). The reaction mixture was stirred at 80° C. for 48 h. The reaction was quenched with NaOH (1N). The layers were separated, and the organic phase was successively washed with NH₄Cl (sat., aq.), Na₂S₂O₃ (10%, aq.), water and brine, and dried (MgSO₄). The solids were removed by filtration and the filtrate was concentrated under reduced pressure to afford 119 (7.63 g, 92%) as a yellow residue.

### Ethyl 2-chloro-3,5-difluoropyridine-4-carboxylate 120

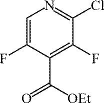 [SMILES: CCOC(=O)c1c(F)cnc(Cl)c1F]

In a sealed tube, a solution of 119 (8.41 g, 41.4 mmol) in POC1₃ (193 mL) was stirred at 80° C. for 20 h. The reaction mixture was concentrated under reduced pressure. The brown residue was taken up in water and EtOAc. The mixture was basified with K₂CO₃ powder and extracted with EtOAc (twice). The combined organic layers were dried (MgSO₄). The solids were removed by filtration and the filtrate was concentrated under reduced pressure to afford 120 (6.57 g, 72%) as a yellow oil.

### Ethyl 3,5-difluoro-2-(4-fluorophenyl)pyridine-4-carboxylate 121

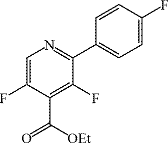 [SMILES: CCOC(=O)c1c(F)cnc(-c2ccc(F)cc2)c1F]

In a sealed tube, a mixture of 120 (5.94 g, 28.8 mmol), 4-fluorophenylboronic acid (5.63 g, 40.2 mmol) and K₂CO₃ (26.8 mL) in DME (53.6 mL) was purged with N₂. Pd(dppf)Cl₂.CH₂Cl₂ (2.19 g, 2.68 mmol) was added and the mixture was purged again with N₂. The reaction mixture was stirred at 80° C. for 4 h. The reaction mixture was diluted with EtOAc and water. The layers were separated and the aqueous phase was extracted with EtOAc (twice). The combined organic layers were dried (MgSO₄). The solids were removed by filtration and the filtrate was concentrated under reduced pressure, then purified by silica column chromatography (heptane/EtOAc, gradient from 49:1 to 4:1) to give 121 (5.59 g, 64%, 86% pure) as a colorless oil.

### 4-(Ethoxycarbonyl)-3/,5-difluoro-2-(4-fluorophenyl)pyridine 1-oxide 122

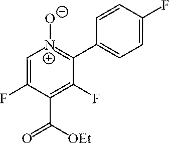 [SMILES: CCOC(=O)c1c(F)c[n+]([O-])c(-c2ccc(F)cc2)c1F]

To a solution of 121 (6.87 g, 21.0 mmol, 86% pure) in DCE (212 mL) was added m-CPBA (19.3 g, 84.0 mmol, 75% pure). The reaction mixture was stirred at 80° C. for 20 h. The reaction was quenched with NaOH (1N, aq.). The organic layer was successively washed with NH₄Cl (sat., aq.), Na₂S₂O₃ (10%, aq.), water and brine, and dried (MgSO₄). The solids were removed by filtration and the filtrate was concentrated under reduced pressure to give 122 (7.03 g) that was used in the next step without further purification.

### Ethyl 2-chloro-3,5-difluoro-6-(4-fluorophenyl)pyridine-4-carboxylate 123

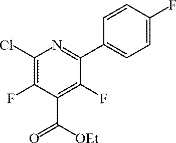 [SMILES: CCOC(=O)c1c(F)c(Cl)nc(-c2ccc(F)cc2)c1F]

In a sealed tube, a solution of 122 in POC1₃ (143 mL) was stirred at 80° C. for 48 h. The reaction mixture was concentrated under reduced pressure. The brown residue was reconstituted in water and EtOAc and the mixture was basified with K₂CO₃ powder. The layers were separated, and the aqueous phase was extracted with EtOAc (twice). The combined organic layers were dried (MgSO₄). The solids were removed by filtration and the filtrate was concentrated under reduced pressure to afford 123 (6.97 g) as a brown oil. The product was used in the next step without further purification.

### 2-f2-Chloro-3,5-Difluoro-6-(4-Fluorophenyl)Pyridin-4-yl]Propan-2-ol 124

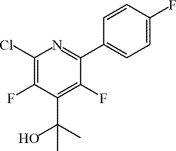 [SMILES: CC(C)(O)c1c(F)c(Cl)nc(-c2ccc(F)cc2)c1F]

CH₃MgBr (3 M in Et₂O, 18.4 mL, 55.2 mmol) was added dropwise to a solution of 123 in anhydrous 2-MeTHF (135 mL) at 0° C. The internal temperature was maintained near 0° C. during the addition. The reaction mixture was stirred at this temperature for 5 h. The reaction mixture was diluted with EtOAc and NH₄Cl (10%, aq.) was added. The layers were separated and the aqueous phase was extracted with EtOAc (twice). The combined organic layers were washed with brine, and dried (MgSO₄). The solids were removed by filtration and the filtrate was concentrated under reduced pressure. The crude mixture was purified by silica column chromatography (heptane/EtOAc, gradient from 95:5 to 7:3) to afford 124 (4.54 g, 68% over 3 steps) as a yellow oil that crystallized on standing.

### 2-F 3,5-Difluoro-2-(4-Fluorophenyl)-6-(3,3,3-Trifluoroprop-1-en-2-yl)Pyridin-4-yl]Propan-2-ol 125

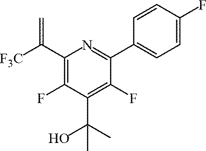 [SMILES: C=C(c1nc(-c2ccc(F)cc2)c(F)c(C(C)(C)O)c1F)C(F)(F)F]

In a sealed tube, a mixture of 124 (4.53 g, 15.0 mmol), 4,4,6-trimethyl-2-(3,3,3-trifluoroprop-1-en-2-yl)-1,3,2-dioxaborinane (4.67 mL, 22.5 mmol) and C_{S2}CO₃ (14.7 g, 45.2 mmol) in H₂O (6.3 mL) and DME (34.5 mL) was purged with N₂. Pd(dppf)Cl₂.CH₂Cl₂ (3.68 g, 4.51 mmol) was added and the mixture was purged again with N₂. The reaction mixture was stirred at 120° C. for 5 h. The reaction mixture was diluted with EtOAc and water. The layers were seperated and the aqueous phase was extracted with EtOAc (twice). The combined organic layers were washed with NaHCOs (sat., aq.) and brine, and dried (MgSO₄). The solids were removed by filtration and the filtrate was, concentrated under reduced pressure. The crude mixture was purified by silica column chromatography (heptane/EtOAc, gradient from 1:0 to 7:3) to afford 125 (4.03 g, 74%).

### T-Butyl 12-[3.5-Difluoro-6-(4-Fluorophenyl)-4-(2-Hydroxypropan-2-yl)Pyridin-2-yl1-3.3.3-Trifluoro-2-Hvdroxypropyl}Carbamate 126

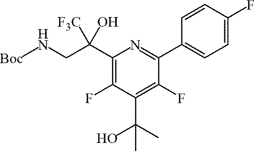 [SMILES: CC(C)(C)OC(=O)NCC(O)(c1nc(-c2ccc(F)cc2)c(F)c(C(C)(C)O)c1F)C(F)(F)F]

K₂O_{S}O₄.2H₂O (1.82 g, 4.94 mmol) and then N-boc-O-tosylhydroxylamine (15.6 g, 54.3 mmol) were added to a solution of 125 (10.5 g, 24.7 mmol, 85% pure) in t-BuOH (280 mL) and H₂O (18 mL). The reaction mixture was stirred at rt for 20 h. The reaction mixture was diluted with water and EtOAc. The layers were separated, and the aqueous phase was extracted with EtOAc (3 times). The combined organic extracts were washed with NaHC03 (sat., aq.), and dried (MgSO₄). The solids were removed by filtration and the filtrate was concentrated under reduced pressure. The crude mixture was purified by silica column chromatography (heptane/EtOAc, gradient from 90:10 to 80:20) to afford two fractions of 126: fraction A (10.4 g, 42%, 49% pure) and fraction B (5.8 g, 44%, 92% pure).

### (-)Amino-2-[3,5-Difluoro-6-(4-Fluorophenyl)-4-(2-Hydroxypropan-2-yl)Pyridin-2-yl]-1,1,1-Trifluoropropan-2-ol 127 and (+)Amino-2-(3,5-Difluoro-6-(4-Fluorophenyl)-4-(2-Hydroxypropan-2-yl)Pyridin-2-yl1-1,1,1-Trifluoropropan-2-ol 128

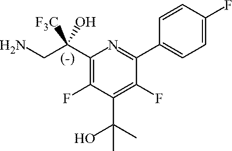

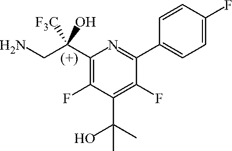

TFA (12 mL, 157 mmol) was added to a solution of 126 (10.4 g, 10.3 mmol, 49% pure) in CH₂C1₂ (150 mL) and the reaction mixture was stirred at rt for 18 h. The reaction mixture was diluted with CH₂C1₂ and poured into NaHC03 (sat., aq.). The layers were separated, and the aqueous phase was extracted with CH₂C1₂ (twice). The combined organic extracts were dried (MgSO₄). The solids were removed by filtration and the filtrate was concentrated under reduced pressure. The crude mixture was combined with another fraction (13.8 mmol) and purified by silica column chromatography (CH₂Cl₂/CH₃OH, gradient from 100:0 to 92:8). The enantiomers were separated by SFC (stationary phase: CHIRALPAK AD-H 5 _{µm} 250 x 30 mm, mobile phase: 85% CO₂, 15% CH₃OH (0.3% i-PrNH₂)) to give 127 (3.64 g, 38%); ¹H NMR (400 MHz, DMSO-d₆) δ ppm 1.35 - 1.79 (m, 2H), 1.65 (s, 6H), 3.12 (d, J=13.4 Hz, 1H), 3.56 (br d, J= 13.4 Hz, 1H), 5.65 (s, 1H), 6.21 - 6.89 (m, 1H), 7.37 (t, J=8.9 Hz, 2H), 7.93 (br dd, J=7.2, 5.6 Hz, 2H); LC-MS (method K): Rt = 1.24 min; mass calcd. for C₁₇H₁₆F₆N₂O₂ 394.1, m/z found 395.2 [M+H]⁺; and 128 (4.35 g, 42%). ¹H NMR (400 MHz, DMSO-d₆) δ ppm 1.41 - 1.75 (m, 2H), 1.65 (s, 6H), 3.12 (br d, J=13.6 Hz, 1H), 3.56 (br d, J=13.7 Hz, 1H), 5.66 (s, 1H), 6.19 - 6.90 (m, 1H), 7.37 (t, J=8.9 Hz, 2H), 7.93 (br dd, J=7.2, 5.7 Hz, 2H); LC-MS (method K): Rt = 1.24 min; mass calcd. for C₁₇H₁₆F₆N₂O₂ 394.1, m/z found 395.2 [M+H]⁺.

### 3.3.11. Synthesis of 140 and 141

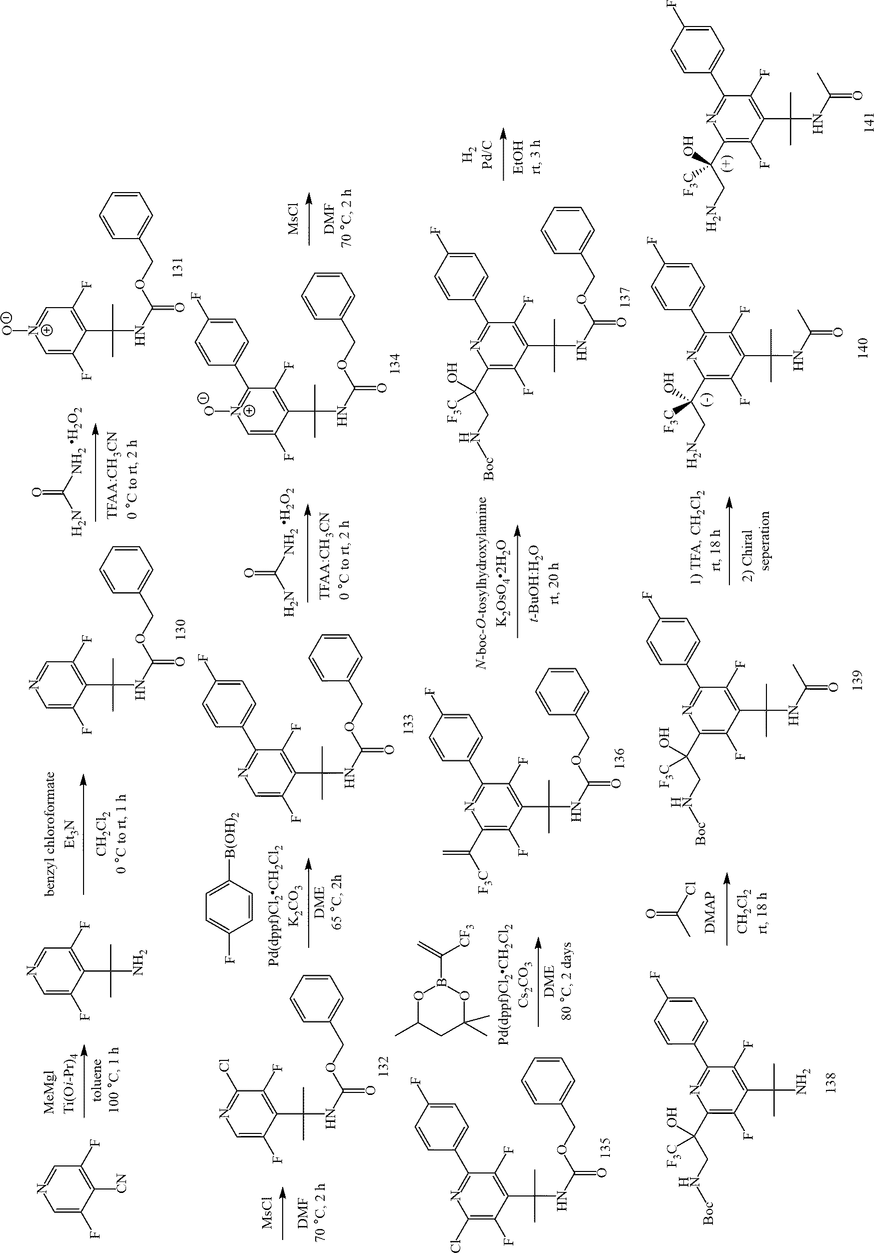

### 2-(3,5-Difluoropyridin-4-yl)propan-2-amine 129

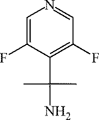

CH₃MgI (3 M in 2-methylTHF, 220 mL, 661 mmol) was added to a solution of 3,5-difluoropyridine-4-carbonitrile (32.5 g, 220 mmol, 95% pure) in toluene (1 L). The reaction mixture was stirred for 30 min and Ti(Oi-Pr)₄ (65.9 mL, 223 mmol) was added. The reaction mixture was stirred at 100° C. for 60 min. The reaction mixture was cooled to rt and the reaction was quenched with Na₂CO₃ (sat., aq.). The mixture was combined with another fraction (143 mmol), filtered over Celite® and washed with EtOAc. The layers were separated, and the aqueous phase was extracted with EtOAc (twice). The combined organic extracts were washed with a solution of brine and water (9:1) and concentrated to dryness under reduced pressure. The crude 129 was used as such in the next step.

### Benzyl (3,5-difluoropyridin-4-yl)propan-2-yllcarbamate 130

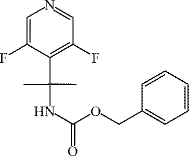 [SMILES: CC(C)(NC(=O)OCc1ccccc1)c1c(F)cncc1F]

To a solution of crude 129 and Et₃N (66.2 mL, 476 mmol) in CH₂Cl₂ (820 mL) at 0° C. was added benzyl chloroformate (44 mL, 310 mmol) dropwise (the internal temperature of the reaction mixture was maintained below 0° C. during the addition). The reaction mixture was stirred for 1 h at rt. The reaction was quenched with water. The layers were separated, and the aqueous phase was extracted with CH₂Cl₂ (twice). The combined organic extracts were dried (MgSO₄). The solids were removed by filtration and the filtrate was concentrated under reduced pressure. The crude mixture was purified by silica column chromatography (heptane/EtOAc, gradient from 90:10 to 70:30) to afford 130 (7.8 g, 9% over 2 steps, 80% pure).

### 4-(((Benzyloxy)carbonyl)amino)propan-2₋yl)-3_{.}5-difluoropyridine 1-oxide 131

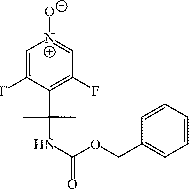 [SMILES: CC(C)(NC(=O)OCc1ccccc1)c1c(F)c[n+]([O-])cc1F]

A mixture of 130 (2.00 g, 5.22 mmol, 80% pure) and urea hydrogen peroxide (1.03 g, 11.0 mmol) in CH₃CN (30 mL) was cooled at 0° C. Trifluoroacetic anhydride (1.45 mL, 10.5 mmol) was slowly added and the reaction mixture was stirred at rt for 2 h. Na₂S₂O₃ (10%, aq.) was added and the mixture was stirred for 15 min. The aqueous phase was extracted with CH₂Cl2 (twice). The combined organic extracts were dried (MgSO₄). The solids were removed by filtration and the filtrate was concentrated under reduced pressure to afford crude 131.

### Benzyl (2-chloro-3,5-difluoropyridin-4-yl)propan-2-yl]carbamate 132

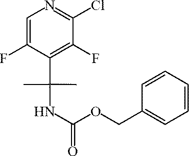 [SMILES: CC(C)(NC(=O)OCc1ccccc1)c1c(F)cnc(Cl)c1F]

Methanesulfonyl chloride (0.67 mL, 8.69 mmol) was added to a solution of crude 131 (500 mg, 0.87 mmol, 56% pure) in DMF (5 mL) at rt. The reaction mixture was stirred at 70° C. for 2 h. The reaction mixture was poured into NaHC03 (sat., aq.) and diluted with EtOAc. The aqueous phase was extracted with EtOAc (twice). The combined organic extracts were dried (MgSO₄). The solids were removed by filtration and the filtrate was concentrated under reduced pressure. The crude mixture was purified by silica column chromatography (CH₂Cl₂/CH₃OH, gradient from 100:0 to 99:1) to afford 132 (270 mg, 78%).

### Benzyl {2-(3,5-difluoro-2-(4-fluorophenyl)pyridin-4-yl1propan-2-yl}carbamate 133

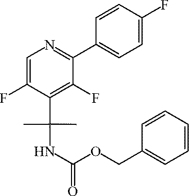 [SMILES: CC(C)(NC(=O)OCc1ccccc1)c1c(F)cnc(-c2ccc(F)cc2)c1F]

In a Schlenk reactor, a mixture of 132 (4.60 g, 13.5 mmol), 4-fluorophenylboronic acid (2.83 g, 20.2 mmol) and K₂CO₃ (2 M in H₂O, 13.5 mL, 27 mmol) in DME (100 mL) was purged with N₂. Pd(dppf)Cl₂.CH₂Cl₂ (2.21 g, 2.70 mmol) was added and the mixture was purged again with N₂. The reaction mixture was stirred at 65° C. for 2 h. The reaction mixture was filtered over Celite® and washed with EtOAc. The filtrate was diluted with EtOAc and brine. The layers were separated, and the aqueous phase was extracted with EtOAc (twice). The combined organic extracts were dried (MgSO₄). The solids were removed by filtration and the filtrate was concentrated under reduced pressure. The crude mixture was purified by silica column chromatography (heptane/EtOAc, gradient from 90:10 to 80:20) to afford 133 (5.34 g, 79%, 80% pure).

### 4-(((Benzvloxy)carbonyl)amino)propan-2-yl)-3.5-difluoro-2-(4-fluorophenyl)pyridine 1-oxide 134

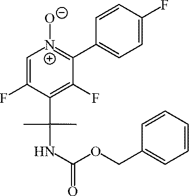 [SMILES: CC(C)(NC(=O)OCc1ccccc1)c1c(F)c[n+]([O-])c(-c2ccc(F)cc2)c1F]

A mixture of 133 (4.30 g, 8.59 mmol, 80% pure) and urea hydrogen peroxide (1.70 g, 18.0 mmol) in CH₃CN (35 mL) was cooled at 0° C. Trifluoroacetic anhydride (2.39 mL, 17.2 mmol) was slowly added. The reaction mixture was stirred at rt for 2 h. The reaction mixture was cooled to 0° C. and additional amount of trifluoroacetic anhydride (1.19 mL, 8.59 mmol) was added. The reaction mixture was stirred at rt for another 2 h. A 10% aqueous solution of Na₂S₂O₃ was added and the mixture was stirred for 15 min. The layers were separated, and the aqueous phase was extracted with CH₂Cl₂ (6 times). The combined organic extracts were dried (MgSO₄). The solids were removed by filtration and the filtrate was concentrated under reduced pressure to afford crude 134.

### Benzyl {2-[2-chloro-3,5-difluoro-6-(4-fluorophenyl)pyridin-4-yl]propan-2-yl}carbamate 135

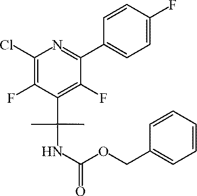 [SMILES: CC(C)(NC(=O)OCc1ccccc1)c1c(F)c(Cl)nc(-c2ccc(F)cc2)c1F]

Methanesulfonyl chloride (6.40 mL, 82.6 mmol) was added to a solution of crude 134 (5.55 g, 8.26 mmol, 62% pure) in DMF (55 mL) at rt. The reaction mixture was stirred at 70° C. for 2 h, then poured into NaHCO₃ (sat., aq.) and diluted with EtOAc. The layers were separated, and the aqueous phase was extracted with EtOAc (twice). The combined organic extracts were washed with brine (3 times), and dried (MgSO₄). The solids were removed by filtration and the filtrate was concentrated under reduced pressure. The crude mixture was combined with another fraction (1.49 mmol) and purified by silica column chromatography (heptane/EtOAc, gradient from 95:5 to 90:10) to afford 135 (5.00 g, 72% pure) as a white solid.

### Benzyl {2-[3,5-difluoro-2-(4-fluorophenyl)-6-(3,3,3-trifluoroprop-1-en-2-yl)pyridin-4-yl]propan-2,-yl}carbamate 136

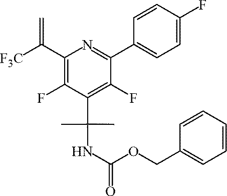 [SMILES: C=C(c1nc(-c2ccc(F)cc2)c(F)c(C(C)(C)NC(=O)OCc2ccccc2)c1F)C(F)(F)F]

In a Schlenk tube, a mixture of 135 (1.00 g, 1.66 mmol, 72% pure), 4,4,6-trimethyl-2-(3,3,3-trifluoroprop-1-en-2-yl)-1,3,2-dioxaborinane (0.69 mL, 3.31 mmol) and C_{S2}CO₃ (1.63 g, 4.99 mmol) in water (1.5 mL) and DME (10 mL) was purged with N₂. Pd(dppf)Cl₂.CH₂Cl₂ (135 mg, 0.17 mmol) was added and the mixture was purged again with N₂. The reaction mixture was stirred at 80° C. for 2 days. The reaction mixture was diluted with EtOAc and water and filtered over Celite^{®}. The layers were separated, and the aqueous phase was extracted with EtOAc (twice). The combined organic extracts were dried (MgSO₄). The solids were removed by filtration and the filtrate was concentrated under reduced pressure. The crude mixture was partially purified by silica column chromatography (heptane/EtOAc, gradient from 95:5 to 85:15) to afford two fractions of 136: fraction A (440 mg, 28%, 53% pure) and fraction B (630 mg, 55%, 71% pure).

### Benzyl {2-[2-{3-[(tert-butoxycarbonyl)amino]-1,1,1-trifluoro-2-hydroxypropan-2-yl}-3,5-difluoro-6-(4-fluorophenyl)pyridin-4-yl]propan-2-yl}carbamate 137

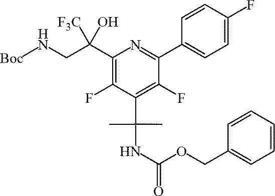 [SMILES: CC(C)(C)OC(=O)NCC(O)(c1nc(-c2ccc(F)cc2)c(F)c(C(C)(C)NC(=O)OCc2ccccc2)c1F)C(F)(F)F]

K₂O_{S}O₄.2H₂O (66.7 mg, 0.18 mmol) and N-boc-O-tosylhydroxylamine (572 mg, 1.99 mmol) were successively added to a solution of 136 (630 mg, 905 µmol, 71% pure) in t-BuOH (10 mL) and water (0.66 mL). The reaction mixture was stirred at rt for 20 h. The reaction mixture was diluted with water and EtOAc. The layers were separated, and the aqueous phase was extracted with EtOAc (3 times). The combined organic extracts were washed with NaHCOs (sat., aq.), and dried (MgSO₄). The solids were removed by filtration and the filtrate was concentrated under reduced pressure. The crude mixture was purified by silica column chromatography (heptane/EtOAc, gradient from 90:10 to 80:20) to afford two fractions of 137: fraction A (420 mg, 35%, 47% pure) and fraction B (50 mg, 7%, 80% pure).

### T-Butyl {2-[4-(2-Aminopropan-2-yl)-3,5-Difluoro-6-(4-Fluorophenyl)Pyridin-2-yl]-3,3,3-Trifluoro-2-Hydroxypropyl}Carbamate 138

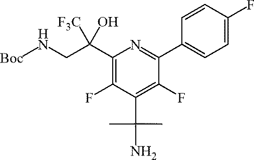 [SMILES: CC(C)(C)OC(=O)NCC(O)(c1nc(-c2ccc(F)cc2)c(F)c(C(C)(C)N)c1F)C(F)(F)F]

A solution of 137 (420 mg, 315 µmol; 47% pure) in EtOH (10 mL) was stirred under N₂ atmosphere. 10% Pd/C (66.9 mg) was added and the reaction mixture was stirred at rt under 5 bar H₂ for 3 h. The reaction mixture was filtered over Celite® and washed with EtOH. The filtrate was concentrated to dryness under reduced pressure. The crude mixture was purified by silica column chromatography (CH₂C1₂/CH₃OH, gradient from 100:0 to 98:2) to afford 138 (103 mg, 66%).

### Tert-Butyl {2-[4-(2-Acetamidopropan-2-yl)-3,3,3-Trifluoro-2-Hydroxypropyl}Carbamate 139

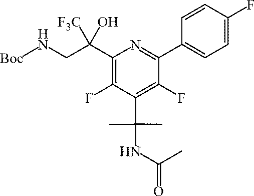 [SMILES: CC(=O)NC(C)(C)c1c(F)c(-c2ccc(F)cc2)nc(C(O)(CNC(=O)OC(C)(C)C)C(F)(F)F)c1F]

At 0° C., acetyl chloride (238 µL, 3.34 mmol) was added to a mixture of 138 (1.10 g, 2.23 mmol) and DMAP (545 mg, 4.46 mmol) in CH₂Cl₂ (15 mL). The reaction mixture was stirred at rt for 18 h. The reaction mixture was diluted with water and CH2Cl₂. The layers were separated and the organic phase was dried (MgSO₄). The solids were removed by filtration and the filtrate was concentrated under reduced pressure. The crude mixture was purified by silica column chromatography (heptane/EtOAc, gradient from 90:10 to 60:40) to afford 139 (1.17 g, 93%).

### N-{2-[(-)-3-Amino-1,1,1-Trifluoro-2-Hydroxypropan-2-yl]-3,5-Difluoro-6-(4-Fluorophenyl)Pyridin-4-yl}Propan-2-yl)Acetamide 140 and {2-|(+)-3-Amino-1,1,1-Trifluoro-2-Hydroxypropan-2-yl1-3,5-Difluoro-6-(4-Fluorophenyl)Pyridin-4-yl}Propan-2-yl)Acetamide 141

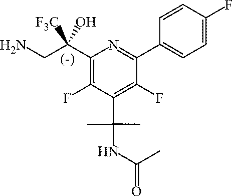

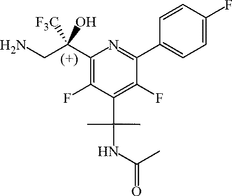

TFA (794 µL, 10.4 mmol) was added to a solution of 139 (1.17 g, 2.08 mmol) in CH₂Cl2 (30 mL) and the reaction mixture was stirred at rt for 18 h. Additional quantity of TFA (794 µL, 10.4 mmol) was added and the reaction mixture was stirred at rt for another 3 h. The reaction mixture was poured into NaHCOs (sat., aq.). The layers were separated, and the aqueous phase was extracted with CH₂Cl₂ (3 times). The combined organic extracts were dried (MgSO₄). The solids were removed by filtration and the filtrate was concentrated under reduced pressure. The enantiomers were separated by SFC (CHIRALPAK AD-H 5 _{µm} 250 x 30 mm, mobile phase: 85% CO₂, 15% i-PrOH (0.3% i-PrNH₂)) to give 140 (378 mg, 41%); ¹H NM+R (400 MHz, DMSO-d₆) δ ppm 1.65 (br dd, J=8.8, 2.6 Hz, 1H), 1.71 (br s, 6H), 1.78 (s, 3H), 3.11 (d, J=13.7 Hz, 1H), 3.52 (br d, J=13.8 Hz, 1H), 6.01 - 7.02 (m, 1H), 7.33 - 7.43 (m, 2H), 7.91 (br dd, J=7.2, 5.6 Hz, 2H), 8.57 (s, 1H); and 141 that was re-purified via chiral SFC (stationary phase: CHIRALPAK AD-H 5 µm 250 x 30 mm, mobile phase: 87% CO₂, 13% i-PrOH (0.3% i-PrNH₂)) to give 141 (359 mg, 40%); ¹H NMR (400 MHz, DMSO-d₆) δ ppm 1.59 - 1.67 (m, 1H), 1.71 (br s, 6H), 1.78 (s, 3H), 3.11 (d, J=13.6 Hz, 1H), 3.52 (br d, J=13.6 Hz, 1H), 6.20 - 6.91 (m, 1H), 7.33 - 7.41 (m, 2H), 7.88 - 7.95 (m, 2H), 8.57 (s, 1H).

### 3.3.12. Synthesis of 143 and 144

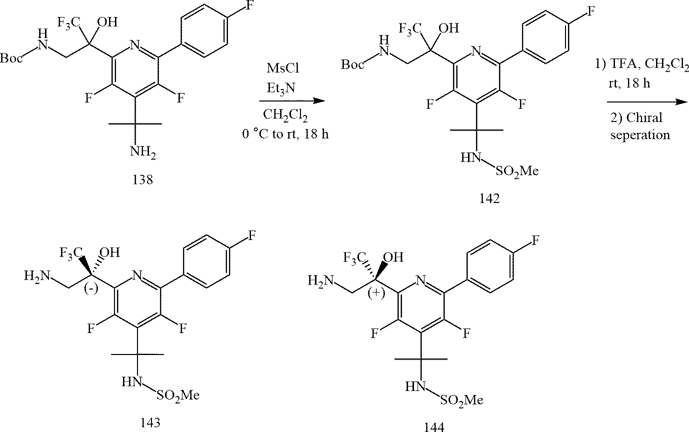

### Tert-Butyl [2-[3,5-Difluoro-6-(4-Fluorophenyl)-4-{2-[(Methanesulfonyl)Amino]Propan-2-yl}Pyridin-2-yl-3,3,3-Trifluoro-2-Hydroxypropyl}Carbamate 142

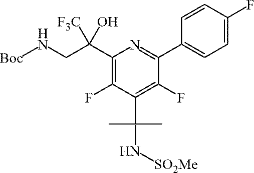 [SMILES: CC(C)(C)OC(=O)NCC(O)(c1nc(-c2ccc(F)cc2)c(F)c(C(C)(C)NS(C)(=O)=O)c1F)C(F)(F)F]

To a solution of 138 (1.10 g, 2.19 mmol) in CH₂Cl₂ (22 mL) at 0° C. was added methanesulfonyl chloride (187 µl, 2.40 mmol) dropwise (the internal temperature of the reaction mixture was maintained between 0 and 3° C. during the addition). Et₃N (304 µL, 2.19 mmol) was added and the reaction mixture was stirred at rt for 18 h. Additional amount of methanesulfonyl chloride (102 µL, 1.31 mmol) and Et₃N (182 µL, 1.31 mmol) were added and the reaction mixture was stirred at rt for another 2 h. Methanesulfonyl chloride (84.8 µL, 1.09 mmol) and Et₃N (182 _{[t}L, 1.31 mmol) were added. The reaction mixture was stirred for 2 h and extra amounts of methanesulfonyl chloride (50.9 _{[t}L, 0.66 mmol) and Et₃N (152 _{[t}L, 1.09 mmol) were added. The reaction mixture was stirred at for another 2 h and the reaction was quenched with NaHCOs (sat., aq.). The mixture was diluted with CH2Cl₂. The layers were separated, and the aqueous phase was extracted with CH₂Cl₂ (twice). The combined organic extracts were dried (MgSO₄). The solids were removed by filtration and the filtrate was concentrated under reduced pressure. The crude mixture was purified by silica column chromatography (heptane/EtOAc, gradient from 90:10 to 60:40) to afford 142 (1.26 g, 90%, 89% pure).

### N-{2-[(-)-3-Amino-1,1,1-Trifluoro-2-Hydroxypropan-2-yl]-3,5-Difluoro-6-(4-Fluorophenyl)Pyridin-4-yl}Propan-2-yl)Methanesulfonamide and 143 A-(2-{2-[(+)-3-Amino-1,1,1-Trifluoro-2-Hydroxypropan-2-y]1-3,5-Difluoro-6-(4-Fluorophenyl)Pyridin-4-yl}Propan-2-vl)Methanesulfonamide 144

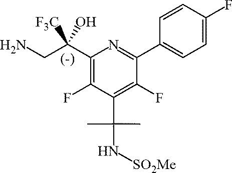

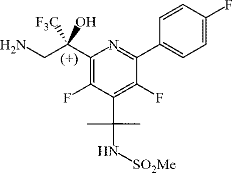

144 TFA (2.23 mL, 29.2 mmol) was added to a solution of 142 (1.25 g, 1.95 mmol) in CH₂Cl2 (34 mL) and the reaction mixture was stirred at rt for 18 h. The reaction mixture was poured into NaHCOs (sat., aq.). The layers were separated and the aqueous phase was extracted with CH₂Cl₂ (3 times). The combined organic extracts were dried (MgSO₄). The solids were removed by filtration and the filtrate was concentrated under reduced pressure. The enantiomers were separated by SFC (stationary phase: Chiralpak IG 5 _{µm} 250 x 20 mm, mobile phase: 90% CO₂, 10% CH₃OH (0.3% i-PrNH₂)) to give 143 (394 mg, 43%); ¹H NMR (400 MHz, DMSO-d₆) δ ppm 1.81 (br d, J=7.7 Hz, 6H), 2.79 - 2.86 (m, 1H), 2.87 (s, 3H), 3.12 (br d, J=13.6 Hz, 1H), 3.54 (br d, J=13.9 Hz, 1H), 6.20 - 6.99 (m, 1H), 7.35 -7.43 (m, 2H), 7.82-8.14 (m, 3H); and 144 (397 mg, 43%); ¹H NMR (400 MHz, DMSO-d₆) δ ppm 1.81 (br d, J=7.7 Hz, 6H), 2.79 - 2.86 (m, 1H), 2.87 (s, 3H), 3.12 (br d, J=13.7 Hz, 1H), 3.54 (br d, J=13.7 Hz, 1H), 6.27 - 6.86 (m, 1H), 7.33 - 7.44 (m, 2H), 7.81 - 8.15 (m, 3H).

### 3.3.13. Synthesis of 148

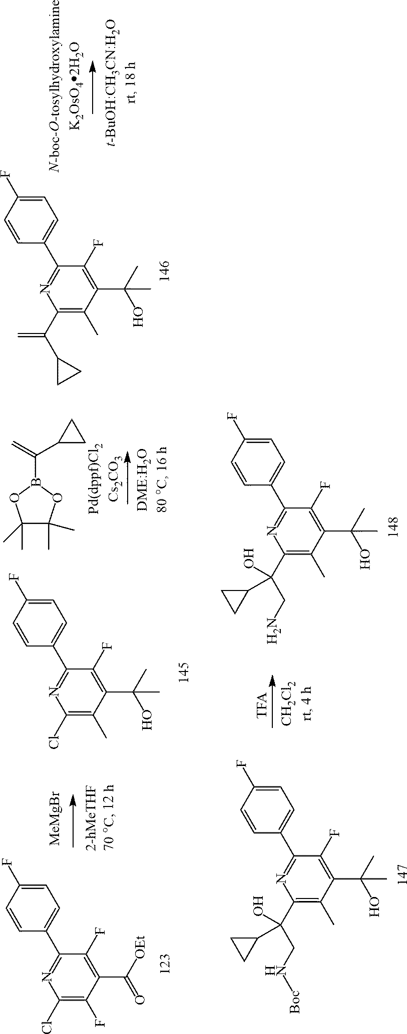

### 2-Chloro-5-fluoro-6-(4-fluorophenyl)-3-methylpyridin-4-y1]propan-2-ol 145

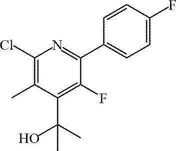 [SMILES: Cc1c(Cl)nc(-c2ccc(F)cc2)c(F)c1C(C)(C)O]

To a solution of 123 (5.05 g, 16.0 mmol) in 2-methylTHF (30 mL) at 0° C. was added CH₃MgBr (1 M in THF, 160 mL, 160 mmol). The reaction mixture was stirred at 70° C. for 12 h, cooled to rt and quenched with NH₄Cl (aq.). The layers were separated, and the aqueous phase was extracted with EtOAc (3 x 150 mL). The combined organic extracts were washed with brine, and dried (Na₂SO₄). The solids were removed by filtration and the filtrate was concentrated under reduced pressure. The residue was purified by silica column chromatography (petroleum ether/EtOAc, gradient from 100:0 to 85:15) to afford 145 (2.2 g, 47%) as a white solid.

### 2-(1-Cyclopropylethenyl)-5-Fluoro-6-(4-Fluorophenyl)-3-Methylpyridin-4-yl]Propan-2-ol 146

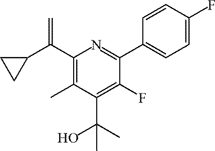 [SMILES: C=C(c1nc(-c2ccc(F)cc2)c(F)c(C(C)(C)O)c1C)C1CC1]

In a Schlenk tube, a mixture of 145 (2.00 g, 6.72 mmol), 2-(1-cyclopropylvinyl)-4,4,5,5-tetramethyl-1,3,2-dioxaborolane (1.56 g, 8.06 mmol) and Cs₂CO₃ (6.57 g, 20.2 mmol) in H₂O (7.11 mL) and DME (35.5 mL) was purged with N₂. PdCl₂(dppf) (246 mg, 0.34 mmol) was added and the mixture was purged again with N₂. The reaction mixture was stirred at 80° C. for 16 h. The mixture was diluted with EtOAc and water, and the layers were separated. The aqueous phase was extracted with EtOAc (twice). The combined organic extracts were dried (MgSO₄). The solids were removed by filtration and the filtrate was concentrated under reduced pressure. The crude mixture was purified by silica column chromatography (heptane/EtOAc, gradient from 100:0 to 75:25) to afford 146 (1.0 g, 45%).

### Tert-Butyl {2-Cyclopropyl-2-[5-Fluoro-6-(4-Fluorophenyl)-4-(2-Hydroxypropan-2-yl)-3-Methylpyridin-2-yl]-2-Hydroxyethyl}Carbamate 147

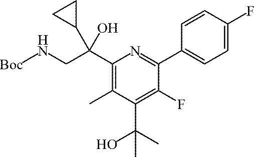 [SMILES: Cc1c(C(O)(CNC(=O)OC(C)(C)C)C2CC2)nc(-c2ccc(F)cc2)c(F)c1C(C)(C)O]

To a solution of 146 (1.00 g, 3.04 mmol) and N-boc-O-tosylhydroxylamine (1.31 g, 4.55 mmol) in t-BuOH (30 mL), CH₃CN (10 mL) and H₂O (5.34 mL) was added K₂O_{S}O₄•2H₂O (224 mg, 0.61 mmol). The reaction mixture was stirred at rt for 18 h. The solvent was removed under reduced pressure and the residual fraction was dissolved in EtOAc and washed with NaHCO₃ (sat., aq.) and brine. The organic layer was dried (MgSO₄). The solids were removed by filtration and the filtrate was concentrated under reduced pressure. The crude mixture was purified by silica column chromatography (heptane/EtOAc, gradient from 100:0 to 50:50) to afford 147 (700 mg, 50%).

### 2-(2-Amino-1-cyclopropyl-1-hydroxyethyl)-5-fluoro-6-(4-fluorophenyl)-3-methylpyridin-4-yl]propan-2-ol 148

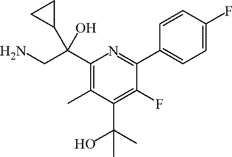 [SMILES: Cc1c(C(O)(CN)C2CC2)nc(-c2ccc(F)cc2)c(F)c1C(C)(C)O]

A solution of 147 (400 mg, 0.87 mmol) in CH₂Cl₂ (10 mL) was treated with TFA (2 mL, 26. mmol) at rt. The reaction mixture was stirred for 4 h and the solvent was removed under reduced pressure. The residue was dissolved in EtOAc and washed with NaHCO₃ (sat., aq.) and water. The organic layer was dried (MgSO₄). The solids were removed by filtration and the filtrate was concentrated under reduced pressure to afford 148 (310 mg, 99%) which was used as such in the next step. LC-MS (method B): Rt = 0.90 min; mass calcd. for C₂₀H₂₄F₂N₂O₂ 362.2, m/z found 363.3 [M+H]⁺.

### 3.3.14. Synthesis of 157 and 158

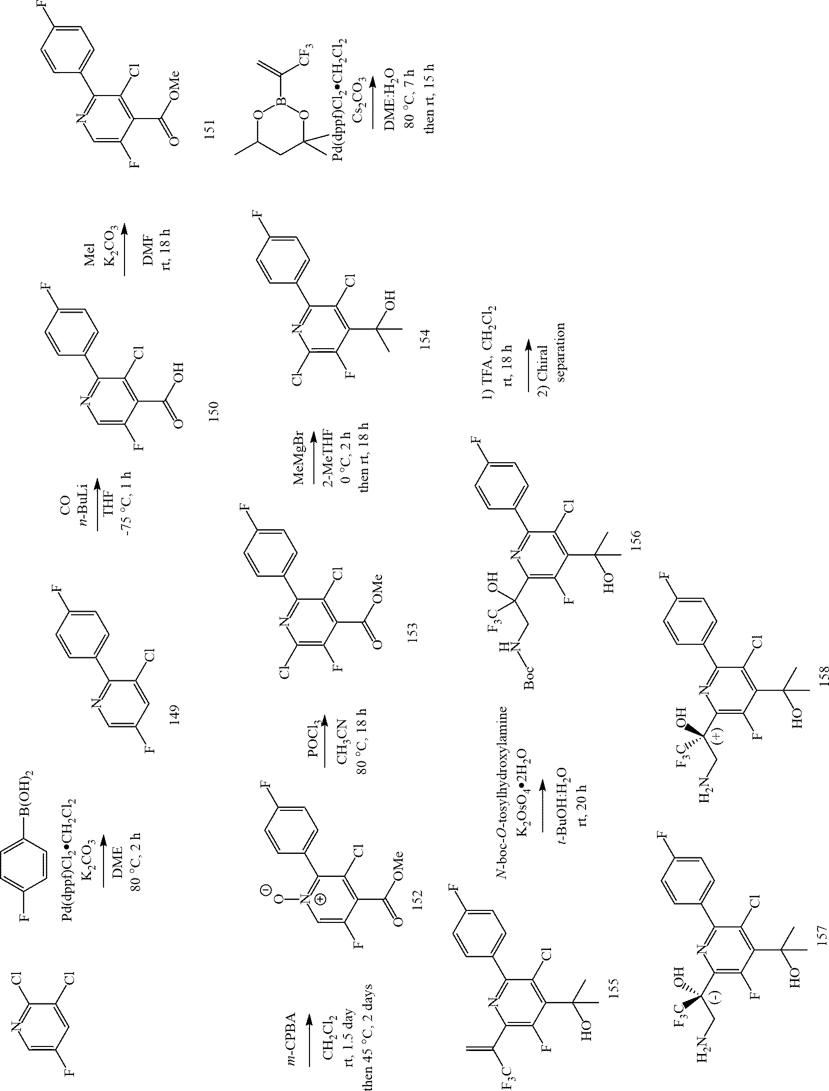

### 3-Chloro-5-fluoro-2-(4-fluorophenyl)pyridine 149

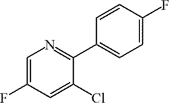 [SMILES: Fc1ccc(-c2ncc(F)cc2Cl)cc1]

A mixture of 2,3-dichloro-5-fluoropyridine (20.0 g, 120 mmol), 4-fluorophenylboronic acid (17.7 g, 127 mmol) and K₂CO₃ (2 M in H₂O, 120 mL) in DME (400 mL) was purged with N₂. Pd(dppf)Cl₂. CH₂Cl₂ (6.89 g, 8.44 mmol) was added and the mixture was purged again with N₂. The reaction mixture was stirred at 80° C. for 2 h. The reaction mixture was cooled to rt and filtered through packed Celite®. The filter cake was washed with EtOAc. The filtrate was diluted with EtOAc and brine. The layers were separated, and the aqueous phase was extracted with EtOAc (twice). The combined organic extracts were dried (MgSO₄). The solids were removed by filtration and the filtrate was concentrated under reduced pressure. The crude mixture was purified by silica column chromatography (heptane/EtOAc, gradient from 95:5 to 80:20) to afford 2 fractions of 149 a pure fraction (16.6 g, 61%) and a fraction containing impurities (9.00 g, 20%, 60% pure).

### 3-Chloro-5-Fluoro-2-(4-Fluorophenyl)Pyridine-4-Carboxylic Acid 150

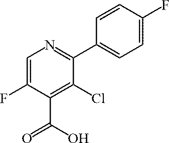 [SMILES: O=C(O)c1c(F)cnc(-c2ccc(F)cc2)c1Cl]

A solution of 149 (108 g, 479 mmol) and n-BuLi (1.6 M in hexane, 330 mL, 528 mmol) in THF (1 L) was stirred at -75° C. for 2 h. CO₂ was bubbled in the reaction mixture for 1 h. Volatiles were evaporated, and the residue was taken up in HCl (3 M aq.). The solids were collected by filtration, washed with water and dried at 70° C. for 50 min to afford 150 (113 g, 88%).

### Methyl 3-Chloro-5-Fluoro-2-(4-Fluorophenyl)Pyridine-4-Carboxylate 151

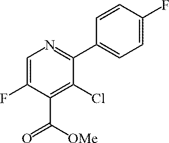 [SMILES: COC(=O)c1c(F)cnc(-c2ccc(F)cc2)c1Cl]

A mixture of 150 (117 g, 434 mmol), CH₃I (32.4 mL, 521 mmol) and K₂CO₃ (132 g, 955 mmol) in DMF (900 mL) was stirred at rt for 18 h. The reaction mixture was diluted with EtOAc and a solution of brine and water (4:1). The layers were separated, and the aqueous phase was extracted with EtOAc (3 times). The combined organic extracts were washed with a solution of brine and water (9:1) (4 times). The organic layer was dried (MgSO₄). The solids were removed by filtration and the filtrate was concentrated under reduced pressure to afford 151 (148 g, quant., 83% pure) as a yellow oil which crystalized on standing.

### Methyl 3-chloro-5-fluoro-2-(4-fluorophenyl)-1-oxo-1λ⁵-pyridine-4-carboxylate 152

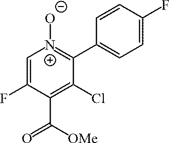 [SMILES: COC(=O)c1c(F)c[n+]([O-])c(-c2ccc(F)cc2)c1Cl]

At 0° C., to a solution of 151 (117 g, 343 mmol, 83% pure) in CH₂Cl₂ (1.3 L) was added m-CPBA (158 g, 686 mmol, 75% pure) portion wise. The reaction mixture was stirred at rt for 24 h. Additional amount of m-CPBA (158 g, 686 mmol, 75% pure) was added portionwise and the reaction mixture was stirred at rt for 6 h, and at 45° C. for 48 h. The reaction was quenched with Na₂S₂CO₃ (sat., aq.). The layers were separated, and the aqueous phase was extracted with CH₂Cl₂ (3 times). The combined organic extracts were washed with a NaOH (1 M, aq.), and dried (MgSO₄). The solids were removed by filtration and the filtrate was concentrated under reduced pressure to afford 152 (119 g, quant., 86% pure) as a yellow solid.

### Methyl 2,5-dichloro-3-fluoro-6-(4-fluorophenyl)pyridine-4-carboxylate 153

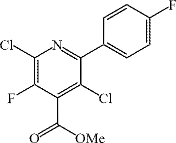 [SMILES: COC(=O)c1c(F)c(Cl)nc(-c2ccc(F)cc2)c1Cl]

To a solution of 152 (88.0 g, 253 mmol, 86% pure) in CH₃CN (880 mL) was added POCl₃ (93.9 mL, 1.01 mol) and the reaction mixture was stirred at 80° C. for 18 h. The reaction mixture was poured dropwise into a solution of water, EtOAc and NaHCO₃ (sat., aq.). The mixture was combined to another fraction (115 mmol). The layers were separated, and the aqueous phase was extracted with EtOAc (twice). The combined organic extracts were dried (MgSO₄). The solids were removed by filtration and the filtrate was concentrated under reduced pressure. The crude mixture was combined with another mixture (43.0 mmol). The residue was diluted with Et₂O and the suspension was stirred at rt for 5 min. The solids were collected by filtration, washed with Et₂O and dried to afford a first crop of 153 (83 g, 64%) as an off-white solid. The filtrate was evaporated, and the residue was dissolved in Et₂O. The suspension was stirred at rt for 5 min, and the solids were collected by filtration, washed with Et₂O and dried to afford a second crop of 153 (28 g, 22%) as an off-white powder. The filtrate was concentrated under reduced pressure and purified by silica column chromatography (heptane/EtOAc, gradient from 95:5 to 90:10) to afford a third crop of 153 (25 g, 11%, 60% pure).

### 2-[2,5-Dichloro-3-fluoro-6-(4-fluorophenyl)pyridin-4-yl]propan-2-ol 154

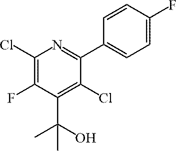 [SMILES: CC(C)(O)c1c(F)c(Cl)nc(-c2ccc(F)cc2)c1Cl]

Under N₂ atmosphere, CH₃MgBr (3 M in Et₂O, 50.3 mL, 151 mmol) was added dropwise to a solution of 153 (16.0 g, 50.3 mmol) in anhydrous 2-MeTHF (250 mL) in an ice bath (the internal temperature was maintained around -2° C.). The reaction mixture was stirred at this temperature for 30 min and at rt for 18 h. The reaction mixture was diluted with EtOAc and NH₄Cl (10%, aq.) was added. The layers were separated, and the aqueous phase was extracted with EtOAc (twice). The combined organic extracts were washed with brine, and dried (MgSO₄). The solids were removed by filtration and the filtrate was concentrated under reduced pressure. The crude mixture was purified by silica column chromatography (heptane/EtOAc, gradient from 95:5 to 85:15) to afford 154 (4.11 g, 26%) as a colorless oil.

### 2-Chloro-5-fluoro-2-(4-fluorophenyl)-6-(3,3,3-trifluoroprop-1-en-2-yl)pyridin-4-yl]propan-2-ol 155

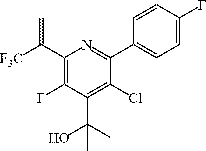 [SMILES: C=C(c1nc(-c2ccc(F)cc2)c(Cl)c(C(C)(C)O)c1F)C(F)(F)F]

A mixture of 154 (15.8 g, 49.7 mmol), 4,4,6-trimethyl-2-(3,3,3-trifluoroprop-1-en-2-yl)-1,3,2-dioxaborinane (20.6 mL, 99.3 mmol) and C_{S2}CO₃ (48.7 g, 150 mmol) in H₂O (35.6 mL) and DME (198 mL) was purged with N₂. Pd(dppf)Cl₂. CH₂Cl₂ (4.06 g, 4.97 mmol) was added and the mixture was purged again with N₂. The reaction mixture was stirred at 80° C. for 7 h and at rt for 15 h. The reaction mixture was diluted with EtOAc and water. The layers were separated, and the aqueous phase was extracted with EtOAc (twice). The combined organic extracts were dried (MgSO₄). The solids were removed by filtration and the filtrate was concentrated under reduced pressure. The crude mixture was purified by silica column chromatography (heptane/EtOAc, gradient from 95:5 to 80:20) to afford 155 (16.7 g, 45%, 50% pure) as an oil.

### T-Butyl {2-[5-Chloro-3-Fluoro-6-(4-Fluorophenyl)-4-(2-Hydroxypropan-2-yl)Pyridin-2-yl]-3,3,3-Trifluoro-2-Hydroxypropyl}Carbamate 156

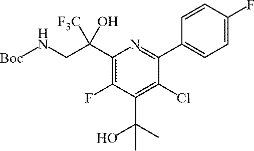 [SMILES: CC(C)(C)OC(=O)NCC(O)(c1nc(-c2ccc(F)cc2)c(Cl)c(C(C)(C)O)c1F)C(F)(F)F]

K₂O_{S}O₄•2H₂O (2.08 g, 5.64 mmol) and N-boc-O-tosylhydroxylamine (17.8 g, 62.0 mmol) were added to a solution of 155 (14.2 g, 28.2 mmol, 75% pure) in t-BuOH (320 mL) and H₂O (20.5 mL). The reaction mixture was stirred at rt for 20 h. The reaction mixture was diluted with water and EtOAc. The mixture was combined with another fraction (19.8 mmol). The layers were separated, and the aqueous phase was extracted with EtOAc (3 times). The combined organic extracts were washed with NaHCO₃ (sat., aq.), and dried (MgSO₄). The solids were removed by filtration and the filtrate was concentrated under reduced pressure. The crude product 156 was used in the next step without further purification.

### (-)Amino-2-[5-ChloroFluoro-6-(4-Fluorophenyl)-4-(2-Hydroxypropan-2-yl)Pyridin-2-yl]-1,1,1-Trifluoropropan-2-ol 157 and (+)Amino-2-[5-ChloroFluoro-6-(4-Fluorophenyl)-4-(2-Hydroxypropan-2-yl)Pyridin-2-yl]-1,1,1-Trifluoropropan-2-ol 158

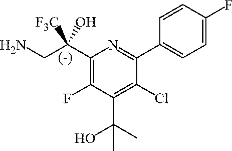

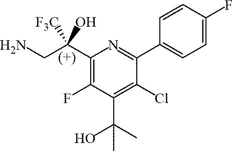

TFA (57 mL, 745 mmol) was added to a solution of 156 in CH₂Cl₂ (710 mL) and the reaction mixture was stirred at rt for 18 h. The reaction mixture was poured slowly into NaHCO₃ (sat., aq.). The layers were separated, and the aqueous phase was extracted with CH₂Cl₂ (twice). The combined organic extracts were dried (MgSO₄). The solids were removed by filtration and the filtrate was concentrated under reduced pressure. The crude mixture was purified by silica column chromatography (CH₂Cl₂/CH₃OH, gradient from 100:0 to 92:8) to afford a mixture of enantiomers (15.8 g, 79% over 2 steps). The enantiomers were separated via chiral SFC (stationary phase: CHIRALPAK AD-H 5 µm 250 x 30 mm, mobile phase: 85% CO₂, 15% CH₃OH (0.6% Et₃N)) to afford 157 (7.8 g, 40%); ¹H NMR (400 MHz, DMSO-d₆) δ ppm 1.58 -1.81 (m, 1H), 1.69 (br s, 6H), 3.08 (d, J=13.6 Hz, 1H), 3.51 (br d, J= 13.7 Hz, 1H), 5.56 (s, 1H), 5.98 - 7.03 (m, 1H), 7.28 - 7.36 (m, 2H), 7.59 - 7.68 (m, 2H);_LC-MS (method K): Rt = 1.26 min; mass calcd. for C₁₇H₁₆ClF₅N₂O₂ 410.1, m/z found 411.2 [M+H]⁺; and 158 (7.38 g, 37%); ¹H NMR (400 MHz, DMSO-d6) δ ppm 1.54 - 1.73 (m, 1H), 1.69 (br s, 6H), 3.08 (br d, J=13.6 Hz, 1H), 3.51 (br d, J=13.4 Hz, 1H), 5.55 (s, 1H), 5.91 - 7.00 (m, 1H), 7.32 (t, J=8.9 Hz, 2H), 7.64 (dd, J=8.6, 5.6 Hz, 2H);_LC-MS (method K): Rt = 1.26 min; mass calcd. for C₁₇H₁₆ClF₅N₂O₂ 410.1, m/z found 411.2 [M+H]⁺.

### 3.3.15. Synthesis of 166

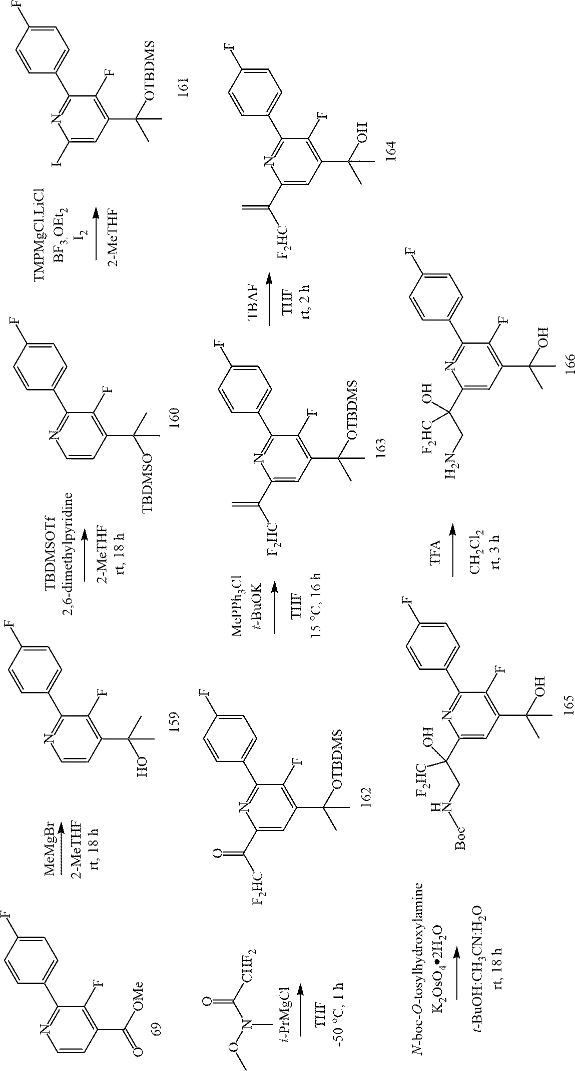

### 2-Fluoro-2-(4-fluorophenyl)pyridin-4-yl]propan-2-ol 159

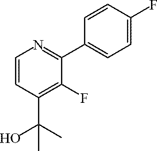 [SMILES: CC(C)(O)c1ccnc(-c2ccc(F)cc2)c1F]

To a solution of 69 (10.0 g, 40.1 mmol) in anhydrous 2-MeTHF (240 mL) at 0° C. under N₂ atmosphere was added CH₃MgBr (3 M, 33.4 mL, 100 mmol). The reaction mixture was stirred at rt for 18 h. the mixture was diluted with EtOAc and quenched with NH₄Cl (10%, aq.). The layers were separated, and the organic phase was dried (MgSO₄). The solids were removed by filtration and the filtrate was concentrated under reduced pressure. The crude mixture was purified by silica column chromatography (heptane/EtOAc, gradient from 95:5 to 70:30) to afford 159 (7.66 g, 77%) as an off-white solid.

### 4-{[Tert-Butyl(Dimethyl)Silyl]Oxy}Propan-2-yl)-3-Fluoro-2-(4-Fluorophenyl)Pyridine 160

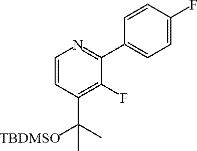 [SMILES: CC(C)(O[Si](C)(C)C(C)(C)C)c1ccnc(-c2ccc(F)cc2)c1F]

t-Butyldimethylsilyl trifluoromethanesulfonate (14.0 mL, 61.0 mmol) was added dropwise to a solution of 159 (7.60 g, 30.5 mmol) and 2,6-dimethylpyridine (10.7 mL, 91.5 mmol) in CH₂Cl₂ (160 mL). The reaction mixture was stirred at rt for 4h. The reaction was diluted with water. The layers were separated, and the organic phase was dried (MgSO₄). The solids were removed by filtration and the filtrate was concentrated under reduced pressure. The crude mixture was purified by silica column chromatography (heptane/EtOAc, gradient from 100:0 to 70:30) to afford 160 (10.3 g, 93%) as a white solid.

### 4-{[Tert-Butyl(Dimethyl)Silyl]Oxy}Propan-2-yl)-3-Fluoro-2-(4-Fluorophenyl)-6-Iodopyridine 161

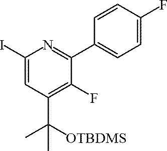 [SMILES: CC(C)(O[Si](C)(C)C(C)(C)C)c1cc(I)nc(-c2ccc(F)cc2)c1F]

To a solution of 160 (2.00 g, 5.50 mmol) in 2-MeTHF was added BF₃.OEt₂ (9.90 mmol) at -10° C. The mixture was stirred for 15 min and 2,2,6,6-tetramethylpiperidinylmagnesium chloride LiCl complex (solution in 2-MeTHF, 16.5 mmol). The mixture was stirred at this temperature for 3.5 h and a solution of I₂ (16.5 mmol) in 2-MeTHF was added while maintaining the temperature of the mixture below 20° C. The reaction mixture was stirred at this temperature for 1.5 h. The reaction was quenched with Na₂CO₃ (aq.) and the mixture warmed to rt. Na₂S₂O₃ (aq.) was added and the mixture was stirred for 30 min and filtered over Celite®. The layers were separated, and the organic phase was concentrated under reduced pressure. The residue was taken up in CH₃OH and concentrated several times under reduced pressure to afford 161.

### 1-(2-{[Tert-Butyl(Dimethyl)Silyl]Oxy}Propan-2-yl)-5-Fluoro-6-(4-Fluorophenyl)Pyridin-2-yl]-2,2-Difluoroethan-1-One 162

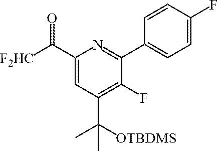 [SMILES: CC(C)(O[Si](C)(C)C(C)(C)C)c1cc(C(=O)C(F)F)nc(-c2ccc(F)cc2)c1F]

A mixture of 161 (5.00 g, 10.2 mmol) and 2,2-difluoro-N-methoxy-N-methylacetamide (1.43 mL, 12.3 mmol) in THF (83 mL) was stirred at -50° C. i-PrMgCl•LiCl (1.3 M in THF, 19.6 mL, 25.5 mmol) was added dropwise. The reaction mixture was stirred at -50° C. for 1 h and warmed to 0° C. The reaction was quenched with NH₄Cl (sat., aq., 10 mL). The layers were separated, and the aqueous phase was extracted with EtOAc. The combined organic extracts were concentrated under reduced pressure. The crude mixture was combined with another fraction (2.04 mmol). The crude mixture was purified by silica column chromatography (heptane/EtOAc, gradient from 98:2 to 95:5) to afford 162 (2.45 g, 85% pure).

### 4-{[Tert-Butyl(Dimethyl)Silyl]Oxy}Propan-2-yl)-6-(3,3-Difluoroprop-1-en-2-yl)-3-Fluoro-2-(4-Fluorophenyl)Pyridine 163

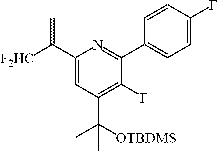 [SMILES: C=C(c1cc(C(C)(C)O[Si](C)(C)C(C)(C)C)c(F)c(-c2ccc(F)cc2)n1)C(F)F]

To a solution of methyltriphenylphosphonium chloride (1.91 g, 6.12 mmol) in THF (100 mL) was added t-BuOK (686 mg, 6.12 mmol). The mixture was stirred at 15° C. for 30 min. A solution of 162 (2.25 g, 5.10 mmol) in THF (25 mL) was added dropwise, and the reaction mixture was stirred at 15° C. for 16 h. Additional amount of t-BuOK (686 mg, 6.12 mmol) and methyltriphenylphosphonium chloride (1.91 g, 6.12 mmol) were added at 15° C. and the reaction mixture was stirred for another 18 h. The reaction mixture was diluted with water (100 mL) and extracted with EtOAc (2 x 300 mL). The combined organic extracts were washed with brine (100 mL) and dried (MgSO₄). The solids were removed by filtration and the filtrate was concentrated under reduced pressure. The residue was purified by silica column chromatography (CH₂Cl₂) to afford 163 (1.2 g, 54%) as a white solid.

### 2-(3,3-Difluoroprop-1-en-2-yl)-3-fluoro-2-(4-fluorophenyl)pyridin-4-yl]propan-2-ol 164

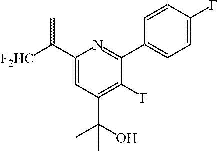 [SMILES: C=C(c1cc(C(C)(C)O)c(F)c(-c2ccc(F)cc2)n1)C(F)F]

A mixture of 163 (1.10 g, 2.50 mmol) and TBAF (1 M in THF, 5.00 mL, 5.00 mmol) in THF (20 mL) was stirred at rt for 2 h. The reaction mixture was diluted with water and EtOAc. The layers were separated, and the organic layer was washed with brine and dried (MgSO₄). The solids were removed by filtration and the filtrate was concentrated under reduced pressure. The crude mixture was purified by silica column chromatography (CH₂Cl₂) to afford 164 (800 mg, 98%).

### Tert-Butyl {3,3-Difluoro-2-[5-Fluoro-4-(2-Hydroxvpropan-2-yl)-6-Phenylpyridin-2-yl]-2-Hydroxypropyl}Carbamate 165

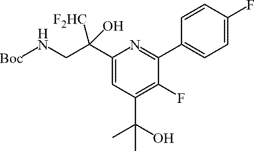 [SMILES: CC(C)(C)OC(=O)NCC(O)(c1cc(C(C)(C)O)c(F)c(-c2ccc(F)cc2)n1)C(F)F]

To a solution of 164 (800 mg, 2.46 mmol) and t-butyl ([(4-methylphenyl)sulfonyl]oxy)carbamate (1.07 g, 3.74 mmol) in t-BuOH (30 mL), CH₃CN (10 mL) and H₂O (4.3 mL) was added K₂O_{S}O_{4•}2H₂O (181 mg, 0.49 mmol). The reaction mixture was stirred at rt for 18 h. The reaction mixture was extracted with EtOAc, washed with water (twice) and brine, and dried (MgSO₄). The solids were removed by filtration and the filtrate was concentrated under reduced pressure. The crude mixture was purified by silica column chromatography (CH₂Cl₂/CH₃OH, gradient from 100:0 to 90:10) to afford 165 (950 mg, 84%).

### 3-Amino-1,1-difluoro-2-[5-fluoro-6-(4-fluorophenyl)-4-(2-hydroxypropan-2-yl)pyridin-2-yl]propan-2-ol 166

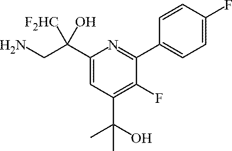 [SMILES: CC(C)(O)c1cc(C(O)(CN)C(F)F)nc(-c2ccc(F)cc2)c1F]

A solution of 165 (940 mg, 2.05 mmol) in CH₂Cl₂ (25 mL) was treated with TFA (5 mL) at rt. The reaction mixture was stirred for 3 h. The solvent was removed under reduced pressure and the residue was dissolved in EtOAc and washed with water. The organic layer was dried (MgSO₄). The solids were removed by filtration and the filtrate was evaporated under reduced pressure to dryness. The crude mixture was purified by silica column chromatography (heptane/EtOAc, gradient from 95:5 to 90:10) to afford 166 (412 mg, 56%) as a white solid. ¹H NMR (400 MHz, DMSO-d₆) δ ppm 7.92 - 7.99 (m, 3H), 7.31 - 7.38 (m, 2H), 6.34 (t, J=54.8 Hz, 1H), 5.89 - 6.12 (m, 1H), 5.60 (s, 1H), 3.08 - 3.14 (m, 1H), 2.99 - 3.05 (m, 1H), 1.55 (s, 6H), 1.32 - 1.61 (m, 2H); LC-MS (method B): Rt = 0.86 min; mass calcd. for C₁₇H₁₈F₄N₂O₂ 358.1, m/z found 359.0 [M+H]⁺.

### 3.3.16. Synthesis of 169 and 170

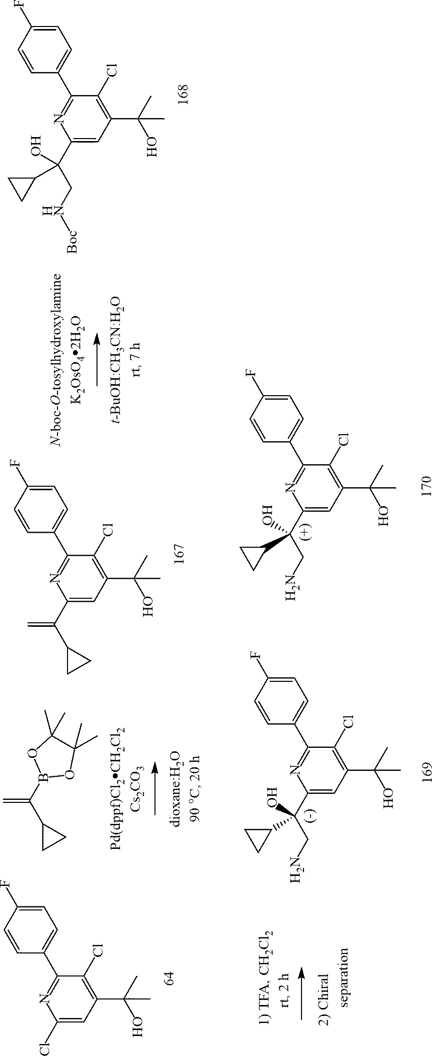

### 2-Chloro-6-(1-cyclopropylethenyl)-2-(4-fluorophenyl)pyridin-4-yl]propan-2-ol 167

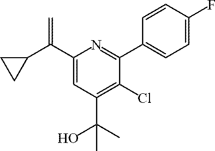 [SMILES: C=C(c1cc(C(C)(C)O)c(Cl)c(-c2ccc(F)cc2)n1)C1CC1]

To a solution 64 (5.33 g, 17.7 mmol) in 1,4-dioxane (76 mL) were added a solution of Cs₂CO₃ (16.2 g, 49.7 mmol) in water (8 mL), 2-(1-cyclopropylvinyl)-4,4,5,5-tetramethyl-1,3,2-dioxaborolane (6.91 g, 35.6 mmol) and Pd(dppf)Cl₂. CH₂Cl₂ (1.52 g, 1.86 mmol) under N₂. The reaction mixture was stirred at 90° C. for 20 h in a sealed reactor. The reaction mixture was cooled to rt, diluted with EtOAc and filtered over Celite®. The filtrate was extracted with EtOAc. The combined organic extracts were washed with water and brine, and dried (MgSO₄). The solids were removed by filtration and the filtrate was concentrated under reduced pressure. The crude mixture was purified by silica column chromatography (CH₂Cl₂) to afford 167 (3.74 g, 64%) as a yellow solid.

### T-Butyl {2-[5-Chloro-6-(4-Fluorophenyl)-4-(2-Hydroxypropan-2-yl)Pyridin-2-yl]-2-Cyclopropyl-2-Hydroxyethyl}Carbamate e 168

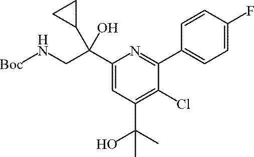 [SMILES: CC(C)(C)OC(=O)NCC(O)(c1cc(C(C)(C)O)c(Cl)c(-c2ccc(F)cc2)n1)C1CC1]

To a solution of 167 (3.67 g, 11.1 mmol) and N-boc-O-tosylhydroxylamine (9.20 g, 32.0 mmol) in t-BuOH (65 mL), CH₃CN (18 mL) and H₂O (18 mL) was added K₂O_{S}O₄•2H₂O (815 mg, 2.21 mmol). The dark mixture was stirred at rt for 7 h. The mixture was extracted with EtOAc. The combined organic extracts were washed with water and brine, dried (MgSO₄), filtered and concentrated under reduced pressure. The crude mixture was purified by flash column chromatography (CH₂Cl₂/(CH₂Cl₂/CH₃OH, gradient from 100:0 to 90:10) to afford 168 (3.72 g, 72%) as an oil.

### 2{6-[(-)-2-Amino-1-Cyclopropyl-1-Hydroxyethyl]-3-Chloro-2-(4-Fluorophenyl)Pyridin-4-yl}-Propan-2-ol 169 and 2{6-[(+)-2-Amino-1-Cyclopropyl-1-Hydroxyethyl]-3-Chloro-2-(4-Fluorophenyl)Pyridin-4-yl}-Propan-2-ol 170

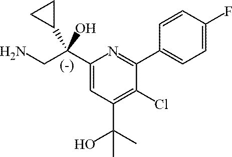

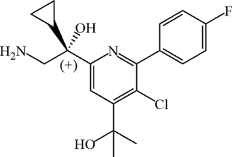

To a solution of 168 (3.70 g, 7.96 mmol) in CH₂Cl₂ (50 mL) was added TFA (6.09 mL, 79.6 mmol). The reaction mixture was stirred at rt for 2 h. The reaction mixture was concentrated under reduced pressure. The residue was diluted with CH₂Cl₂ and the solution was washed with NaHCO₃ (sat., aq., twice) and water. The desired product precipitated and was collected by filtration. ¹H NMR (400 MHz, DMSO-d6, 27° C.) δ ppm 0.06-0.15 (m, 1H), 0.24-0.38 (m, 2H), 0.42-0.50 (m, 1H), 1.18-1.50 (m, 3H), 1.64 (d, J=5.3 Hz, 6H), 2.81 (br d, J=13.2 Hz, 1H), 3.14 (br d, J=13.0 Hz, 1H), 4.83 (br s, 1 H), 5.54 (s, 1H), 7.28 (t, J=8.9 Hz, 2H), 7.61-7.69 (m, 2H), 8.06 (s, 1H). The enantiomers were separated via SFC (stationary phase: Daicel Chiralpak AD_H 5 µm 300 g, mobile phase: heptane (0.2% i-PrNH₂)/EtOH (0.2% i-PrNH₂), gradient from 100:0 to 95:5) to afford 169 (800 mg); [α]_{D}²⁰ -38.93 (c 0.614, DMF); and 170 (800 mg); [α]_{D}²⁰ +38.0 (c 0.25, DMF).

### 3.3.17. Synthesis of 175 and 176

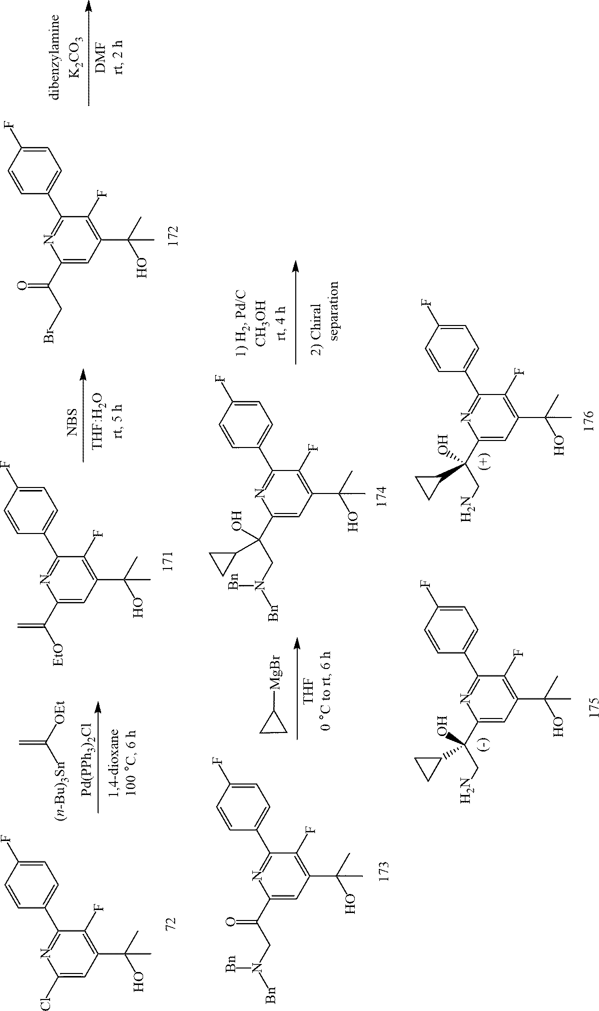

### 2-(1-Ethoxyethenyl)-3-fluoro-2-(4-fluorophenyl)pyridin-4-yl]propan-2-ol 171

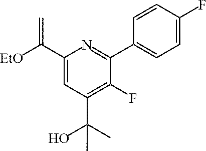 [SMILES: C=C(OCC)c1cc(C(C)(C)O)c(F)c(-c2ccc(F)cc2)n1]

Into a microwave vial equipped with a magnetic stir bar were added tributyl(1-ethoxyvinyl)tin (6.75 mL, 19.4 mmol) and 72 (5.00 g, 17.6 mmol) in 1,4-dioxane (15 mL). Pd(PPh₃)₂Cl₂ (1.24 g, 1.76 mmol) was added and the reaction mixture was stirred at 100° C. for 6 h. The reaction mixture was cooled to rt and filtered through Celite®. The filter cake was washed with 1,4-dioxane (3 times) and the filtrate was concentrated under reduced pressure. The crude mixture was purified by silica column chromatography (heptane/EtOAc, gradient from 100:0 to 0:100) to afford 171 (1.68 g, 30%).

### 2-Bromo[5-fluoro-6-(4-fluorophenyl)-4-(2-hydroxypropan-2-yl)pyridin-2-yl]ethanone 172

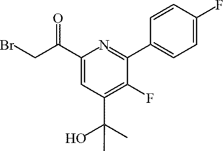 [SMILES: CC(C)(O)c1cc(C(=O)CBr)nc(-c2ccc(F)cc2)c1F]

To a solution of 171 (1.68 g, 5.26 mmol) in THF (14 mL) and water (4 mL) was added NBS (0.94 g, 5.26 mmol) at 0° C. The reaction mixture warmed to rt and was stirred for 5 h. The reaction mixture was diluted with water and the mixture was extracted with EtOAc (3 x 200 mL). The combined organic layers were washed with NaHCO₃ (sat., aq.) and dried (MgSO₄). The solids were removed by filtration and the filtrate was concentrated under reduced pressure to afford 172 (crude) as a yellow solid. The crude was used in the next step without purification.

### 2-(Dibenzylamino)-1-[5-fluoro-6-(4-fluorophenyl)-4-(2-hydroxypropan-2-yl)pyridin-2-yl]ethan-1-one 173

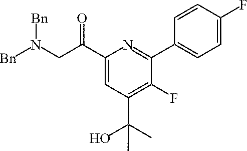 [SMILES: CC(C)(O)c1cc(C(=O)CN(Cc2ccccc2)Cc2ccccc2)nc(-c2ccc(F)cc2)c1F]

Crude 172 (11 g) was added to a suspension of K₂CO₃ (4.50 g, 32.7 mmol) and dibenzylamine (6 mL, 31.2 mmol) in DMF (40 mL). The reaction mixture was stirred at rt for 2 h. The reaction mixture was diluted with water (600 mL) and partitioned with EtOAc (4 x 200 mL). The organic layers were combined and dried (MgSO₄). The solids were removed by filtration and the filtrate was passed through packed alumina and concentrated under reduced pressure. The crude mixture was purified by silica column chromatography (heptane/EtOAc, gradient from 100:0 to 0:100) to afford 173 (1.38 g) as a yellow oil.

### 2-{6-Cyclopropyl-2-(dibenzylamino)-1-hydroxyethyl]-3-fluoro-2-(4-fluorophenyl)pyridin-4-yl}propan-2-ol 174

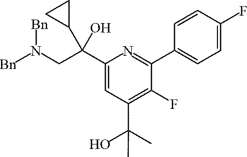 [SMILES: CC(C)(O)c1cc(C(O)(CN(Cc2ccccc2)Cc2ccccc2)C2CC2)nc(-c2ccc(F)cc2)c1F]

Into a microwave vial equipped with a magnetic stir bar were added 173 (6.68 g, 13.7 mmol, not pure) in anhydrous THF (100 mL). The vial was sealed and cooled to 0° C. Then, cyclopropylmagnesium bromide (1 M in 2-MeTHF, 70 mL, 70 mmol) was added. The reaction was allowed to reach rt over 3 h. An additional amount of cyclopropylmagnesium bromide (1 M in 2-MeTHF, 27 mL, 27 mmol) was added at rt. The reaction mixture was stirred for 3 h. The reaction mixture was diluted with CH₃OH (5 mL), and NaHCO₃ (sat., aq.) was added. The layers were separated, and the aqueous phase was extracted with EtOAc (5 x 200 mL). The combined organic layers were dried (MgSO₄). The solids were removed by filtration and the filtrate was concentrated under reduced pressure. The crude mixture was purified by silica column chromatography (heptane/EtOAc, gradient from 100:0 to 80:20) to afford 174 (2.83 g, 39%) as a yellow oil.

### 2-{6-[(-)-2-Amino-1-cyclopropyl-1-hydroxyethyl]-3-fluoro-2-(4-fluorophenyl)pyridin-4-yl)propan-2-ol 175 and 2-{6-[(+)-2-amino-1-cyclopropyl-1-hydroxyethyl]-3-fluoro-2-(4-fluorophenyl)pyridin-4-yl}propan-2-ol 176

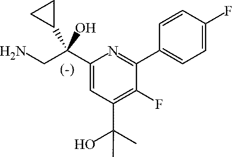

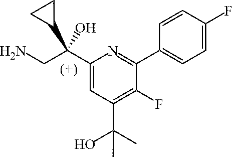

Into an erlenmeyer flask were added a solution of 174 (2.83 g, not pure) in CH₃OH (100 mL). The flask was sparged with N₂ and 10%Pd/C (700 mg) was added. The flask was sealed and exposed to H₂. After 4 h at rt, the reaction mixture was filtered through Celite®. The filtrate was concentrated under reduced pressure. The enantiomers were separated by SFC (stationary phase: Chiralpak Daicel IC 20 x 250 mm, mobile phase: CO₂, EtOH + 0.4% i-PrNH₂) to afford 175 (507 mg, 27%); LC-MS (method C): Rt = 1.55 min; mass calcd. for C₁₉H₂₂F₂N₂O₂ 348.2, m/z found 349 [M+H]⁺; [α]_{D}²⁰ -34.7 (c 0.36, DMF); and 176 (503 mg, 27%); LC-MS (method C): Rt = 1.56 min; mass calcd. for C₁₉H₂₂F₂N₂O₂ 348.2, m/z found 349 [M+H]⁺; [α]_{D}²⁰ +199.2 (c 0.125, DMF).

### 3.3.18. Synthesis of 182

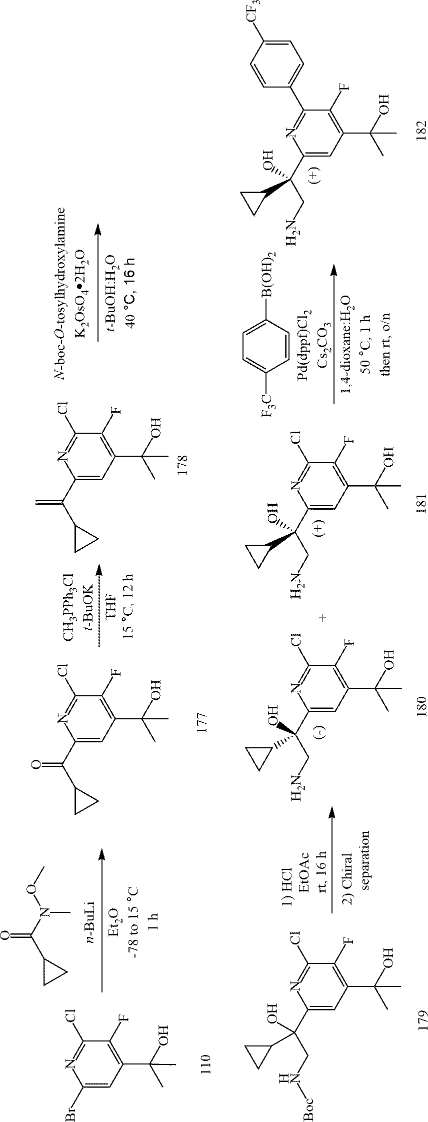

### Chloro-5-fluoro-4-(2-hydroxypropan-2-yl)pyridin-2-yl](cyclopropyl)methanone 177

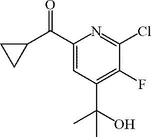 [SMILES: CC(C)(O)c1cc(C(=O)C2CC2)nc(Cl)c1F]

To a solution of 110 (50.0 g, 168 mmol) in Et₂O (900 mL) was added n-BuLi (2.5 M in hexanes, 134 mL, 335 mmol) at -78° C. The reaction mixture was stirred at -78° C. for 10 min and a solution of cyclopropanecarboxylic acid N-methoxy-N-methylamide (32.5 g, 251 mmol) in Et₂O (100 mL) was added. The reaction mixture was stirred for 10 min at -78° C. and at 15° C. for 1 h. NH₄Cl (sat., aq., 100 mL) was added. The layers were separated, and the aqueous phase was extracted with EtOAc (2 x 200 mL). The combined extracts were washed with water (100 mL) and brine (100 mL) and concentrated under reduced pressure. The residue was purified by silica column chromatography (petroleum ether/EtOAc, gradient from 100:0 to 90:10) to afford 177 (170 g, 98%) as a yellow oil.

### 2-Chloro-6-(1-cyclopropylethenyl)-3-fluoropyridin-4-yl]propan-2-ol 178

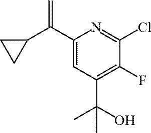 [SMILES: C=C(c1cc(C(C)(C)O)c(F)c(Cl)n1)C1CC1]

To a solution of methyl(triphenyl)phosphonium bromide (156 g, 437 mmol) in THF (500 mL) was added t-BuOK (49.0 g, 437 mmol) and the mixture was stirred at 15° C. for 30 min. To the mixture was added dropwise a solution of 177 (93.8 g, 364.1 mmol) in THF (200 mL) and the reaction mixture was stirred at 15° C. for 12 h. The reaction mixture was diluted with water (200 mL) and the aqueous phase was extracted with EtOAc (2 x 300 mL). The combined organic extracts were washed with water (200 mL) and brine (200 mL), and dried (Na₂SO₄). The solids were removed by filtration and the filtrate was concentrated under reduced pressure. The residue was purified by silica column chromatography (petroleum ether/EtOAc, gradient from 100:0 to 90:10) to afford 178 (70 g, 75 %) as a yellow solid.

### Tert-Butyl {2-(6-Chloro-5-Fluoro-4-(2-Hydroxypropan-2-yl)Pyridin-2-yl]-2-Cyclopropyl-2-Hydroxyethyl}Carbamate 179

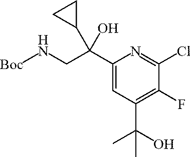 [SMILES: CC(C)(C)OC(=O)NCC(O)(c1cc(C(C)(C)O)c(F)c(Cl)n1)C1CC1]

To a solution of 178 (88.0 g, 310 mmol) in t-BuOH (800 mL) and H₂O (160 mL) was added N-Boc-O-tosylhydroxylamine (107 g, 372 mmol). K₂O_{S}O₄•2H₂O (17.2 g, 46.5 mmol) was added and the reaction mixture was stirred at 40° C. for 16 h. The reaction mixture was diluted with water (200 mL) and the layers were separated. The aqueous phase was extracted with EtOAc (2 x 300 mL). The combined organic extracts were washed with water (200 mL) and brine (200 mL), and dried (Na₂SO₄). The solids were removed by filtration and the filtrate was concentrated under reduced pressure. The residue was purified by silica column chromatography (petroleum ether/EtOAc, gradient from 100:0 to 50:50) to afford 179 (60 g, 50%) as a yellow oil.

### 2-{6-[(-)-2-Amino-1-Cyclopropyl-1-Hydroxyethyl]-2-Chloro-3-Fluoropyridin-4-yl}Propan-2-ol 180 and 2-{6-[(+)-2-Amino-1-Cyclopropyl-1-Hydroxyethyl]-2-Chloro-3-Fluoropyridin-4-yl}Propan-2-ol 181

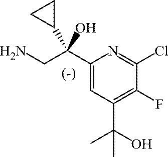

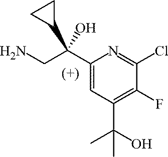

To a solution of 179 (60.0 g, 154 mmol) in EtOAc (200 mL) was added HCl (4 M in EtOAc, 300 mL). The white suspension was stirred at rt for 16 h. The reaction mixture was poured into petroleum ether (1.5 L) and the solid was collected by filtration. The solid was basified with NaOH (0.5 M, aq., 1.2 L) and extracted with a mixture of CH₂Cl₂ and CH₃OH (10:1, 2 x 1.3 L). The combined organic extracts were dried (Na₂SO₄). The solids were removed by filtration and the filtrate was concentrated under reduced pressure. The residue was triturated in petroleum ether and CH₂Cl₂ (10: 1, 400 mL), and collected by filtration to afford a mixture of enantiomers (21 g, 45%, 95% purity) as white solid. The enantiomers were separated via SFC (stationary phase: Chiralpak Diacel AD 20 x 250 mm, mobile phase: CO₂, i-PrOH + 0.4% i-PrNH2) to afford 180; [α]_{D}²⁰ -32.6 (c 0.31, DMF); and 181; [α]_{D}²⁰ +28.9 (c 0.35, DMF).

### 2-{6-[(+)-2-Amino-1-cyclopropyl-1-hydroxyethyl]-3-fluoro-2-[4-(trifluoromethyl)phenyl]pyridin-4-yl}propan-2-ol 182

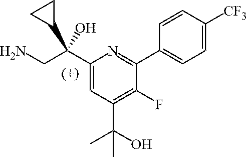

To a mixture of 181 (2.89 g, 10.0 mmol) and 4-(trifluoromethyl)phenylboronic acid (2.28 g, 12.0 mmol) in 1,4-dioxane (90 mL) was added a solution of C_{S2}CO₃ (7.20 g, 22.0 mmol) in water (10 mL). The mixture was degassed for 15 min with N₂. PdCl₂(dppf) (368 mg, 0.50 mmol) was added and the reaction mixture was stirred at 50° C. for 1 h. The reaction mixture was cooled to rt and stirred overnight. The residue was dissolved in CH₂Cl₂. The solution was dried (MgSO₄). The solids were removed by filtration and the filtrate was concentrated under reduced pressure. The crude mixture was purified by silica column chromatography (CH₂Cl₂/7M NH₃ in CH₃OH, 95/5). The residue was triturated in DIPE and the precipitate was collected by filtration and dried under vacuum to afford 182 (1.93 g, 48%). ¹H NMR (400 MHz, DMSO-d6) δ ppm 0.08 - 0.16 (m, 1H), 0.25 - 0.40 (m, 2H), 0.41 - 0.51 (m, 1H), 1.28 - 1.36 (m, 1H), 1.56 (d, J=4.9 Hz, 6H), 2.87 (d, J=13.0 Hz, 1H), 3.20 (d, J=13.0 Hz, 1H), 4.87 (s, 1H), 5.59 (s, 1H), 7.88 (d, J=8.1 Hz, 2H), 7.93 (d, J=5.7 Hz, 1H), 8.11 (d, J=8.1 Hz, 2H); LC-MS (method H): Rt = 1.90 min; mass calcd. for C₂₀H₂₂F₄N₂O₂ 398.2, m/z found 399.3 [M+H]⁺.

### 3.3.19. Synthesis of 183

### 2-{6-[(+)-2-Amino-1-cyclopropyl-1-hydroxyethyl]-2-(4-chlorophenyl)-3-fluoropyridin-4-yl}propan-2-ol 183

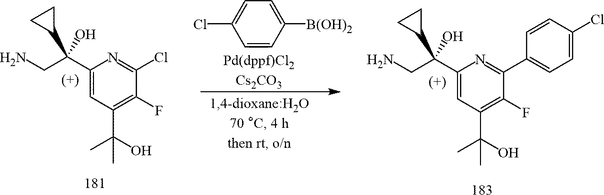

To a mixture of 181 (2.89 g, 10 mmol) and 4-chlorophenylboronic acid (2.00 g, 12.79 mmol) in 1,4-dioxane (90 mL) was added a solution of C_{S2}CO₃ (7.20 g, 22.0 mmol) in water (10 mL). The mixture was degassed for 15 min with N₂. PdCl₂(dppf) (368 mg, 0.50 mmol) was added and the reaction mixture was stirred at 70° C. for 4 h. The mixture was cooled to rt and stirred overnight under N₂. The residue was dissolved in CH₂Cl₂ and dried (MgSO₄). The solids were removed by filtration and the filtrate was evaporated under reduced pressure. The crude was purified by silica column chromatography (CH₂Cl₂/7M NH₃ in CH₃OH, 95/5). The crude was triturated in DIPE. The precipitate was collected by filtration and dried under vacuum to afford 183 (2.2 g, 60%). LC-MS (method H): Rt = 1.76 min; mass calcd. for C₁₉H₂₂ClFN₂O₂ 364.1, m/z found 365.1 [M+H]⁺.

### 3.3.20. Synthesis of 184

### 2-{6-[(+)-2-Amino-1-cyclopropyl-1-hydroxyethyl]-2-(4-cyclopropylphenyl)-3-fluoropyridin-4-yl}propan-2-ol 184

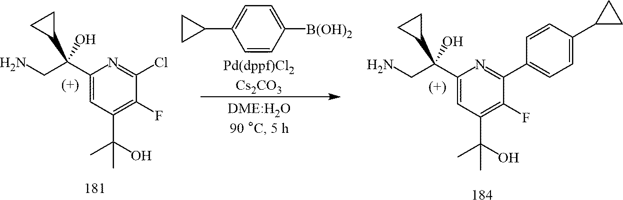

A microwave vial was charged with 181 (0.25 g, 0.87 mmol), 4-cyclopropylphenylboronic acid (168 mg, 1.04 mmol), C_{S2}CO₃ (846 mg, 2.60 mmol), 1,4-dioxane (5 mL), and water (1 mL). The vial was sealed and degassed with N₂. Pd(dppf)Cl₂ (31.7 mg, 43.3 µmol) was added and the vial was sealed. The reaction mixture was shacked at 90° C. for 5 h. The mixture was diluted with CH₂Cl₂ and partitioned with water. The organic layer was dried (MgSO₄). The solids were removed by filtration and the filtrate was concentrated under reduced pressure. The crude mixture was purified by silica column chromatography (CH₂Cl₂/7M NH₃ in CH₃OH, gradient from 100:0 to 90:10) to afford 184 (195 mg, 43%, 71% pure). LC-MS (method G): Rt = 1.96 min; mass calcd. for C₂₂H₂₇FN₂O₂ 370.2, m/z found 371.2 [M+H]⁺.

### 3.3.21. Synthesis of 185

### 2-{6-[(+)-2-Amino-1-cyclopropyl-1-hydroxyethyl]-3-fluoro-2-(3-fluorophenyl)pyridin-4-yl}propan-2-ol 185

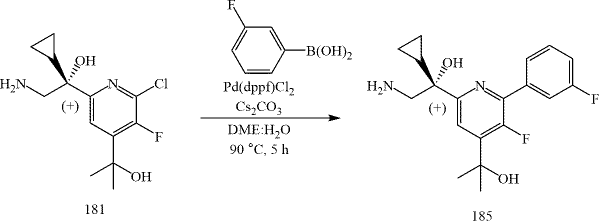

A microwave vial was charged with 181 (0.25 g, 0.86 mmol), 3-fluorophenylboronic acid (144 mg, 1.03 mmol), C_{S2}CO₃ (840 mg, 2.58 mmol), 1,4-dioxane (5 mL), and water (1 mL). The vial was sealed and degassed with N₂. Pd(dppf)Cl₂ (31.4 mg, 43.0 µmol) was added and the vial was sealed. The reaction mixture was shacked at 90° C. for 5 h. The mixture was diluted with CH₂Cl₂ and partitioned with water. The organic layer was dried (MgSO₄). The solids were removed by filtration and the filtrate was concentrated under reduced pressure. The crude mixture was purified by silica column chromatography (CH₂Cl₂/7M NH₃ in CH₃OH, gradient from 100:0 to 90:10) to afford 185 (190 mg, 53%, 83% pure). LC-MS (method G): Rt = 1.71 min; mass calcd. for C₁₉H₂₂F₂N₂O₂ 348.2, m/z found 349.2 [M+H]⁺.

### 3.3.22. Synthesis of 186

### 2-{6-[(+)-2-Amino-1-cyclopropyl-1-hydroxyethyl]-2-(3,4-difluorophenyl)-3-fluoropyridin-4-yl}propan-2-ol 186

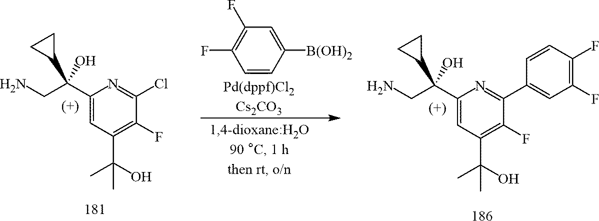

To a mixture of 181 (3.46 g, 12.0 mmol) and 3,4-difluorophenylboronic acid (2.27 g, 14.4 mmol) in 1,4-dioxane (90 mL) was added a solution of C_{S2}CO₃ (8.60 g, 26.4 mmol) in water (10 mL). The mixture was degassed for 15 min with N₂. PdCl₂(dppf) (460 mg, 0.62 mmol) was added and the reaction mixture was stirred at 90° C. for 1 h. The reaction mixture was cooled to rt and stirred overnight. The residue was dissolved in CH₂Cl₂. The solution was dried (MgSO₄). The solids were removed by filtration and the filtrate was concentrated under reduced pressure. The crude mixture was purified by silica column chromatography (CH₂Cl₂/7M NH₃ in CH₃OH, 95/5). The residue was triturated in DIPE, and the precipitate was collected by filtration and dried under vacuum to afford 186 (2.55 g, 58%). ¹H NMR (400 MHz, DMSO-d6) δ ppm 0.07 - 0.15 (m, 1H), 0.24 - 0.39 (m, 2H), 0.43 - 0.51 (m, 1H), 1.27 - 1.36 (m, 1H), 1.54 (d, J=5.3 Hz, 6H), 2.86 (d, J=13.0 Hz, 1H), 3.19 (d, J=13.0 Hz, 1H), 4.85 (s, 1H), 5.57 (s, 1H), 7.57 (dt, J=10.6, 8.5 Hz, 1H), 7.76 - 7.82 (m, 1H), 7.88 (d, J=5.7 Hz, 1H), 7.94 (ddd, J=11.7, 8.2, 1.6 Hz, 1H); LC-MS (method H): Rt = 1.76 min; mass calcd. for C₁₉H₂₁F₃N₂O₂ 366.2, m/z found 367.3 [M+H]⁺.

### 3.3.23. Synthesis of 189 and 190

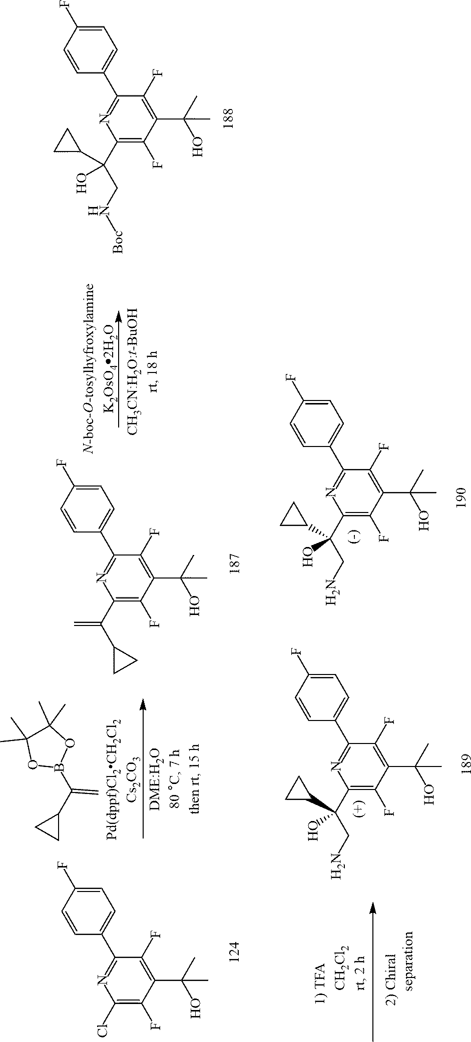

### 2-(1-Cyclopropylethenyl)-3,5-difluoro-6-(4-fluorophenyl)pyridin-4-yl]propan-2-ol 187

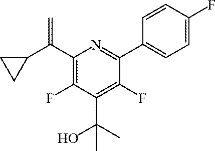 [SMILES: C=C(c1nc(-c2ccc(F)cc2)c(F)c(C(C)(C)O)c1F)C1CC1]

In a Schlenk tube, a mixture of 124 (1.00 g, 3.15 mmol), 2-(1-cyclopropylvinyl)-4,4,5,5-tetramethyl-1,3,2-dioxaborolane (611 mg, 3.15 mmol) and C_{S2}CO₃ (3.09 g, 9.49 mmol) in water (2.6 mL) and DME (16.6 mL) was purged with N₂. Pd(dppf)Cl₂. CH₂Cl₂ complex (129 mg, 0.16 mmol) was added and the mixture was purged again with N₂. The reaction mixture was stirred at 80° C. for 7 h then at rt for 15 h. The reaction mixture was diluted with EtOAc and water. The layers were separated, and the aqueous phase was extracted with EtOAc (twice). The combined organic extracts were dried (MgSO₄). The solids were removed by filtration and the filtrate was evaporated under reduced pressure. The crude was purified by silica column chromatography (heptane/EtOAc, 75:25) to afford 187 (0.93 g, 89%) as an orange oil.

### Tert-Butyl {2-Cyclopropyl-2-[3,5-Difluoro-6-(4-Fluorophenyl)-4-(2-Hydroxypropan-2-yl)Pyridin-2-yl]-2-Hydroxyethyl}Carbamate 188

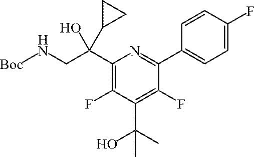 [SMILES: CC(C)(C)OC(=O)NCC(O)(c1nc(-c2ccc(F)cc2)c(F)c(C(C)(C)O)c1F)C1CC1]

To a mixture of 187 (20.0 mg, 60.0 mmol) and N-boc-O-tosylhydroxylamine (26.2 g, 91.2 mmol) in t-BuOH (450 mL), CH₃CN (140 mL) and water (105 mL) was added K₂O_{S}O₄•2H₂O (2.21 g, 6.00 mmol). The reaction mixture was stirred at rt for 18 h. The solvent was removed under reduced pressure. The residue was dissolved in EtOAc and washed with NaHCO₃ (sat., aq.) and brine. The organic layer was dried (MgSO₄). The solids were removed by filtration and the filtrate was concentrated under reduced pressure to afford crude 188.

### (-){2-[2-Amino-1-Cyclopropyl-1-Hydroxyethyl]-3,5-Difluoro-6-(4-Fluorophenyl)Pyridin-4-yl}Propanol 189 and (+){2-[2-Amino-1-Cyclopropyl-1-Hydroxyethyl]-3,5-Difluoro-6-(4-Fluorophenyl)Pyridin-4-yl}Propanol 190

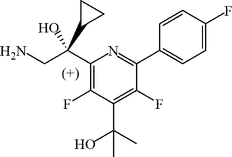

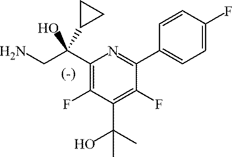

To a solution of 188 (27.0 g, 57.9 mmol) in CH₂Cl₂ (350 mL) was added TFA (50 mL). The reaction mixture was stirred at rt for 2 h. The mixture was concentrated under reduced pressure. The crude mixture was diluted with CH₂Cl₂ and the solution was washed with NaHCO₃ (sat., aq.). The precipitate was removed by filtration and the filtrate was dried (MgSO₄). The solids were removed by filtration and the filtrate was concentrated under reduced pressure. The residue was dissolved in EtOAc, dried and evaporated under reduced pressure. The mixture was combined with other fractions (3.47 mmol, 53.6 mmol and 57.9 mmol). The crude was purified by silica column chromatography (CH₂Cl₂/CH₃OH/NH₃, 97:3)) to afford the racemic mixture. The enantiomers were separated by SFC (Daicel 300 gr OD-H 5um, mobile phase: 93% CO₂, 7% CH₃OH + 0.4% i-PrNH2) to afford 189 (13.2 g, 21%); [α]_{D}²⁰ -6.57 (c 1.05, DMF); and 190 (13.6 g, 21%). [α]_{D}²⁰ +7.53 (c 0.425, DMF).

### 3.3.24. Synthesis of 195 and 196

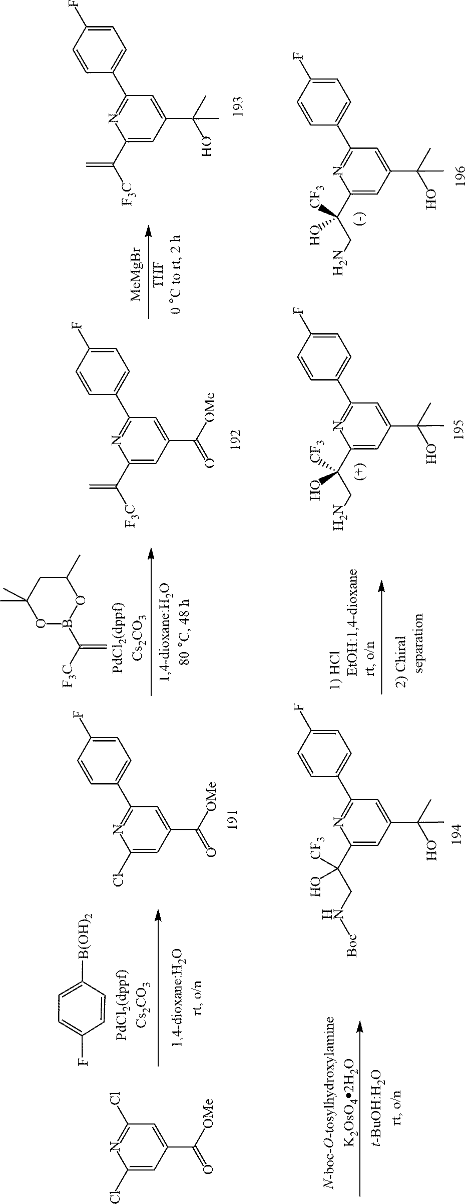

### Methyl 2-chloro-6-(4-fluorophenyl)pyridine-4-carboxylate 191

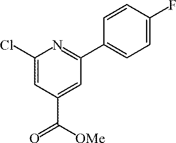 [SMILES: COC(=O)c1cc(Cl)nc(-c2ccc(F)cc2)c1]

To a mixture of degassed 1,4-dioxane (1.3 L) and water (68 mL) were added methyl 2,6-dichloropyridine-4-carboxylate (25.6 g, 124 mmol), C_{S2}CO₃ (61.7 g, 189 mmol), 4-fluorophenylboronic acid (17.0 g, 121 mmol) and PdCl₂(dppf) (6.87 g, 9.39 mmol). The reaction mixture was stirred at rt overnight. The mixture was filtered over decalite and washed with EtOAc. The filtrate was concentrated under reduced pressure to afford crude 191 which was used in the next step without further purification.

### Methyl 2-(4-fluorophenyl)-6-(3,3,3-trifluoroprop-1-en-2-yl)pyridine-4-carboxylate 192

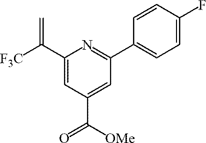 [SMILES: C=C(c1cc(C(=O)OC)cc(-c2ccc(F)cc2)n1)C(F)(F)F]

To a degassed mixture of 1,4-dioxane (1.06 L) and water (56 mL) were added 191 (24.0 g, 55.1 mmol, 61% pure), C_{S2}CO₃ (26.0 g, 80.0 mmol), 4,4,6-trimethyl-2-(3,3,3-trifluoroprop-1-en-2-yl)-1,3,2-dioxaborinane (12.4 g, 55.6 mmol) and PdCl₂(dppf) (3.76 g, 5.14 mmol). The reaction mixture was stirred at 80° C. for 2 days. The mixture was filtered over decalite and washed with EtOAc. The filtrate was concentrated under reduced pressure. The reaction was not finished, so the crude was re-engaged into the reaction. Degassed 1,4-dioxane and water were added, followed by C_{S2}CO₃ (26.0 g, 80.0 mmol), 4,4,6-trimethyl-2-(3,3,3-trifluoroprop-1-en-2-yl)-1,3,2-dioxaborinane (12.4 g, 55.6 mmol) and PdCl₂(dppf) (3.76 g, 5.14 mmol). The reaction mixture was stirred at 80° C. for 2 days. The mixture was filtered over decalite and washed with EtOAc. The filtrate was concentrated under reduced pressure. The crude was purified by silica column chromatography (heptane/CH₂Cl₂, gradient from 70:30 to 30:70) to afford 192 (18.8 g, 38% over 2 steps, 95% pure).

### 2-(4-Fluorophenyl)-6-(3,3,3-trifluoroprop-1-en-2-yl)pyridin-4-yl]propan-2-ol 193

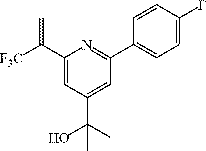 [SMILES: C=C(c1cc(C(C)(C)O)cc(-c2ccc(F)cc2)n1)C(F)(F)F]

A solution of 192 (15.7 g, 45.9 mmol, 95% pure) in THF (400 mL) was stirred under N₂ atmosphere and cooled to 0° C. CH₃MgBr (3.4 M in 2-methylTHF, 40.5 mL, 138 mmol) was added dropwise and the reaction mixture was stirred for 1 h at 0° C., then at rt for 2 h. The mixture was carefully diluted with EtOAc and NH₄Cl (aq.) was added. The layers were separated, and the aqueous phase was extracted with EtOAc. The combined organic extracted were washed with brine and dried (MgSO₄). The solids were removed by filtration and the filtrate was evaporated under reduced pressure. The residue was purified over a silica column (heptane/CH₂Cl₂, gradient from 1:1 to 0:1) to afford 193 (13.8 g, 93%).

### Tert-Butyl {3,3,3-Trifluoro-2-[6-(4-Fluorophenyl)-4-(2-Hydroxypropan-2-yl)Pyridin-2-yl]-2-Hvdroxypropyl}Carbamate 194

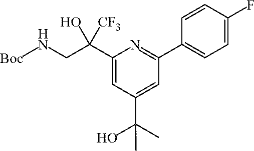 [SMILES: CC(C)(C)OC(=O)NCC(O)(c1cc(C(C)(C)O)cc(-c2ccc(F)cc2)n1)C(F)(F)F]

A solution of 193 (13.8 g, 42.42 mmol) and A-boc-O-tosylhydroxylamine (24.4 g, 84.8 mmol) in t-BuOH (360 mL) and water (40 mL) was stirred at rt. K₂O_{S}O₄•2H₂O (0.78 g, 2.12 mmol) was added and the reaction mixture was stirred at rt overnight. The mixture was concentrated under reduced pressure. the residue was dissolved in CH₂Cl₂ and the solution was washed with water. The organic layer was dried (MgSO₄). The solids were removed by filtration and the filtrate was evaporated under reduced pressure. The crude mixture was purified over a silica column (CH₂Cl₂) to afford 194 (19.4 g, quant.).

### (+)Amino-1,1,1-Trifluoro-2-[6-(4-Fluorophenyl)-4-(2-Hydroxypropan-2-yl)Pyridin-2-yl]Propan-2-ol 195 and (-)Amino-1,1,1-Trifluoro-2-[6-(4-Fluorophenyl)-4-(2-Hydroxypropan-2-yl)Pyridin-2-yl]Propan-2-ol 196

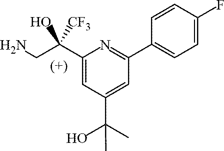

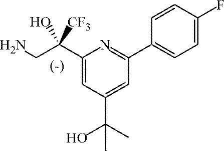

A solution of 194 (19.3 g, 42.0 mmol) in 1,4-dioxane (250 mL) and ethanol (25 mL) was stirred at rt. HCl (4 M in 1,4-dioxane, 52.5 mL, 210 mmol) was added dropwise and the reaction mixture was stirred overnight at rt. The mixture was evaporated under reduced pressure and diluted with ice water. The solution was alkalized with Na₂CO₃ (sat., aq.). the aqueous phase was extracted with CH₂Cl₂ (twice). The combined organic extracted were dried (MgSO₄). The solids were removed by filtration and the filtrate was concentrated under reduced pressure. The residue was triturated in DIPE and CH₃CN (9:1). The solid was collected by filtration and dried under vacuum to afford a mixture of enantiomers (11.3 g). The enantiomers were separated by SFC (stationary phase: Chiralpak Daicel IG 20 x 250 mm, mobile phase: CO₂, EtOH + 0.4% i-PrNH₂) to afford 195 (5.6 g, 37%) LC-MS (method C): Rt = 1.78 min; mass calcd. for C₁₇H₁₈F₄N₂O₂ 358.1, m/z found 359.1 [M+H]⁺and 196 (5.7 g, 38%) LC-MS (method C): Rt = 1.78 min; mass calcd. for C₁₇H₁₈F₄N₂O₂ 358.1, m/z found 359.2 [M+H]⁺.

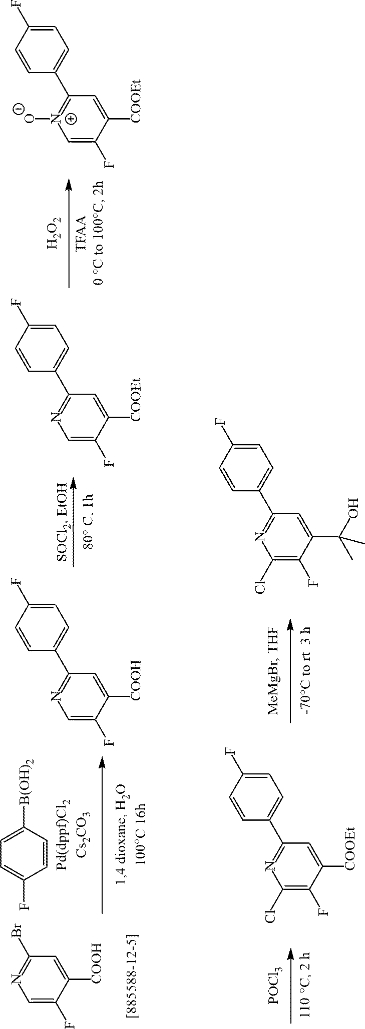

2-bromo-5-fluoroisonicotinic acid (10.0 g, 45.5 mmol), (4-fluorophenyl)boronic acid (7.63 g, 54.5 mmol), C_{S2}CO₃ (44.4 g, 136 mmol) were suspended in H₂O (60 mL) and 1,4-dioxane (240 mL). The mixture was sparged with Ar for 5 minutes and then treated with Pd(dppf)Cl₂ (3.33 g, 4.55 mmol). The mixture was sparged with Ar for another 5 min and then stirred at 100° C. for 16 hours. The reaction mixture was filtered through a pad of Celite® and the pad washed with ethyl acetate (200 mL) and H₂O (120 mL). The layers were separated and the aqueous layer was adjusted to pH = 4 with 1 N HCl. The suspension isolated via filtration. The filter cake was washed with H₂O (50 mL X 3) before drying under reduced pressure to afford 5-fluoro-2-(4-fluorophenyl)isonicotinic acid (9.0 g, 82%).

Thionyl chloride (11.7 mL, 160 mmol) was added to a solution consisting of 5-fluoro-2-(4-fluorophenyl)isonicotinic acid (9.0 g, 38 mmol) and ethanol (100 mL). The resultant mixture was refluxed at 80° C. for 1 hour. The reaction mixture was concentrated to dryness under reduced pressure to give the crude product. The residue was extracted with ethyl acetate (60 mL *2) after adding a solution sat. NaHCO₃ (60 mL). The combined organic extracts were dried over anhydrous Na₂SO₄, filtered, and concentrated to dryness under reduced pressure to give the crude product, which was purified by column chromatography (eluent: petroleum ether: ethyl acetate = 10:1) to afford ethyl 5-fluoro-2-(4-fluorophenyl)isonicotinate (9.2 g, 91%) as a colourless oil.

H₂O₂ (21.0 mL, 209 mmol, 30% purity) was added dropwise to a solution consisting of ethyl 5-fluoro-2-(4-fluorophenyl)isonicotinate (7.0 g, 27 mmol) and TFAA (50 mL) at 0° C. The resultant mixture was stirred at 100° C. for 2 hours. The reaction mixture was poured into sat.Na₂SO₃ (aq, 150 mL), extracted with ethyl acetate (150 mLx 3).

The combined organic extracts were dried over anhydrous Na₂SO₄, filtered, and concentrated to dryness under reduced pressure to give the crude product, which was purified by column chromatography (eluent: petroleum ether: ethyl acetate = 10:1 to 4:1) to afford 4-(ethoxycarbonyl)-5-fluoro-2-(4-fluorophenyl)pyridine 1-oxide (4.5 g, 60%) as a white solid and recovered starting material ethyl 5-fluoro-2-(4-fluorophenyl)isonicotinate (4.0 g, 56%).

A solution consisting of 4-(ethoxycarbonyl)-5-fluoro-2-(4-fluorophenyl)pyridine 1-oxide (4.0 g, 14 mmol) and POCl₃ (25 mL) was stirred at 110° C. for 2 hours. Then about 15 mL of phosphorus oxychloride was removed by distillation.

The resulting mixture was cooled to room-temperature and dropped-wise into water (70 mL) and stirred for 15 min. Saturated ammonium hydroxide solution (about 20 mL) was added to adjust the pH to 7. The resultant mixture was extracted with ethyl acetate (80 mL x 2). The combined organic extracts were dried over anhydrous Na₂SO₄, filtered, and concentrated to dryness under reduced pressure to give the crude product, which was purified by column chromatography (eluent: petroleum ether: ethyl acetate = 10:1 to 4:1) to afford the 4-(ethoxycarbonyl)-5-fluoro-2-(4-fluorophenyl)pyridine 1-oxide (4.2 g, 90%) as a white solid.

Methyl magnesium bromide (18 mL, 54 mmol, 3 M in Et₂O) was added to a solution consisting of 4-(ethoxycarbonyl)-5-fluoro-2-(4-fluorophenyl)pyridine 1-oxide (4.0 g, 13 mmol) in dry THF (30 mL) at -70° C. (dry ice/ethanol). After addition, the reaction mixture was allowed to warm to room-temperature for 3 hours.The reaction mixture was poured into sat. NH₄Cl (100 mL) and extracted with ethyl acetate (100 mL x 2). The combined organic extracts were dried over anhydrous Na₂SO₄, filtered, and concentrated to dryness under reduced pressure to give the crude product, which was purified by column chromatography (eluent: petroleum ether: ethyl acetate = 10:1) to afford 2-(2-chloro-3-fluoro-6-(4-fluorophenyl)pyridin-4-yl)propan-2-ol (4.0 g, 97%) as a light-yellow solid.

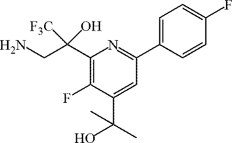 [SMILES: CC(C)(O)c1cc(-c2ccc(F)cc2)nc(C(O)(CN)C(F)(F)F)c1F]

3-amino-1,1,1-trifluoro-2-(3-fluoro-6-(4-fluorophenyl)-4-(2-hydroxypropan-2-yl)pyridin-2-yl)propan-2-ol has been obtained from 2-(2-chloro-3-fluoro-6-(4-fluorophenyl)pyridin-4-yl)propan-2-ol following a similar route to the one described for synthetizing compound 81 and 82.

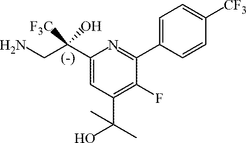

(-)-3-amino-1,1,1-trifluoro-2-(5-fluoro-4-(2-hydroxypropan-2-yl)-6-(4-(trifluoromethyl)phenyl)pyridin-2-yl)propan-2-ol has been obtained from compound 113 and 4-(trifluoromethyl)phenylboronic acid following a similar route to the one described for synthetizing compound 114

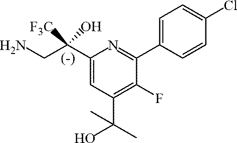

(-)-3-amino-2-(6-(4-chlorophenyl)-5-fluoro-4-(2-hydroxypropan-2-yl)pyridin-2-yl)-1,1,1-trifluoropropan-2-ol has been obtained from compound 113 and 4-chloro phenylboronic acid following a similar route to the one described for synthetizing compound 114

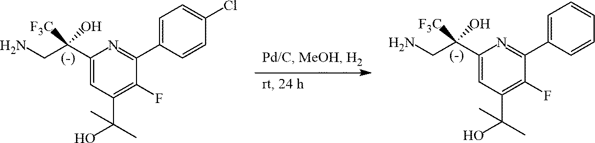

Into a 250 mL Erlenmeyer flask was placed 200 mg of the amine, 100 mL methanol, and approx. 54 mg of 10% Pd/C under nitrogen. The mixture stirred under hydrogen atmosphere for 24h. LCMS shows full conversion to product. The reaction mixture was filtered thought celite, and the solvent was removed under reduced pressure to afford a colourless oil (-)-3-amino-2-(6-phenyl)-5-fluoro-4-(2-hydroxypropan-2-yl)pyridin-2-yl)-1,1,1-trifluoropropan-2-ol (150 mg, 82% yield) which was used as such in the next step.

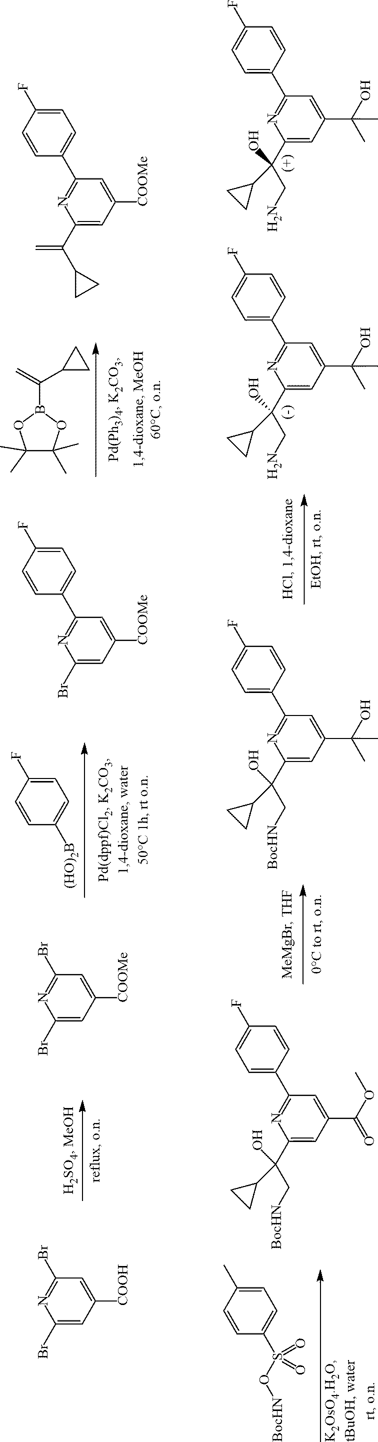

To a solution of 2,6-dibromopyridine-4-carboxylic acid (50 g, 178 mmol) in methanol (250 mL) H₂SO₄ (1 mL, 18.76 mmol) was added dropwise and the mixture was refluxed overnight. The mixture was evaporated, taken up in water, neutralised with sodium bicarbonate and three times extracted with dichloromethane. The organic layer was once washed with water, dried over MgSO₄, filtered and evaporated to afford 2,6-dibromopyridine-4-carboxylate (49.9 g , 95%)

A mixture of methyl 2,6-dibromopyridine-4-carboxylate (14.7 g, 50 mmol), 4-fluorophenylboronic acid (7 g, 50 mmol), K₂CO₃ (27.6 g, 200 mmol) and tetrabutylammonium bromide (1.63 g, 5 mmol) was stirred in 1,4-dioxane (150 mL) and water (50 mL). Degassing with nitrogen was done for fifteen minutes and then [1,1′-Bis-(diphenylphosphino)ferrocene] dichloropalladium (II) (740 mg, 0.1 mmol) was added to the mixture. The vessel was closed and stirred at 50° C. for one hour and overnight at ambient temperature. The mixture was evaporated and the residue was diluted with ice water, extracted two times with ethylacetate and the organic layer was dried over MgSO₄, filtered and evaporated again. The residue was purified over silica gel column chromarography. with heptane/dichloromethane 100/0 to 50/50 as gradient. The corresponding fractions were evaporated. The first residue was triturated in diisopropylether. The white precipitate was collected by filtration and dried in vacuo (3 g, 18% yield). This filtrate was evaporated together with the second impure part that came from column. This fraction was further purified by Prep HPLC yielding to 6.2 g (39% yield) of the desired compound methyl 2-bromo-6-(4-fluorophenyl)isonicotinate.

To a solution of methyl 2-bromo-6-(4-fluorophenyl)isonicotinate (4.6 g, 14.8 mmol) in 1,4-dioxane (40 mL), MeOH (10 mL) under N₂ atmosphere was added 2-(1-cyclopropylvinyl)-4,4,5,5-tetramethyl-1,3,2-dioxaborolane (2.9 g, 14.8 mmol), K₂CO₃ (4.1 g, 29.7 mmol) and Pd(PPh₃)4 (343 mg, 0.3 mmol). The mixture was stirred at 60° C. overnight in a closed vessel. After cooling the mixture was diluted with ethylacetate and twice washed with brine. The organic layer was dried over MgSO₄, filtered and evaporated. The residue was purified over silica column chromatography with heptane/dichloromethane 1/0 to 1/1 as a gradient. The corresponding fractions were evaporated to afford methyl 2-(1-cyclopropylvinyl)-6-(4-fluorophenyl)isonicotinate (3.3 g, 74% yield)

(tert-butoxycarbonylamino) 4-methylbenzenesulfonate (6.4 g, 22.2 mmol) was added to a solution of methyl 2-(1-cyclopropylvinyl)-6-(4-fluorophenyl)isonicotinate (3.3 g, 11.1 mmol) in t-BuOH (50 mL) and water, distilled (5 mL). After this potassium osmate (VI) dihydrate (82 mg, 0.22 mmol) was added to the solution. The solution was allowed to stir overnight at room temperature. The mixture was diluted with ethyl acetate and the solution was washed with water and brine. The organic layer was dried with MgSO₄, filtered and evaporated.

A purification was performed via Prep HPLC (Stationary phase: RP XBridge Prep C18 OBD-10 µm,50×150 mm, Mobile phase: 0.25% NH₄HCO₃ solution in water, CH₃CN) yielding to methyl 2-(1-cyclopropylvinyl)-6-(4-fluorophenyl)isonicotinate (1.4 g, 29% yield). A solution of methyl 2-(1-cyclopropylvinyl)-6-(4-fluorophenyl)isonicotinate (1.4 g, 3.25 mmol) in THF (30 mL) was stirred under nitrogen and cooled on an ice ethanol bath.

Methylmagnesiumbromide (2.87 mL, 3.4 M, 9.77 mmol) was added dropwise and stirring was done for one hour on the cooling bath and overnight at room temperature. The mixture was carefully diluted with ethylacetate and then decomposed with NH₄Cl solution in water and ice. The layers were separated and the waterlayer was extracted one more time with ethylacetate. The combined organic layers were once washed with brine, dried over MgSO₄, filtered and evaporated.

A purification was performed via Prep HPLC (Stationary phase: RP XBridge Prep C18 OBD-10 µm,50×150 mm, Mobile phase: 0.25% NH₄HCO₃ solution in water, CH₃CN) yielding tert-butyl (2-cyclopropyl-2-(6-(4-fluorophenyl)-4-(2-hydroxypropan-2-yl)pyridin-2-yl)-2-hydroxyethyl)carbamate (558 mg, yield 39% yield).

A solution of tert-butyl (2-cyclopropyl-2-(6-(4-fluorophenyl)-4-(2-hydroxypropan-2-yl)pyridin-2-yl)-2-hydroxyethyl)carbamate (558 mg, 1.3 mmol) in 1,4-dioxane (5 mL) and ethanol (5 mL) was stirred at room temperature. HCl (4 M in dioxane) (3.24 mL, 4 M, 12.96 mmol) was added dropwise and stirring was continued overnight at ambient temperature. The mixture was evaporated, dissolved in water, alkalised with Na₂CO₃ solution and two times extracted with dichloromethane. The organic layer was dried over MgSO₄, filtered and evaporated. The residue was purified over a RediSep column with dichloromethane/methanol-NH₃ 98/2 to 95/5 as gradient. The corresponding fractions were evaporated to afford 2-(2-(2-amino-1-cyclopropyl-1-hydroxyethyl)-6-(4-fluorophenyl)pyridin-4-yl)propan-2-ol (430 mg, 100% yield)

The enantiomers were separated by Prep SFC (Stationary phase: Chiralpak Daicel IC 20 x 250 mm, Mobile phase: CO₂, EtOH—iPrOH (50-50) + 0.4% iPrNH2) yielding to (-)-2-(2-(2-amino-1-cyclopropyl-1-hydroxyethyl)-6-(4-fluorophenyl)pyridin-4-yl)propan-2-ol (167 mg , 39% yield) and (+)-2-(2-(2-amino-1-cyclopropyl-1-hydroxyethyl)-6-(4-fluorophenyl)pyridin-4-yl)propan-2-ol (193 mg, 45% yield) .

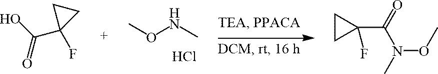

To a solution of 1-fluorocyclopropanecarboxylic acid (10 g, 96.1 mmol) in dry DCM (450 mL) was added dry Et₃N (41.7 mL, 0.7 g/mL, 288.2 mmol) and the mixture was stirred for 2 min. Then solid N,O-dimethylhydroxylamine hydrochloride (11.2 g, 115.3 mmol) was added and the mixture was stirred for 2 min. Finally, 1-Propanephosphonic anhydride (50 W/W% solution in EtOAc) (111 mL, 1.1 g/mL, 191.9 mmol) was added dropwise and the reaction mixture was stirred at rt for 16 h. The RM was poured out in sat NaHCO₃ solution (500 mL), the organic layer was separated, the aqueous phase was extracted two times more with DCM. The combined organic layers were dried over MgSO₄ and evaporated, yielding 1-fluoro-N-methoxy-N-methylcyclopropane-1-carboxamide (13.8 g, yield 98%) as a yellow oil.

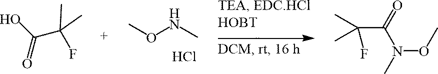

To a solution of 2-fluoro-2-methylpropanoic acid (10 g, 94 mmol) in dichloromethane (500 mL) was added 1-(3-dimethylaminopropyl)-3-ethylcarbodiimide hydrochloride (21 g, 113 mmol), 1-hydroxybenzotriazole (15.2 g, 113 mmol,) and triethylamine (47.7 g, 471 mmol). N,O-dimethylhydroxylamine hydrochloride (11 g, 122 mmol) was added into the mixture. The reaction mixture was stirred at r.t. for 16 h. cooling down to r.t., the resulting mixture was filtered. The filtrate was washed with 2 M HCl solution(3 x 200 mL). Then the mixture was washed with a saturated sodium bicarbonate solution (3 x 200 mL). The organic layers were combined, dried over anhydrous sodium sulfate, filtered and concentrated to afford the 2-fluoro-N-methoxy-N,2-dimethylpropanamide crude 2-fluoro-N-methoxy-N,2-dimethylpropanamide as a yellow oil (10.6 g, 75% yield).

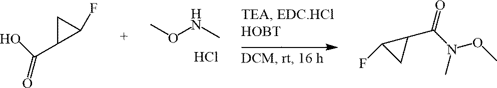

2-Fluoro-N-methoxy-N-methylcyclopropane-1-carboxamide have been prepared following a similar route to the one described for synthetizing 2-fluoro-N-methoxy-N,2-dimethyl-propanamide crude 2-fluoro-N-methoxy-N,2-dimethylpropanamide starting from 2-fluorocyclopropane-1-carboxylic acid.

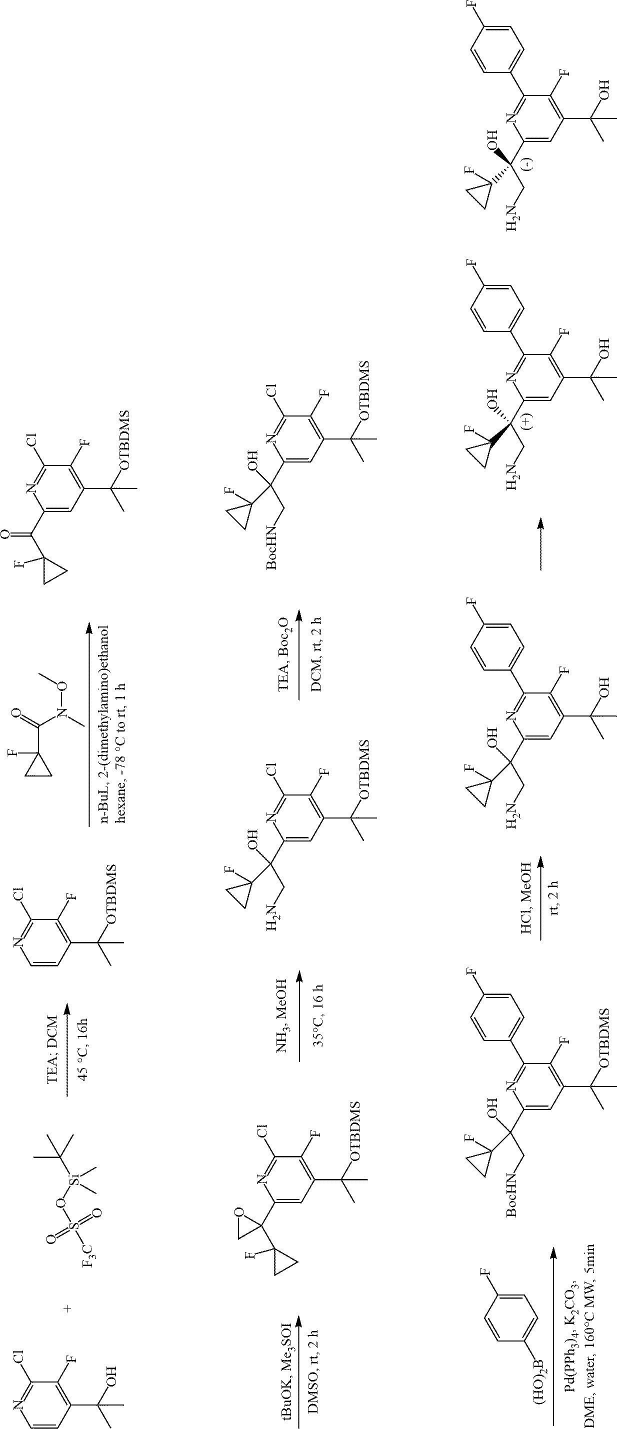

To a solution of 2-(2-chloro-3-fluoropyridin-4-yl)propan-2-ol (49 g, 258 mmol) in dichloromethane (800 mL) was added triethylamine (65 g, 646 mmol,) and trifluoromethanesulfonic acid tert-butyldimethylsilyl ester (102 g, 387 mmol,). Then the reaction mixture was stirred at 45° C. for 16 h. After cooling down to r.t., the reaction was quenched with water (600 mL). The resulting mixture was extracted with dichloromethane (3 x 800 mL). The organic layers were combined, dried over anhydrous sodium sulfate, filtered and concentrated. The residue obtained was purified by column chromatography (0-20% ethyl acetate/petroleum ether) to afford 4-(2-(tert-butyldimethylsilyloxy)propan-2-yl)-2-chloro-3-as a yellow oil (59 g, 75 yield).

A solution of 2-(dimethylamino)ethanol (17.6 g, 197 mmol) in hexane (500 mL) was cooled at -5° C. and n-BuLi (157 mL, 395 mmol) was added dropwise under a nitrogen atmosphere. After 30 min at 0° C. , the solution was cooled at -78° C. and a solution of 4-(2-(tert-butyldimethylsilyloxy)propan-2-yl)-2-chloro-3-fluoropyridine (20 g, 65.8 mmol) in hexane (60 mL) was added dropwise. After 1 h of stirring a deep rust coloured solution was observed. Then a solution of 1-fluoro-N-methoxy-N-methylcyclopropane-1-carboxamide (38.8 g, 263 mmol) in THF (100 mL) was introduced dropwise. After addition, the reaction medium was allowed to warm slowly to r.t. (1 h). The mixture was quenched by water (1000 mL) and extracted by EtOAc (500 mL x 3). The organic phase was dried over Na₂SO₄ and concentrated under reduced pressure. The crude product was purified by silica gel chromatography (petroleum ether : EtOAc ═ 50 : 1) to afford (4-(2-((tert-butyldimethylsilyl)oxy)propan-2-yl)-6-chloro-5-fluoropyridin-2-yl)(1-fluorocyclopropyl)methanone ( 18 g, yield:70% ) as a yellow oil.

Trimethylsulfoxonium iodide ( 11.2 g, 50.8 mmol) was added to the solution of Potassium tert-butoxide (5.7 g, 50.8 mmol) in DMSO (300 mL). The reaction mixture was stirred at rt for 0.5 h. Then (4-(2-((tert-butyldimethylsilyl)oxy)propan-2-yl)-6-chloro-5-fluoropyridin-2-yl)(1-fluorocyclopropyl)methanone (18 g, 46 mmol) was added to the mixture and stirred another 2 h. The mixture was added with water (200 mL) and extracted by EtOAc (300 mL x 3).The organic phase was dried over Na₂SO₄ and concentrated under reduced pressure to afford 4-(2-((tert-butyldimethylsilyl)oxy)propan-2-yl)-2-chloro-3-fluoro-6-(2-(1-fluorocyclopropyl)oxiran-2-yl)pyridine ( 18 g, yield : 97% ) as a yellow oil.

4-((Tert-butyldimethylsilyl)oxy)propan-2-yl)-2-chloro-3-fluoro-6-(1-fluorocyclopropyl)oxiran-2-yl)pyridine (18 g, 44.6 mmol) was dissolved in NH₃ in MeOH (300 mL). The mixture was stirred at 35° C. for 16 h. The mixture was concentrated under reduced pressure. The crude product was purified by silica gel chromatography (petroleum ether : EtOAc = 10 : 1) to afford 2-amino-1-(4-(2-((tert-butyldimethylsilyl)oxy)propan-2-yl)-6-chloro-5-fluoropyridin-2-yl)-1-(1-fluorocyclopropyl)ethan-1-ol ( 10 g, yield: 53 % ) as a yellow solid.

Di-tert-butyl dicarbonate ( 4.7 mL, 22.2 mmol), Triethylamine ( 7 mL, 50.5 mmol,) has been added to a solution of 2-amino-1-(4-(2-((tert-butyldimethylsilyl)oxy)propan-2-yl)-6-chloro-5-fluoropyridin-2-yl)-1-(1-fluorocyclopropyl)ethan-1-ol ( 8.5 g, 20.2 mmol), in dichloromethane (100 mL). The mixture was stirred at room temperature for 2 h. The mixture was concentrated under reduced pressure to afford tert-butyl (2-(4-(2-((tert-butyldimethylsilyl)oxy)propan-2-yl)-6-chloro-5-fluoropyridin-2-yl)-2-(1-fluorocyclopropyl)-2-hydroxyethyl)carbamate (10 g, yield: 95%).

Tetrakis(triphenylphosphine)palladium (0.88 g, 0.77 mmol) was added to a solution of tert-butyl (2-(4-(2-((tert-butyldimethylsilyl)oxy)propan-2-yl)-6-chloro-5-fluoropyridin-2-yl)-2-(1-fluorocyclopropyl)-2-hydroxyethyl)carbamate (4 g, 7.7 mmol), (4-fluorophenyl)boronic acid (1.6 g, 11.5 mmol), potassium carbonate (2.65 g, 19.2 mmol) in DME (36 mL) and water (12 mL). The mixture was stirred at 160° C. for 5 min under microwave. The mixture was added with water (50 mL) and extracted by EtOAc (70 mL x 3). The organic phase was dried over Na₂SO₄ and concentrated under reduced pressure. The crude product was purified by silica gel chromatography (petroleum ether : EtOAc = 50 : 1) to afford tert-butyl (2-(4-(2-((tert-butyldimethylsilyl)oxy)propan-2-yl)-5-fluoro-6-(4-fluorophenyl)pyridin-2-yl)-2-(1-fluorocyclopropyl)-2-hydroxyethyl)carbamate (8 g) as a yellow oil.

Hydrochloric acid (80 mL) has been added to a solution of tert-butyl (2-(4-(2-((tert-butyldimethylsilyl)oxy)propan-2-yl)-5-fluoro-6-(4-fluorophenyl)pyridin-2-yl)-2-(1-fluorocyclopropyl)-2-hydroxyethyl)carbamate (10 g, 17.2 mmol ) in Methanol (40 mL). The mixture was stirred at room temperature for 2 h. Themixture was concentrated under reduced pressure and 40 mL of water was added. The mixture was and extracted by DCM (30 mL x 3). The organic phase was concentrated under reduced pressure to afford 2-(6-(2-amino-1-(1-fluorocyclopropyl)-1-hydroxyethyl)-3-fluoro-2-(4-fluorophenyl)pyridin-4-yl)propan-2-ol (6 g, yield : 95%).

The racemates of 2-(6-(2-amino-1-(1-fluorocyclopropyl)-1-hydroxyethyl)-3-fluoro-2-(4-fluorophenyl)pyridin-4-yl)propan-2-ol ( 6.5 g, 17.7 mmol) were separated by Prep-Chiral-HPLC with the following conditions: Column: CHIRAL ART Cellulose-SB S-5um 50*250mm, 50 mm*250 mm 5 um; Mobile Phase A:CO2, Mobile Phase B:MEOH(2 mM NH3-MEOH); Flow rate: 150 mL/min; Gradient:24% B; 220 nm; RT1:4.89; RT2:5.67; Injection Volumn:4 ml; Number Of Runs:38; to afford : (-)2-(6-(2-amino-1-(1-fluorocyclopropyl)-1-hydroxyethyl)-3-fluoro-2-(4-fluorophenyl)pyridin-4-yl)propan-2-ol (first eluting isomer): (2.8 mg, 42 %) as a yellow solid. e.e = 100%. [α] = -6° (589 nm, 23.6° C., 5 mg in 10 mL MeOH), and (+) 2-(6-(2-amino-1-(1-fluorocyclopropyl)-1-hydroxyethyl)-3-fluoro-2-(4-fluorophenyl)pyridin-4-yl)propan-2-ol (second eluting isomer): (3.2 g, 45%) as a yellow solid. e.e = 98.44%. [α] = +6° (589 nm, 23.6° C., 5 mg in 10 mL MeOH)

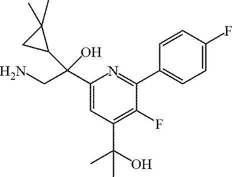 [SMILES: CC(C)(O)c1cc(C(O)(CN)C2CC2(C)C)nc(-c2ccc(F)cc2)c1F]

2-(6-(2-Amino-1-(2,2-dimethylcyclopropyl)-1-hydroxyethyl)-3-fluoro-2-(4-fluorophenyl)-pyridin-4-yl)propan-2-ol have been prepared following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol using N-methoxy-N,2,2-trimethyl-cyclopropanecarboxamide.

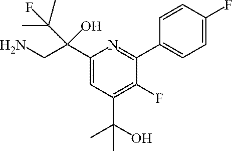 [SMILES: CC(C)(O)c1cc(C(O)(CN)C(C)(C)F)nc(-c2ccc(F)cc2)c1F]

1-Amino-3-fluoro-2-(5-fluoro-6-(4-fluorophenyl)-4-(2-hydroxypropan-2-yl)pyridin-2-yl)-3-methylbutan-2-ol have been prepared following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol using 2-fluoro-N-methoxy-N,2-dimethylpropanamide crude 2-fluoro-N-methoxy-N,2-dimethylpropanamide.

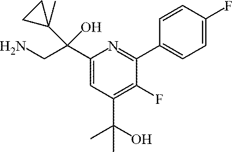 [SMILES: CC(C)(O)c1cc(C(O)(CN)C2(C)CC2)nc(-c2ccc(F)cc2)c1F]

2-(6-(2-Amino-1-hydroxy-1-(1-methylcyclopropyl)ethyl)-3-fluoro-2-(4-fluorophenyl)pyridin-4-yl)propan-2-ol has been prepared following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol using N-methoxy-N,1-dimethylcyclopropane-carboxamide.

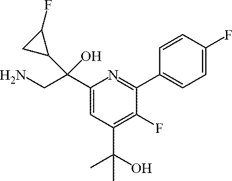 [SMILES: CC(C)(O)c1cc(C(O)(CN)C2CC2F)nc(-c2ccc(F)cc2)c1F]

2-(6-(2-Amino-1-(2-fluorocyclopropyl)-1-hydroxyethyl)-3-fluoro-2-(4-fluorophenyl)pyridin-4-yl)propan-2-ol have been prepared following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol using 2-fluoro-N-methoxy-N-methylcyclopropane-1-carboxamide.

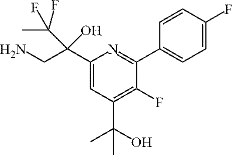 [SMILES: CC(C)(O)c1cc(C(O)(CN)C(C)(F)F)nc(-c2ccc(F)cc2)c1F]

1-Amino-3,3-difluoro-2-(5-fluoro-6-(4-fluorophenyl)-4-(2-hydroxypropan-2-yl)pyridin-2-yl)butan-2-ol have been prepared following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol using 2,2-difluoro-N-methoxy-N-methylpropanamide

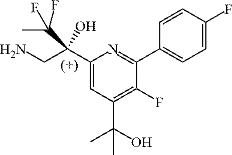

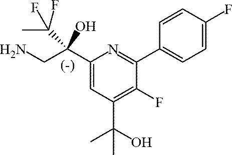

The racemates of 1-amino-3,3-difluoro-2-(5-fluoro-6-(4-fluorophenyl)-4-(2-hydroxypropan-2-yl)pyridin-2-yl)butan-2-ol (300 mg, 0.806 mmol) was separated by Prep-Chiral-HPLC with the following conditions: Column: Reg-AD Column, 4.6*100 mm 5 um; Mobile Phase A:Hex ( 0.1%DEA): EtOH=90:10 , Mobile Phase B:; Flow rate: 1 mL/min; Gradient:0 B to 0 B in min; nm; RT1:; RT2:; Injection Volumn: ml;

(-)-1Amino-3,3-difluoro-2-(5-fluoro-6-(4-fluorophenyl)-4-(2-hydroxypropan-2-yl)pyridin-2-yl)butan-2-ol1 (first eluting isomer): (113 mg, 37 %) as a white solid. e.e = 99.574%. [α] = -18° (589 nm, 24.2° C., 5 mg in 10 mL MeOH).

(+) 1-amino-3,3-difluoro-2-(5-fluoro-6-(4-fluorophenyl)-4-(2-hydroxypropan-2-yl)pyridin-2-yl)butan-2-ol (second eluting isomer): (119 mg, 39%) as a white solid. e.e = 99.440%. [α] = +18° (589 nm, 24.6° C., 5 mg in 10 mL MeOH)

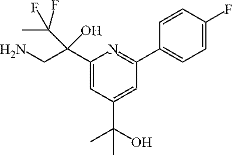 [SMILES: CC(C)(O)c1cc(-c2ccc(F)cc2)nc(C(O)(CN)C(C)(F)F)c1]

1-Amino-3,3-difluoro-2-(5-fluoro-6-(4-fluorophenyl)-4-(2-hydroxypropan-2-yl)pyridin-2-yl)butan-2-ol have been prepared following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol starting from 2-(2-chloropyridin-4-yl)propan-2-ol and using 2,2-difluoro-N-methoxy-N-methylpropanamide

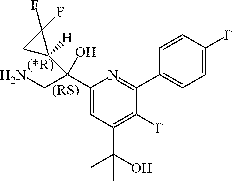

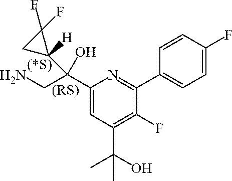

(RS)-2-(6-(2-amino-1-((*S)-2,2-difluoro-1-methylcyclopropyl)-1-hydroxyethyl)-3-fluoro-2-(4-fluorophenyl)pyridin-4-yl)propan-2-ol and (RS)-2-(6-(2-amino-1-((*R)-2,2-difluoro-1-methylcyclopropyl)-1-hydroxyethyl)-3-fluoro-2-(4-fluorophenyl)pyridin-4-yl)propan-2-ol have been obtained following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol starting from using 2,2-difluoro-N-methoxy-N-methylcyclopropane-1-carboxamide. Note : A separation of diastereaoisomers has been performed before deprotection of the amino and hydroxy group.

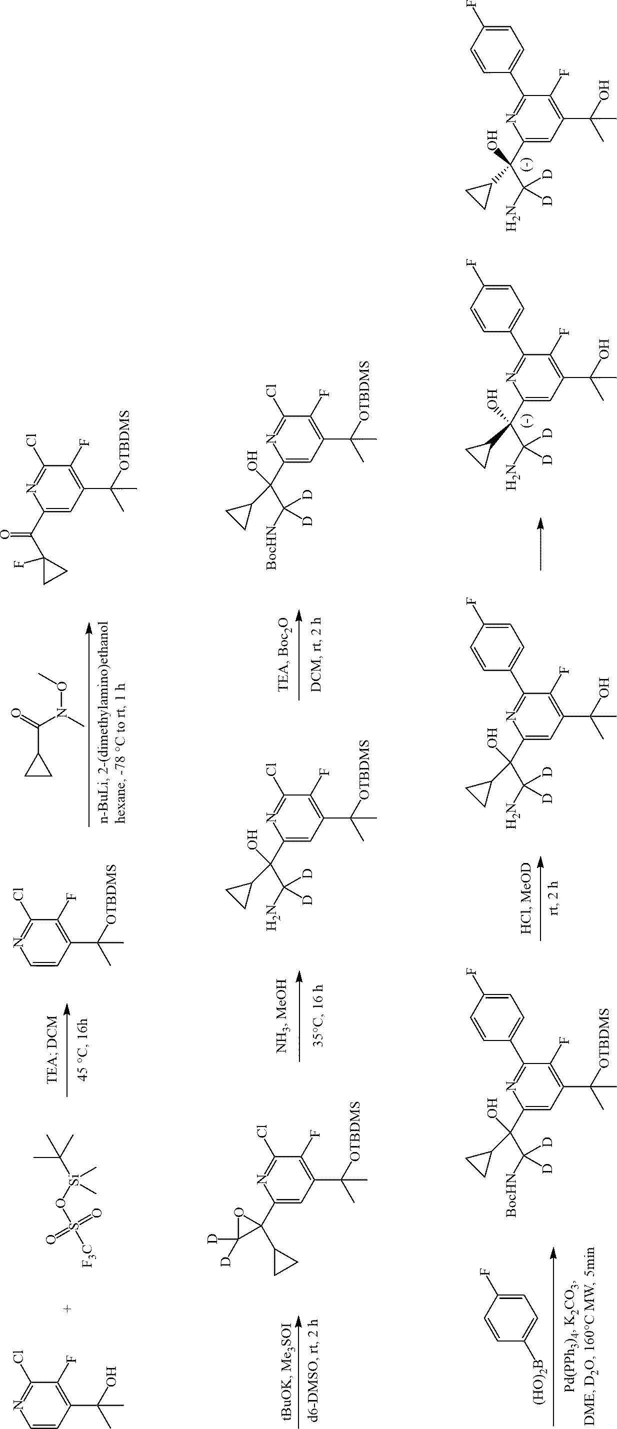

2-(6-(2-amino-1-cyclopropyl-1-hydroxyethyl-2,2-d2)-3-fluoro-2-(4-fluorophenyl)pyridin-4-yl)propan-2-ol have been prepared following a similar route to the one described for synthetizing 2-(6-(2-amino-1-(1-fluorocyclopropyl)-1-hydroxyethyl)-3-fluoro-2-(4-fluorophenyl)pyridin-4-yl)propan-2-ol using N-methoxy-N-methylcyclopropanecarboxamide.

Note : in step 3 : DMSO has been replaced by DMSO-d₆, in step 6: water has been replaced by D2O and in step 7, methanol has been replaced by deuterated methanol.

The enantiomers of 2-(6-(2-amino-1-cyclopropyl-1-hydroxyethyl-2,2-d2)-3-fluoro-2-(4-fluorophenyl)pyridin-4-yl)propan-2-ol (340 mg, 0.970 mmol) were separated by Prep-Chiral-HPLC with the following conditions: column: Chrialpak AD-H, 2 x 25 cm, 5 um; Mobile Phase A: Hex (8 mmol/L NH3. MeOH )--HPLC, Mobile Phase B: EtOH--HPLC; Flow rate: 20 mL/min; Gradient: 5% B to 5% B in 21 min; 220/254 nm; Rt1: 10.004 min; Rt2: 15.561 min; Injection Volumn: 0.5 ml; Number of Runs: 12; (-)-2-(6-(2-amino-1-cyclopropyl-1-hydroxyethyl-2,2-d2)-3-fluoro-2-(4-fluorophenyl)pyridin-4-yl)propan-2-ol (125.6 mg, 36%) as light yellow solid. [α]= -10° (589 nm, 24.2° C., 5 mg in 10 mL MeOH).

(+)(6-(2-amino-1-cyclopropyl-1-hydroxyethyl-2,2-d2)-3-fluoro(4-fluorophenyl)pyridin-4-yl)propan (114.9 mg, 33%) as light yellow solid. [α]= +10° (589 nm, 24.2° C., 5 mg in 10 mL MeOH).

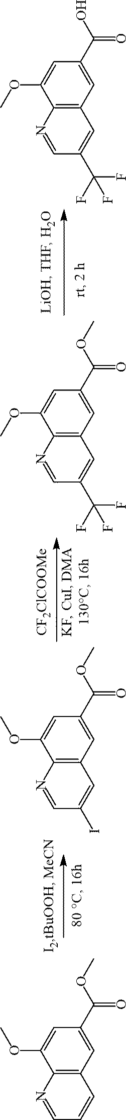

I2 (5.3 g, 21 mmol), ethyl 8-methoxyquinoline-6-carboxylate (3.0 g, 14 mmol), t-BuOOH (14.0 g, 109 mmol, 70%), and CH₃CN (50 mL) was added to a 100 mL round-bottomed flask. The resultant mixture was stirred at 80° C. for 16 hours. The reaction mixture was poured into sat. Na₂SO₃ (200 mL), extracted with ethyl acetate (100 mL x 3), the combined organic extracts were dried over anhydrous Na₂SO₄, filtered, and concentrated to dryness under reduced pressure to afford the crude product, which was purified by FCC (eluent: petroleum ether: ethyl acetate = 1:0 to 3:1, petroleum ether: ethyl acetate = 3:1, Rf=0.7) to afford methyl 3-iodo-8-methoxyquinoline-6-carboxylate, LCMS: as a white solid (3.5 g, 73% yield)

Methyl 2-chloro-2,2-difluoroacetate (5.9 g, 41 mmol) was added to a solution consisting of methyl 3-iodo-8-methoxyquinoline-6-carboxylate (3.5 g, 10 mmol), KF (1.2 g, 21 mmol), CuI (3.9 g, 21 mmol) and dimethylacetamide (60 mL). The mixture was stirred at 130° C. for 16 hours before cooling to room-temperature. The reaction mixture was poured into sat. NaCl (200 mL), extracted with ethyl acetate (80 mL x 3), the combined organic extracts were dried over anhydrous Na₂SO₄, filtered, and concentrated to dryness under reduced pressure to afford the crude product, which was purified by flash column chromatography (eluent: petroleum ether: ethyl acetate = 1:0 to 3:1, petroleum ether: ethyl acetate = 3:1, Rf=0.7) to afford methyl 8-methoxy-3-(trifluoromethyl)quinoline-6-carboxylate as a brown solid (2.5 g, 73% purity, 63% yield)

LiOH.H₂O (551 mg, 13.1 mmol) was added into to a 0° C. (ice/water) mixture consisting of methyl 8-methoxy-3-(trifluoromethyl)quinoline-6-carboxylate (2.5 g, 8.8 mmol), THF (15 mL) and H₂O (15 mL). The resultant mixture was stirred at room-temperature for 2 hours. The reaction mixture was acidified to pH = 5 by addition ofHCl.

The mixture was concentrated to dryness under reduced pressure to afford the crude product, which was purified by preparative HPLC with a Phenomenex Synergi Max-RP 250*50 mm*10 um (eluent: 25% to 55% (v/v) water (0.1%TFA)-ACN to afford title product. The product was suspended in water (50 mL), the mixture frozen using dry ice/acetone, and then lyophilized to dryness to afford 8-methoxy-3-(trifluoromethyl)quinoline-6-carboxylic acid as a yellow solid (910 mg, 39% yield).

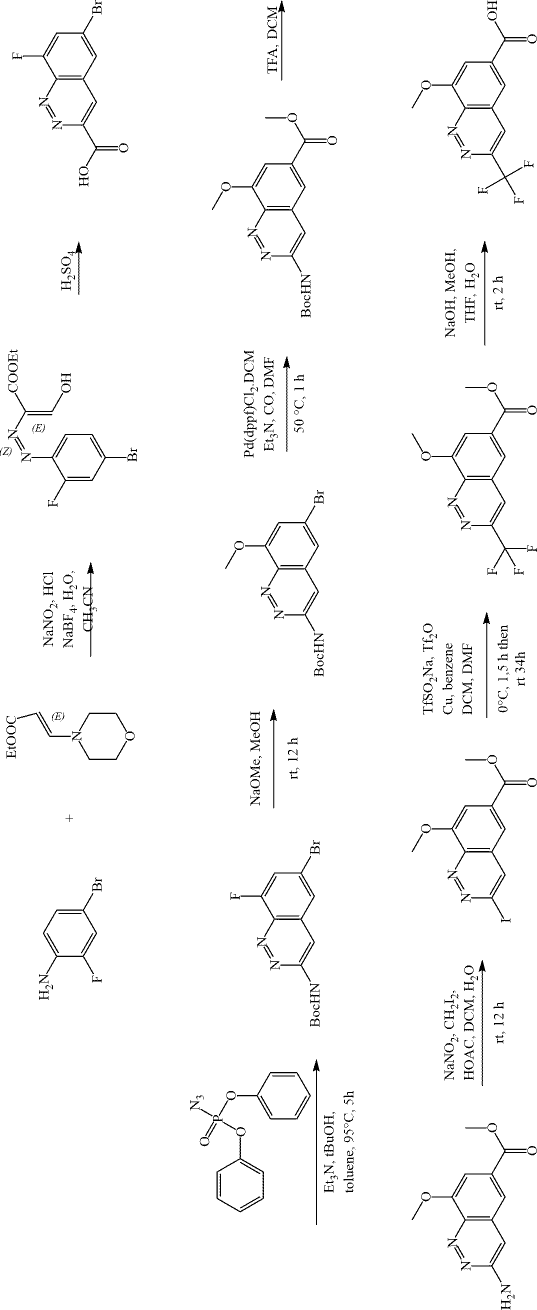

To a cooled (0° C.) solution of 4-bromo-2-fluoro-aniline (22 g, 115.8 mmol) in H₂O (250 mL) was treated with HCl (12 M, 28 mL) and NaNO2 (9.6 g, 138.9 mmol) After 40 min at 0° C., HCl (12 M, 30 mL ) and sodium tetrafluoroborate (50.8 g, 463 mmol) was added. After stirring at 0° C. for 40 min, the intermediate diazonium was filtered, A solution of crude product in MeCN (200 mL) was treated with ethyl-3-morpholinoprop-2-enoate (21.4 g, 115 mmol) ,The mixture was stirred at 25° C. for 10 hr. The mixture was concentrated. The residue was purified by column chromatography (SiO2, Petroleum ether/Ethyl acetate=50/1 to 5:1) to afford 2-(4-bromo-2-fluorophenyl)diazenyl)-3-hydroxyacrylate (20 g, 54%) as yellow solid was obtained .

To a solution ethyl 2-(4-bromo-2-fluorophenyl)diazenyl)-3-hydroxyacrylate (12 g ,36.2 mmol) was added H₂SO₄ (100 mL) stirred at 100° C. for 8 h. The mixture was quenched by water (1000 mL) then extracted with ethyl acetate (800 mL*3) .The organic phase was concentrated under vacuum to give brown solid .The brown solid (8 g ,crude) was used for the next step directly without purification

A solution of 6-bromo-8-fluorocinnoline-3-carboxylic acid (10 g, 37 mmol) in tert-butanol (120 mL) was added diphenyl phosphorazidate (12.2 g, 44.4 mmol) and triethylamine (8.2 g, 81.4 mmol), the mixture was heated at 95° C. for 5 h. After cooling down to rt, the solvent was removed under reduced pressure and the residue obtained was purified by silica gel chromatography (0-10% ethyl acetate/petroleum ether) to afford the tert-butyl (6-bromo-8-fluorocinnolin-3-yl)carbamate yellow solid (3 g, 24% yield).

To a solution of tert-butyl (6-bromo-8-fluorocinnolin-3-yl)carbamate (2.4 g, 7 mmol) in MeOH (50 mL) was treated with NaOMe (1.1 g, 21 mmol) the mixture was stirred at rt for 12 h. The reaction was quenched with water (20 mL). The resulting mixture was extracted with ethyl acetate (3 x 70 mL). The organic layers were combined, dried over anhydrous sodium sulfate, filtered, and concentrated to provide tert-butyl (6-bromo-8-methoxycinnolin-3-yl)carbamate (0.86 g, 35%) as a yellow solid.

To solution of tert-butyl (6-bromo-8-methoxycinnolin-3-yl)carbamate (0.85 g, 2.4 mmol) in MeOH (20 mL) was treated with 1,1′-bis(diphenylphosphino)ferrocene-palladium(II)dichloride dichloromethane complex (196.0 mg, 0.24 mmol) and Et₃N (0.49 g, 4.8 mmol), the mixture was stirred under CO (1 atm) at 50° C. for 1 h. After cooling to r.t., the solvent was removed in vacuo and the residue was purified by silica gel chromatography (0-20% ethyl acetate/petroleum ether) to provide the methyl 3-((tert-butoxycarbonyl)amino)-8-methoxycinnoline-6-carboxylate (630 mg, 79% yield) as a yellow solid.

To a solution of methyl 3-((tert-butoxycarbonyl)amino)-8-methoxycinnoline-6-carboxylate (0.63 g, 1.9 mmol) in DCM (15 mL) was treated with TFA (4.3 g, 38 mmol), the mixture was stirred at rt for 12 h. The solvent was removed under reduced pressure and the crude product was applied onto C18 (5-60% MeCN/H₂O(0.05% NH₄HCO₃)) provided methyl 3-amino-8-methoxycinnoline-6-carboxylate (400 mg, 90 % yield) as a yellow solid.

To a solution of methyl 3-amino-8-methoxycinnoline-6-carboxylate (326.5 mg, 1.4 mmol) in DCM (8 mL) and H₂O (8 mL)was treated with CH₂I₂ (750 mg, 2.8 mmol), NaNO₂ (483 mg, 7 mmol) and HOAc (1.68 g, 28 mmol), the mixture was stirred at rt for 12 h. The solvent was removed under reduced pressure and the crude was purified by silica gel chromatography (0-50% ethyl acetate/petroleum ether) to provide methyl 3-iodo-8-methoxycinnoline-6-carboxylate (180 mg, 38% yield) as a yellow solid .

Under a nitrogen atmosphere, benzene (520.5 mg, 6.66 mmol) and Tf₂O (844.2 mg, 3.0 mmol) were added into a suspension of TfSO₂Na (212.2 mg, 1.4 mmol) in DCM (8 mL), which was well cooled by ice-bath. After stirring at 0° C. for 1.5 h, the reaction mixture was warmed to rt and allowed to react for 34 h. Then the reaction mixture was diluted with DCM, washed successively with saturated aqueous NaHCO₃ and NaCl and dried over Na₂SO₄, the solvent was removed under reduced pressure and get [Ph₂SCF₃][OTf] as a yellow solid. In a 10 mL sealed tube, methyl 3-iodo-8-methoxycinnoline-6-carboxylate (234 mg, 0.68 mmol) and [Ph₂SCF₃][OTf] (550 mg,1.36 mmol) were dissolved in DMF (8 mL), Cu (130 mg, 2.04 mmol) was added. The reaction mixture was stirred at 70° C. for 12 h. After cooling down to rt, the solvent was removed under reduced pressure and the crude was purified by silica gel chromatography (0-50% ethyl acetate/petroleum ether) to provide the target product (27 mg, 13% yield) as a yellow solid.

To a solution of methyl 8-methoxy-3-(trifluoromethyl)cinnoline-6-carboxylate (278 mg, 0.97 mmol) in MeOH (10 mL),THF (10 ml) and H₂O (2 mL) was treated with NaOH (0.155 g, 3.9 mmol), the reaction mixture was stirred at rt for 2 h. The reaction was neutralized with 1 N HCl (4 mL) and extracted with ethyl acetate (3 X 20 mL), the combined organic layer was washed with brine and dried over anhydrous Na₂SO₄, the solvent was removed under reduced pressure and the crude product was applied onto C18 (5-60% MeCN/H₂O (0.05% HCl)) provide the target product (65.4 mg, 25% yield) as a light yellow solid .

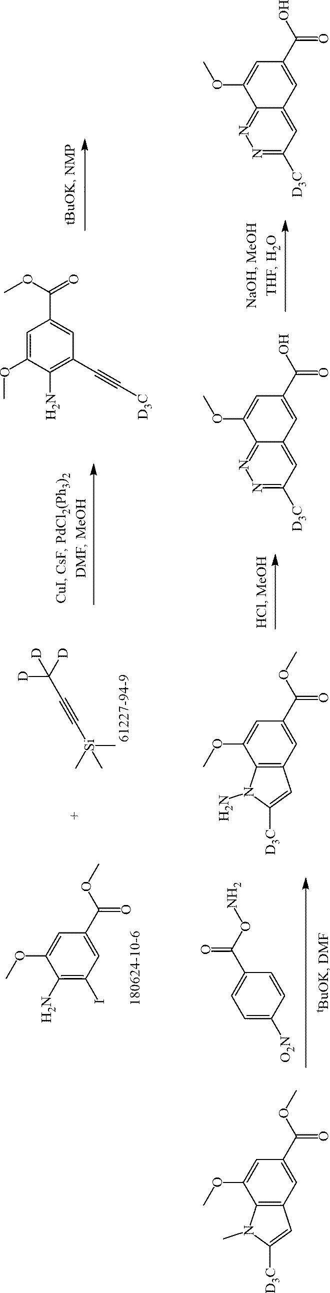

To a solution of methyl 4-amino-3-iodo-5-methoxybenzoate (8 g, 26.051 mmol) in DMF (80 mL) and MeOH (20 mL) was added trimethyl(prop-1-yn-1-yl-d3)silane (6.006 g, 52.103 mmol), CuI (0.992 mg, 5.210 mmol), CsF (11.8 mg, 78 mmol), PdCl₂(PPh₃)₂ (0.91 g, 1.303 mmol). The resulting mixture was maintained under nitrogen and stirred at 30° C. for 30 min. The mixture was filtered through a celite pad and was washed with CH₂Cl₂ (200 mL X 1). The filtrate was concentrated under reduced pressure to remove MeOH and CH₂Cl₂. The residue was poured into ice water. The precipitated solid was filtered and was washed with water (200 mL). The solid was dissolved in CH₂Cl₂ (200 mL).The combined organic layer was washed with saturated brine (200 mL X 6). The organic layer was dried over anhydrous sodium sulfate, filtered and concentrated. The residue was dried over vacuum to afford methyl 4-amino-3-methoxy-5-(prop-1-yn-1-yl-d3)benzoate as a brown solid (5 g, 87% yield).

Methyl 4-amino-3-methoxy-5-(prop-1-yn-1-yl-d3)benzoate (6 g, 27 mmol, 1 eq) was dissolved in 1-methyl-2-pyrrolidinone (120 mL). The resultant mixture was purged with N2 then potassium tert-butoxide (7.573 g, 67 mmol) was added. The mixture was purged again with N₂ then stirred at 30° C. for 4 h. A saturated aqueous solution of NH₄Cl was added to the crude. The resulting mixture was extracted with ethyl acetate (3 x 100 mL). The organic layers were combined, dried over anhydrous sodium sulfate, filtered and concentrated. The residue obtained was purified by silica gel chromatography (0-10% ethyl acetate/petroleum ether) to afford the methyl 7-methoxy-2-(methyl-d3)-1H-indole-5-carboxylate as a yellow solid (3.95 g, 66% yield). Methyl 7-methoxy-2-(methyl-d3)-1H-indole-5-carboxylatee (1.18 g, 5.3 mmol) was dissolved in DMF (30 mL). The resultant mixture was purged with N₂. Then potassium tert-butoxide (1.19 g, 10.6 mmol) was added in the mixture. After that O-(4-nitrobenzoyl)hydroxylamine (1.9 g, 10.6 mmol) was added to the mixture. The mixture was purged again with N2 then stirred at r.t. for 4 h. A saturated aqueous solution of NH₄Cl was added to the crude. The resulting mixture was extracted with ethyl acetate (3 x 30 mL). The organic layers were combined, dried over anhydrous sodium sulfate, filtered and concentrated. The residue obtained was purified by silica gel chromatography (0-30% ethyl acetate/petroleum ether) to afford the methyl 1-amino-7-methoxy-2-(methyl-d3)-1H-indole-5-carboxylate as a yellow solid (700 mg, 56 % yield).

To a solution of methyl 1-amino-7-methoxy-2-(methyl-d3)-1H-indole-5-carboxylate (700 mg, 2.950 mmol) in methanol (18 mL) was added 4 M hydrogen chloride solution in methanol (6 mL). The resulting mixture was heated to 80° C. for 16 h. After cooling down to r.t., the mixture was filtrated and concentrated. The mixture was dissolved with dichloromethane. A saturated aqueous solution of NaHCO₃ was added to the crude. The organic layers were combined, dried over anhydrous sodium sulfate, filtered and concentrated. The residue obtained was purified by silica gel chromatography (0-50% ethyl acetate/petroleum ether) to afford the methyl 8-methoxy-3-(methyl-d3)cinnoline-6-carboxylate as a yellow solid (520 mg, 75% yield).

To a solution of methyl 8-methoxy-3-(methyl-d3)cinnoline-6-carboxylate (510 mg, 2.2 mmol) in methanol (10 mL) and tetrahydrofuran (10 mL) was added sodium hydroxide (346 mg, 8.6 mmol, 4 eq) and D₂O (2 mL). The mixture was stirred at 80° C. for 4 h. The mixture was filtrated and concentrated. The residue obtained was purified by flash column chromatography (C18: H2O(0.05%):MeCN=1:1) to afford the 8-methoxy-3-(methyl-d3)cinnoline-6-carboxylic acid as a yellow solid (287.1 mg, 58% yield).

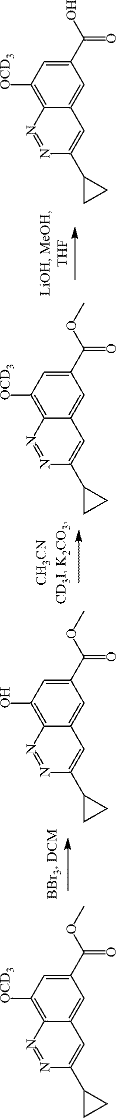

To a solution of methyl 3-cyclopropyl-8-methoxycinnoline-6-carboxylate (5 g, 19.35 mmol) in dichloromethane (80 mL) was added boron tribromide (14.55 g, 58.08 mmol) at 0° C. The mixture was stirred at r.t. for 2 h. The mixture was filtrated and concentrated afford the methyl 3-cyclopropyl-8-hydroxycinnoline-6-carboxylate crude as a yellow solid (4.3 g, 91 % yield).

Methyl 3-cyclopropyl-8-hydroxycinnoline-6-carboxylate (1 g, 4.09 mmol and potassium carbonate (1.69 g, 12.3 mmol) were added into a round bottom flask. The mixture was purged with N₂. After that acetonitrile (40 mL) was added. The mixture was purged again with N2 then iodomethane-D3 (2.37 g, 16.37 mmol) was added. The mixture was purged again with N2 then stirred at 40° C. for 2 h. The resulting mixture was extracted with dichloromethane (3 x 50 mL). The organic layers were combined, dried over anhydrous sodium sulfate, filtered and concentrated. The residue obtained was purified by silica gel chromatography (0-50% ethyl acetate/petroleum ether) to afford the methyl 3-cyclopropyl-8-(methoxy-d3)cinnoline-6-carboxylate as a yellow solid (400 mg, 37% yield).

To a solution of methyl methyl 3-cyclopropyl-8-(methoxy-d3)cinnoline-6-carboxylate (1.07 g, 4.09 mmol) in methanol (8 mL) and tetrahydrofuran (8 mL) was added lithium hydroxide (0.29 g, 12.29 mmol) and H₂O (1.6 mL). The mixture was stirred at r.t. for 1 h. The mixture was filtrated and concentrated. The residue obtained was purified by silica gel chromatography (0-50% H₂O/ACN) to afford the 3-cyclopropyl-8-(methoxy-d3)cinnoline-6-carboxylic acid as a yellow solid (351 mg, 70.767 % yield).

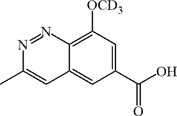

8-(Methoxy-d3)-3-methylcinnoline-6-carboxylic acid has been prepared following a similar procedure to the one use to make 3-cyclopropyl-8-(methoxy-d3)cinnoline-6-carboxylic acid

### 4. Syntheses of Final Products

The final compounds were synthesized according to one of the following procedures: A, B, C or D.

### Procedure A

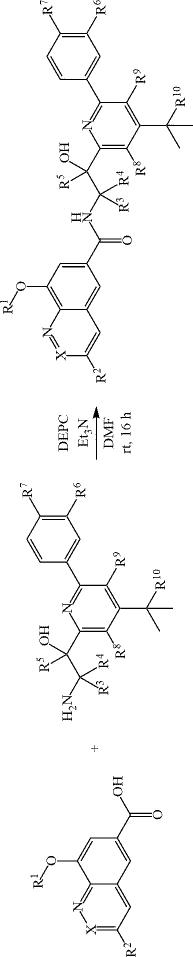

In a vial, to a mixture of amine (1 equiv.) and carboxylic acid (1.5 equiv.) in anhydrous DMF (0.027 M) was added Et₃N (3 equiv.) followed by DEPC (2 equiv.) The vial was sealed and stirred at rt for 16 h. The solution was then submitted for purification.

### Procedure B

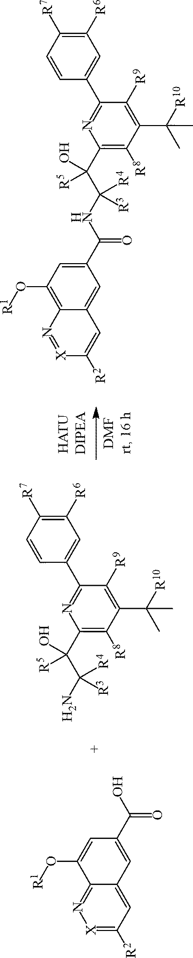

To a mixture of acid carboxylic (1 equiv.) and amine (1 equiv.) in DMF (0.17 M) was added DIPEA (2 equiv.) followed by HATU (1 equiv.) portionwise. The reaction mixture was stirred at rt for few hours. The reaction was diluted with water and stirred at rt for another 2 h. The precipitate was collected by filtration and dried under vacuum. Then a purification was eventually performed.

### Procedure C

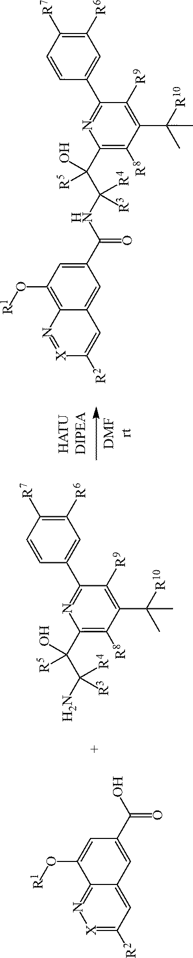

To a mixture of carboxylic acid (1 equiv.) and amine (1.1 equiv) in anhydrous DMF (0.10 M) were added HATU (1.5 equiv.) and DIPEA (2 equiv.). The reaction mixture was stirred at rt for few hours. The mixture was diluted with EtOAc and NaHCO₃ (10%, aq.). The layers were separated, and the aqueous phase was extracted with EtOAc (twice). The combined organic extracts were washed with brine (3 times) and dried (MgSO₄). The solids were removed by filtration and the filtrate was concentrated under reduced pressure. Purification of the crude mixture delivered the desired compound.

### Procedure D

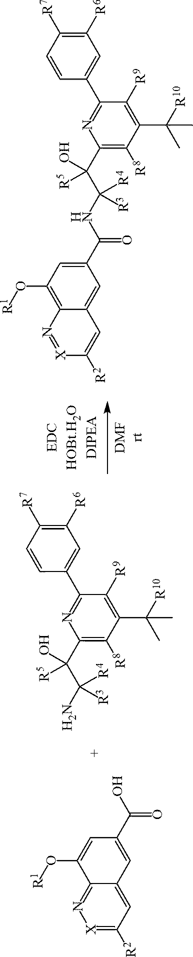

A mixture of carboxylic acid (1 equiv.), amine (1 equiv.), EDC (1 equiv.), HOBt.H₂O (1 equiv.) and DIPEA (2 equiv.) in DMF (0.05 M) was stirred at rt for few hours. The reaction was diluted with H₂O, brine, NaHCO₃ (sat., aq.) and EtOAc. The layers were separated, and the aqueous phase was extracted with EtOAc. The combined organic extracts were washed with a mixture of KHSO₄ and brine (1:1), then with brine (4 times). The organic layer was dried (MgSO₄). The solids were removed by filtration and the filtrate was concentrated under reduced pressure. Purification of the crude mixture delivered the desired compound.

### 4.1. Quinoline Products

### (-)-N-{2-[5-Chloro-6-(4-Fluorophenyl)-4-(2-Hydroxypropan-2-yl)Pyridin-2-yl]-3,3,3-Trifluoro-2-Hydroxypropyl}-8-Methoxy-3-Methylquinoline-6-Carboxamide 200

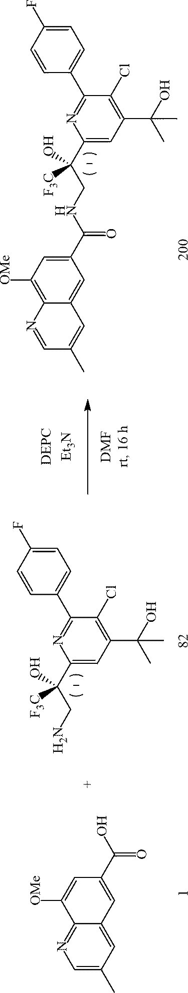

200 (121 mg. 80%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 1.58 (s, 3H), 1.65 (s, 3H), 2.49 (br s, 3H), 3.96 (s, 3H), 4.11 (dd, J=14.1, 5.5 Hz, 1H), 4.22 (dd, J=14.1, 6.2 Hz, 1H), 5.70 (s, 1H), 7.26 - 7.34 (m, 2H), 7.35 (d, J=1.5 Hz, 1H), 7.44 (s, 1H), 7.68 - 7.74 (m, 2H), 7.78 (d, J=1.5 Hz, 1H), 8.06 (dd, J=2.1, 1.0 Hz, 1H), 8.34 (s, 1H), 8.73 (t, J=5.9 Hz, 1H), 8.77 (d, J=2.0 Hz, 1H); LC-MS (method B): Rt = 1.12 min; mass calcd. for C₂₉H₂₆ClF₄N₃O₄ 591.0, m/z found 592.3 [M+H]⁺; [α]_{D}²⁰ -62.55° (c 0.267, DMF).

### (-)-N-{2-[3,5-difluoro-6-(4-fluorophenyl)-4-(2-hydroxypropan-2-yl)pyridin-2-yl)-3,3,3-trifluoro-2-hydroxypropyl}-8-methoxy-3-methylquinoline-6-carboxamide 201

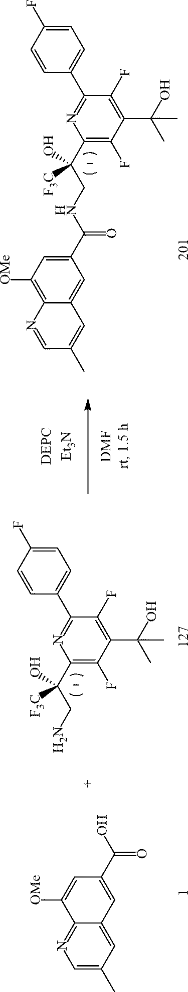

201 (74 mg. 49%) was synthesized according to procedure A. The purification was performed via reverse phase HPLC (stationary phase: Kromasil C18 100A 5 µm (Eka Nobel), mobile phase: NH₄HCO₃ (0.25% in H₂O)/ CH₃OH, gradient from 80:20 to 0:100). ¹H NMR (400 MHz, DMSO-d₆) δ ppm 1.61 (s, 6H), 2.46 (s, 3H), 3.90 (s, 3H), 4.07 (br dd, J=14.0, 5.2 Hz, 1H), 4.41 (br dd, J=13.8, 6.7 Hz, 1H), 5.61 (s, 1H), 7.07 (s, 1H), 7.27 (d, J=1.5 Hz, 1H), 7.30 - 7.42 (m, 2H), 7.72 (d, J=1.5 Hz, 1H), 7.93 (dd, J=7.5, 5.5 Hz, 2H), 7.99 (s, 1H), 8.57 (br t, J=5.9 Hz, 1H), 8.74 (d, J=2.0 Hz, 1H); LC-MS (method B): Rt 1.04 min; mass calcd. for C₂₉H₂₅F₆N₃O₄ 593.0, m/z found 594.0 [M+H]⁺; [α]_{D}²⁰ -25.43° (c 0.260, DMF).

### (-)-N-[5-chloro-3-fluoro-6-(4-fluorophenyl)-4-hydroxypropan-2-yl)pyridin-2-yl]-3,3,3-trifluoro-2-hydroxypropyl}-8-methoxy-3-methylquinoline-6-carboxamide 202

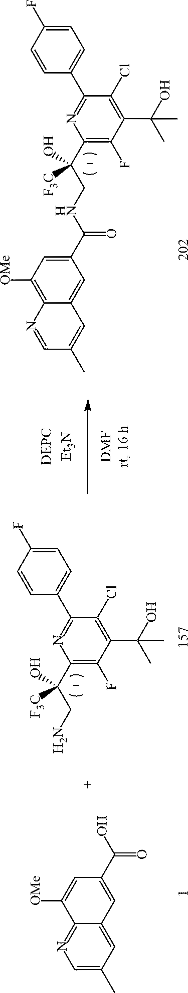

202 (130 mg. 88%) was synthesized according to procedure A. The purification was performed via reverse phase HPLC (stationary phase: 250 g, YMC Tri-Art, mobile phase: NH₄HCO₃ (0.25% in H₂O)/CH₃CN, gradient from 65:35 to 35:65). ¹H NMR (400 MHz, DMSO-d₆) δ ppm 1.65 (s, 3H), 1.66 (s, 3H), 2.48 (s, 3H), 3.93 (s, 3H), 4.03 (br dd, J=13.9, 5.1 Hz, 1H), 4.41 (br dd, J=14.0, 6.7 Hz, 1H), 5.60 (s, 1H), 6.97 (br s, 1H), 7.24 - 7.31 (m, 3H), 7.53 - 7.61 (m, 2H), 7.71 (d, J=1.5 Hz, 1H), 8.04 (d, J=1.1 Hz, 1H), 8.57 (t, J=6.1 Hz, 1H), 8.76 (d, J=2.2 Hz, 1H); LC-MS (method G): Rt 2.05 min; mass calcd. for C₂₉H₂₅ClF₅N₃O₄ 609.2, m/z found 610.2 [M+H]⁺; [α]_{D}²⁰ -42.09° (c 0.269, DMF).

### (+)-N-{3,3-Difluoro-2-[5-Fluoro-6-(4-Fluorophenyl)-4-(2-Hydroxypropan-2-yl)Pyridin-2-yl]-2-Hydroxypropyl}-8-Methoxy-3-Methylquinoline-6-Carboxamide 203 and (-)-N-{3,3-Difluoro-2-[5-Fluoro-6-(4-Fluorophenyl)-4-(2-Hydroxypropan-2-yl)Pyridin-2-yl]-2-Hydroxypropyl}-8-Methoxy-3-Methylquinoline-6-Carboxamide 204

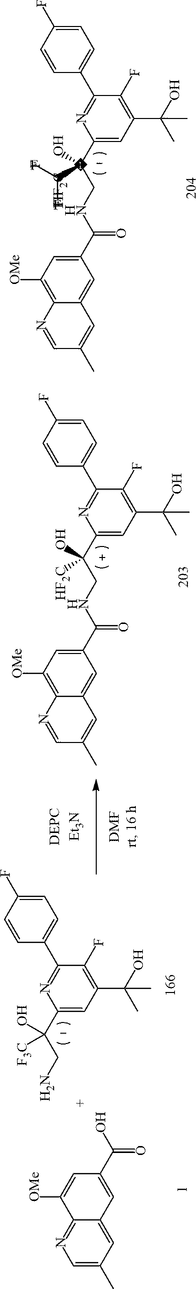

203 (174 mg. 75%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆) δ ppm 1.46 (s, 3H), 1.54 (s, 3H), 2.48 (s, 3H), 3.82 (br dd, J=13.8, 5.4 Hz, 1H), 3.93 (s, 3H), 3.95 - 4.06 (m, 1H), 5.62 (s, 1H), 6.58 (s, 1H), 6.64 (t, J=54.6 Hz, 1H), 7.22 - 7.32 (m, 3H), 7.72 (d, J=1.5 Hz, 1H), 7.90 - 7.99 (m, 3H), 8.04 (d, J=5.5 Hz, 1H), 8.54 (t, J=6.1 Hz, 1H), 8.75 (d, J=2.2 Hz, 1H); LC-MS (method B): Rt 1.00 min; mass calcd. for C₂₉H₂₇F₄N₃O₄ 557.0, m/z found 558.0 [M+H]⁺. A second purification was performed via normal phase HPLC (stationary phase: Daicel Chiralpak IC, 250 g, 5 µm, mobile phase: heptane/EtOH, 90: 10) to afford 203 (37 mg, 16%); [α]_{D}²⁰ +78.54° (c 0.261, DMF); and 204 (20 mg, 9%).

### (-)-N-{2-[5-Chloro-6-(4-Fluorophenyl)-4-(2-Hydroxypropan-2-yl)Pyridin-2-yl]-2-Cyclopropyl-2-Hydroxyethyl}-8-Methoxy-3-Methylquinoline-6-Carboxamide 205

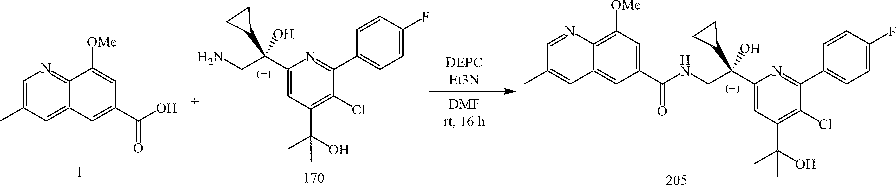

205 (115 mg. 74%) was synthesized according to procedure A. A purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.10 - 0.18 (m, 1H), 0.26 - 0.33 (m, 1H), 0.34 - 0.42 (m, 1H), 0.52 - 0.60 (m, 1H), 1.46 - 1.53 (m, 1H), 1.56 (s, 3H), 1.64 (s, 3H), 3.34 - 3.40 (m, 3H), 3.79 - 3.86 (m, 1H), 3.89 - 3.99 (m, 1H), 3.96 (s, 3H), 5.56 (br s, 1H), 5.57 (br s, 1H), 7.23 - 7.30 (m, 2H), 7.34 (d, J=1.5 Hz, 1H), 7.66 - 7.72 (m, 2H), 7.74 (d, J=1.5 Hz, 1H), 8.02 (dd, J=2.0, 0.9 Hz, 1H), 8.15 (s, 1H), 8.57 (t, J=5.9 Hz, 1H), 8.76 (d, J=2.0 Hz, 1H); LC-MS (method F): Rt = 2.51 min; mass calcd. for C₃₁H₃₁ClFN₃O₄ 563.0, m/z found 564.0 [M+H]⁺; [α]_{D}²⁰ -97.84° (c 0.254, DMF).

### (-)-N-{Cyclopropyl-2-[5-Fluoro-6-(4-Fluorophenyl)-4-Hydroxypropan-2-yl)Pyridin-2-yl]-2-Hydroxyethyl}-8-Methoxy-3-Methylquinoline-6-Carboxamide 206

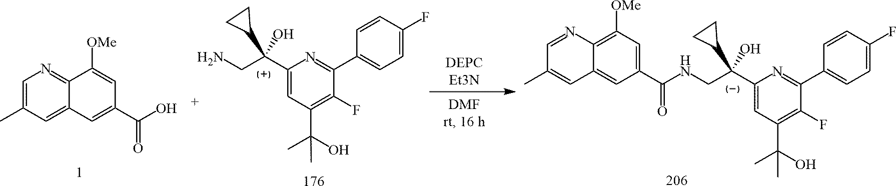

206 (123 mg. 78%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.11 - 0.19 (m, 1H), 0.26 - 0.34 (m, 1H), 0.36 - 0.45 (m, 1H), 0.54 - 0.62 (m, 1H), 1.48 (s, 3H), 1.54 (s, 3H), 1.55 - 1.60 (m, 1H), 2.48 (s, 3H), 3.88 (dd, J=13.4, 55 Hz, 1H), 3.92 - 3.99 (m, 1H), 3.95 (s, 3H), 5.56 (s, 2H), 7.28 - 7.37 (m, 3H), 7.76 (d, J=1.8 Hz, 1H), 7.94 (d, J=5.5 Hz, 1H), 7.96 - 8.02 (m, 3H), 8.55 (t, J=5.9 Hz, 1H), 8.76 (d, J=2.0 Hz, 1H); LC-MS (method F): Rt = 2.50 min; mass calcd. for C₃₁H₃₁F₂N₃O₄ 547.0, m/z found 548.4 [M+H]⁺; [α]_{D}²⁰ -32.65° (c 0.269, DMF)

### (-)-N-Cyclopropyl-2-{5-Fluoro-4-(2-Hydroxypropan-2-yl)-6-[4-(Trifluoromethyl)Phenyl]pyridin-2-yl}-2-Hydroxyethyl]-8-Methoxy-3-Methylquinoline-6-Carboxamide 207

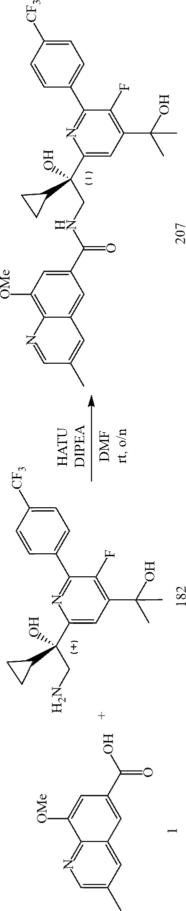

207 (141 mg. 47%) was synthesized according to procedure B. The precipitate was purified via silica column chromatography (CH₂Cl₂/CH₃OH, gradient from 99:1 to 95:5). The residue was crystallized from DIPE and CH₃CN (10:1) and the precipitate was collected by filtration and dried under vacuum. ¹H NMR (400 MHz, DMSO-d₆) δ ppm 0.10 - 0.21 (m, 1H), 0.27 - 0.36 (m, 1H), 0.37 - 0.46 (m, 1H), 0.54 - 0.64 (m, 1H), 1.48 (s, 3H), 1.55 (s, 3H), 1.56 - 1.63 (m, 1H), 2.47 (s, 3H), 3.90 (dd, J=13.4, 5.3 Hz, 1H), 3.95 (s, 3H), 3.95 - 4.00 (m, 1H), 5.58 (s, 1H), 5.60 (s, 1H), 7.35 (d, J=1.6 Hz, 1H), 7.78 (d, J=1.6 Hz, 1H), 7.85 (d, J=8.5 Hz, 2H), 8.01 (d, J=5.7 Hz, 1H), 8.04 (dd, J=2.0, 0.8 Hz, 1H), 8.15 (d, J=8.1 Hz, 2H), 8.55 (t, J=5.9 Hz, 1H), 8.76 (d, J=2.0 Hz, 1H); LC-MS (method H): Rt = 2.13 min; mass calcd. for C₃₂H₃₁F₄N₃O₄ 597.2, m/z found 598.5 [M+H]⁺; [α]_{D}²⁰ -41.28° (c 0.312, DMF).

### (-)-N-{2-[6-(4-chlorophenyl)-5-fluoro-4-(2-hydroxypropan-2-yl)pyridin-2-yl]-2-cyclopropyl-2-hydroxyethyl}-8-methoxy-3-methylquinoline-6-carboxamide 208

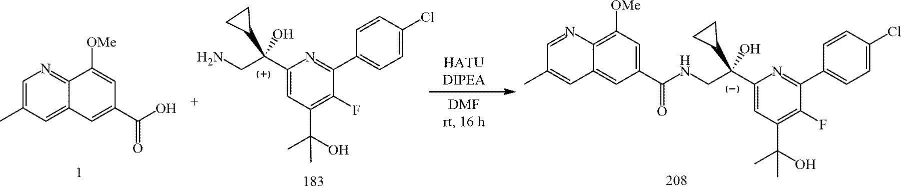

208 (220 mg. 78%) was synthesized according to procedure B. A purification was performed via SFC (stationary phase: Chiralpak Daicel IC 20 x 250 mm, mobile phase: CO₂, EtOH + 0.4% i-PrNH₂). ¹H NMR (400 MHz, DMSO-d₆) δ ppm 8.76 (d, J=2.0 Hz, 1H), 8.56 (t, J=5.9 Hz, 1H), 7.95 - 8.02 (m, 4H), 7.77 (d, J=1.6 Hz, 1H), 7.53 - 7.59 (m, 2H), 7.35 (d, J=1.6 Hz, 1H), 5.57 (s, 1H), 5.56 (s, 1H), 3.85 - 4.00 (m, 2H), 3.95 (s, 3H), 2.48 (s, 3H), 1.56 - 1.62 (m, 1H), 1.54 (s, 3H), 1.48 (s, 3H), 0.54 - 0.62 (m, 1H), 0.37 - 0.46 (m, 1H), 0.27 - 0.35 (m, 1H), 0.11 - 0.20 (m, 1H); LC-MS (method C): Rt = 2.16 min; mass calcd. for C₃₁H₃₁ClFN₃O₄ 563.2, m/z found 564.2 [M+H]⁺; [α]_{D}²⁰ -47.5° (c 0.53, DMF).

### (-)-N-{2-Cyclopropyl-2-[6-(3,4-Difluorophenyl)-5-Fluoro-4-(2-Hydroxypropan-2-yl)Pyridin-2-yl]-2-Hydroxyethyl}-8-Methoxy-3-Methylquinoline-6-Carboxamide 209

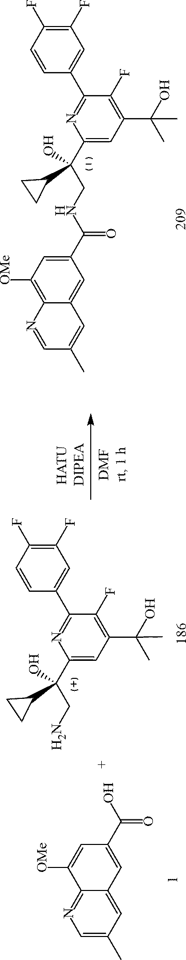

209 (121 mg. 43%) was synthesized according to procedure B. The precipitate was dissolved in CH₂Cl₂ and the solution was washed with water (twice) and dried (MgSO₄). The solids were removed by filtration and the filtrate was evaporated under reduced pressure. The residue was crystallized from DIPE and CH₃CN (5:1) and the white precipitate was collected by filtration and dried under vacuum. ¹H NMR (400 MHz, DMSO-d₆) δ ppm 0.11 - 0.20 (m, 1H), 0.26 - 0.34 (m, 1H), 0.37 - 0.46 (m, 1H), 0.55 - 0.63 (m, 1H), 1.47 (s, 3H), 1.54 (s, 3H), 1.55 - 1.60 (m, 1H), 2.48 (s, 3H), 3.87 (dd, J=13.4, 5.3 Hz, 1H), 3.95 (s, 3H), 3.99 (dd, J=13.4, 6.5 Hz, 1H), 5.57 (s, 1H), 5.58 (s, 1H), 7.34 (d, J=1.6 Hz, 1H), 7.51 - 7.59 (m, 1H), 7.76 (d, J=1.6 Hz, 1H), 7.78 -7.83 (m, 1H), 7.95 - 8.04 (m, 3H), 8.53 (t, J=5.9 Hz, 1H), 8.76 (d, J=2.4 Hz, 1H); LC-MS (method H): Rt = 2.08 min; mass calcd. for C₃₁H₃₀F₃N₃O₄ 565.2, m/z found 566.4 [M+H]⁺; [α]_{D}²⁰ -41.25° (c 0.414, DMF).

### (-)-N-{2-[5-Chloro-6-(4-Fluorophenyl)-4-(2-Hydroxypropan-2-yl)Pyridin-2-yl]-3,3,3-Trifluoro-2-Hydroxypropyl}-2-Fluoro-8-Methoxy-3-Methylquinoline-6-Carboxamide 210

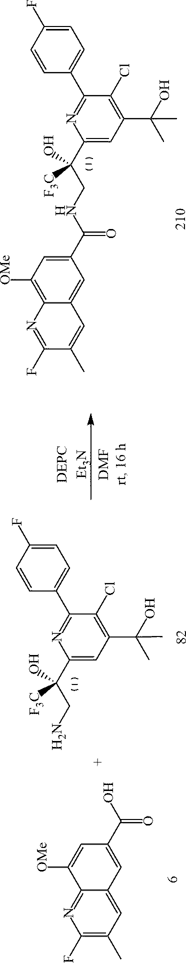

210 (117 mg. 75%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 1.57 (s, 3H), 1.65 (s, 3H), 2.41 (s, 3H), 3.96 (s, 3H), 4.12 (dd, J=14.0, 5.6 Hz, 1H), 4.21 (dd, J=13.8, 6.3 Hz, 1H), 5.70 (s, 1H), 7.26 - 7.35 (m, 2H), 7.39 - 7.46 (m, 2H), 7.66 -7.75 (m, 2H), 7.86 (d, J=1.5 Hz, 1H), 8.30 - 8.36 (m, 2H), 8.75 (t, J=5.8 Hz, 1H); LC-MS (method B): Rt = 1.20 min; mass calcd. for C₂₉H₂₅ClF₅N₃O₄ 609.0, m/z found 610.3 [M+H]⁺; [α]_{D}²⁰ -73.01° (c 0.257, DMF).

### (-)-N-{2-[3,5-Difluoro-6-(4-Fluorophenyl)-4-(2-Hydroxypropan-2-yl)Pyridin-2-yl]-3,3,3-Trifluoro-2-Hydroxypropyl}-2-Fluoro-8-Methoxy-3-Methylquinoline-6-Carboxamide 211

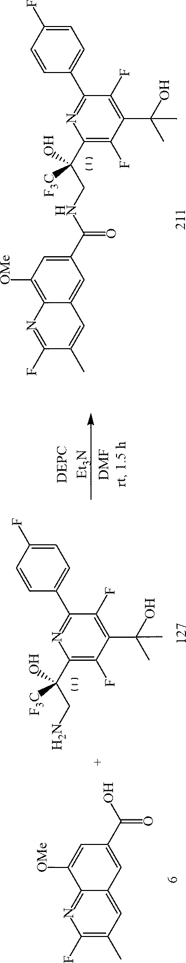

211 (77 mg. 50%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆) δ ppm 1.61 (s, 6H), 2.38 (s, 3H), 3.90 (s, 3H), 4.08 (dd, J=14.0, 5.2 Hz, 1H), 4.40 (dd, J=13.9, 6.8 Hz, 1H), 5.60 (s, 1H), 7.04 (s, 1H), 7.30 - 7.39 (m, 3H), 7.80 (d, J=1.5 Hz, 1H), 7.92 (dd, J=7.5, 5.5 Hz, 2H), 8.26 (d, J=10.1 Hz, 1H), 8.58 (br t, J=5.9 Hz, 1H); LC-MS (method B): Rt 1.12 min; mass calcd. for C₂₉H₂₄F₇N₃O₄ 611.0, m/z found 612.0 [M+H]⁺; [α]_{D}²⁰ = -21.58° (c 0.260, DMF).

### (-)-N-{2-[5-Chloro-3-Fluoro-6-(4-Fluorophenyl)-4-(2-Hydroxypropan-2-yl)Pyridin-2-yl]-3,3,3-Trifluoro-2-Hydroxypropyl}-2-Fluoro-8-Methoxy-3-Methylquinoline-6-Carboxamide 212

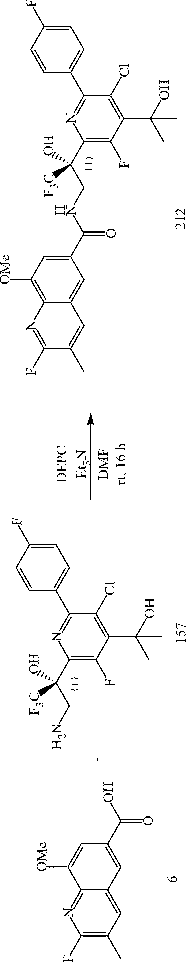

212 (120 mg. 78%) was synthesized according to procedure A. The purification was performed via reverse phase HPLC (stationary phase: 250 g, YMC Tri-Art, mobile phase: NH₄HCO₃ (0.25% in H₂O)/CH₃CN, gradient from 65:35 to 35:65). ¹H NMR (400 MHz, DMSO-d₆) δ ppm 1.65(s, 3H), 1.66 (s, 3H), 2.40 (s, 3H), 3.92 (s, 3H), 4.04 (dd, J=14.0, 5.0 Hz, 1H), 4.40 (dd, J=13.8, 6.7 Hz, 1H), 5.53 (s, 1H), 6.95 (s, 1H), 7.22 - 7.33 (m, 2H), 7.35 (d, J=1.3 Hz, 1H), 7.53 - 7.62 (m, 2H), 7.81 (d, J=1.5 Hz, 1H), 8.31 (d, J=9.9 Hz, 1H), 8.59 (br t, J=5.9 Hz, 1H); LC-MS (method G): Rt 2.16 min; mass calcd. for C₂₉H₂₄ClF₆N₃O₄ 627.2, m/z found 628.2 [M+H]⁺; [α]_{D}²⁰ -47.06° (c 0.264, DMF).

### (+)-N-{3,3-Difluoro-2-[5-Fluoro-6-(4-Fluorophenyl)-4-(2-Hydroxypropan-2-yl)Pyridin-2-yl]-2-Hydroxypropyl}-2-Fluoro-8-Methoxy-3-Methylquinoline-6-Carboxamide 213 and (-)-N-{3,3-Difluoro-2-[5-Fluoro-6-(4-Fluorophenyl)-4-(2-Hydroxypropan-2-yl)Pyridin-2-yl]-2-Hydroxypropyl}-2-Fluoro-8-Methoxy-3-Methylquinoline-6-Carboxamide 214

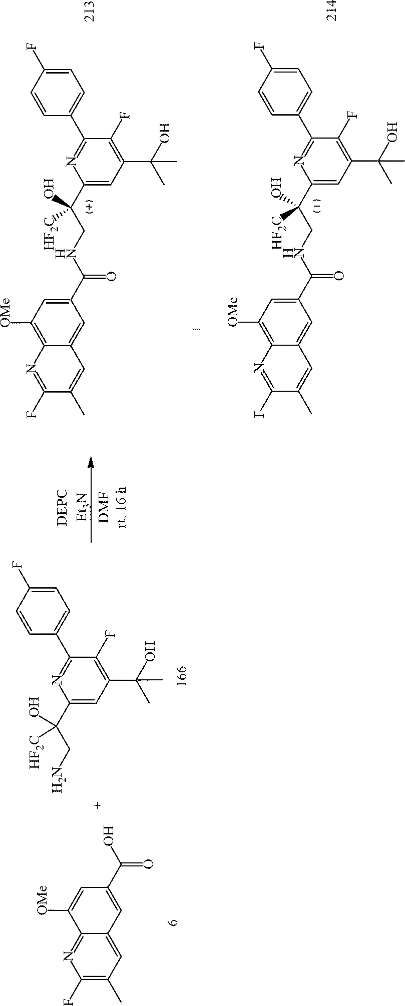

213 (56 mg. 31%) and 214 (50 mg, 28%) were synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆) δ ppm 8.58 (t, J=6.2 Hz, 1H), 8.20 (d, J=10.1 Hz, 1H), 8.03 (d, J=5.5 Hz, 1H), 7.93 (dd, J=7.6, 5.6 Hz, 2H), 7.80 (d, J=1.5 Hz, 1H), 7.38 (d, J=1.5 Hz, 1H), 7.20 - 7.30 (m, 2H), 6.63 (t, J=54.6 Hs, 1H), 6.59 (s, 1H), 5.61 (s, 1H), 3.95 - 4.04 (m, 1H), 3.92 (s, 3H), 3.78 - 3.87 (m, 1H), 2.40 (s, 3H), 1.53 (s, 3H), 1.45 (s, 3H): LC-MS (method B): Rt 1.08 min; mass calcd. for C₂₉H₂₆F₅N₃O₄ 575.0, m/z found 573.3 [M+H]⁺. A second purification was performed via silica column chromatography (heptane/EtOH, 90:10) delivered 213 (56 mg, 31%); [α]_{D}²⁰ +83.74° (c 0.268, DMF); and 214 (50 mg, 28%); [α]_{D}²⁰ -77.37° (c 0.259, DMF).

### (-)-N-[5-Chloro-6-(4-Fluorophenyl)-4-Hydroxypropan-2-yl)Pyridin-2-yl]-2-Cyclopropyl-2-Hydroxyethyl}-2-Fluoro-8-Methoxy-3-Methylquinoline-6-Carboxamide 215

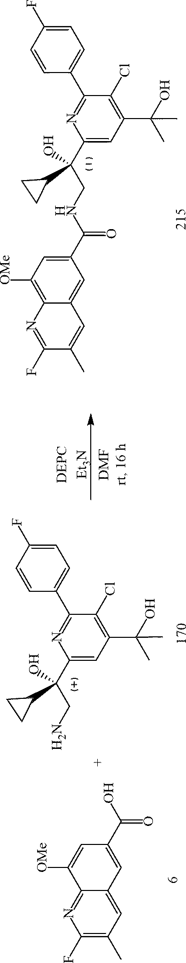

215 (119 mg. 75%) was synthesized according to procedure A and purified by reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.10 - 0.19 (m, 1H), 0.25 - 0.33 (m, 1H), 0.34 - 0.43 (m, 1H), 0.52 - 0.60 (m, 1H), 1.45 - 1.54 (m, 1H), 1.56 (s, 3H), 1.63 (s, 3H), 2.40 (s, 3H), 3.85 (dd, J=13.4, 5.5 Hz, 1H), 3.89 - 3.94 (m, 1H), 3.95 (s, 3H), 5.52 (s, 1H), 5.57 (s, 1H), 7.22 - 7.30 (m, 2H), 7.43 (d, J=1.3 Hz, 1H), 7.64 - 7.73 (m, 2H), 7.82 (d, J=1.5 Hz, 1H), 8.14 (s, 1H), 8.29 (d, J=10.1 Hz, 1H), 8.59 (t, J=5.9 Hz, 1H); LC-MS (method F): Rt = 2.53 min; mass calcd. for C₃₁H₃₀ClF₂N₃O₄ 581.0, m/z found 582.4 [M+H]⁺; [α]_{D}²⁰ -7.84° (c 0.268, DMF).

### (-)-N-{2-Cyclopropyl-2-[5-Fluoro-6-(4-Fluorophenyl)-4-(2-Hydroxypropan-2-yl)Pyridin-2-yl]-2-Hydroxyethyl}-2-Fluoro-8-Methoxy-3-Methylquinoline-6-Carboxamide 216

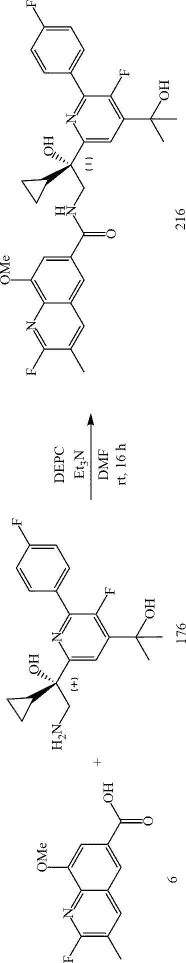

216 (110 mg. 68%) was synthesized according to procedure A then purified via reverse phase HPLC (Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.10 - 0.20 (m, 1H), 0.25 - 0.34 (m, 1H), 0.34 - 0.46 (m, 1H), 0.53 - 0.61 (m, 1H), 1.47 (s, 3H), 1.51 - 1.61 (m, 4H), 2.40 (s, 3 H), 3.85 - 3.99 (m, 5H), 5.55 (s, 1H), 5.56 (s, 1H), 7.27 - 7.36 (m, 2H), 7.43 (d, J=1.5 Hz, 1H), 7.84 (d, J=1.5 Hz, 1H), 7.94 (d, J=5.7 Hz, 1H), 7.98 (dd, J=7.5, 5.5 Hz, 2H), 8.27 (d, J=10.3 Hz, 1H), 8.58 (t, J=5.8 Hz, 1H); LC-MS (method F): Rt = 2.53 min; mass calcd. for C₃₁H₃₀F₃N₃O₄ 565.0, m/z found 566.4 [M+H]⁺; [α]_{D}²⁰ -30.36° (c 0.263, DMF).

### N-[(-)Cyclopropyl{5-Fluoro-4-(2-Hydroxypropanyl)-6-[4-(Trifluoromethyl)Phenyl]-Pyridinyl}Hydroxyethyl]Fluoro-8-Methoxy-3-Methylquinoline-6-Carboxamide 217

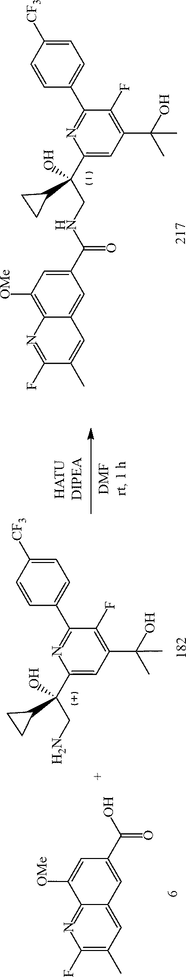

217 (270 mg. 88%) was synthesized according to procedure B. A purification was performed via SFC (stationary phase: Chiralpak Diacel AD 20 x 250 mm, mobile phase: CO₂, EtOH + 0.4% i-PrNH₂). ¹H NMR (400 MHz, DMSO-d₆) δ ppm 8.57 (t, J=5.9 Hz, 1H), 8.30 (d, J=10.2 Hz, 1H), 8.14 (d, J=8.1 Hz, 2H), 8.01 (d, J=5.7 Hz, 1H), 7.81 - 7.87 (m, 3H), 7.43 (d, J=1.2 Hz, 1H), 5.60 (s, 1H), 5.55 (s, 1H), 3.87 - 3.99 (m, 2H), 3.94 (s, 3H), 2.39 (s, 3H), 1.57 - 1.63 (m, 1H), 1.55 (s, 3H), 1.48 (s, 3H), 0.55 - 0.64 (m, 1H), 0.38 - 0.46 (m, 1H), 0.27 - 0.36 (m, 1H), 0.12 - 0.22 (m, 1H); LC-MS (method C): Rt 2.31 min; mass calcd. for C₃₂H₃₀F₅N₃O₄ 615.2, m/z found 616.2 [M+H]⁺; [α]_{D}²⁰ -33.5° (c 0.41, DMF).

### (-)-N-{2-Cyclopropyl-2-[6-(3,4-Difluorophenyl)-5-Fluoro-4-(2-Hydroxypropan-2-yl)Pyridin-2-yl]-2-Hydroxyethyl}-2-Fluoro-8-Methoxy-3-Methylquinoline-6-Carboxamide 218

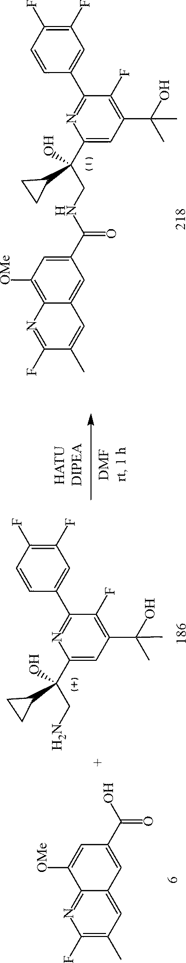

218 (192 mg. 66%) was synthesized according to procedure B. A purification was performed via preparatory HPLC (stationary phase: RP XBridge Prep C18 OBD-10µm, 50x150mm, mobile phase: NH₄HCO₃ (0.25% in H₂O)/CH₃CN).¹H NMR (400 MHz, DMSO-d₆) δ ppm 0.10 - 0.21 (m, 1H), 0.25 - 0.34 (m, 1H), 0.37 - 0.46 (m, 1H), 0.55 - 0.63 (m, 1H), 1.47 (s, 3H), 1.54 (s, 3H), 1.55 - 1.61 (m, 1H), 2.40 (s, 3H), 3.84 - 3.91 (m, 1H), 3.94 (s, 3H), 3.95 - 4.01 (m, 1H), 5.55 (s, 1H), 5.58 (s, 1H), 7.43 (d, J=1.2 Hz, 1H), 7.51 - 7.60 (m, 1H), 7.77 - 7.83 (m, 1H), 7.85 (d, J=1.2 Hz, 1H), 7.94 - 8.02 (m, 2H), 8.29 (dd, J=10.2, 0.8 Hz, 1H), 8.57 (t, J=5.9 Hz, 1H); LC-MS (method A): Rt 9.45 min; mass calcd. for C₃₁H₂₉F₄N₃O₄ 583.2, m/z found 584.2 [M+H]⁺; [α]_{D}²⁰ -36.01° (c 0.281, DMF).

### (-)-N-{2-[5-Chloro-6-(4-Fluorophenyl)-4-(2-Hydroxypropan-2-yl)Pyridin-2-yl]-3,3,3-Trifluoro-2-Hydroxypropyl}-8-(Cyclopropyloxy)-3-Methylquinoline-6-Carboxamide 219

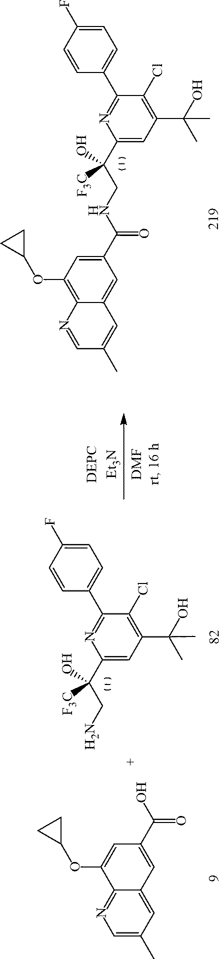

219 (120 mg. 76%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆,) δ ppm 0.72 - 0.81 (m, 2H), 0.81 - 0.90 (m, 2H), 1.58 (s, 3H), 1.65 (s, 3H), 2.48 (s, 3H), 3.99 - 4.05 (m, 1H), 4.11 (dd, J=13.9, 5.3 Hz, 1H), 4.19 - 4.28 (m, 1H), 5.68 (s, 1H), 7.26 -7.33 (m, 2H), 7.43 (s, 1H), 7.68 (d, J=1.5 Hz, 1H), 7.69 - 7.74 (m, 2H), 7.78 (d, J=1.5 Hz, 1H), 8.02 - 8.06 (m, 1H), 8.34 (s, 1H), 8.70 (t, J=5.9 Hz, 1H), 8.76 (d, J=2.2 Hz, 1H); LC-MS (method D): Rt 2.48 min; mass calcd. for C₃₁H₂₈ClF₄N₃O₄ 617.0, m/z found 618.0 [M+H]⁺; [α]_{D}²⁰ -73.66° (c 0.253, DMF).

### (-)8-(Cyclopropyloxy)-3-Methyl-N-{(2S)-3,3,3-Trifluoro-2-[5-Fluoro-6-(4-Fluorophenyl)-4-(2-Hydroxypropan-2-yl)Pyridin-2-yl]-2-Hydroxypropyl}Quinoline-6-Carboxamide 272

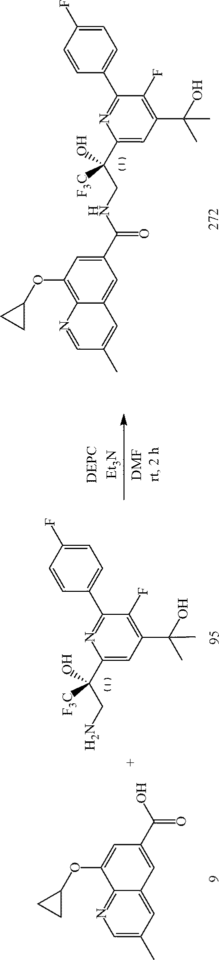

272 (121 mg, 76%) was synthesized according to procedure A. The purification was performed via reverse phase HPLC (stationary phase: Kromasil C18 100A 5 um (Eka Nobel), mobile phase: NH₄HCO₃ (0.25% in H₂O)/CH₃CN, gradient from 70:30 to 20:80). ¹H NMR (400 MHz, DMSO-d₆) δ ppm 0.71 - 0.80 (m, 2H), 0.80 - 0.90 (m, 2H), 1.49 (s, 3H), 1.56 (s, 3H), 2.48 (s, 3H), 4.00 (tt, J=6.0, 2.9 Hz, 1H), 4.12 - 4.20 (m, 1H), 4.24 - 4.31 (m, 1H), 5.67 (s, 1H), 7.32 - 7.39 (m, 2H), 7.40 (s, 1H), 7.67 (d, J=1.5 Hz, 1H), 7.81 (d, J=1.5 Hz, 1H), 7.98 - 8.04 (m, 3H), 8.14 (d, J=5.3 Hz, 1H), 8.68 (t, J=5.9 Hz, 1H), 8.75 (d, J=2.2 Hz, 1H); LC-MS (method G): Rt 2.16 min; mass calcd. for C₃₁H₂₈F₅N₃O₄ 601.2, m/z found 602.0 [M+H]⁺; [α]_{D}²⁰ -167° (c 0.3, DMF).

### (-)(Cyclopropyloxy)-N-{2-[3,5-Difluoro-6-(4-Fluorophenyl)-4-(2-Hydroxypropan-2-yl)Pyridin-2-yl]-3,3,3-Trifluoro-2-Hydroxypropyl}-3-Methylquinoline-6-Carboxamide 220

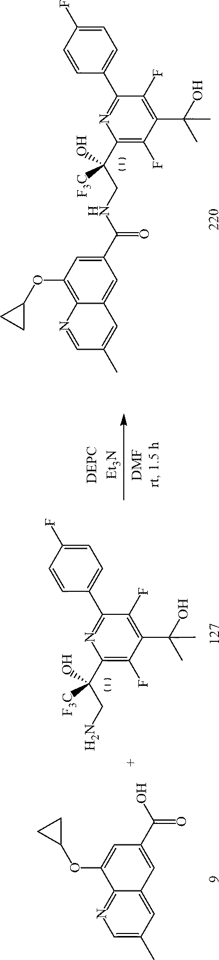

220 (95 mg. 60%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.69 - 0.76 (m, 2H), 0.76 - 0.83 (m, 2H), 1.61 (s, 6H), 2.46 (s, 3H), 3.90 - 3.97 (m, 1H), 4.07 (dd, J=13.8, 5.0 Hz, 1H), 4.41 (dd, J=13.8, 6.5 Hz, 1H), 5.60 (s, 1H), 7.08 (s, 1H), 7.30 - 7.39 (m, 2H), 7.58 (d, J=1.5 Hz, 1H), 7.73 (d, J=1.5 Hz, 1H), 7.93 (dd, J=7.4, 5.6 Hz, 2H), 7.97 (d, J=0.9 Hz, 1H), 8.54 (t, J=5.9 Hz, 1H), 8.73 (d, J=2.0 Hz, 1H); LC-MS (method B): Rt 1.10 min; mass calcd. for C₃₁H₂₇F₆N₃O₄ 619.0, m/z found 620.0 [M+H]⁺; [α]_{D}²⁰ -26.64° (c 0.259, DMF).

### (-)-N-{2-[5-Chloro-3-Fluoro-6-(4-Fluorophenyl)-4-(2-Hydroxypropan-2-yl)Pyridin-2-yl]-3,3,3-Trifluoro-2-Hydroxypropyl}-8-(Cyclopropyloxy)-3-Methylquinoline-6-Carboxamide 221

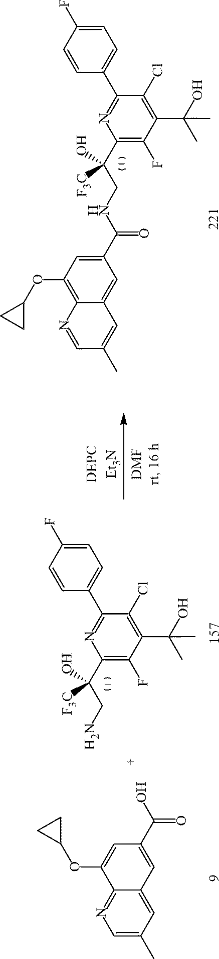

221 (110 mg, 71%) was synthesized according to procedure A. The purification was performed via reverse phase HPLC (stationary phase: 250 g YMC Tri-Art, mobile phase: NH₄HCO₃ (0.25% in H₂O)/CH₃CN, gradient from 65:35 to 35:65). ¹H NMR (400 MHz, DMSO-d₆) δ ppm 0.71 -0.86 (m, 4H), 1.66 (s, 3H), 1.67 (s, 3H), 2.48 (s, 3H), 3.94 - 4.1 (m, 1H), 4.04 (dd, J=13.9, 5.1 Hz, 1H), 4.40 (dd, J=13.8, 6.7 Hz, 1H), 5.53 (s, 1H), 6.95 (s, 1H), 7.23 - 7.31 (m, 2H), 7.53 -7.64 (m, 3H), 7.74 (d, J=1.5 Hz, 1H), 8.02 (d, J=1.1 Hz, 1H), 8.54 (t, J=6.1 Hz, 1H), 8.75 (d, J=2.0 Hz, 1H); LC-MS (method G): Rt 2.14 min; mass calcd. for C₃₁H₂₇ClF₅N₃O₄ 635.2, m/z found 636.2 [M+H]⁺; [α]_{D}²⁰ -46.32° (c 0.272, DMF).

### (-)-A-{2-[5-Chloro-6-(4-Fluorophenyl)-4-(2-Hydroxypropan-2-yl)Pyridin-2-yl]-2-Cyclopropyl-2-Hydroxyethyl}-8-(Cyclopropyloxy)-3-Methylquinoline-6-Carboxamide 222

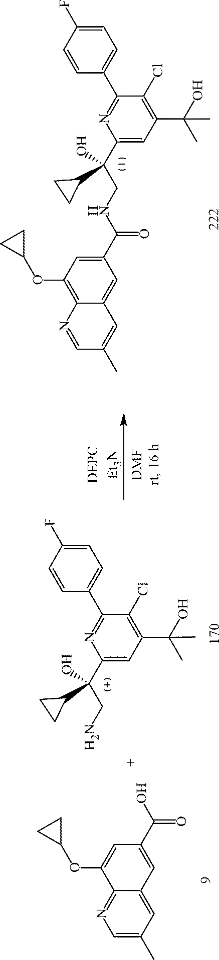

222 (115 mg, 71%) was synthesized according to procedure A. The purification was performed via reverse phase HPLC (stationary phase: Kromasil C18, 100A, 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.10 - 0.19 (m, 1H), 0.26 - 0.34 (m, 1H), 0.35 - 0.43 (m, 1H), 0.53 - 0.61 (m, 1H), 0.72 - 0.89 (m, 4H), 1.45 - 1.53 (m, 1H), 1.57 (s, 3H), 1.64 (s, 3H), 2.48 (s, 3H), 3.86 (dd, J=13.4, 5.3 Hz, 1H), 3.94 (dd, J=13.4, 6.4 Hz, 1H), 3.99 - 4.05 (m, 1H), 5.55 (s, 1H), 5.57 (s, 1H), 7.23 - 7.30 (m, 2H), 7.66 - 7.73 (m, 3H), 7.74 (d, J=1.8 Hz, 1H), 8.00 (dd, J=1.9, 1.0 Hz, 1H), 8.16 (s, 1H), 8.54 (t, J=5.8 Hz, 1H), 8.75 (d, J=2.2 Hz, 1H); LC-MS (method F): Rt = 2.55 min; mass calcd. for C₃₃H₃₃ClFN₃O₄ 589.0, m/z found 590.4 [M+H]⁺; [α]_{D}²⁰ -82.15° (c 0.261, DMF).

### (-)-N-{2-Cyclopropyl-2-[5-Fluoro-6-(4-Fluorophenyl)-4-(2-Hydroxypropan-2-yl)Pyridin-2-yl]-2-Hydroxyethyl}-8-(Cyclopropyloxy)-3-Methylquinoline-6-Carboxamide 223

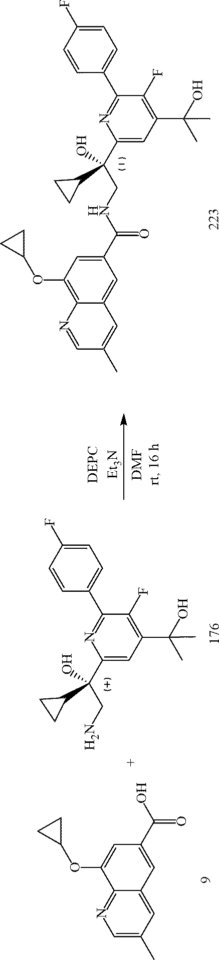

223 (112 mg. 68%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.11 - 0.20 (m, 1H), 0.26 - 0.34 (m, 1H), 0.36 - 0.46 (m, 1H), 0.54 - 0.63 (m, 1H), 0.70 - 0.80 (m, 2H), 0.80 - 0.88 (m, 2H), 1.48 (s, 3H), 1.51 - 1.60 (m, 4H), 2.47 (s, 3H), 3.85 - 3.92 (m, 1H), 3.93 - 4.04 (m, 2H), 5.55 (br s, 1H), 5.57 (br s, 1H), 7.26 - 7.37 (m, 2H), 7.67 (d, J=1.8 Hz, 1H), 7.77 (d, J=1.5 Hz, 1H), 7.92 - 8.03 (m, 4H), 8.52 (t, J=5.7 Hz, 1H), 8.74 (d, J=2.0 Hz, 1H); LC-MS (method F): Rt = 2.54 min; mass calcd. for C₃₃H₃₃F₂N₃O₄ 573.0, m/z found 574.5 [M+H]⁺; [α]_{D}²⁰ -29.64° (c 0.253, DMF).

### (-)-N-Cyclopropyl-2-{5-Fluoro-4-(2-Hydroxypropan-2-yl)-6-[4-(Trifluoromethyl)Phenyl]-Pyridin-2-yl}-2-Hydroxyethyl]-8-(Cyclopropyloxy)-3-Methylquinoline-6-Carboxamide 224

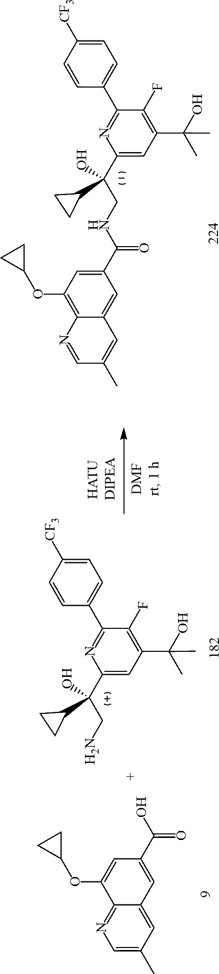

224 (301 mg. 97%) was synthesized according to procedure B. ¹H NMR (400 MHz, DMSO-d₆) δ ppm 0.12 - 0.21 (m, 1H), 0.27 - 0.36 (m, 1H), 0.38 - 0.47 (m, 1H), 0.56 - 0.65 (m, 1H), 0.70 -0.89 (m, 4H), 1.48 (s, 3H), 1.53 - 1.61 (m, 4H), 2.47 (s, 3H), 3.91 (dd, J=13.4, 5.3 Hz, 1H), 3.94 - 4.03 (m, 2H), 5.56 (s, 1H), 5.60 (s, 1H), 7.67 (d, J=1.6 Hz, 1H), 7.79 (d, J=1.2 Hz, 1H), 7.85 (d, J=8.5 Hz, 2H), 8.00 - 8.04 (m, 2H), 8.15 (br d, J=8.1 Hz, 2H), 8.51 (t, J=5.7 Hz, 1H), 8.74 (d, J=2.0 Hz, 1H); LC-MS (method H): Rt = 2.25 min; mass calcd. for C₃₄H₃₃F₄N₃O₄ 623.2, m/z found 624.5 [M+H]⁺; [α]_{D}²⁰ -38.23° (c 0.293, DMF).

### (-)-N-{2-Cyclopropyl-2-[6-(3,4-Difluorophenyl)-5-Fluoro-4-(2-Hydroxypropan-2-yl)Pyridin-2-yl]-2-Hydroxyethyl}-8-(Cyclopropyloxy)-3-Methylquinoline-6-Carboxamide 225

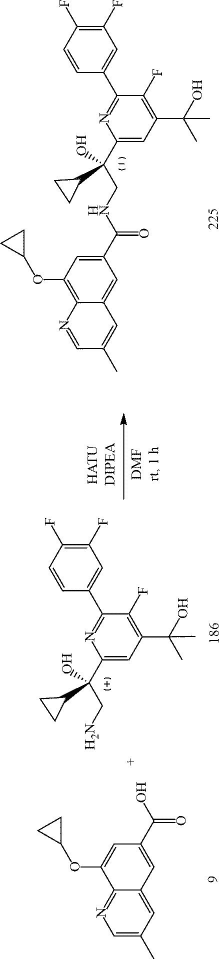

225 (290 mg. 98%) was synthesized according to procedure B. ¹H NMR (400 MHz, DMSO-d₆) δ ppm 0.10 - 0.21 (m, 1H), 0.26 - 0.34 (m, 1H), 0.38 - 0.46 (m, 1H), 0.56 - 0.64 (m, 1H), 0.70 -0.80 (m, 2H), 0.81 - 0.88 (m, 2H), 1.47 (s, 3H), 1.52 - 1.60 (m, 4H), 2.47 (s, 3H), 3.87 (dd, J=13.4, 5.3 Hz, 1H), 3.96 - 4.04 (m, 2H), 5.56 (s, 1H), 5.58 (s, 1H), 7.55 (dt, J=10.6, 8.5 Hz, 1H), 7.66 (d, J=1.6 Hz, 1H), 7.77 (d, J=2.0 Hz, 1H), 7.78 - 7.84 (m, 1H), 7.94 - 8.04 (m, 3H), 8.50 (t, J=5.9 Hz, 1H), 8.74 (d, J=2.0 Hz, 1H); LC-MS (method H): Rt = 2.15 min; mass calcd. for C₃₃H₃₂F₃N₃O₄ 591.2, m/z found 592.5 [M+H]⁺; [α]_{D}²⁰ -32.28° (c 0.361, DMF).

### (-)-N-{2-[5-Chloro-6-(4-Fluorophenyl)-4-(2-Hydroxypropan-2-yl)Pyridin-2-yl]-2-Cyclopropyl-2-Hydroxyethyl}-3-(Difluoromethyl)-8-Methoxyquinoline-6-Carboxamide226

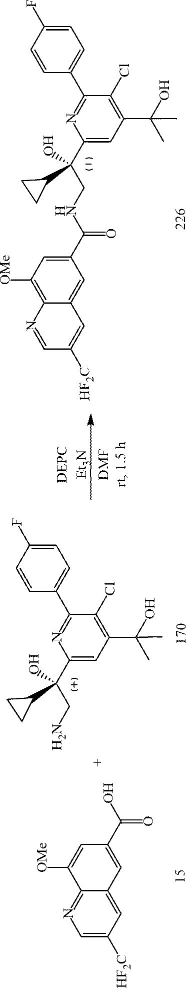

226 (126 mg. 77%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆) δ ppm 9.06 (d, J=2.0 Hz, 1H), 8.62 (t, J=5.9 Hz, 1H), 8.58 (d, J=1.8 Hz, 1H), 8.15 (s, 1H), 7.98 (d, J=1.5 Hz, 1H), 7.66 - 7.71 (m, 2H), 7.52 (d, J=1.3 Hz, 1H), 7.34 (t, J=55., 1H), 7.21 - 7.28 (m, 2H), 5.58 (s, 1H), 5.51 (s, 1H), 4.00 (s, 3H), 3.90 - 3.96 (m, 1H), 3.82 - 3.89 (m, 1H), 1.63 (s, 3H), 1.56 (s, 3H), 1.47 - 1.54 (m, 1H), 0.53 - 0.62 (m, 1H), 0.35 - 0.43 (m, 1H), 0.26 - 0.34 (m, 1H), 0.11 - 0.19 (m, 1H); LC-MS (method B): Rt = 1.09 min; mass calcd. for C₃₁H₂₉ClF₃N₃O₄ 599.0, m/z found 600.0 [M+H]⁺; [α]_{D}²⁰ -9.94° (c 0.251, DMF).

### N-{(-)Cyclopropyl[5-Fluoro-6-(4-Fluorophenyl)-4-(2-Hydroxypropanyl)Pyridinyl]Hydroxyethyl}-3-(Difluoromethyl)-8-Methoxyquinoline-6-Carboxamide 227

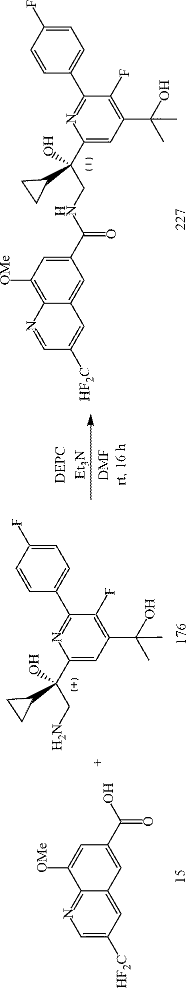

227 (255 mg. 60%) was synthesized according to procedure A. The purification was performed via reverse phase HPLC (stationary phase: 250 g, YMC Tri-Art, mobile phase: NH₄HCO₃ (0.25% in H₂O)/CH₃CN, gradient from 65:35 to 35:65). ¹H NMR (400 MHz, DMSO-d₆) δ ppm 9.05 (d, J=2.0 Hz, 1H), 8.60 (t, J=5.9 Hz, 1H), 8.56 (d, J=1.8 Hz, 1H), 7.93 - 8.02 (m, 4H), 7.52 (d, J=1.5 Hz, 1H), 7.33 (t, J=55.1 Hz, 1H), 7.27 - 7.34 (m, 2H), 5.56 (s, 1H), 5.51 (s, 1H), 3.99 (s, 3H), 3.87 - 3.98 (m, 2H), 1.55 - 1.61 (m, 1H), 1.54 (s, 3H), 1.47 (s, 3H), 0.55 - 0.63 (m, 1H), 0.37 - 0.46 (m, 1H), 0.27 - 0.35 (m, 1H), 0.12 - 0.20 (m, 1H); LC-MS (method D): Rt = 2.44 min; mass calcd. for C₃₁H₂₉F₄N₃O₄ 583.0, m/z found 584.0 [M+H]⁺; [α]_{D}²⁰ -32.4° (c 0.25, DMF).

### (-)Cyclopropyl-N-{2-[3,5-Difluoro-6-(4-Fluorophenyl)-4-(2-Hydroxypropan-2-yl)Pyridin-2-yl]-3,3,3-Trifluoro-2-Hydroxypropyl}-8-Methoxyquinoline-6-Carboxamide 228

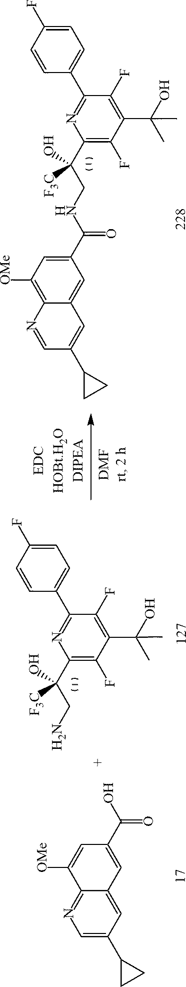

228 (61 mg. 49%) was synthesized according to procedure D. The crude mixture was purified by silica column chromatography (CH₂Cl₂/CH₃OH, gradient from 100:0 to 98:2). The residue was diluted in EtOH and evaporated under reduced pressure (3 times). ¹H NMR (400 MHz, DMSO-d₆) δ ppm 8.73 (d, J=2.1 Hz, 1H), 8.54 (br t, J=5.9 Hz, 1H), 7.93 (br dd, J=7.2, 5.9 Hz, 2H), 7.82 (d, J=1.8 Hz, 1H), 7.69 (d, J=1.0 Hz, 1H), 7.36 (t, J=8.8 Hz, 2H), 7.23 (s, 1H), 7.05 (s, 1H), 5.63 (s, 1H), 4.40 (br dd, J=13.7, 6.7 Hz, 1H), 4.07 (br dd, J=14.2, 4.8 Hz, 1H), 3.90 (s, 3H), 2.09 -2.18 (m, 1H), 1.60 (s, 6H), 1.06 - 1.11 (m, 2H), 0.81 - 0.88 (m, 2H); LC-MS (method I): Rt = 3.05 min; mass calcd. for C₃₁H₂₇F₆N₃O₄ 619.2, m/z found 620.2 [M+H]⁺; [α]_{D}²⁰ -22.31° (c 0.26, DMF).

### 4.2. Cinnolines

### (-)-N-{2-[5-Chloro-6-(4-Fluorophenyl)-4-(2-Hydroxypropan-2-yl)Pyridin-2-yl]-2-Hydroxypropyl}-8-Methoxy-3-Methylcinnoline-6-Carboxamide 229

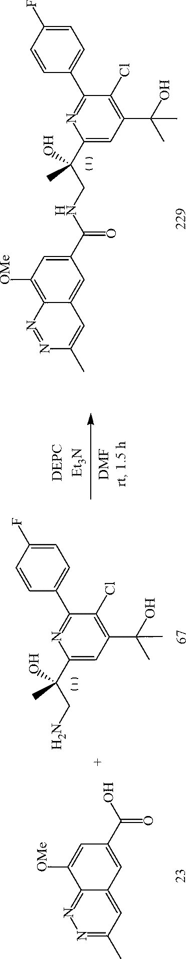

229 (73 mg. 46%) was synthesized according to procedure A and was purified via reverse phase HPLC (stationary phase: Kromasil C18 100A 5 µm (Eka Nobel), mobile phase: NH₄HCO₃ (0.25% in H₂O)/CH₃CN, gradient from 85:15 to 25:75). ¹H NMR (400 MHz, DMSO-d₆) δ ppm 1.51 (s, 3H), 1.57 (s, 3H), 1.63 (s, 3H), 2.88 (s, 3H), 3.64 - 3.78 (m, 2H), 4.08 (s, 3H), 5.58 (s, 1H), 5.71 (s, 1H), 7.20 - 7.28 (m, 2H), 7.43 (d, J=1.3 Hz, 1H), 7.60 - 7.69 (m, 2H), 7.79 (d, J=1.5 Hz, 1H), 7.91 (s, 1H), 8.24 (s, 1H), 8.60 (t, J=6.1 Hz, 1H); LC-MS (method B): Rt 0.91 min; mass calcd. for C₂₈H₂₈ClFN₄O₄ 538.0, m/z found 539.3 [M+H]⁺; [α]_{D}²⁰ -37.31° (c 0.26, DMF).

### (+)-N-{2-5-Chloro-6-(4-Fluorophenyl)-4-(2-Hydroxypropan-2-yl)Pyridin-2-yl]-2-Hydroxypropyl}-8-Methoxy-3-Methylcinnoline-6-Carboxamide 230

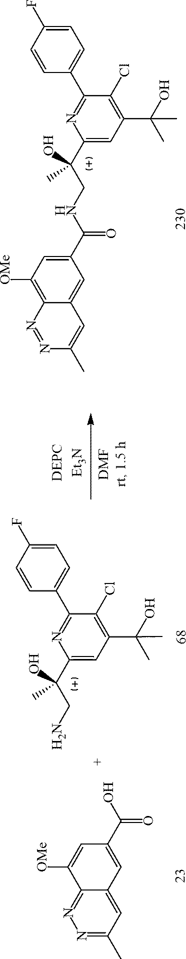

230 (63 mg. 38%) was synthesized according to procedure A and purified via reverse phase HPLC (stationary phase: 250 g YMC Tri-Art, mobile phase: NH₄HCO₃ (0.25% in H₂O)/CH₃CN, gradient from 65:35 to 35:65).¹H NMR (400 MHz, DMSO-d₆) δ ppm 1.51 (s, 3H), 1.57 (s, 3H), 1.63 (s, 3H), 2.88 (s, 3H), 3.63 - 3.77 (m, 2H), 4.08 (s, 3H), 5.58 (s, 1H), 5.72 (s, 1H), 7.20 - 7.28 (m, 2H), 7.43 (d, J=1.3 Hz, 1H), 7.61 - 7.68 (m, 2H), 7.79 (d, J=1.5 Hz, 1H), 7.91 (s, 1H), 8.24 (s, 1H), 8.60 (t, J=6.2 Hz, 1H); LC-MS (method D): Rt 2.31 min; mass calcd. for C₂₈H₂₈ClFN₄O₄ 538.0, m/z found 539 [M+H]⁺; [α]_{D}²⁰ +36.01° (c 0.256, DMF).

### (-)-N-{2-[5-Chloro-6-(4-Fluorophenyl)-4-(2-Hydroxypropan-2-yl)Pyridin-2-yl]-2-Hydroxy-3-Methylbutyl}-8-Methoxy-3-Methylcinnolme-6-Carboxamide 231

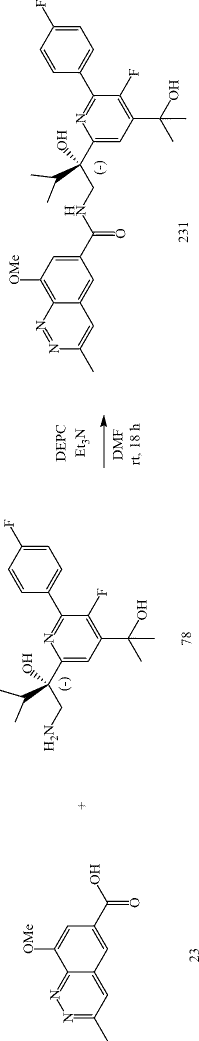

231 (398 mg. 79%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 85:15 to 20:80). ¹H NMR (400 MHz, DMSO-d₆) δ ppm 8.40 (dd, J=6.4, 4.8 Hz, 1H), 7.92 - 8.00 (m, 3H), 7.79 (s, 1H), 7.63 (d, J=1.3 Hz, 1H), 7.26 - 7.33 (m, 3H), 5.56 (s, 1H), 5.54 (s, 1H), 3.96 - 4.09 (m, 1H), 4.02 (s, 3H), 3.73 (dd, J=13.3, 4.5 Hz, 1H), 2.86 (s, 3H), 2.45 (quin, J=6.9 Hz, 1H), 1.53 (s, 3H), 1.44 (s, 3H), 1.02 (d, J=6.6 Hz, 3H), 0.72 (d, J=6.8 Hz, 3H); LC-MS (method G): Rt 1.93 min; mass calcd. for C₃₀H₃₂F₂N₄O₄ 536.2, m/z found 537.2 [M+H]⁺; [α]_{D}²⁰ -82.1° (c 0.525, DMF).

### (-)-N-{2-[5-Chloro-6-(4-Fluorophenyl)-4-(2-Hydroxypropan-2-yl)Pyridin-2-yl]-3,3,3-Trifluoro-2-Hydroxypropyl}-8-Methoxy-3-Methylcinnoline-6-Carboxamide 232

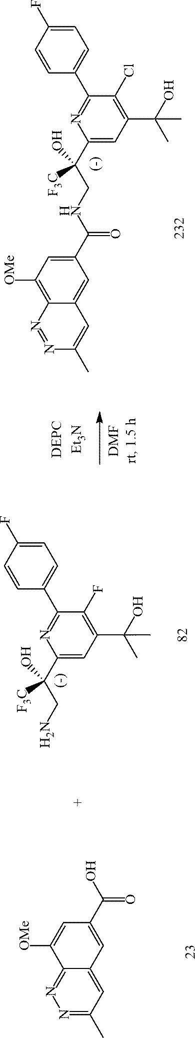

232 (122 mg. 81%) was synthesized according to procedure A. The purification was performed via reverse phase HPLC (Kromasil C18 100A 5 µm (Eka Nobel), mobile phase: NH₄HCO₃ (0.25% in H₂O)/CH₃CN, gradient from 70:30 to 10:90).¹H NMR (400 MHz, DMSO-d₆) δ ppm 1.57 (s, 3H), 1.65 (s, 3H), 2.87 (s, 3H), 4.05 - 4.15 (m, 4H), 4.18 - 4.26 (m, 1H), 5.69 (s, 1H), 7.25 - 7.34 (m, 3H), 7.39 (d, J=1.3 Hz, 1H), 7.65 - 7.73 (m, 2H), 7.76 (d, J=1.5 Hz, 1H), 7.95 (s, 1H), 8.33 (s, 1H), 8.80 (t, J=5.7 Hz, 1H); LC-MS (method B): Rt 1.10 min; mass calcd. for C₂₈H₂₅ClF₄N₄O₄ 592.0, m/z found 593.0 [M+H]⁺; [α]_{D}²⁰ -67.53° (c 0.270, DMF).

### (+)-N-{2-[5-Chloro-6-(4-Fluorophenyl)-4-{2-[(Methanesulfonyl)Amino]Propan-2-yl}Pyridin-2-yl]-3,3,3-Trifluoro-2-Hydroxypropyl}-8-Methoxy-3-Methylcinnoline-6-Carboxamide 233

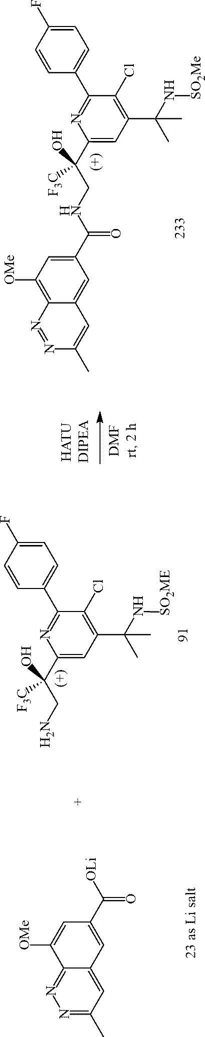

233 (54 mg. 47%) was synthesized according to procedure C. The crude mixture was purified by silica column chromatography (CH₂Cl₂/CH₃OH, gradient from 100:0 to 99:1). A second purification was performed via reverse phase (stationary phase: YMC-actus Triart C18 10 µm 30 x 150 mm, mobile phase: NH₄HCO₃ (0.25% in H₂O)/CH₃CN, gradient from 65:35 to 45:55). The residue was co-evaporated with EtOH (3 times) and dried under vacuum at 50° C. for 5 h. ¹H NMR (500 MHz, DMSO-d6, 30° C.) δ ppm 1.75 (s, 3H), 1.78 (s, 3H), 2.84 (s, 3H), 2.87 (s, 3H), 4.04 - 4.11 (m, 4H), 4.27 (br dd, J=13.9, 6.3 Hz, 1H), 7.26 - 7.34 (m, 3H), 7.39 (s, 1H), 7.69 (dd, J=8.5, 5.7 Hz, 2H), 7.74 (s, 2H), 7.95 (d, J=3.8 Hz, 2H), 8.74 (t, J=5.5 Hz, 1H); LC-MS (method I): Rt = 2.69 min; mass calcd. for C₂₉H₂₈ClF₄N₅O₅S 669.1, m/z found 670.3 [M+H]⁺; [α]_{D}²⁰ +64.75° (c 0.278, DMF).

### (-)Methoxy-3-Methyl-N-{3,3,3-Trifluoro-2-[5-Fluoro-6-(4-Fluorophenyl)-4-(2-Hydroxypropan-2-yl)Pyridin-2-yl]-2-Hydroxypropyl}Cinnoline-6-Carboxamide 234

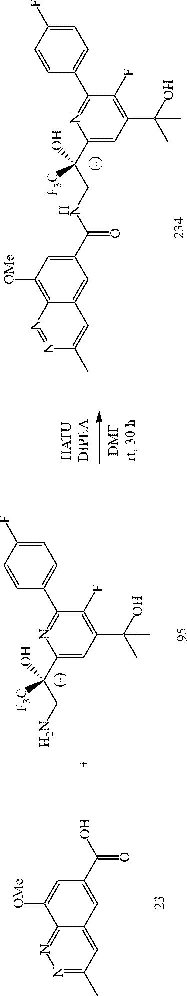

234 (1.7 g. 83%) was synthesized according to procedure B. The reaction was quenched by the addition of water and the mixture was stirred for few hours. The supernatant was removed. The resulting solid was triturated in water and collected by filtration. The residue was purified by silica column chromatography (EtOAc). ¹H NMR (400 MHz, DMSO-d₆) δ ppm 1.49 (s, 3H), 1.56 (s, 3H), 2.87 (s, 3H), 4.07 (s, 3H), 4.16 (dd, J=14.0, 5.6 Hz, 1H), 4.26 (dd, J=14.0, 6.2 Hz, 1H), 5.67 (s, 1H), 7.29 (s, 1H), 7.32 - 7.38 (m, 2H), 7.39 (d, J=1.3 Hz, 1H), 7.78 (d, J=1.5 Hz, 1H), 7.93 (s, 1H), 8.00 (dd, J=7.6, 5.6 Hz, 2H), 8.13 (d, J=5.5 Hz, 1H), 8.78 (t, J=5.9 Hz, 1H); LC-MS (method B): Rt = 1.01 min; mass calcd. for C₂₈H₂₅F₅N₄O₄ 576.0, m/z found 577.3 [M+H]⁺; [α]_{D}²⁰ -46.67° (c 0.09 DMF).

### (-)-N-{2-[4-(2-Acetamidopropan-2-yl)-5-Fluoro-6-(4-Fluorophenyl)Pyridin-2-yl]-3,3,3-Trifluoro-2-Hydroxypropyl}-8-Methoxy-3-Methylcinnoline-6-Carboxamide 235 and (+)-N-{2-[4-(2-Acetamidopropan-2-yl)-5-Fluoro-6-(4-Fluorophenyl)Pyridin-2-y1]-3,3,3-Trifluoro-2-Hydroxypropyl}-8-Methoxy-3-Methylcinnoline-6-Carboxamide 236

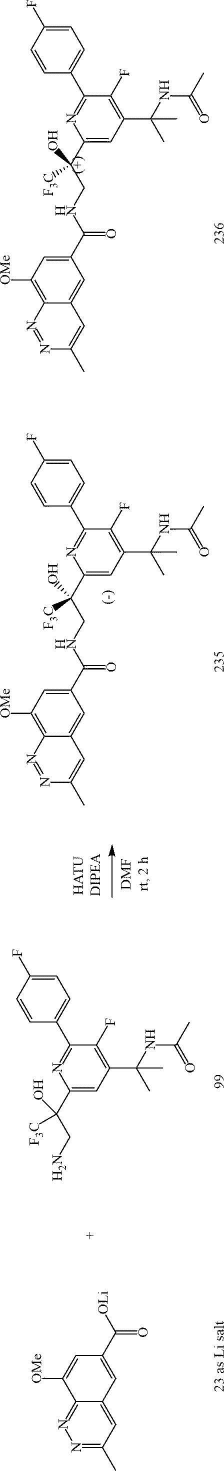

235 (38 mg. 26%) and 236 (35 mg, 24%) were synthesized according to procedure C. The crude mixture was purified by silica column chromatography (CH₂Cl₂/CH₃OH, gradient from 100:0 to 96:4). The residue was diluted with EtOH and evaporated under reduced pressure (twice). A second purification was performed by chiral SFC (stationary phase: CHIRALPAK AD-H 5 µm 250 x 30 mm, mobile phase: 80% CO₂, 20% EtOH) to give 235 (52 mg); ¹H NMR (500 MHz, DMSO-d₆, 31° C.) δ ppm 1.61 (s, 3H), 1.62 (s, 3H), 1.83 (s, 3H), 2.86 (s, 3H), 4.06 (s, 3H), 4.10 (dd, J=13.9, 5.4 Hz, 1H), 4.29 (br dd, J=13.9, 6.6 Hz, 1H), 7.24 (s, 1H), 7.31 - 7.37 (m, 2H), 7.39 (d, J=1.3 Hz, 1H), 7.71 - 7.77 (m, 2H), 7.97 (br dd, J=8.0, 5.8 Hz, 2H), 7.99 (s, 1H), 8.35 (s, 1H), 8.71 (br t, J=5.8 Hz, 1H); LC-MS (method I): Rt = 2.68 min; mass calcd. for C₃₀H₂₈F₅N₅O₄ 617.2, m/z found 618.4 [M+H]⁺; [α]_{D}²⁰ -74.23° (c 0.26, DMF); and 236 (51 mg); ¹H NMR (500 MHz, DMSO-d₆, 31° C.) δ ppm 1.61 (br s, 3H), 1.62 (br s, 3H), 1.83 (s, 3H), 2.86 (s, 3H), 4.06 (s, 3H), 4.10 (dd, J=14.1, 5.5 Hz, 1H), 4.29 (dd, J=14.1, 6.3 Hz, 1H), 7.25 (br s, 1H), 7.31 - 7.37 (m, 2H), 7.39 (d, J=1.3 Hz, 1H), 7.72 - 7.76 (m, 2H), 7.97 (dd, J=8.0, 5.8 Hz, 3H), 7.99 (s, 1H), 8.35 (s, 1H), 8.72 (br t, J=5.7 Hz, 1H); LC-MS (method I): Rt = 2.68 min; mass calcd. for C₃₀H₂₈F₅N₅O₄ 617.2, m/z found 618.4 [M+H]⁺; [α]_{D}²⁰ +87.31° (c 0.26, DMF).

### (-)Methoxy-3-Methyl-N-{3,3,3-Trifluoro-2-[5-Fluoro-6-(4-Fluorophenyl)-4-{2-[(MethaneSulfonyl)Amino]propan-2-yl}Pyridin-2-yl]-2-Hydroxypropyl}Cinnoline-6-Carboxamide 237

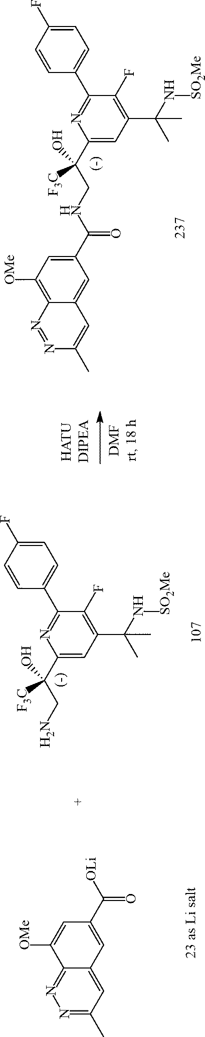

237 (80 mg. 64%) was synthesized according to procedure C. The crude mixture was purified by silica column chromatography (CH₂Cl₂/CH₃OH, gradient from 100:0 to 98:2). The crude was diluted with EtOH and evaporated under reduced pressure (twice). A second purification was performed by reverse phase (spherical C18, 25 µm, 40 g YMC-ODS-25, mobile phase: NH₄HCO₃ (0.25% in H₂O)/CH₃CN, gradient from 50:50 to 10:90). The residue was diluted with EtOH and evaporated under reduced pressure (twice). ¹H NMR (400 MHz, DMSO-d₆) δ ppm 1.70 (s, 6H), 2.86 (s, 3H), 2.87 (s, 3H), 4.07 (s, 3H), 4.14 (br dd, J=13.8, 4.4 Hz, 1H), 4.25 - 4.33 (m, 1H), 7.31 - 7.42 (m, 4H), 7.77 (s, 1H), 7.85 (br s, 1H), 7.92 - 7.96 (m, 2H), 8.00 (br dd, J=7.6, 6.1 Hz, 2H), 8.78 (br t, J=5.7 Hz, 1H); LC-MS (method I): Rt = 2.71 min; mass calcd. for C₂₉H₂₈F₅N₅O₅S 653.2, m/z found 654.3 [M+H]⁺; [α]_{D}²⁰ -78.89° (c 0.27, DMF).

### (+)Methoxy-3-Methyl-N-{3,3,3-Trifluoro-2-[5-Fluoro-6-(4-Fluoro-3-Methylphenyl)-4-(2-Hydroxypropan-2-yl)Pyridin-2-yl]-2-Hydroxypropyl}Cinnoline-6-Carboxamide 238

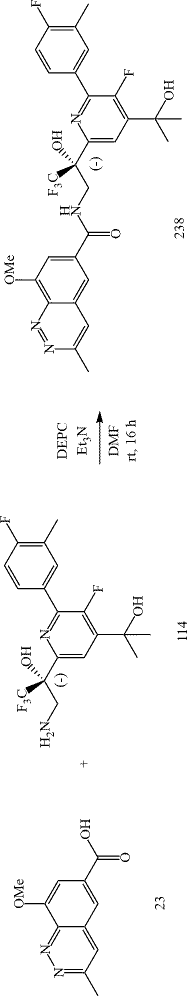

238 (93 mg. 51%) was synthesized according to procedure A. The purification was performed via reverse phase HPLC (Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆) δ ppm 1.49 (s, 3H), 1.55 (s, 3H), 2.26 (s, 3H), 2.86 (s, 3H), 4.06 (s, 3H), 4.13 (br dd, J=14.0, 5.4 Hz, 1H), 4.28 (br dd, J=14.0, 6.1 Hz, 1H), 5.66 (s, 1H), 7.22 - 7.43 (m, 3H), 7.74 - 7.86 (m, 3H), 7.91 (s, 1H), 8.11 (d, J=5.3 Hz, 1H), 8.80 (br t, J=5.7 Hz, 1H); LC-MS (method G): Rt 2.04 min; mass calcd. for C₂₉H₂₇F₅N₄O₄ 590.2, m/z found 591.2 [M+H]⁺; [α]_{D}²⁰ -79.08° (c 0.502, DMF).

### (-)-N-{2-[6-(3,4-Difluorophenyl)-5-Fluoro-4-(2-Hydroxypropan-2-yl)Pyridin-2-yl]-3,3,3-Trifluoro-2-Hydroxypropyl}-8-Methoxy-3-Methylcinnoline-6-Carboxamide 239

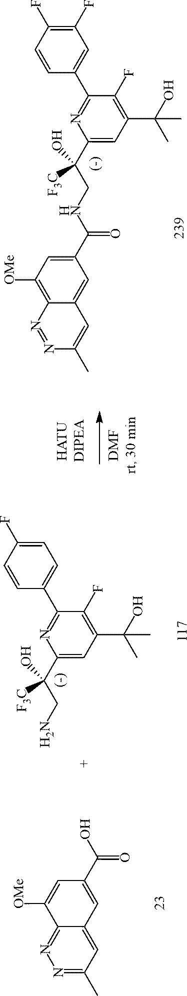

239 (71 mg. 47%) was synthesized according to procedure B. The reaction was quenched by the addition of water. The mixture was stirred for few hours and the surnatant was removed. The solid was triturated in water and collected by filtration. The solid was purified via SFC (Chiralpak Daicel IC 20 x 250 mm, mobile phase: CO₂, EtOH + 0.4% i-PrNH₂). ¹H NMR (400 MHz, DMSO-d₆) δ ppm 1.47 (s, 3H), 1.55 (s, 3H), 2.86 (s, 3H), 4.06 (s, 3H), 4.11 (dd, J=13.9, 5.5 Hz, 1H), 4.31 (dd, J=13.8, 6.3 Hz, 1H), 5.68 (s, 1H), 7.31 (s, 1H), 7.38 (d, J=1.3 Hz 1H), 7.58 (dt, J=10.6, 8.6 Hz, 1H), 7.78 (d, J=1.3 Hz, 1H), 7.79 - 7.85 (m, 1H), 7.95 (s, 1H), 7.98 (ddd, J=11.9, 8.1, 1.8 Hz, 1H), 8.15 (d, J=5.3 Hz, 1H), 8.77 (t, J=5.9 Hz, 1H); LC-MS (method H): Rt = 1.99 min; mass calcd. for C₂₈H₂₄F₆N₄O₄ 594.2, m/z found 594.4 [M+H]⁺; [α]_{D}²⁰ -97.85° (c 0.255, DMF).

### (-)-N-{2-[3,5-Difluoro-6-(4-Fluorophenyl)-4-(2-Hydroxypropan-2-yl)Pyridin-2-yl]-3,3,3-Trifluoro-2-Hydroxypropyl}-8-Methoxy-3-Methylcinnoline-6-Carboxamide 240

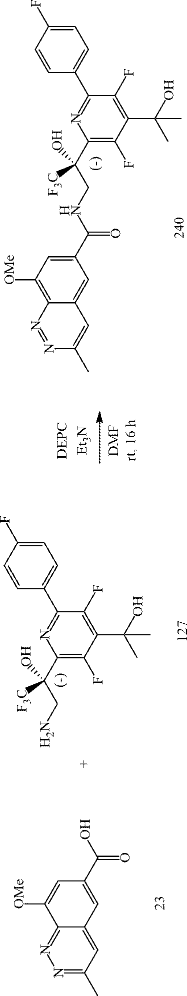

240 (220 mg. 58%) was synthesized according to procedure A. The purification was performed via reverse phase HPLC (Kromasil C18, 100A, 5 µm (Eka Nobel), mobile phase: NH₄HCO₃ (0.25% in H₂O)/CH₃CN, gradient from 90:10 to 30:70). ¹H NMR (400 MHz, DMSO-d₆) δ ppm 1.62 (s, 6H), 2.85 (s, 3H), 4.02 (s, 3H), 4.09 (dd, J=14.0, 5.2 Hz, 1H), 4.39 (dd, J=13.8, 6.7 Hz, 1H), 5.60 (s, 1H), 7.00 (s, 1H), 7.31 (d, J=1.3 Hz, 1H), 7.32 - 7.38 (m, 2H), 7.71 (d, J=1.5 Hz, 1H), 7.89 (s, 1H), 7.92 (dd, J=7.4, 5.6 Hz, 2H), 8.69 (t, J=6.1 Hz, 1H); LC-MS (method D): Rt 2.31 min; mass calcd. for C₂₈H₂₄F₆N₄O₄ 594.0, m/z found 595.0 [M+H]⁺; [α]_{D}²⁰ -25.19° (c 0.27, DMF).

### (-)-N-{2-[4-(2-Acetamidopropan-2-yl)-3,5-Difluoro-6-(4-Fluorophenyl)Pyridin-2-yl]-3,3,3-Trifluoro-2-Hydroxypropyl}-8-Methoxy-3-Methylcinnoline-6-Carboxamide 241

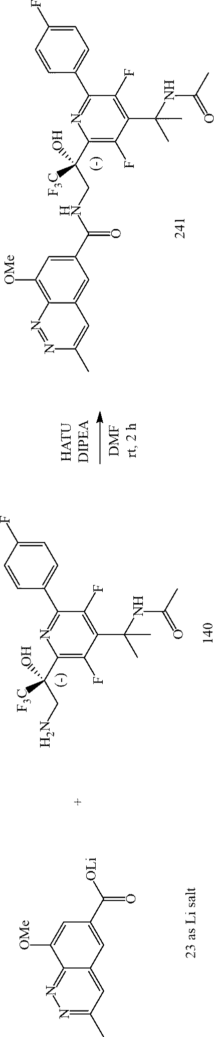

241 (78 mg, 54%) was synthesized according to procedure C. The crude mixture was purified by silica column chromatography (CH₂Cl₂/CH₃OH, gradient from 100:0 to 99:1). The crude was co-evaporated with EtOH (3 times) and dried under vacuum at 60° C. for 18 h. A second purification was performed by reverse phase (stationary phase: YMC-actus Triart C18 10 µm 30 x150 mm, 40 g, mobile phase: NH₄HCO₃ (0.25% in H₂O)/CH₃CN, gradient from 75:25 to 35:65). ¹H NMR (400 MHz, DMSO-d₆) δ ppm 1.67 (br s, 3H), 1.69 (br s, 3H), 1.76 (s, 3H), 2.85 (s, 3H), 4.02 (s, 3H), 4.03 - 4.09 (m, 1H), 4.44 (br dd, J=13.9, 6.7 Hz, 1H), 7.00 (s, 1H), 7.29 - 7.39 (m, 3H), 7.72 (d, J=1.1 Hz, 1H), 7.87 - 7.95 (m, 3H), 8.57 (s, 1H), 8.66 (br t, J=5.9 Hz, 1H); LC-MS (method I): Rt = 2.72 min; mass calcd. for C₃₀H₂₇F₆N₅O₄ 635.2, m/z found 636.5 [M+H]⁺; [α]_{D}²⁰ = -27.18° (c 0.287, DMF).

### (+)-N-{2-[4-(2-Acetamidopropan-2-yl)-3,5-Difluoro-6-(4-Fluorophenyl)Pyridin-2-yl]-3,3,3-Trifluoro-2-Hydroxypropyl}-8-Methoxy-3-Methylcinnoline-6-Carboxamide 242

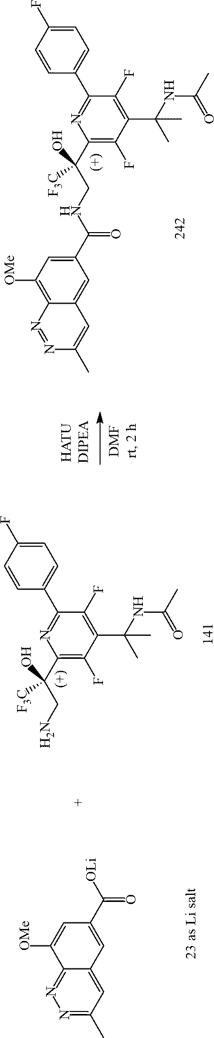

242 (81 mg. 55%) was synthesized according to procedure C. The crude mixture was purified by silica column chromatography (CH₂Cl₂/CH₃OH, gradient from 100:0 to 99:1). The residue was co-evaporated with EtOH (3 times) and dried under vacuum at 50° C. for 5 h. A second purification was performed by reverse phase (stationary phase: YMC-actus Triart C18 10 µm 30 x 150 mm, 40 g, mobile phase: NH₄HCO₃ (0.25% in H₂O)/CH₃CN, gradient from 75:25 to 35:65). ¹H NMR (400 MHz, DMSO-d₆) δ ppm 1.67 (br s, 3H), 1.69 (br s, 3H), 1.76 (s, 3H), 2.85 (s, 3H), 4.02 (s, 3H), 4.03 - 4.09 (m, 1H), 4.44 (br dd, J=13.6, 6.9 Hz, 1H), 7.00 (s, 1H), 7.31 (d, J=0.6 Hz, 1H), 7.32 - 7.39 (m, 2H), 7.72 (d, J=0.7 Hz, 1H), 7.87 - 7.96 (m, 3H), 8.57 (s, 1H), 8.66 (br t, J=5.9 Hz, 1H); LC-MS (method I): Rt = 2.72 min; mass calcd. for C₃₀H₂₇F₆N₅O₄ 635.2, m/z found 636.5 [M+H]⁺; [α]_{D}²⁰ +22° (c 0.300, DMF).

### (-)-N-{2-[3,5-Difluoro-6-(4-Fluorophenyl)-4-{2-[(Methanesulfoyl)Amino]Propan-2-yl}Pyridin-2-yl]-3,3,3-Trifluoro-2-Hydroxypropyl}-8-Methoxy-3-Methylcinnoline-6-Carboxamide 243

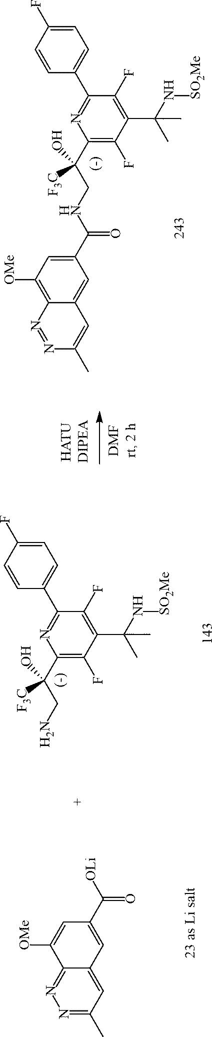

243 (78 mg, 55%) was synthesized according to procedure C. The crude mixture was purified by silica column chromatography (CH₂Cl₂/CH₃OH, gradient from 100:0 to 99:1). The residue was co-evaporated with EtOH (3 times) and dried under vacuum at 50° C. for 5 h. A second purification was performed by reverse phase (stationary phase: YMC-actus Triart C18 10 µm 30 x150 mm, 40 g, mobile phase: NH₄HCO₃ (0.25% in H₂O)/CH₃CN, gradient from 75:25 to 35:65). ¹H NMR (400 MHz, DMSO-d₆) δ ppm 1.78 (br s, 6H), 2.85 (s, 3H), 2.87 (s, 3H), 4.02 (s, 3H), 4.12 (br dd, J=13.8, 4.5 Hz, 1H), 4.39 (dd, J=13.3, 6.0 Hz, 1H), 7.06 (br s, 1H), 7.32 (d, J=1.0 Hz, 1H), 7.33 - 7.40 (m, 2H), 7.71 (d, J=1.1 Hz, 1H), 7.87 - 8.01 (m, 4H), 8.70 (t, J=5.8 Hz, 1H); LC-MS (method I): Rt = 2.73 min; mass calcd. for C₂₉H₂₇F₆N₅O₅S 671.2, m/z found 672.5 [M+H]⁺; [α]_{D}²⁰ -21.72° (c 0.29, DMF).

### (+)-N-{2-[3,5-Difluoro-6-(4-Fluorophenyl)-4-{2-[(Methanesulfonyl)Amino]Propan-2-yl}Pyridin-2-yl]-3,3,3-Trifluoro-2-Hydroxypropyl}-8-Methoxy-3-Methylcinnoline-6-Carboxamide 244

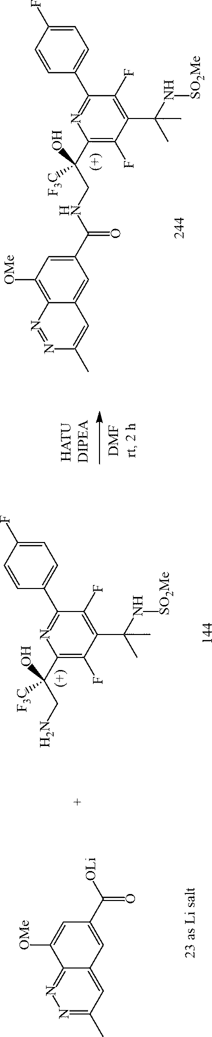

244 (74 mg. 52%) was synthesized according to procedure C. The crude mixture was purified by silica column chromatography (CH₂Cl₂/CH₃OH, gradient from 100:0 to 99:1). The residue was co-evaporated with EtOH (3 times) and dried under vacuum at 50° C. for 5 h. A second purification was performed by reverse phase (stationary phase: YMC-actus Triart C18 10 µm 30 x 150 mm, 40 g, mobile phase: NH₄HCO₃ (0.25% in H₂O)/CH₃CN, gradient from 75:25 to 35:65). ¹H NMR (400 MHz, DMSO-d₆) δ ppm 1.78 (br s, 6H), 2.85 (s, 3H), 2.87 (s, 3H), 4.02 (s, 3H), 4.08 - 4.16 (m, 1H), 4.39 (br dd, J=14.2, 5.4 Hz, 1H), 6.99 - 7.14 (m, 1H), 7.32 (d, J=1.0 Hz, 1H), 7.34 - 7.40 (m, 2H), 7.72 (d, J=1.0 Hz, 1H), 7.87 - 8.01 (m, 4H), 8.64 - 8.75 (m, 1H); LC-MS (method I): Rt = 2.73 min; mass calcd. for C₂₉H₂₇F₆N₅O₅S 671.2, m/z found 672.5 [M+H]⁺; [α]_{D}²⁰ +14.8° (c 0.25, DMF).

### (+)-N-{Cyclopropyl-2-[5-Fluoro-6-(4-Fluorophenyl)-4-Hydroxypropan-2-yl)-3-Methylpyridin-2-yl]-2-Hydroxyethyl}-8-Methoxy-3-Methylcinnoline-6-Carboxamide 245 and (-)-N-{Cyclopropyl-2-[5-Fluoro-6-(4-Fluorophenyl)-4-Hydroxypropan-2-yl)-3-Methylpyridin-2-yl]-2-Hydroxyethyl}-8-Methoxy-3-Methylcinnoline-6-Carboxamide 246

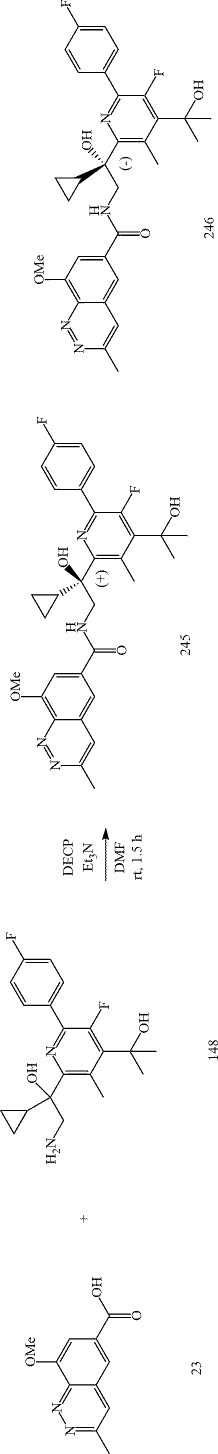

The racemic product was synthesized according to procedure A and purified via reverse phase HPLC (stationary phase: Kromasil C18 100A 5 µm (Eka Nobel), mobile phase: NH₄HCO₃ (0.25% in H₂O)/CH₃CN, gradient from 70:30 to 20:80). ¹H NMR (400 MHz, DMSO-d₆) δ ppm 8.51 (t, J=5.9 Hz, 1H), 7.85 (dd, J=7.5, 5.7 Hz, 2H), 7.75 (s, 1H), 7.64 (d, J=1.3 Hz, 1H), 7.24 -7.31 (m, 3H), 5.91 (s, 1H), 5.32 (s, 1H), 4.12 (dd, J=13.2, 6.6 Hz, 1H), 3.99 (s, 3H), 3.83 (dd, J=13.4, 5.3 Hz, 1H), 2.91 (s, 3H), 2.84 (s, 3H), 1.67 - 1.75 (m, 1H), 1.65 (d, J=4.4 Hz, 3H), 1.60 (d, J=2.6 Hz, 3H), 0.59 - 0.67 (m, 1H), 0.37 - 0.47 (m, 1H), 0.19 - 0.34 (m, 2H); LCMS (method B): Rt 1.08 min; mass calcd. for C₃₁H₃₂F₂N₄O₄ 562.0, m/z found 563.0 [M+H]⁺. Then the isomers were separated by SFC (stationary phase: Daicel Chiralpak IC 250 g, 5 µm, mobile phase: heptane/EtOH, 60:40) to afford 245 (88 mg. 18%) and 246 (97 mg, 20%).

### (-)-N-{2-[5-Chloro-3-Fluoro-6-(4-Fluorophenyl)-4-(2-Hydroxypropan-2-yl)Pyridin-2-yl]-3,3,3-Trifluoro-2-Hydroxypropyl}-8-Methoxy-3-Methylcinnoline-6-Carboxamide 247

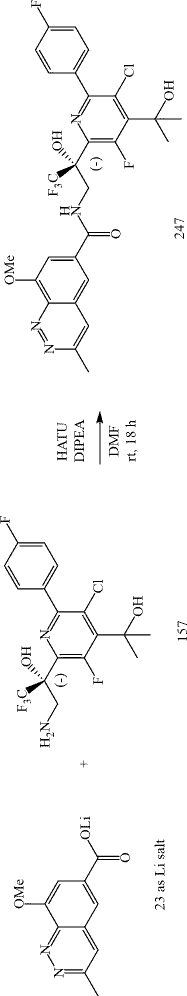

247 (108 mg. 88%) was synthesized according to procedure C. The crude mixture was purified by silica column chromatography (CH₂Cl₂/CH₃OH, gradient from 100:0 to 99:1). The residue was co-evaporated with EtOH (3 times) and dried under vacuum at 50° C. for 3 h. ¹H NMR (400 MHz, DMSO-d₆) δ ppm 1.66 (br s, 6H), 2.86 (s, 3H), 4.00 - 4.10 (m, 4H), 4.37 (br dd, J=13.5, 6.5 Hz, 1H), 5.54 (s, 1H), 6.93 (s, 1H), 7.23 - 7.33 (m, 3H), 7.54 - 7.61 (m, 2H), 7.72 (d, J=1.0 Hz, 1H), 7.94 (s, 1H), 8.72 (t, J=6.1 Hz, 1H); LC-MS (method I): Rt = 2.83 min; mass calcd. for C₂₈H₂₄ClF₅N₄O₄ 610.1, m/z found 611.3 [M+H]⁺; [α]_{D}²⁰ -44.56° (c 0.285, DMF).

### (+)-N-{2-[5-Chloro-3-Fluoro-6-(4-Fluorophenyl)-4-(2-Hydroxypropan-2-yl)Pyridin-2-yl]-3,3,3-Trifluoro-2-Hydroxypropyl}-8-Methoxv-3-Methylcinnoline-6-Carboxamide 248

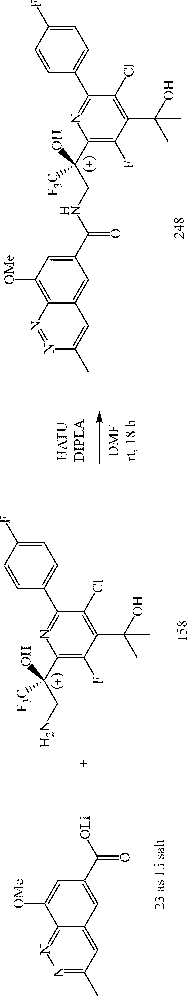

248 (112 mg. 91%) was synthesized according to procedure C. The crude mixture was purified by silica column chromatography (CH₂Cl₂/CH₃OH, gradient from 100:0 to 99:1). The residue was co-evaporated with EtOH (3 times) and dried under vacuum at 50° C. for 3 h. ¹H NMR (400 MHz, DMSO-d₆) δ ppm 1.66 (br s, 6H), 2.86 (s, 3H), 4.01 - 4.09 (m, 4H), 4.32 - 4.42 (m, 1H), 5.54 (s, 1H), 6.93 (s, 1H), 7.24 - 7.33 (m, 3H), 7.53 - 7.62 (m, 2H), 7.72 (d, J=1.0 Hz, 1H), 7.94 (s, 1H), 8.72 (t, J=6.0 Hz, 1H); LC-MS (method I): Rt = 2.83 min; mass calcd. for C₂₈H₂₄ClF₅N₄O₄ 610.1, m/z found 611.3 [M+H]⁺; [α]_{D}²⁰ +45.69° (c 0.267, DMF).

### (+)-N-{3,3-Difluoro-2-[5-Fluoro-6-(4-Fluorophenyl)-4-(2-Hydroxypropan-2-yl)Pyridin-2-yl]-2-Hydroxypropyl}-8-Methoxy-3-Methylcinnoline-6-Carboxamide 249 and (-)-N-{3,3-Difluoro-2-[5-Fluoro-6-(4-Fluorophenyl)-4-(2-Hydroxypropan-2-yl)Pyridin-2-yl]-2-Hydroxypropyl}-8-Methoxy-3-Methylcinnoline-6-Carboxamide 250

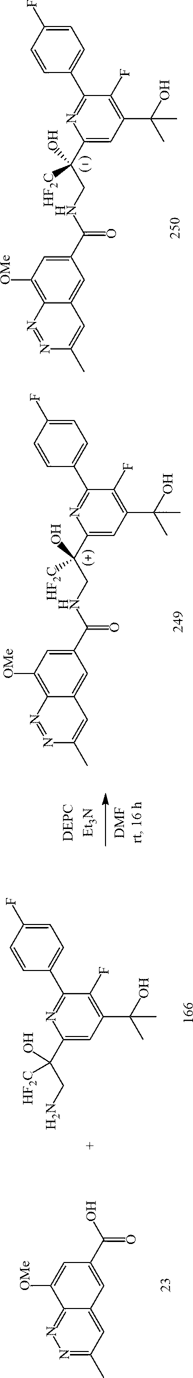

249 (74 mg. 32%) and 250 (78 mg, 33%) were synthesized according to procedure A. A purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80) to afford a racemic mixture. ¹H NMR (400 MHz, DMSO-d₆) δ ppm 8.69 (t, J=6.1 Hz, 1H), 8.04 (d, J=5.5 Hz, 1H), 7.93 (dd, J=7.6, 5.6 Hz, 2H), 7.82 (s, 1H), 7.71 (d, J=1.3 Hz, 1H), 7.34 (d, J=1.3 Hz, 1H), 7.19 - 7.30 (m, 2H), 6.66 (t, J=54.8 Hz, 1H), 6.56 (s, 1H), 5.62 (s, 1H), 3.96 - 4.11 (m, 4H), 3.78 - 3.86 (m, 1H), 2.86 (s, 3H), 1.54 (s, 3H), 1.45 (s, 3H); LC-MS (method B): Rt 0.93 min; mass calcd. for C₂₈H₂₆F₄N₄O₄ 558.0, m/z found 559.0 [M+H]⁺. The enantiomers were separated by SFC (stationary phase: Daicel Chiralpak IC 250 g, 5 µm, mobile phase: heptane/EtOH 86:14) afforded 249; [α]_{D}²⁰ +82.53 (c 0.261, DMF); and 250; [α]_{D}²⁰ -73.76° (c 0.263, DMF).

### N-{(-)[5-Chloro-6-(4-Fluorophenyl)-4-(2-Hydroxypropanyl)Pyridinyl]CyclopropylHydroxyethyl}-8-Methoxy-3-Methylcinnoline-6-Carboxamide 251

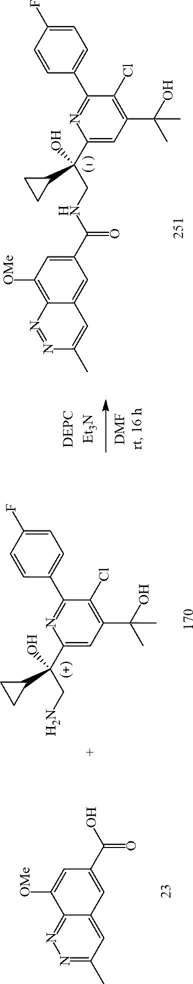

251 (292 mg. 63%) was synthesized according to procedure A. The purification was performed via reverse phase HPLC (Kromasil C18 100A 5 µm (Eka Nobel), mobile phase: NH₄HCO₃ (0.25% in H₂O)/CH₃CN, gradient from 85:15 to 25:75). ¹H NMR (400 MHz, DMSO-d₆) δ ppm 0.10 - 0.21 (m, 1H), 0.27 - 0.35 (m, 1H), 0.35 - 0.43 (m, 1H), 0.53 - 0.62 (m, 1H), 1.47 - 1.54 (m, 1H), 1.56 (s, 3H), 1.64 (s, 3H), 2.87 (s, 3 H), 3.81 - 3.96 (m, 2H), 4.07 (s, 3H), 5.44 (s, 1H), 5.57 (s, 1H), 7.21 - 7.30 (m, 2H), 7.39 (d, J=1.3 Hz, 1H), 7.65 - 7.71 (m, 2H), 7.74 (d, J=1.3 Hz, 1H), 7.91 (s, 1H), 8.15 (s, 1H), 8.66 (t, J=5.8 Hz, 1H); LC-MS (method B): Rt 1.00 min; mass calcd. for C₃₀H₃₀ClFN₄O₄ 564.0, m/z found 565.4 [M+H]⁺; [α]_{D}²⁰ -5.13° (c 0.526, DMF).

### (-)-N-{2-Cyclopropyl-2-[5-Fluoro-6-(4-Fluorophenyl)-4-(2-Hydroxypropan-2-yl)Pyridin-2-yl]-2-Hydroxyethyl}-8-Methoxy-3-Methylcinnoline-6-Carboxamide 252

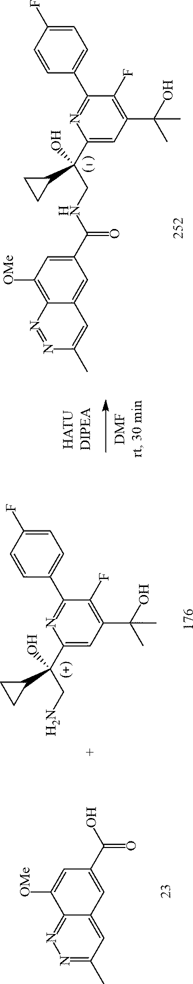

252 (288 mg. 73%) was synthesized according to procedure B. The reaction was quenched by the addition of water and the mixture was stirred for few hours. The surnatant was removed. The resulting solid was triturated in water and filtered off. The residue was purified by silica column chromatography (EtOAc). ¹H NMR (400 MHz, DMSO-d₆) δ ppm 0.12 - 0.21 (m, 1H), 0.28 -0.36 (m, 1H), 0.37 - 0.46 (m, 1H), 0.55 - 0.63 (m, 1H), 1.48 (s, 3H), 1.54 (s, 3H), 1.56 - 1.62 (m, 1H), 2.86 (s, 3H), 3.86 - 3.98 (m, 2H), 4.06 (s, 3H), 5.43 (s, 1H), 5.56 (s, 1H), 7.27 - 7.35 (m, 2H), 7.40 (d, J=1.3 Hz, 1H), 7.76 (d, J=1.3 Hz, 1H), 7.89 (s, 1H), 7.92 - 8.01 (m, 3H), 8.64 (t, J=5.9 Hz, 1H); LC-MS (method B): Rt = 1.00 min; mass calcd. for C₃₀H₃₀F₂N₄O₄ 548.0, m/z found 549.3 [M+H]⁺; [α]_{D}²⁰ -30.92° (c 0.255, DMF).

### (-)-N-Cyclopropyl-2-{5-Fluoro-4-(2-Hydroxypropan-2-yl)-6-[4-(Trifluoromethyl)Phenyl]-Pyridin-2-yl}-2-Hydroxyethyl]-8-Methoxy-3-Methylcinnoline-6-Carboxamide 253

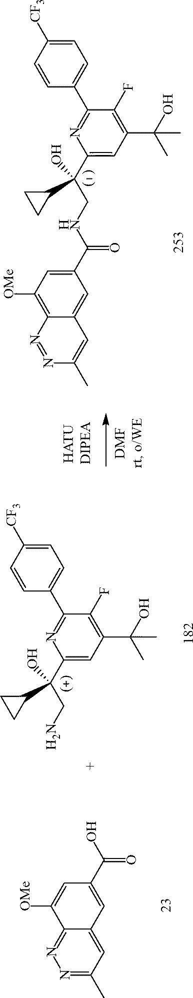

253 (199 mg. 67%) was synthesized according to procedure B. The crude was purified by silica column chromatography (CH₂Cl₂/CH₃OH, gradient from 99:1 to 95:5). The residue was triturated in DIPE. The solids were collected by filtration and dried under vacuum. ¹H NMR (400 MHz, DMSO-d₆) δ ppm 0.13 - 0.22 (m, 1H), 0.28 - 0.37 (m, 1H), 0.38 - 0.48 (m, 1H), 0.56 - 0.64 (m, 1H), 1.48 (s, 3H), 1.55 (s, 3H), 1.58 - 1.65 (m, 1H), 2.86 (s, 3H), 3.87 - 3.98 (m, 2H), 4.06 (s, 3H), 5.45 (s, 1H), 5.61 (s, 1H), 7.39 (d, J=1.6 Hz, 1H), 7.77 (d, J=1.6 Hz, 1H), 7.84 (d, J=8.5 Hz, 2H), 7.92 (s, 1H), 8.01 (d, J=5.7 Hz, 1H), 8.14 (d, J=8.1 Hz, 2H), 8.65 (t, J=5.9 Hz, 1H); LC-MS (method H): Rt = 2.08 min; mass calcd. for C₃₁H₃₀F₄N₄O₄ 598.2, m/z found 599.4 [M+H]⁺; [α]_{D}²⁰ -37.77° (c 0.349, DMF).

### (-)-N-{2-Cyclopropyl-2-[6-(4-Cyclopropylphenyl)-5-Fluoro-4-(2-Hydroxypropan-2-yl)Pyridin-2-yl]-2-Hydroxyethyl}-8-Methoxy-3-Methylcinnoline-6-Carboxamide 254

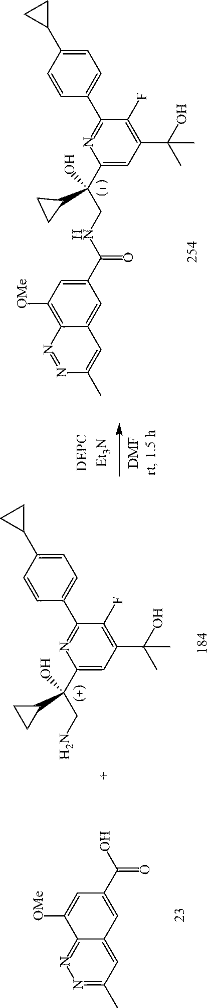

254 (129 mg. 43%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 60:40 to 20:80). A second purification was performed via reverse phase HPLC (stationary phase: Kromasil C18 100A 5 µm (Eka Nobel), mobile phase: NH₄HCO₃ (0.25% in H₂O)/CH₃OH, gradient from 50:50 to 10:90). ¹H NMR (400 MHz, DMSO-d₆) δ ppm 8.66 (t, J=5.8 Hz, 1H), 7.90 (d, J=5.7 Hz, 1H), 7.87 (s, 1H), 7.79 (dd, J=8.1, 1.3 Hz, 2H), 7.76 (d, J=1.3 Hz, 1H), 7.39 (d, J=1.3 Hz, 1H), 7.12 - 7.17 (m, 2H), 5.54 (s, 1H), 5.42 (s, 1H), 4.05 (s, 3H), 3.84 - 3.96 (m, 2H), 2.87 (s, 3H), 1.91 - 2.00 (m, 1H), 1.55 - 1.61 (m, 1H), 1.54 (s, 3H), 1.48 (s, 3H), 0.96 - 1.03 (m, 2H), 0.66 - 0.71 (m, 2H), 0.54 -0.61 (m, 1H), 0.36 - 0.45 (m, 1H), 0.27 - 0.35 (m, 1H), 0.12 - 0.21 (m, 1H); LCMS (method B): Rt 1.13 min; mass calcd. for C₃₃H₃₅FN₄O₄ 570.0, m/z found 571 [M+H]⁺; [α]_{D}²⁰ -34.45° (c 0.508, DMF).

### (-)-N-{2-Cyclopropyl-2-[5-Fluoro-6-(3-Fluorophenyl)-4-(2-Hydroxypropan-2-yl)Pyridin-2-yl]-2-Hydroxyethyl}-8-Methoxy-3-Methylcinnoline-6-Carboxamide 255

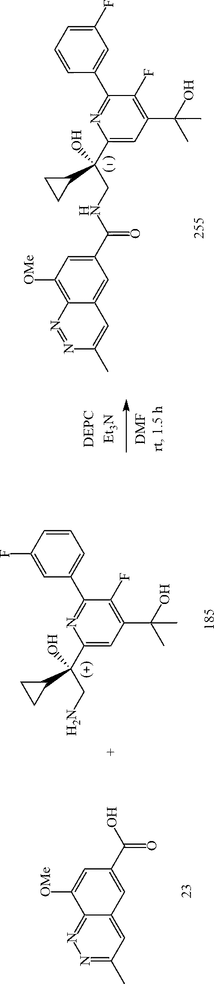

255 (75 mg. 25%) was synthesized according to procedure A then purified via reverse phase HPLC (stationary phase: Kromasil C18 100A 5 µm (Eka Nobel), mobile phase: NH₄HCO₃ (0.25% in H₂O)/CH₃CN, gradient from 60:40 to 20:80). ¹H NMR (400 MHz, DMSO-d₆) δ ppm 8.66 (t, J=5.8 Hz, 1H), 7.98 (d, J=5.7 Hz, 1H), 7.91 (s, 1H), 7.69 - 7.80 (m, 3H), 7.48 - 7.56 (m, 1H), 7.39 (d, J=1.3 Hz, 1H), 7.29 (td, J=8.6, 2.0 Hz, 1H), 5.64 (s, 1H), 5.51 (s, 1H), 4.05 (s, 3H), 3.94 - 4.01 (m, 1H), 3.84 - 3.91 (m, 1H), 2.86 (s, 3H), 1.55 - 1.63 (m, 1H), 1.54 (s, 3H), 1.47 (s, 3H), 0.54 - 0.64 (m, 1H), 0.37 - 0.48 (m, 1H), 0.28 - 0.36 (m, 1H), 0.12 - 0.23 (m, 1 H); LC-MS (method B): Rt 1.03 min; mass calcd. for C₃₀H₃₀F₂N₄O₄ 548.0, m/z found 549.0 [M+H]⁺; [α]_{D}²⁰ -34.36° (c 0.521, DMF).

### (-)-N-Cyclopropyl-2-(6-(3,4-Difluorophenyl)-5-Fluoro-4-Hydroxypropan-2-yl)Pyridin-2-yl]-2-Hydroxyethyl}-8-Methoxy-3-Methylcinnoline-6-Carboxamide 256

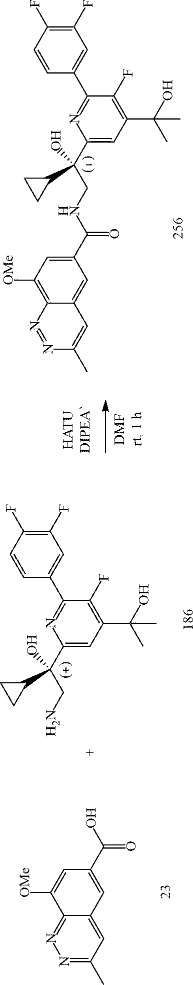

256 (163 mg. 58%) was synthesized according to procedure B. The crude was purified by silica column chromatography (CH₂Cl₂/CH₃OH, gradient from 99:1 to 95:5). The residue was crystallized from DIPE and CH₃CN (1:1) and collected by filtration and dried under vacuum. ¹H NMR (400 MHz, DMSO-d₆) δ ppm 0.12 - 0.22 (m, 1H), 0.27 - 0.35 (m, 1H), 0.38 - 0.46 (m, 1H), 0.56 - 0.64 (m, 1H), 1.47 (s, 3H), 1.54 (s, 3H), 1.55 - 1.62 (m, 1H), 2.86 (s, 3H), 3.88 (dd, J=13.4, 5.7 Hz, 1H), 3.98 (dd, J=13.4, 6.5 Hz, 1H), 4.06 (s, 3H), 5.45 (s, 1H), 5.58 (s, 1H), 7.39 (d, J=1.2 Hz, 1H), 7.54 (dt, J=10.6, 8.5 Hz, 1H), 7.76 (d, J=1.2 Hz, 1H), 7.77 - 7.83 (m, 1H), 7.92 (s, 1H), 7.94 - 8.01 (m, 2H), 8.63 (t, J=5.9 Hz, 1H); LC-MS (method H): Rt = 1.91 min; mass calcd. for C₃₀H₂₉F₃N₄O₄ 566.2, m/z found 567.4 [M+H]⁺; [α]_{D}²⁰ -40.47° (c 0.551, DMF).

### N-{(-)Cyclopropyl[3,5-Difluoro-6-(4-Fluorophenyl)-4-(2-Hydroxypropanyl)Pyridinyl]Hydroxyethyl}-8-Methoxy-3-Methylcinnoline-6-Carboxamide 257

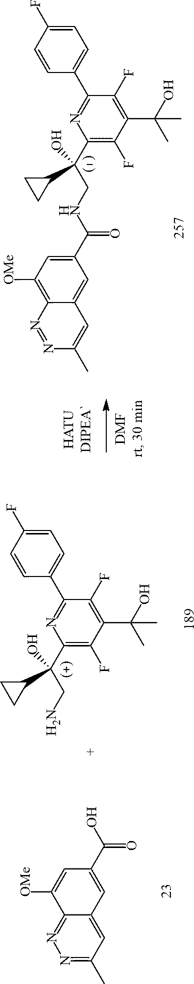

257 (50 mg. 65%) was synthesized according to procedure B. A purification was performed via reverse phase HPLC (stationary phase: Kromasil C18 100A 5 µm (Eka Nobel), mobile phase: NH₄HCO₃ (0.25% in H₂O)/CH₃CN, gradient from 70:30 to 20:80). ¹H NMR (400 MHz, DMSO-d₆) δ ppm 8.55 (dd, J=6.9, 5.2 Hz, 1H), 7.81 - 7.87 (m, 2H), 7.78 (s, 1H), 7.62 (d, J=1.5 Hz, 1H), 7.25 - 7.32 (m, 2H), 7.24 (d, J=1.5 Hz, 1H), 5.57 (s, 1H), 5.32 (s, 1H), 4.20 (dd, J=13.3, 7.4 Hz, 1H), 3.97 (s, 3H), 3.63 (dd, J=13.5, 4.7 Hz, 1H), 2.84 (s, 3H), 1.65 - 1.73 (m, 1H), 1.63 (s, 6H), 0.65 - 0.74 (m, 1H), 0.41 - 0.50 (m, 1H), 0.20 - 0.34 (m, 2H), LC-MS (method B): Rt = 1.03 min; mass calcd. for C₃₀H₂₉F₃N₄O₄ 566.0, m/z found 567.0 [M+H]⁺.

### N-{(-)Cyclopropyl[3,5-Difluoro-6-(4-Fluorophenyl)-4-(2-Hydroxypropanyl)Pyridinyl]Hydroxyethyl}-8-Methoxy-3-Methylcinnoline-6-Carboxamide 258

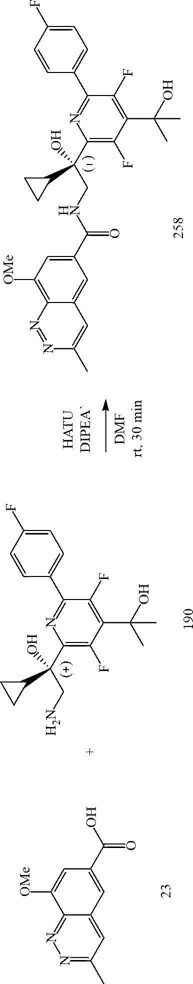

258 (60 mg. 78%) was synthesized according to procedure B. A purification was performed via reverse phase HPLC (stationary phase: Kromasil, C18, 100A, 5 µm (Eka Nobel), mobile phase: NH₄HCO₃ (0.25% in H₂O)/CH₃OH, gradient from 50:50 to 10:90). ¹H NMR (400 MHz, DMSO-d₆) δ 8.55 (t, J=1.0 Hz, 1H), 7.84 (dd, J=7.5, 5.5 Hz, 2H), 7.78 (s, 1H), 7.62 (d, J=1.3 Hz, 1H), 7.25 - 7.33 (m, 2H), 7.24 (d, J=1.1 Hz, 1H), 5.57 (br s, 1H), 5.32 (s, 1H), 4.15 - 4.25 (m, 1H), 3.97 (s, 3H), 3.54 - 3.68 (m, 1H), 2.83 (s, 3H), 1.66 - 1.73 (m, 1H), 1.63 (s, 6H), 0.66 - 0.77 (m, 1H), 0.40 - 0.50 (m, 1H), 0.18 - 0.34 (m, 2H); LC-MS (method B): Rt = 1.02 min; mass calcd. for C₃₀H₂₉F₃N₄O₄ 566.0, m/z found 567.0 [M+H]⁺; [α]_{D}²⁰ -14.2° (c 0.65, DMF).

### (-)-N-{2-Cyclopropyl-2-[6-(3,4-Difluorophenyl)-4-(2-Hydroxypropan-2-yl)Pyridin-2-yl]-2-Hydroxyethyl}-8-Methoxy-3-Methylcinnoline-6-Carboxamide 259

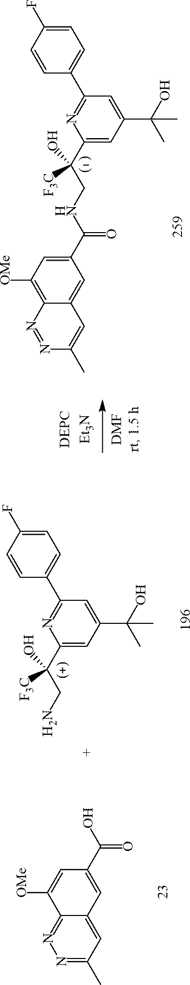

259 (117 mg. 50%) was synthesized according to procedure A and purified by reverse phase HPLC (stationary phase: Kromasil C18 100A 5 µm (Eka Nobel), mobile phase: NH₄HCO₃ (0.25% in H₂O)/CH₃CN, gradient from 60:40 to 20:80). ¹H NMR (400 MHz, DMSO-d₆) δ ppm 8.79 (t, J=5.9 Hz, 1H), 8.16 - 8.25 (m, 2H), 7.97 (d, J=1.3 Hz, 1H), 7.91 (s, 1H), 7.88 (d, J=1.1 Hz, 1H), 7.77 (d, J=1.3 Hz, 1H), 7.37 (d, J=1.3 Hz, 1H), 7.30 - 7.36 (m, 2H), 7.22 (s, 1H), 5.37 (s, 1H), 4.32 (dd, J=14.0, 6.5 Hz, 1H), 4.14 (dd, J=13.9, 5.3 Hz, 1H), 4.03 (s, 3H), 2.85 (s, 3H), 1.45 (s, 6H); LC-MS (method B): Rt = 0.99 min; mass calcd. for C₂₈H₂₆F₄N₄O₄ 558.0, m/z found 559.0 [M+H]⁺; [α]_{D}²⁰ -81.9° (c 0.525, DMF).

### (-)-N-{2-Cyclopropyl-2-[5-Fluoro-6-(4-Fluorophenyl)-4-(2-Hydroxypropan-2-yl)Pyridin-2-yl]-2-Hydroxyethyl}-8-(Difluoromethoxy)-3-Methylcinnoline-6-Carboxamide 260

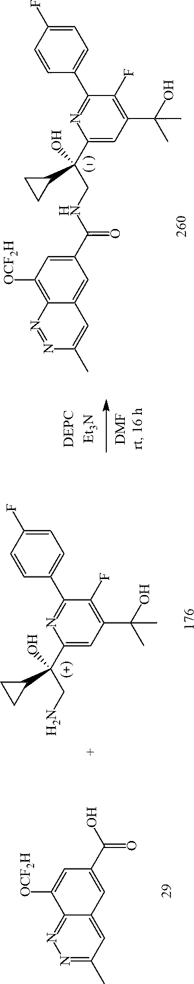

260 (99 mg. 59%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 85:15 to 20:80). ¹H NMR (400 MHz, DMSO-d₆) δ ppm 0.10 - 0.21 (m, 1H), 0.26 - 0.44 (m, 2H), 0.53 - 0.63 (m, 1H), 1.48 (s, 3H), 1.54 (s, 3H), 1.50 - 1.61 (m, 2H), 2.92 (s, 3H), 3.92 (d, J=5.9 Hz, 2H), 5.40 (s, 1H), 5.56 (s, 1H), 7.27 - 7.34 (m, 2H), 7.60 (t, J=73.7 Hz, 1H), 7.79 (s, 1H), 7.97 (br dd, J=7.5, 5.7 Hz, 2H), 8.05 (s, 1H), 8.17 (d, J=1.5 Hz, 1H), 8.73 (t, J=6.1 Hz, 1H); LC-MS (method B): Rt 1.07 min; mass calcd. for C₃₀H₂₈F₄N₄O₄ 584.0, m/z found 585.3 [M+H]⁺; [α]_{D}²⁰ -25.75° (c 0.268, DMF).

### (-)-N-{2-[5-Chloro-6-(4-Fluorophenyl)-4-(2-Hydroxypropan-2-yl)Pyridin-2-yl]-3,3,3-Trifluoro-2-Hydroxypropyl}-3-Methyl-8-(Trifluoromethoxy)Cinnoline-6-Carboxamide 261

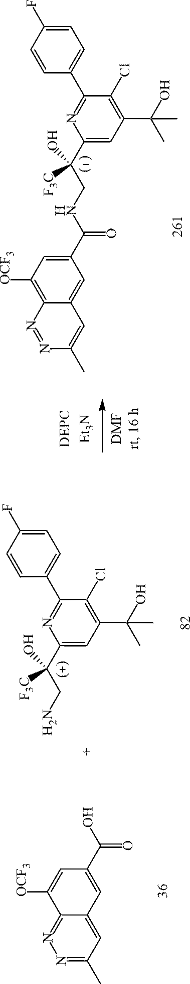

261 (111 mg. 67%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆) δ ppm 1.57 (s, 3H), 1.64 (s, 3H), 2.95 (s, 3H), 4.07 - 4.25 (m, 2H), 5.67 (s, 1H), 7.22 - 7.32 (m, 3H), 7.64 - 7.72 (m, 2H), 7.99 (d, J=1.3 Hz, 1H), 8.18 (s, 1H), 8.32 (s, 1H), 8.36 (d, J=1.5 Hz, 1H), 8.92 (t, J=5.6 Hz, 1H); LC-MS (method B): Rt 1.17 min; mass calcd. for C₂₈H₂₂ClF₇N₄O₄ 646.0, m/z found 647.0 [M+H]⁺; [α]_{D}²⁰ -25.75° (c 0.268, DMF).

### (-)(Cyclopropyloxy)-N-{2-[3,5-Difluoro-6-(4-Fluorophenyl)-4-(2-Hydroxypropan-2-yl)Pyridin-2-yl]-3,3,3-Trifluoro-2-Hydroxypropyl}-3-Methylcinnoline-6-Carboxamide 262

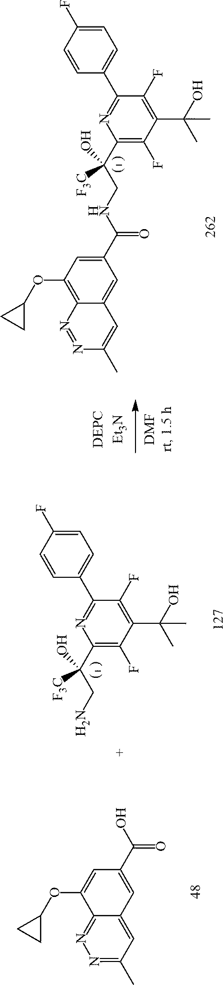

262 (96 mg, 61%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆) δ ppm 0.77 - 0.83 (m, 2H), 0.83 - 0.89 (m, 2H), 1.62 (s, 6H), 2.84 (s, 3H), 4.02 - 4.13 (m, 2H), 4.40 (br dd, J=13.8, 6.1 Hz, 1H), 5.61 (s, 1H), 7.04 (s, 1H), 7.31 - 7.38 (m, 2H), 7.61 (d, J=1.5 Hz, 1H), 7.74 (d, J=1.5 Hz, 1H), 7.87 (s, 1H), 7.92 (dd, J=7.5, 5.5 Hz, 2H), 8.70 (br t, J=5.7 Hz, 1H); LC-MS (method B): Rt 1.04 min; mass calcd. for C₃₀H₂₆F₆N₄O₄ 620.2, m/z found 621.3 [M+H]⁺; [α]_{D}²⁰ -27.61° (c 0.268, DMF).

### (-)-N-{2-[5-Chloro-3-Fluoro-6-(4-Fluorophenyl)-4-(2-Hydroxypropan-2-yl)Pyridin-2-yl]-3,3,3-Trifluoro-2-Hydroxypropyn-8-(Cyclopropyloxy)-3-Methylcinnoline-6-Carboxamide 263

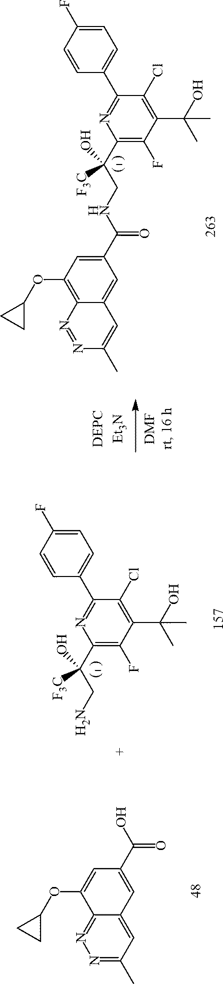

263 (123 mg, 79%) was synthesized according to procedure A. The purification was performed via reverse phase HPLC (stationary phase: Kromasil C18 100A 5 µm (Eka Nobel), mobile phase: NH₄HCO₃ (0.25% in H₂O)/ CH₃CN, gradient from 80:20 to 20:80). ¹H NMR (400 MHz, DMSO-d6) δ ppm 0.78 - 0.93 (m, 4H), 1.67 (br s, 6H), 2.86 (s, 3H), 4.01 - 4.15 (m, 2H), 4.38 (br dd, J=13.8, 6.3 Hz, 1H), 5.53 (s, 1H), 6.93 (s, 1H), 7.22 - 7.31 (m, 2H), 7.54 - 7.61 (m, 2H), 7.63 (d, J=1.5 Hz, 1H), 7.74 (d, J=1.5 Hz, 1H), 7.92 (s, 1H), 8.70 (br t, J=5.8 Hz, 1H); LC-MS (method G): Rt 2.04 min; mass calcd. for C₃₀H₂₆ClF₅N₄O₄ 636.2, m/z found 637.2 [M+H]⁺; [α]_{D}²⁰ -50.1° (c 0.256, DMF).

### (-)-N-{2-[5-Chloro-6-(4-Fluorophenyl)-4-(2-Hydroxypropan-2-yl)Pyridin-2-yl]-2-Cyclopropyl-2-Hydroxyethyl}-8-(Cyclopropyloxy)-3-Methylcinnoline-6-Carboxamide 264

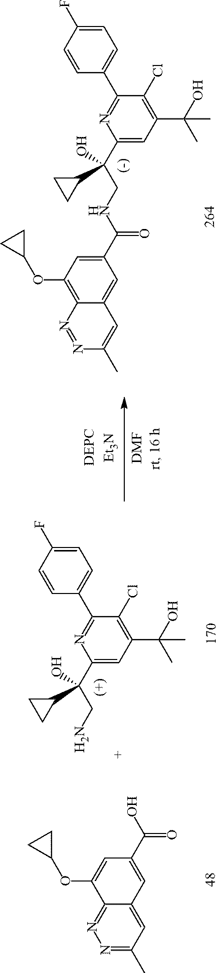

266 (101 mg. 62%) was synthesized according to procedure A. A purification was performed via reverse phase HPLC (stationary phase: Kromasil C18, 100A, 5 µm (Eka Nobel), mobile phase: NH₄HCO₃ (0.25% in H₂O)/CH₃OH, gradient from 80:20 to 0:100). ¹H NMR (400 MHz, DMSO-d₆) δ ppm 0.10 - 0.20 (m, 1H), 0.24 - 0.34 (m, 1H), 0.34 - 0.44 (m, 1H), 0.52 - 0.62 (m, 1H), 0.71 - 0.80 (m, 2H), 0.81 - 0.91 (m, 2H), 1.44 - 1.53 (m, 1H), 1.57 (s, 3H), 1.64 (s, 3H), 2.48 (s, 3H), 3.84 (dd, J=13.5, 5.3 Hz, 1H), 3.93 (dd, J=13.4, 6.4 Hz, 1H), 3.98 - 4.05 (m, 1H), 5.55 (s, 1H), 7.20 - 7.32 (m, 2H), 7.65 - 7.73 (m, 3H), 7.74 (d, J=1.5 Hz, 1H), 7.98 - 8.02 (m, 1H), 8.15 (s, 1H), 8.55 (t, J=5.9 Hz, 1H), 8.75 (d, J=2.2 Hz, 1H); LC-MS (method F): Rt = 2.55 min; mass calcd. for C₃₂H₃₂ClFN₄O₄ 590.0, m/z found 590.4 [M]⁺; [α]_{D}²⁰ -85.6° (c 0.253, DMF).

### (-)-N-{2-Cyclopropyl-2-[5-Fluoro-6-(4-Fluorophenyl)-4-(2-Hydroxypropan-2-yl)Pyridin-2-yl]-2-Hydroxyethyl}-8-(Cyclopropyloxy)-3-Methylcinnoline-6-Carboxamide 265

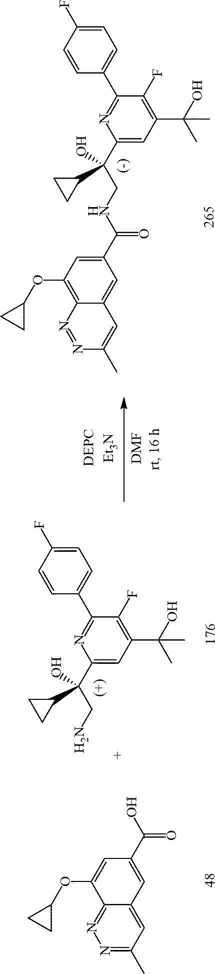

265 (125 mg. 76%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d6) δ ppm -0.06 - 0.05 (m, 1H), 0.09 - 0.18 (m, 1H), 0.19 - 0.30 (m, 1H), 0.37 - 0.47 (m, 1H), 0.61 - 0.78 (m, 4H), 1.30 (s, 3H), 1.36 (s, 3H), 1.38 - 1.44 (m, 1H), 2.68 (s, 3H), 3.68 - 3.83 (m, 2H), 3.91 - 3.98 (m, 1H), 5.25 (s, 1H), 5.38 (s, 1H), 7.08 - 7.18 (m, 2H), 7.53 (d, J=1.5 Hz, 1H), 7.59 (d, J=1.5 Hz, 1H), 7.69 (s, 1H), 7.74 - 7.85 (m, 3H), 8.44 (t, J=5.8 Hz, 1H); LC-MS (method G): Rt 2.02 min; mass calcd. for C₃₂H₃₂F₂N₄O₄ 574.2, m/z found 575.2 [M+H]⁺; [α]_{D}²⁰ -32.75° (c 0.256, DMF).

### (-)(Difluoromethyl)-8-Methoxy-N-{3,3,3-Trifluoro-2-[5-Fluoro-6-(4-Fluorophenyl)-4-(2-Hydroxypropan-2-yl)Pyridin-2-yl]-2-Hydroxypropyl}Cinnoline-6-Carboxamide 266

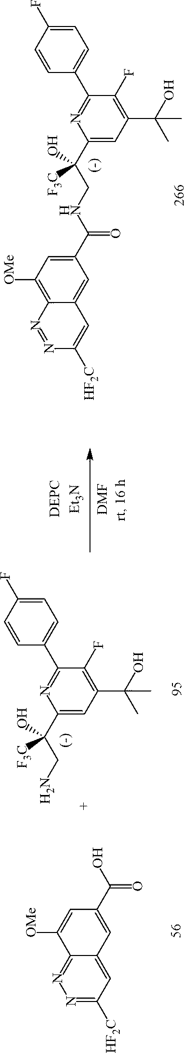

266 (142 mg. 87%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 70:30 to 0:100). ¹H NMR (400 MHz, DMSO-d6) δ ppm 8.83 (t, J=5.9 Hz, 1H), 8.54 (s, 1H), 8.12 (d, J=5.3 Hz, 1H), 8.03 (d, J=1.5 Hz, 1H), 7.99 (dd, J=7.6, 5.6 Hz, 2H), 7.60 (d, J 1.1 Hz, 1H), 7.55 (t, J=54.4 Hz, 1H), 7.30 - 7.38 (m, 2H), 7.28 (s, 1H), 5.67 (s, 1H), 4.25 - 4.31 (m, 1H), 4.16 (dd, J=13.9, 5.7 Hz, 1H), 4.11 (s, 3H), 1.55 (s, 3H), 1.48 (s, 3H); LC-MS (method B): Rt 1.09 min; mass calcd. for C₂₈H₂₃F₇N₄O₄ 612.0, m/z found 613.0 [M+H]⁺; [α]_{D}²⁰ -82.05° (c 0.254, DMF).

### (-)-N-{2-[5-Chloro-6-(4-Fluorophenyl)-4-(2-Hydroxypropan-2-yl)Pyridin-2-yl]-3,3,3-Trifluoro-2-Hydroxypropyl}-3-(Difluoromethyl)-8-Methoxycinnoline-6-Carboxamide 267

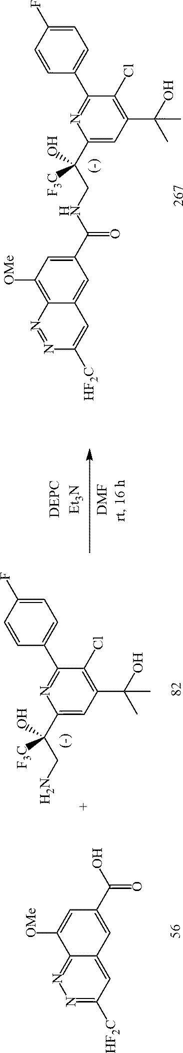

267(137 mg. 86%) was synthesized according to procedure A. A purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d₆, 81° C.) δ ppm 8.58 (br t, J=5.8 Hz, 1H), 8.45 (s, 1H), 8.31 (s, 1H), 7.96 (d, J=1.3 Hz, 1H), 7.64 - 7.69 (m, 2H), 7.57 (d, J=1.3 Hz, 1H), 7.48 (t, J=54.5 Hz, 1H), 7.19 - 7.26 (m, 2H), 6.94 -7.10 (m, 1H), 4.26 (dd, J=14.2, 6.5 Hz, 1H), 4.13 (s, 3H), 4.07 - 4.12 (m, 1H), 1.65 (s, 3H), 1.60 (s, 3H); LC-MS (method E): Rt 2.13 min; mass calcd. for C₂₈H₂₃ClF₆N₄O₄ 628.0, m/z found 629.0 [M+H]⁺; [α]_{D}²⁰ -46.74° (c 0.261, DMF).

### N-{(-)[5-Chloro-6-(4-Fluorophenyl)-4-(2-Hydroxypropanyl)Pyridinyl]CyclopropylHydroxyethyl}-3-(Difluoromethyl)-8-Methoxycinnoline-6-Carboxamide 268

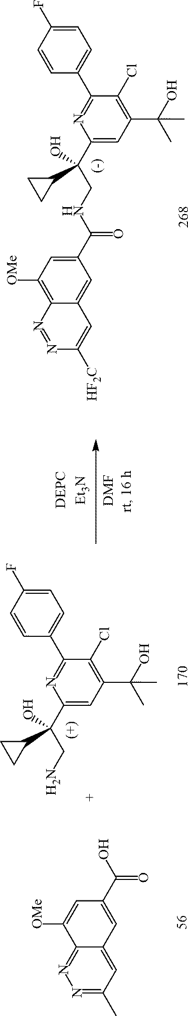

268 (121 mg. 72%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃OH, gradient from 80:20 to 20:80). ¹H NMR (400 MHz, DMSO-d6) δ ppm 8.71 (t, J=5.9 Hz, 1H), 8.54 (s, 1H), 8.15 (s, 1H), 8.00 (d, J=1.3 Hz, 1H), 7.65 - 7.71 (m, 2H), 7.60 (d, J=1.3 Hz, 1H), 7.56 (t, J=54.4 Hz, 1H), 7.20 - 7.27 (m, 2H), 5.59 (s, 1H), 5.41 (s, 1H), 4.13 (s, 3H), 3.84 - 3.96 (m, 2H), 1.64 (s, 3H), 1.56 (s, 3H), 1.49 - 1.55 (m, 1H), 0.55 - 0.63 (m, 1H), 0.36 - 0.45 (m, 1H), 0.28 - 0.36 (m, 1H), 0.12 - 0.21 (m, 1H); LC-MS (method B): Rt 1.10 min; mass calcd. for C₃₀H₂₈ClF₃N₄O₄ 600.0, m/z found 601.0 [M+H]⁺; [α]_{D}²⁰ -42.93° (c 0.410, DMF).

### (-)-N-{2-Cyclopropyl-2-[5-Fluoro-6-(4-Fluorophenyl)-4-(2-Hydroxypropan-2-yl)Pyridin-2-yl]-2-Hydroxyethyn-3-(Difluoromethyl)-8-Methoxycinnoline-6-Carboxamide 269

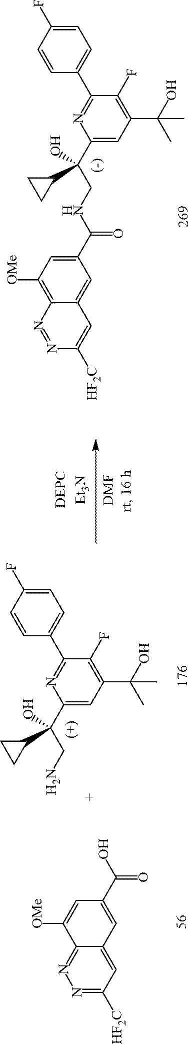

269 (121 mg. 72%) was synthesized according to procedure A. The purification was performed via reverse phase HPLC (stationary phase: Kromasil C18 100A 5 µm (Eka Nobel), mobile phase: NH₄HCO₃ (0.25% in H₂O)/CH₃CN, gradient from 80:20 to 20:80). ¹H NMR (400 MHz, DMSO-d6) δ ppm 8.71 (t, J=5.9 Hz, 1H), 8.53 (s, 1H), 8.02 (d, J 1.1 Hz, 1H), 7.93 - 8.00 (m, 3H), 7.60 (d, J 1.1 Hz, 1H), 7.55 (t, J 54.4 Hz, 1H), 7.26 - 7.33 (m, 2H), 5.58 (s, 1H), 5.42 (s, 1H), 4.11 (s, 3H), 3.89 - 3.98 (m, 2H), 1.56 - 1.63 (m, 1H), 1.54 (s, 3H), 1.47 (s, 3H), 0.56 - 0.64 (m, 1H), 0.38 - 0.46 (m, 1H), 0.28 - 0.36 (m, 1H), 0.13 - 0.22 (m, 1H); LC-MS (method E): Rt 2.08 min; mass calcd. for C₃₀H₂₈F₄N₄O₄ 584.0, m/z found 585.0 [M+H]⁺; [α]_{D}²⁰ -24.46° (c 0.254, DMF).

### (-)-N-{2-Cyclopropyl-2-[6-(3,4-Difluorophenyl)-5-Fluoro-4-(2-Hydroxypropan-2-yl)Pyridin-2-yl]-2-Hydroxyethyl}-3-(Difluoromethyl)-8-Methoxycinnoline-6-Carboxamide 270

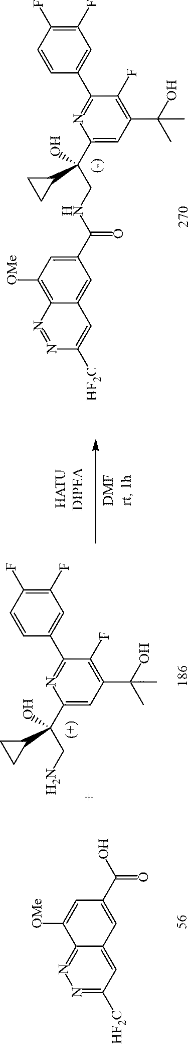

270 (153 mg. 51%) was synthesized according to procedure B. The precipitate was dissolved in CH₂Cl₂ and washed with water (twice). The organic layer was dried (MgSOa). The solids were removed by filtration and the filtrate was evaporated under reduced pressure. The residue was crystallized from DIPE and CH₃CN (5:1), collected by filtration and dried under vacuum. ¹H NMR (400 MHz, DMSO-d6) δ ppm 8.68 (t, J=6.1 Hz, 1H), 8.53 (s, 1H), 8.02 (d, J=1.2 Hz, 1H), 7.92 - 8.00 (m, 2H), 7.77 - 7.83 (m, 1H), 7.60 (d, J=1.2 Hz, 1H), 7.48 - 7.56 (m, 1H), 7.55 (t, J=54.3 Hz, 1H), 5.59 (s, 1H), 5.41 (s, 1H), 4.11 (s, 3H), 3.96 - 4.02 (m, 1H), 3.86 - 3.93 (m, 1H), 1.56 - 1.64 (m, 1H), 1.54 (s, 3H), 1.47 (s, 3H), 0.58 - 0.65 (m, 1H), 0.39 - 0.47 (m, 1H), 0.28 -0.37 (m, 1H), 0.14 - 0.22 (m, 1H); LC-MS (method H): Rt 2.06 min; mass calcd. for C₃₀H₂₇F₅N₄O₄ 602.2, m/z found 603.2 [M+H]⁺; [α]_{D}²⁰ -34.65° (c 0.329, DMF).

### 3-Cyclopropyl-N-{(-)-2-Cyclopropyl-2-[5-Fluoro-6-(4-Fluorophenyl)-4-(2-Hydroxypropan-2-yl)Pyridin-2-yl]-2-Hydroxyethyl)-8-Methoxycinnoline-6-Carboxamide 271

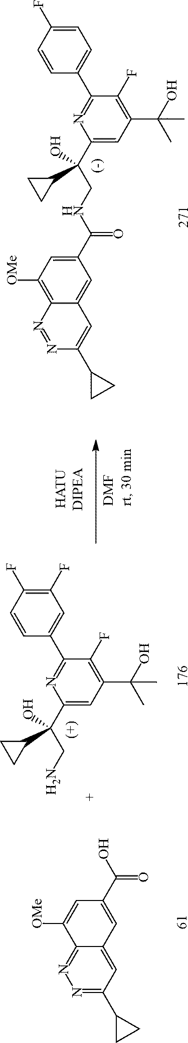

271 (270 mg. 16%) was synthesized according to procedure A. The reaction was quenched with ice and water, and the mixture was stirred for 15 min. The precipitate was collected by filtration and washed with water. The residual fraction was dissolved in EtOAc and dried (MgSOi). The solids were removed by filtration and the filtrate was concentrated under reduced pressure. The crude product was crystallized from EtOAc, the precipitate was collected by filtration, washed with EtOAc and dried under vacuum at 65° C. The product was purified via reverse phase HPLC (stationary phase: Kromasil C18 100A 5 µm (Eka Nobel), mobile phase: NH₄HCO₃ (0.25% in H₂O)/CH₃OH, gradient from 90:10 to 20:80). ¹H NMR (400 MHz, DMSO-d6) δ ppm 8.62 (t, J=6.1 Hz, 1H), 7.98 (dd, J=7.5, 5.5 Hz, 2H), 7.94 (d, J=5.7 Hz, 1H), 7.85 (s, 1H), 7.74 (d, J=1.3 Hz, 1H), 7.35 (d, J=1.5 Hz, 1H), 7.27 - 7.34 (m, 2H), 5.56 (s, 1H), 5.44 (s, 1H), 4.04 (s, 3H), 3.86 - 3.98 (m, 2H), 1.55 - 1.61 (m, 1H), 1.54 (s, 3H), 1.47 (s, 3H), 1.14 - 1.24 (m, 5H), 0.53 -0.63 (m, 1H), 0.36 - 0.45 (m, 1H), 0.25 - 0.35 (m, 1H), 0.11 - 0.21 (m, 1H); [α]_{D}²⁰ -32.40° (c 0.250, DMF).

The following compounds 273-336 were prepared using methods analogous to those described in the preceding examples.

### (-)Fluoro-8-methoxy-3-methyl-n-(3,3,3-trifluoro(5-fluoro-6-(4-fluorophenyl)-4-(2-hydroxypropanyl)pyridinyl)hydroxypropyl)quinoline-6-carboxamide 273

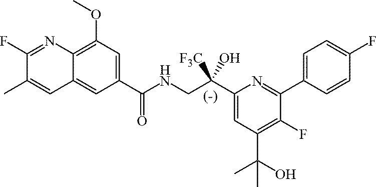

¹H NMR (400 MHz, DMSO-d6) δ 8.74 (t, J=5.72 Hz, 1H), 8.31 (d, J=9.90 Hz, 1H), 8.12 (d, J=5.50 Hz, 1H), 8.00 (dd, J=5.61, 7.59 Hz, 2H), 7.87 (d, J=1.54 Hz, 1H), 7.43 (d, J=1.32 Hz, 1H), 7.38-7.42 (m, 1H), 7.27-7.38 (m, 2H), 5.66 (s, 1H), 4.05-4.30 (m, 2H), 3.95 (s, 3H), 2.40 (s, 3H), 1.55 (s, 3H), 1.49 (s, 3H)
LC-MS (RT: 1.17, MW = 594 [M+H]⁺, METHOD L)
OR = -84.86 ° (589 nm, c 0.185 w/v %, DMF, 20° C.)

### (-)Methoxy-3-Methyl-N-(3,3,3-Trifluoro-2-(5-Fluoro-4-(2-Hydroxypropan-2-yl)-6-(4-(Trifluoromethyl)Phenyl)pyridin-2-yl)-2-Hydroxypropyl)Cinnoline-6-Carboxamide 274

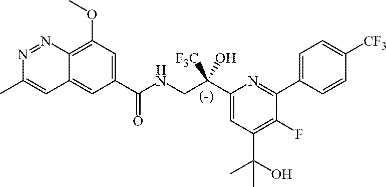

¹H NMR (400 MHz, DMSO-d6) 6 ppm 1.53 (s, 6 H) 2.86 (s, 3 H) 4.06 (s, 3 H) 4.13 - 4.31 (m, 2 H) 5.71 (s, 1 H) 7.33 (s, 1 H) 7.39 (d, J=1.32 Hz, 1 H) 7.78 (d, J=1.54 Hz, 1 H) 7.88 (d, J=8.36 Hz, 2 H) 7.95 (s, 1 H) 8.15 (d, J=8.14 Hz, 2 H) 8.20 (d, J=5.50 Hz, 1 H) 8.78 (t, J=6.05 Hz, 1 H)
LC-MS (RT: 1.13, MW = 626 [M+H]⁺, METHOD L)
OR = -114.73 ° (589 nm, c 0.2545 w/v %, DMF, 20° C.)
119.14° C. (DSC: From 30 to 400° C. at 10° C./min 50 ml N₂)

### (+)-N-cyclopropyl-2-(5-fluoro-6-(4-fluorophenyl)-4-hydroxypropan-2-yl)pyridin-2-yl)-2-hydroxyethyl)-8-methoxy-3-methylcinnoline-6-carboxamide 275 (enantiomer of Compound 252

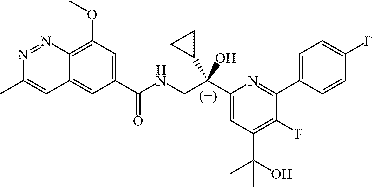

1H NMR (400 MHz, DMSO-d6) 6 8.65 (t, J=6.10 Hz, 1H), 7.95-8.00 (m, 2H), 7.94 (d, J=5.70 Hz, 1H), 7.89 (s, 1H), 7.76 (d, J=1.22 Hz, 1H), 7.39 (d, J=1.22 Hz, 1H), 7.28-7.36 (m, 2H), 5.56 (s, 1H), 5.45 (s, 1H), 4.06 (s, 3H), 3.87-3.97 (m, 2H), 2.86 (s, 3H), 1.55-1.61 (m, 1H), 1.54 (s, 3H), 1.47 (s, 3H), 0.55-0.62 (m, 1H), 0.27-0.45 (m, 2H), 0.12-0.20 (m, 1H)
LC-MS(RT: 1.06, MW = 549 [M+H]⁺, , METHOD L)
OR = +29.89 ° (589 nm, c 0.261 w/v %, DMF, 20° C.)

### (-)-N-{2-[5-Chloro-6-(4-Fluorophenyl)-4-{2-[(Methanesulfonyl)Amino]Propan-2-yl}Pyridin-2-yl]-3,3,3-Trifluoro-2-Hydroxypropyl}-8-Methoxy-3-Methylcinnoline-6-Carboxamide 276 (Enantiomer of 233)

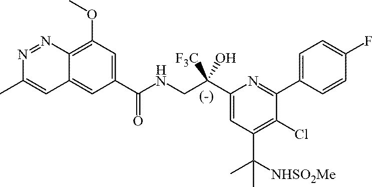

LC-MS (RT: 2.69, MW = 670 [M+H]⁺, Method: method I)
OR = -54.81 ° (589 nm, c 0.27 w/v %, DMF, 20° C.)

### (-)-N-(5-chloro-6-(4-fluorophenyl)-4-hydroxypropan-2-yl)pyridin-2-yl)-3,3,3-trifluoro-2-hydroxypropyl)-8-methoxy-3-(trifluoromethyl)cinnoline-6-carboxamide 277

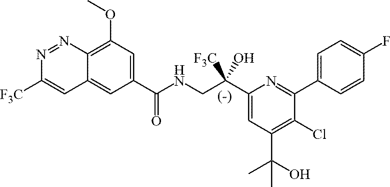

¹H NMR (400 MHz, DMSO-d6) 6 8.98-9.12 (m, 1H), 8.87 (s, 1H), 8.33 (s, 1H), 8.07 (d, J=1.54 Hz, 1H), 7.65-7.77 (m, 3H), 7.48-7.64 (m, 1H), 7.15-7.37 (m, 2H), 5.68 (s, 1H), 4.15 (s, 3H), 4.08-4.30 (m, 2H), 1.64 (s, 3H), 1.56 (s, 3H)

LC-MS (RT: 1.15, MW = 647 [M+H]⁺, METHOD L)

### (-)-N-(6-(4-chlorophenyl)-5-fluoro-4-hydroxypropan-2-yl)pyridin-2-yl)-2-cyclopropyl-2-hydroxyethyl)-8-methoxy-3-methylcinnoline-6-carboxamide 278

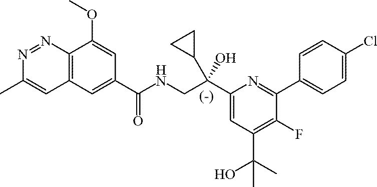

1H NMR (400 MHz, DMSO-d6) 6 8.65 (t, J=5.94 Hz, 1H), 7.91-8.00 (m, 3H), 7.88 (s, 1H), 7.75 (d, J=1.32 Hz, 1H), 7.54 (d, J=7.65 Hz, 2H), 7.39 (d, J=1.32 Hz, 1H), 5.58 (s, 1H), 5.44 (s, 1H), 4.06 (s, 3H), 3.84-3.98 (m, 2H), 2.87 (s, 3H), 1.55-1.62 (m, 1H), 1.54 (s, 3H), 1.47 (s, 3H), 0.54-0.63 (m, 1H), 0.36-0.45 (m, 1H), 0.27-0.36 (m, 1H), 0.12-0.21 (m, 1H)

OR = -37.99 ° (589 nm, c 0.5475 w/v %, DMF, 20° C.)

### (RS)Methoxy-3-Methyl-N-(3,3,3-Trifluoro-2-(3-Fluoro-6-(4-Fluorophenyl)-4-(2-Hydroxypropan-2-yl)Pyridin-2-yl)-2-Hydroxypropyl)Cinnoline-6-Carboxamide 279

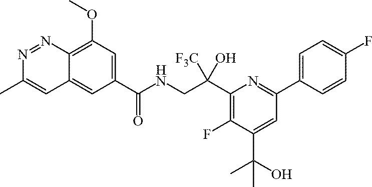 [SMILES: COc1cc(C(=O)NCC(O)(c2nc(-c3ccc(F)cc3)cc(C(C)(C)O)c2F)C(F)(F)F)cc2cc(C)nnc12]

¹H NMR (400 MHz, DMSO-d6, 27° C.) δ ppm 8.69 (t, J=6.1 Hz, 1 H) 8.10 - 8.21 (m, 3 H) 7.83 (s, 1 H) 7.66 (d, J=1.3 Hz, 1 H) 7.31 - 7.41 (m, 2 H) 7.25 (d, J=1.3 Hz, 1 H) 7.04 (s, 1 H) 5.63 (s, 1 H) 4.49 - 4.62 (m, 1 H) 3.99 - 4.05 (m, 1 H) 3.98 (s, 3 H) 2.83 (s, 3 H) 1.50 (s, 6 H)

LC-MS(RT: 1.01, Area %: 98.37, MW: 576.00, BPM1: 577, BPM2: 577, METHOD L)

### (+)Methoxy-3-Methyl-N-(3.3.3-Trifluoro-2-(3-Fluoro-6-(4-Fluorophenyl)-4-(2-Hydroxypropan-2-yl)Pyridm-2-yl)-2-Hydroxypropyl)Cinnoline-6-Carboxamide 280

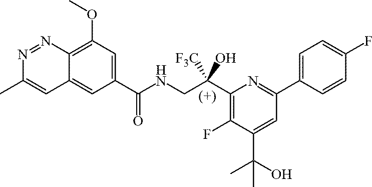

¹H NMR (400 MHz, DMSO-d₆, 27° C.) δ ppm 8.69 (t, J=6.1 Hz, 1 H) 8.10 - 8.21 (m, 3 H) 7.83 (s, 1 H) 7.66 (d, J=1.3 Hz, 1 H) 7.31 - 7.41 (m, 2 H) 7.25 (d, J=1.3 Hz, 1 H) 7.04 (s, 1 H) 5.63 (s, 1 H) 4.49 - 4.62 (m, 1 H) 3.99 - 4.05 (m, 1 H) 3.98 (s, 3 H) 2.83 (s, 3 H) 1.50 (s, 6 H)

### (-)Methoxy-3-Methyl-N-(3,3,3-Trifluoro-2-(3-Fluoro-6-(4-Fluorophenyl)-4-(2-Hydroxypropan-2-yl)Pyridin-2-yl)-2-Hydroxypropyl)Cinnoline-6-Carboxarmide 281

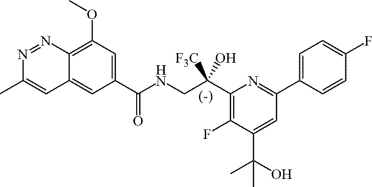

¹H NMR (400 MHz, DMSO-d₆, 27° C.) δ ppm 8.69 (t, J=6.1 Hz, 1 H) 8.10 - 8.21 (m, 3 H) 7.83 (s, 1 H) 7.66 (d, J=1.3 Hz, 1 H) 7.31 - 7.41 (m, 2 H) 7.25 (d, J=1.3 Hz, 1 H) 7.04 (s, 1 H) 5.63 (s, 1 H) 4.49 - 4.62 (m, 1 H) 3.99 - 4.05 (m, 1 H) 3.98 (s, 3 H) 2.83 (s, 3 H) 1.50 (s, 6 H)

LC-MS(RT: 1.01, Area %: 98.37, MW: 576.00, BPM1: 577, BPM2: 577, METHOD L)

### (-)-N-(5-chloro-6-(4-fluorophenyl)-4-hydroxypropan-2-yl)pyridin-2-yl)-2-cyclopropyl-2-hydroxyethyl)-3-cyclopropyl-8-methoxycinnoline-6-carboxamide 282

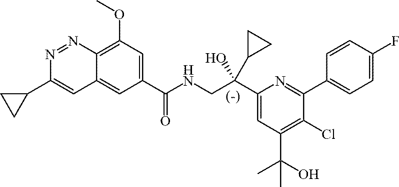

1H NMR (400 MHz, DMSO-d6) δ ppm 0.06 - 0.65 (m, 4 H) 1.10 - 1.28 (m, 4 H) 1.48 - 1.54 (m, 1 H) 1.56 (s, 3 H) 1.64 (s, 3 H) 3.79 - 3.98 (m, 2 H) 4.06 (s, 3 H) 5.43 (s, 1 H) 5.58 (s, 1 H) 7.25 (t, J=8.88 Hz, 2 H) 7.35 (d, J=1.25 Hz, 1 H) 7.62 - 7.71 (m, 2 H) 7.72 (d, J=1.25 Hz, 1 H) 7.87 (s, 1 H) 8.15 (s, 1 H) 8.64 (s, 1 H)
LC-MS (RT: 9.32, MW = 591 [M+H]⁺, METHOD M)
OR = -13.45 ° (589 nm, c 0.4685 w/v %, DMF, 20° C.)

### (-)Cyclopropyl-N-(2-(5-Fluoro-6-(4-Fluorophenyl)-4-(2-Hydroxypropan-2-yl)Pyridin-2-yl)-2-HydroxyMethylbutyl)-8-Methoxycinnoline-6-Carboxamide 283

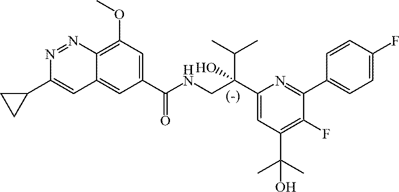

1H NMR (400 MHz, DMSO-d6) δ 8.37 (br t, J=5.72 Hz, 1H), 7.92-7.98 (m, 3H), 7.76 (s, 1H), 7.62 (d, J=1.54 Hz, 1H), 7.26-7.32 (m, 2H), 7.25 (d, J=1.32 Hz, 1H), 5.54 (d, J=11.22 Hz, 2H), 4.00 (s, 3H), 3.95-4.00 (m, 1H), 3.74 (dd, J=4.51, 13.31 Hz, 1H), 2.40-2.48 (m, 2H), 1.52 (s, 3H), 1.44 (s, 3H), 1.12-1.24 (m, 4H), 1.02 (d, J=6.82 Hz, 3H), 0.72 (d, J=6.82 Hz, 3H).
LC-MS (RT: 2.13, MW = 577 [M+H]⁺, METHOD Q)
OR = -58.31 ° (589 nm, c 0.5265 w/v %, DMF, 20° C.)

### (-)-N-cyclopropyl-2-(5-fluoro-6-(4-fluorophenyl)-4-hydroxypropan-2-yl)pyridin-2-yl)-2-hydroxyethyl)-8-methoxy-3-(trifluoromethyl)cinnoline-6-carboxamide 284

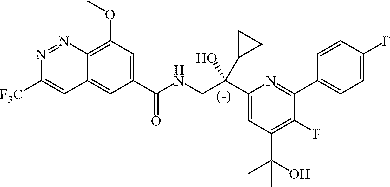

¹H NMR (400 MHz, DMSO-d6) δ ppm 8.81 (s, 1 H) 8.75 (t, J=6.1 Hz, 1 H) 8.06 (d, J=1.5 Hz, 1 H) 7.92 - 8.00 (m, 3 H) 7.69 (d, J=1.3 Hz, 1 H) 7.20 - 7.34 (m, 2 H) 5.57 (s, 1 H) 5.41 (s, 1 H) 4.13 (s, 3 H) 3.94 (d, J=5.9 Hz, 2 H) 1.57 - 1.63 (m, 1 H) 1.54 (s, 3 H) 1.48 (s, 3 H) 0.55 - 0.65 (m, 1 H) 0.38 - 0.48 (m, 1 H) 0.26 - 0.36 (m, 1 H) 0.12 - 0.24 (m, 1 H)
LC-MS (RT: 1.15, MW = 603 [M+H]⁺, METHOD L)
OR = -15.03 ° (589 nm, c 0.2595 w/v %, DMF, 20° C.)

### (S)(Difluoromethyl)-N-(2-(5-Fluoro-6-(4-Fluorophenyl)-4-(2-Hydroxypropan-2-yl)Pyridin-2-yl)-2-HydroxyMethylbutyl)-8-Methoxycinnoline-6-Carboxamide 285

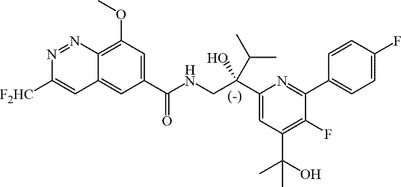

1H NMR (400 MHz, DMSO-d6) δ 8.42-8.48 (m, 2H), 7.90-7.99 (m, 4H), 7.50 (d, J=1.32 Hz, 1H), 7.55 (t, J=54.36 Hz, 1H), 7.27 (t, J=8.25 Hz, 2H), 5.56 (s, 1H), 5.51 (s, 1H), 4.07 (s, 3H), 4.01 (dd, J=7.04, 13.42 Hz, 1H), 3.76 (dd, J=4.73, 13.31 Hz, 1H), 2.41-2.49 (m, 1H), 1.52 (s, 3H), 1.44 (s, 3H), 1.03 (d, J=6.82 Hz, 3H), 0.73 (d, J=6.82 Hz, 3H)
LC-MS(RT: 2.10, MW = 587 [M+H]⁺, METHOD Q)
OR = -66.99 ° (589 nm, c 0.521 w/v %, DMF, 20° C.)

### (-)Cyclopropyl-8-Methoxy-N-(3,3,3-Trifluoro-2-(5-Fluoro-6-(4-Fluorophenyl)-4-(2-Hydroxypropan-2-yl)Pyridin-2-yl)-2-Hydroxypropyl)Cinnoline-6-Carboxamide 286

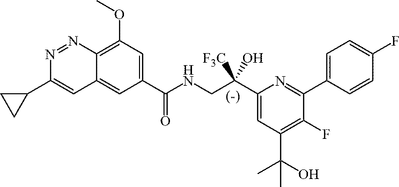

¹H NMR (400 MHz, DMSO-d6, 27° C.) δ ppm 8.76 (t, J=5.9 Hz, 1 H) 8.12 (d, J=5.3 Hz, 1 H) 7.99 (dd, J=7.5, 5.5 Hz, 2 H) 7.88 (s, 1 H) 7.75 (d, J=1.5 Hz, 1 H) 7.31 - 7.40 (m, 3 H) 7.29 (s, 1 H) 5.67 (s, 1 H) 4.06 - 4.33 (m, 2 H) 4.05 (s, 3 H) 2.42 - 2.49 (m, 1 H) 1.55 (s, 3 H) 1.48 (s, 3 H) 1.12 - 1.24 (m, 4 H)
LC-MS (RT: 2.08, MW = 602 [M+H]⁺, METHOD S)
OR = -92.8 ° (589 nm, c 0.264 w/v %, DMF, 20° C.)

### (-)-N-cyclopropyl-2-(3,5-difluoro-6-(4-fluorophenyl)-4-hydroxypropan-2-yl)pyridin-2-yl)-2-hydroxyethyl)-3-(difluoromethyl)-8-methoxycinnoline-6-carboxamide 287

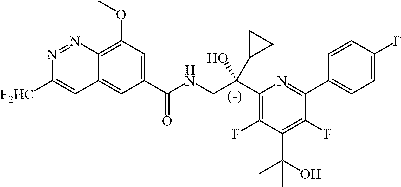

¹H NMR (400 MHz, DMSO-d6) δ 8.62 (t, J=6.90 Hz, 1H), 8.42 (s, 1H), 7.88 (d, J=1.32 Hz, 1H), 7.76-7.87 (m, 2H), 7.45 (d, J=1.32 Hz, 1H), 7.53 (t, J=54.36 Hz, 1H), 7.21-7.31 (m, 2H), 5.57 (s, 1H), 5.32 (s, 1H), 4.03 (s, 3H), 3.60-4.29 (m, 2H), 1.67-1.75 (m, 1H), 1.64 (s, 6H), 0.65-0.76 (m, 1H), 0.40-0.53 (m, 1H), 0.19-0.36 (m, 2H)
LC-MS (RT: 1.07, MW = 603 [M+H]⁺, METHOD L)
OR -9.06 ° (589 nm, c 0.508 w/v %, DMF, 20° C.)

### (-)Cyclopropyl-N-(2-Cyclopropyl-2-(3,5-Difluoro-6-(4-Fluorophenyl)-4-(2-Hydroxypropan-2-yl)Pyridin-2-yl)-2-Hydroxyethyl)-8-Methoxycinnoline-6-Carboxamide 288

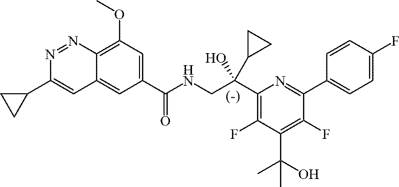

¹H NMR (400 MHz, DMSO-d6) δ 8.52 (t, J=6.80 Hz, 1H), 7.85 (dd, J=5.50, 7.48 Hz, 2H), 7.74 (s, 1H), 7.59 (d, J=1.32 Hz, 1H), 7.24-7.32 (m, 2H), 7.20 (d, J=1.54 Hz, 1H), 5.56 (s, 1H), 5.31 (s, 1H), 3.96 (s, 3H), 3.57-4.28 (m, 2H), 2.40-2.48 (m, 1H), 1.65-1.73 (m, 1H), 1.63 (s, 6H), 1.10-1.22 (m, 4H), 0.64-0.74 (m, 1H), 0.39-0.50 (m, 1H), 0.18-0.35 (m, 2H)
LC-MS (RT: 1.10, MW = 593 [M+H]⁺, METHOD L)
OR = -10.87 ° (589 nm, c 0.5245 w/v %, DMF, 20° C.)

### (-)Cyclopropyl-N-(2-Cyclopropyl-2-(5-Fluoro-6-(4-Fluorophenyl)-4-(2-Hydroxypropan-2-yl)Pyridin-2-yl)-2-Hydroxyethyl)-8-(Methoxy-d3)Cinnoline-6-Carboxamide 289

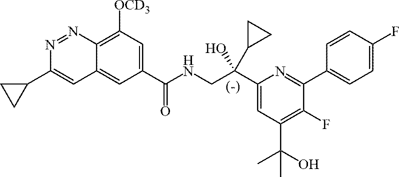

¹H NMR (400 MHz, DMSO-d6) δ 8.62 (t, J=5.94 Hz, 1H), 7.98 (dd, J=5.50, 7.48 Hz, 2H), 7.94 (d, J=5.72 Hz, 1H), 7.85 (s, 1H), 7.74 (d, J=1.32 Hz, 1H), 7.35 (d, J=1.54 Hz, 1H), 7.18-7.34 (m, 2H), 5.56 (s, 1H), 5.44 (s, 1H), 3.84-3.99 (m, 2H), 2.41-2.48 (m, 1H), 1.55-1.62 (m, 1H), 1.54 (s, 3H), 1.47 (s, 3H), 1.12-1.26 (m, 4H), 0.54-0.64 (m, 1H), 0.36-0.48 (m, 1H), 0.25-0.34 (m, 1H), 0.11-0.23 (m, 1H)
LC-MS (RT: 1.10, MW = 578 [M+H]⁺, METHOD L)
OR = -31.66 ° (589 nm, c 0.537 w/v %, DMF, 20° C.)

### (-)-N-cyclopropyl-2-(5-fluoro-6-(4-fluorophenyl)-4-hydroxypropan-2₋yl)pyridin-2-yl)-2-hydroxyethyl)-8-(methoxy-d3)-3-methylcinnoline-6-carboxamide 290

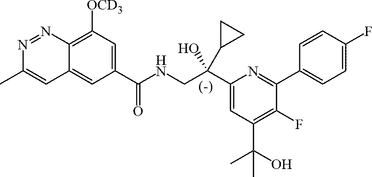

¹H NMR (400 MHz, DMSO-d6) δ 8.65 (t, J=1.00 Hz, 1H), 7.98 (dd, J=5.50, 7.48 Hz, 2H), 7.94 (d, J=5.72 Hz, 1H), 7.89 (s, 1H), 7.76 (d, J=1.54 Hz, 1H), 7.39 (d, J=1.54 Hz, 1H), 7.26-7.36 (m, 2H), 5.56 (br s, 1H), 5.45 (br s, 1H), 3.83-3.99 (m, 2H), 2.86 (s, 3H), 1.55-1.62 (m, 1H), 1.54 (s, 3H), 1.47 (s, 3H), 0.54-0.65 (m, 1H), 0.35-0.48 (m, 1H), 0.26-0.34 (m, 1H), 0.11-0.22 (m, 1H)
LC-MS(RT: 1.02, MW = 552 [M+H]⁺, METHOD L)
OR = -29.64 ° (589 nm, c 0.523 w/v %, DMF, 20° C.)

### (-)Cyclopropoxy-3-Methyl-N-(3,3,3-Trifluoro-2-(6-(4-Fluorophenyl)-4-(2-Hydroxypropan-2-yl)Pyridin-2-yl)-2-Hydroxypropyl)Cinnoline-6-Carboxamide 291

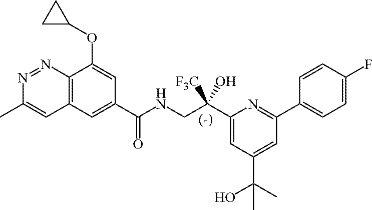

1HNMR (400 MHz, METHANOL-d4) δ 8.14 (t, J=6.55 Hz, 2H), 7.98 (d, J=1.32 Hz, 2H), 7.86 (s, 1H), 7.64 (br s, 2H), 7.20 (t, J=8.20 Hz, 2H), 4.61-4.69 (m, 1H), 4.02 (br d, J=13.86 Hz, 2H), 2.90 (br s, 3H), 1.55 (s, 3H), 1.55 (s, 3H), 0.89 (br s, 4H)

LC-MS(RT: 2.01, MW = 585 [M+H]⁺, METHOD Q)

### (-)-N-(3,3-difluoro-2-(5-fluoro-6-(4-fluorophenyl)-4-(2-hydroxypropan-2-yl)pyridin-2-yl)-2-hydroxypropyl)-3-(difluoromethyl)-8-methoxycinnoline-6-carboxamide 292

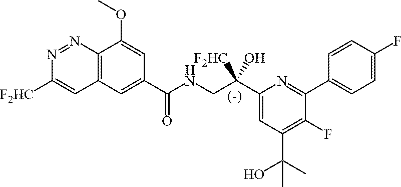

¹H NMR (400 MHz, DMSO-d6) δ 8.75 (t, J=6.16 Hz, 1H), 8.45 (s, 1H), 8.04 (d, J=5.50 Hz, 1H), 7.96 (d, J=1.32 Hz, 1H), 7.92 (dd, J=5.50, 7.70 Hz, 2H), 7.55 (s, 1H), 7.39-7.72 (m, 1H), 7.17-7.27 (m, 2H), 6.55 (s, 1H), 6.51-6.89 (m, 1H), 5.63 (s, 1H), 4.09 (s, 3H), 3.79-4.05 (m, 2H), 1.54 (s, 3H), 1.45 (s, 3H)
LC-MS (RT: 1.01, MW = 595 [M+H]⁺, METHOD L)
OR -68.52 ° (589 nm, c 0.27 w/v %, DMF, 20° C.),

### (-)Cyclopropyl-N-(3,3-Difluoro-2-(5-Fluoro-6-(4-Fluorophenyl)-4-(2-Hydroxypropan-2-yl)Pyridin-2-yl)-2-Hydroxypropyl)-8-Methoxycinnoline-6-Carboxamide 293

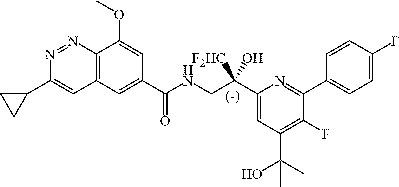

¹H NMR (400 MHz, DMSO-d6) δ 8.66 (t, J=6.16 Hz, 1H), 8.03 (d, J=5.72 Hz, 1H), 7.93 (dd, J=5.50, 7.48 Hz, 2H), 7.79 (s, 1H), 7.69 (d, J=1.54 Hz, 1H), 7.30 (d, J=1.1.54 Hz, 1H), 7.19-7.28 (m, 2H), 6.55 (s, 1H), 6.48-6.83 (m, 1H), 5.62 (s, 1H), 4.02 (s, 3H), 3.77-4.00 (m, 2H), 2.43-2.48 (m, 1H), 1.54 (s, 3H), 1.45 (s, 3H), 1.13-1.25 (m, 4H)
LC-MS(RT: 1.02, MW = 585 [M+H]⁺, METHOD L)
OR = -85.16 ° (589 nm, c 0.256 w/v %, DMF, 20° C.)

### N-((-)(2,2-dimethylcyclopropyl)(5-fluoro-6-(4-fluorophenyl)-4-(2-hydroxypropanyl)pyndmyl)hydroxyethyl)-8-methoxy-3-methylcmnoline-6-carboxamide 294

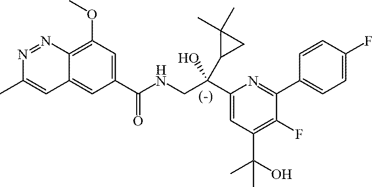

¹H NMR (400 MHz, DMSO-d6 ) d 8.72 (t, J=6.05 Hz, 1H), 7.92-8.05 (m, 3H), 7.87 (s, 1H), 7.78 (d, J=1.54 Hz, 1H), 7.41 (d, J=1.32 Hz, 1H), 7.26-7.35 (m, 2H), 5.55 (s, 1H), 5.46 (s, 1H), 4.07 (s, 3H), 3.69-3.91 (m, 2H), 2.87 (s, 3H), 1.52 (s, 6H), 1.39-1.47 (m, 1H), 0.96 (s, 3H), 0.79 (s, 3H), 0.69-0.76 (m, 1H), 0.38-0.46 (m, 1H)

LC-MS (RT: 1.12, MW =577 [M+H]⁺, METHOD L)

### N-((+)(2,2-dimethylcyclopropyl)(5-fluoro-6-(4-fluorophenyl)-4-(2-hydroxypropanyl)pyridinyl)hydroxyethyl)-8-methoxy-3-methylcinnoline-6-carboxamide 295

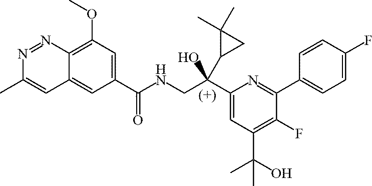

¹H NMR (400 MHz, DMSO-d6 ) δ 8.73 (t, J=5.94 Hz, 1H), 7.94-8.02 (m, 3H), 7.87 (s, 1H), 7.78 (d, J=1.54 Hz, 1H), 7.41 (d, J=1.32 Hz, 1H), 7.26-7.37 (m, 2H), 5.55 (s, 1H), 5.47 (s, 1H), 4.07 (s, 3H), 3.68-3.90 (m, 2H), 2.87 (s, 3H), 1.52 (s, 6H), 1.44 (dd, J=5.83, 8.91 Hz, 1H), 0.96 (s, 3H), 0.79 (s, 3H), 0.69-0.76 (m, 1H), 0.42 (dd, J=3.85, 8.69 Hz, 1H)

LC-MS (RT: 1.12, MW = 577 [M+H]⁺, METHOD L)

### (-)-N-(5-fluoro-6-(4-fluorophenyl)-4-hydroxypropan-2-yl)pyridin-2-yl)-2-(1-fluorocyclopropyl)-2-hydroxyethyl)-8-methoxy-3-methylcinnoline-6-carboxamide 296

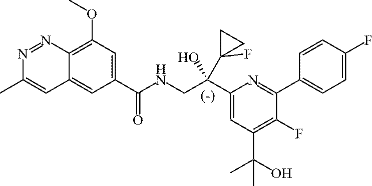

¹H NMR (400 MHz, DMSO-d6) δ 8.64 (t, J=5.72 Hz, 1H), 8.03 (d, J=5.50 Hz, 1H), 7.99 (dd, J=5.61, 7.59 Hz, 2H), 7.92 (s, 1H), 7.78 (d, J=1.54 Hz, 1H), 7.40 (d, J=1.32 Hz, 1H), 7.28-7.37 (m, 2H), 6.18 (s, 1H), 5.61 (s, 1H), 4.08-4.29 (m, 2H), 4.06 (s, 3H), 2.86 (s, 3H), 1.55 (s, 3H), 1.51 (s, 3H), 0.88-1.14 (m, 4H)
LC-MS(RT: 1.85, MW = 567 [M+H]⁺, METHOD O)
OR = -28.35 ° (589 nm, c 0.2575 w/v %, DMF, 20° C.)

### (+)-N-(5-fluoro-6-(4-fluorophenyl)-4-hydroxypropan-2-yl)pyridm-2-yl)-2-(l-fluorocyclopropyl)-2-hydroxyethyl)-8-methoxy-3-methylcinnoline-6-carboxamide 297

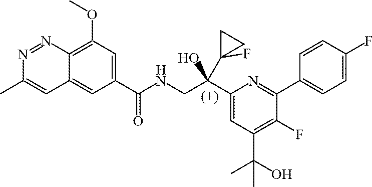

LC-MS(RT: 0.99, MW = 567 [M+H]⁺, METHOD L)
OR = +28.51 ° (589 nm, c 0.2525 w/v %, DMF, 20° C.)

### (-)-N-(5-fluoro-6-(4-fluorophenyl)-4-hydroxypropan-2-yl)pyridin-2-yl)-2-(1-methylcyclopropyl)-2-hydroxyethyl)-8-methoxy-3-methylcinnoline-6-carboxamide 298

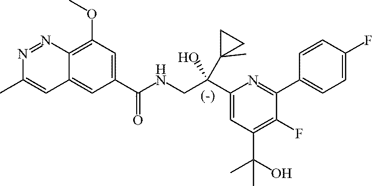

1H NMR (400 MHz, DMSO-d6 )δ8.51 (t, J=5.61 Hz, 1H), 7.96-8.03 (m, 3H), 7.95 (d, J=5.72 Hz, 1H), 7.81 (d, J=1.32 Hz, 1H), 7.42 (d, J=1.32 Hz, 1H), 7.27-7.39 (m, 2H), 5.66 (s, 1H), 5.58 (s, 1H), 4.07 (s, 3H), 3.99-4.36 (m, 2H), 2.87 (s, 3H), 1.56 (s, 3H), 1.52 (s, 3H), 1.04 (s, 3H), 0.90-0.99 (m, 1H), 0.77-0.86 (m, 1H), 0.20-0.29 (m, 1H), 0.00-0.06 (m, 1H)

LC-MS (RT: 1.93, MW = 563 [M+H]⁺, METHOD O)

### (+)-N-(5-fluoro-6-(4-fluorophenyl)-4-hydroxypropan-2-yl)pyridin-2-yl)-2-(1-methylcyclopropyl)-2-hydroxyethyl)-8-methoxy-3-methylcinnoline-6-carboxamide 299

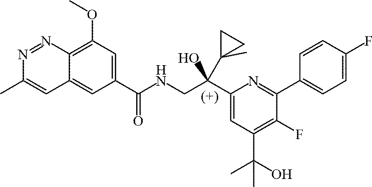

1H NMR (400 MHz, DMSO-d6 )δ8.51 (t, J=5.61 Hz, 1H), 7.96-8.02 (m, 3H), 7.95 (d, J=5.50 Hz, 1H), 7.80 (d, J=1.32 Hz, 1H), 7.42 (d, J=1.32 Hz, 1H), 7.27-7.39 (m, 2H), 5.65 (s, 1H), 5.58 (s, 1H), 4.07 (s, 3H), 4.00-4.32 (m, 2H), 2.87 (s, 3H), 1.56 (s, 3H), 1.52 (s, 3H), 1.04 (s, 3H), 0.91-1.00 (m, 1H), 0.81-0.85 (m, 1H), 0.20-0.28 (m, 1H), 0.01-0.07 (m, 1H)

LC-MS(RT: 1.93, MW = 563 [M+H]⁺, METHOD O)

### N-((*S)(5-Fluoro-6-(4-Fluorophenyl)-4-(2-Hydroxypropanyl)Pyridinyl)((1*S,2*R)Fluorocyclopropyl)Hydroxyethyl)-8-Methoxy-3-Methylcinnoline-6-Carboxamide 300

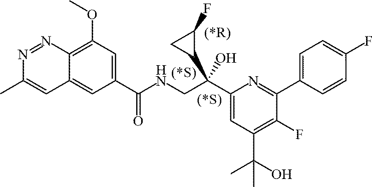

¹H NMR (400 MHz, DMSO-d6) δ 8.66 (t, J=5.94 Hz, 1H), 7.93-8.02 (m, 3H), 7.86 (s, 1H), 7.75 (d, J=, 1H), 7.38 (d, J=1.32 Hz, 1H), 7.26-7.35 (m, 2H), 5.67 (s, 1H), 5.58 (s, 1H), 4.42-4.70 (m, 1H), 4.05 (s, 3H), 3.75-3.89 (m, 2H), 2.86 (s, 3H), 2.08-2.25 (m, 1H), 1.55 (s, 3H), 1.47 (s, 3H), 1.00-1.13 (m, 2H)

SFC (RT: 5.50, Method SFC)

### N-((*S)(5-Fluoro-6-(4-Fluorophenyl)-4-(2-Hydroxypropanyl)Pyridinyl)((1*S.2*S)Fluorocyclopropyl)Hydroxyethyl)-8-Methoxy-3-Methylcinnoline-6-Carboxamide 302

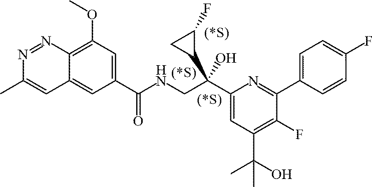

¹H NMR (400 MHz, DMSO-d6) δ 8.76 (t, J=5.94 Hz, 1H), 7.93-8.03 (m, 2H), 7.87-7.92 (m, 2H), 7.79 (d, J=1.32 Hz, 1H), 7.41 (d, J=1.54 Hz, 1H), 7.23-7.37 (m, 2H), 5.68 (s, 1H), 5.57 (s, 1H), 4.67-4.93 (m, 1H), 4.06 (s, 3H), 3.84-4.05 (m, 2H), 2.87 (s, 3H), 2.01-2.16 (m, 1H), 1.54 (s, 3H), 1.47 (s, 3H)
SFC (RT: 5.71, Area %: 100.00, Method SFC)
OR = -22 ° (589 nm, c 0.2 w/v %, DMF, 20° C.)

### N-((*R)(5-Fluoro-6-(4-Fluorophenyl)-4-(2-Hydroxypropanyl)Pyridinyl)((1*R,2*S)Fluorocyclopropyl)Hydroxyethyl)-8-Methoxy-3-Methylcinnoline-6-Carboxamide 301

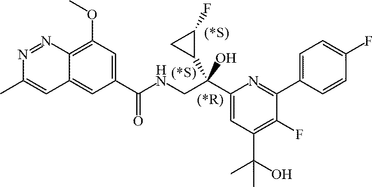

LC-MS (RT: 1.88, MW = 567 [M+H], METHOD R)
SFC (RT: 5.38, Area %: 100.00, Method SFC)

### N-((*R)(5-Fluoro-6-(4-Fluorophenyl)-4-(2-Hydroxypropanyl)Pyridinyl)((l*R, 2*R)Fluorocyclopropyl)Hydroxyethyl)-8-Methoxy-3-Methylcinnoline-6-Carboxamide 301 303

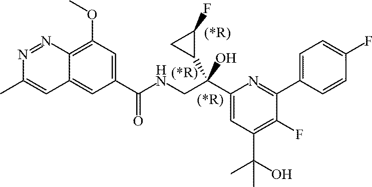

LC-MS(RT: 1.90, MW = 567 [M+H]⁺, METHOD R)
SFC (RT: 5.17, Area %: 100.00, Method SFC)

### (-)-N-cyclopropyl-2-(6-(4-fluorophenyl)-4-hydroxypropan-2-yl)pyridin-2-yl)-2-hydroxyethyl)-8-methoxy-3-methylcinnoline-6-carboxamide 304

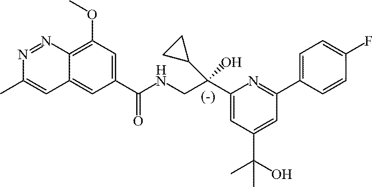

1H NMR (400 MHz, DMSO-d6) δ ppm 0.07 - 0.68 (m, 4 H) 1.45 (d, J=1.05 Hz, 6 H) 1.61 (ddd, J=8.15, 5.33, 3.03 Hz, 1 H) 2.85 (s, 3 H) 3.80 - 4.11 (m, 5 H) 5.28 (s, 1 H) 5.46 (s, 1 H) 7.30 (t, J=8.88 Hz, 2 H) 7.38 (d, J=1.25 Hz, 1 H) 7.83 (d, J=1.25 Hz, 1 H) 7.86 (s, 1 H) 8.10 - 8.24 (m, 2 H) 8.63 (t, J=5.80 Hz, 1 H)
LC-MS (RT: 8.50, MW = 531 [M+H]⁺, METHOD M)
OR = -23.92 ° (589 nm, c 0.418 w/v %, DMF, 20° C.)

### (+)-N-cyclopropyl-2-(6-(4-fluorophenyl)-4-hydroxypropan-2-yl)pyridin-2-yl)-2-hydroxyethyl)-8-methoxy-3-methylcinnoline-6-carboxamide 305

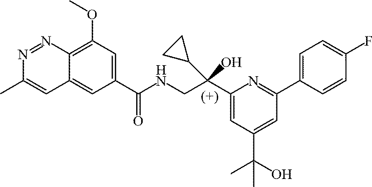

1H NMR (400 MHz, DMSO-d6) δ ppm 0.06 - 0.64 (m, 4 H) 1.45 (d, J=1.05 Hz, 6 H) 1.55 - 1.65 (m, 1 H) 2.85 (s, 1 H) 3.86 - 4.06 (m, 5 H) 5.19 - 5.34 (m, 1 H) 5.28 (s, 1 H) 5.45 (s, 1 H) 7.30 (t, J=8.88 Hz, 2 H) 7.37 (d, J=1.36 Hz, 1 H) 7.72 (d, J=1.36 Hz, 1 H) 7.74 (d, J=1.46 Hz, 1 H) 7.82 (d, J=1.36 Hz, 1 H) 7.86 (s, 1 H) 8.14 - 8.21 (m, 2 H) 8.63 (t, J=5.75 Hz, 1 H)
LC-MS (RT: 8.49, MW = 531 [M+H]⁺, METHOD M)
OR = +25.25 ° (589 nm, c 0.404 w/v %, DMF, 20° C.)

### (-)-N-fluoro-2-(6-(4-fluorophenyl)-4-(2-hydroxypropan-2-yl)pyridin-2-yl)-2-hydroxy-3-methylbutyl)-8-methoxy-3-methylcinnoline-6-carboxamide 306

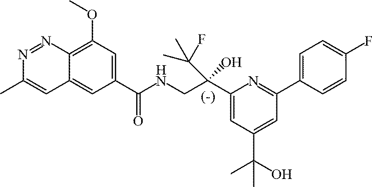

1H NMR (400 MHz, DMSO-d6 ) δ 8.36 (t, J=6.80 Hz, 1H), 8.03 (d, J=5.28 Hz, 1H), 7.98 (dd, J=5.61, 7.59 Hz, 2H), 7.91 (s, 1H), 7.66 (d, J=1.32 Hz, 1H), 7.31-7.38 (m, 2H), 7.27 (d, J=1.32 Hz, 1H), 5.95 (s, 1H), 4.33-4.46 (m, 1H), 4.01 (s, 3H), 3.85-3.94 (m, 1H), 2.85 (s, 3H), 1.50 (s, 3H), 1.48 (s, 3H), 1.44 (d, J=1.00 Hz, 3H), 1.37 (d, J=22.01 Hz, 3H)
LC-MS(RT: 1.89, MW = 569 [M+H]⁺, METHOD O)
OR = -91.04 ° (589 nm, c 0.2735 w/v %, DMF, 20° C.)

### (+)-N-fluoro-2-(6-(4-fluorophenyl)-4-(2-hydroxypropan-2-yl)pyridin-2-yl)-2-hydroxy-3-methylbutyl)-8-methoxy-3-methylcinnoline-6-carboxamide 307

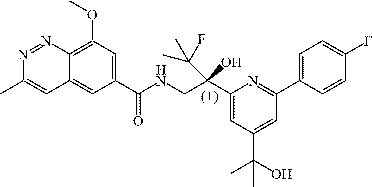

1H NMR (400 MHz, DMSO-d6 ) δ 8.34 (dd, J=4.73, 6.71 Hz, 1H), 8.03 (d, J=5.50 Hz, 1H), 7.98 (dd, J=5.50, 7.48 Hz, 2H), 7.89 (s, 1H), 7.65 (d, J=1.32 Hz, 1H), 7.30-7.39 (m, 2H), 7.27 (d, J=1.32 Hz, 1H), 5.97 (br s, 1H), 5.56 (br s, 1H), 4.33-4.43 (m, 1H), 4.01 (s, 3H), 3.86-3.96 (m, 1H), 2.84 (s, 3H), 1.50 (s, 3H), 1.48 (s, 3H), 1.44 (d, J=22.89 Hz, 3H), 1.37 (d, J=22.23 Hz, 3H)
LC-MS(RT: 1.89, MW = 569 [M+H]⁺, METHOD O)
OR = +79.7 ° (589 nm, c 0.2685 w/v %, DMF, 20° C.)

### (-)Cyclopropoxy-N-(3,3-Difluoro-2-(5-Fluoro-6-(4-Fluorophenyl)-4-(2-Hydroxypropan-2-yl)Pyridin-2-yl)-2-Hydroxypropyl)-3-Methylcinnoline-6-Carboxamide 308

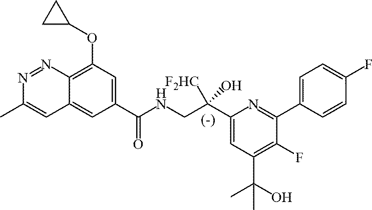

¹H NMR (400 MHz, DMSO-d6) δ 8.65 (t, J=6.16 Hz, 1H), 8.04 (d, J=5.50 Hz, 1H), 7.93 (dd, J=5.50, 7.70 Hz, 2H), 7.78 (s, 1H), 7.72 (d, J=1.54 Hz, 1H), 7.64 (d, J=1.32 Hz, 1H), 7.23-7.32 (m, 2H), 6.54 (s, 1H), 6.49-6.81 (m, 1H), 5.62 (s, 1H), 4.10 (tt, J=3.05, 5.97 Hz, 1H), 3.75-4.04 (m, 2H), 2.86 (s, 3H), 1.54 (s, 3H), 1.46 (s, 3H), 0.85-0.95 (m, 2H), 0.76-0.85 (m, 2H)
LC-MS(RT: 1.84, MW = 585 [M+H]⁺, METHOD O)
OR = -76.16 ° (589 nm, c 0.2705 w/v %, DMF, 20° C.)

### (-)-N-(3,3-difluoro-2-(6-(4-fluorophenyl)-4-(2-hydroxypropan-2-yl)pyridin-2-yl)-2-hydroxybutyl)-8-methoxy-3-methylcinnoline-6-carboxamide 309

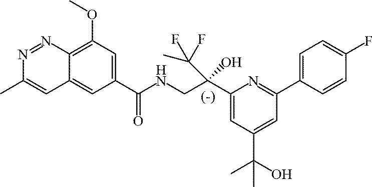

1HNMR (400 MHz, DMSO-d6 ) δ 8.53 (dd, J=4.40, 7.26 Hz, 1H), 8.16-8.25 (m, 2H), 7.92 (d, J=1.32 Hz, 1H), 7.83 (s, 1H), 7.79 (s, 1H), 7.65 (d, J=1.32 Hz, 1H), 7.30-7.36 (m, 2H), 7.25 (d, J=1.32 Hz, 1H), 6.57 (s, 1H), 5.32 (s, 1H), 4.39-4.49 (m, 1H), 3.98 (s, 3H), 3.90-3.96 (m, 1H), 2.83 (s, 3H), 1.60-1.77 (m, 3H), 1.43 (s, 6H)
LC-MS (RT: 1.76, MW = 555 [M+H]⁺, METHOD O)
OR = -51.85 ° (589 nm, c 0.216 w/v %, DMF, 20° C.)

### (+)-N-(3,3-difluoro-2-(6-(4-fluorophenyl)-4-(2-hydroxypropan-2-yl)pyridin-2-yl)-2-hydroxybutyl)-8-methoxy-3-methylcinnoline-6-carboxamide 310

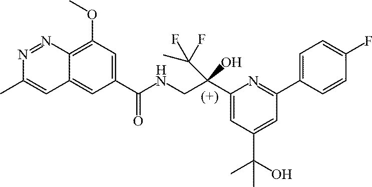

1H NMR (400 MHz, DMSO-d6 ) δ 8.53 (dd, J=4.40, 7.04 Hz, 1H), 8.15-8.25 (m, 2H), 7.92 (d, J=1.10 Hz, 1H), 7.83 (s, 1H), 7.79 (s, 1H), 7.65 (d, J=1.32 Hz, 1H), 7.33 (t, J=8.80 Hz, 2H), 7.25 (d, J=1.10 Hz, 1H), 6.57 (s, 1H), 5.32 (s, 1H), 4.45 (dd, J=7.26, 13.64 Hz, 1H), 3.98 (s, 3H), 3.93 (dd, J=4.29, 13.75 Hz, 1H), 2.83 (s, 3H), 1.66 (t, J=19.48 Hz, 3H), 1.43 (s, 6H)
LC-MS(RT: 1.75, MW = 555 [M+H]⁺, METHOD O)
OR = +45.83 ° (589 nm, c 0.24 w/v %, DMF, 20° C.)

### (-)Methoxy-3-(Methyl-d3)-N-(3,3,3-Trifluoro-2-(5-Fluoro-6-(4-Fluorophenyl)-4-(2-Hydroxypropan-2-yl)Pyridin-2-yl)-2-Hydroxypropyl)Cinnoline-6-Carboxamide 311

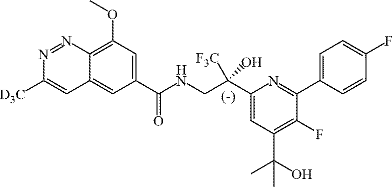

¹H NMR (400 MHz, DMSO-d6) δ 8.79 (t, J=5.94 Hz, 1H), 8.12 (d, J=5.28 Hz, 1H), 7.99 (dd, J=5.72, 7.48 Hz, 2H), 7.93 (s, 1H), 7.78 (d, J=1.54 Hz, 1H), 7.39 (d, J=1.32 Hz, 1H), 7.32-7.38 (m, 2H), 7.29 (s, 1H), 5.67 (s, 1H), 4.11-4.30 (m, 2H), 4.06 (s, 3H), 1.55 (s, 3H), 1.48 (s, 3H)
LC-MS (RT: 1.92, MW = 579 [M+H]⁺, METHOD O)
OR = -90.21 ° (589 nm, c 0.235 w/v %, DMF, 20° C.)

### (-)(Difluoromethoxy)-3-Methyl-N-(3.3.3-Trifluoro-2-(5-Fluoro-6-(4-Fluorophenyl)-4-(2-Hydroxypropan-2-yl)Pyridin-2-yl)-2-Hydroxypropyl)Cinnoline-6-Carboxamide 312

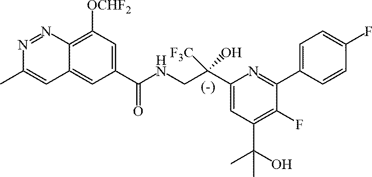

¹H NMR (400 MHz, DMSO-d6) δ 8.83 (t, J=6.05 Hz, 1H), 8.18 (d, J=1.32 Hz, 1H), 8.11 (d, J=5.28 Hz, 1H), 8.09 (s, 1H), 7.98 (dd, J=5.72, 7.70 Hz, 2H), 7.77 (s, 1H), 7.60 (br t, J=73.62 Hz, 1H), 7.30-7.38 (m, 2H), 7.23 (s, 1H), 5.66 (s, 1H), 4.12-4.31 (m, 2H), 2.92 (s, 3H), 1.55 (s, 3H), 1.49 (s, 3H)

LC-MS (RT: 1.13, MW = 613 [M+H]⁺, METHOD L)

### (-)-N-(3,3-difluoro-2-(5-fluoro-6-(4-fluorophenyl)-4-(2-hydroxypropan-2-yl)pyridin-2-yl)-2-hydroxybutyl)-8-methoxy-3-methylcinnoline-6-carboxamide 313

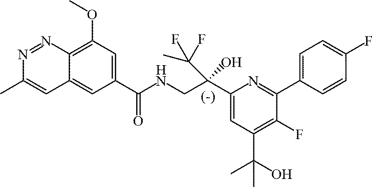

¹H NMR (400 MHz, DMSO-d6) δ 8.56 (t, J=5.80 Hz, 1H), 8.07 (d, J=5.28 Hz, 1H), 7.99 (dd, J=5.61, 7.59 Hz, 2H), 7.89 (s, 1H), 7.71 (d, J=1.54 Hz, 1H), 7.29-7.38 (m, 3H), 6.55 (s, 1H), 5.61 (s, 1H), 4.03 (s, 3H), 3.96-4.38 (m, 2H), 2.85 (s, 3H), 1.67 (t, J=19.59 Hz, 3H), 1.53 (s, 3H), 1.49 (s, 3H)
LC-MS (RT: 1.01, MW = 573 [M+H]⁺, METHOD L)
OR = -78.85 ° (589 nm, c 0.208 w/v %, DMF, 20° C.)

### (+)-N-(3,3-difluoro-2-(5-fluoro-6-(4-fluorophenyl)-4-(2-hydroxypropan-2-yl)pyridin-2-yl)-2-hydroxybutyl)-8-methoxy-3-methylcinnoline-6-carboxamide 314

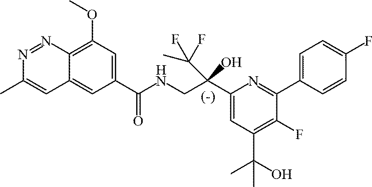

1H NMR (400 MHz, DMSO-d6 ) δ 8.56 (t, J=5.80 Hz, 1H), 8.07 (d, J=5.50 Hz, 1H), 7.94-8.02 (m, 2H), 7.89 (s, 1H), 7.71 (d, J=1.32 Hz, 1H), 7.27-7.40 (m, 3H), 6.55 (s, 1H), 5.61 (s, 1H), 4.03 (s, 3H), 3.95-4.38 (m, 2H), 2.85 (s, 3H), 1.61-1.75 (m, 3H), 1.53 (s, 3H), 1.49 (s, 3H)
LC-MS (RT: 1.01, MW = 573 [M+H]⁺, METHOD L)
OR: +92.75 ° (589 nm, c 0.207 w/v %)

### (+)-N-cyclopropyl-2-(5-fluoro-6-(4-fluorophenyl)-4-hydroxypropan-2-yl)pyridin-2-yl)-2-hydroxyethyl-1,1-d2)-8-methoxy-3-methylcinnoline-6-carboxamide 315

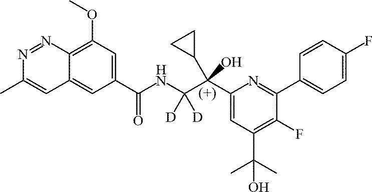

1H NMR (400 MHz, DMSO-d6) δ ppm 0.12 - 0.20 (m, 1 H), 0.27 - 0.34 (m, 1 H), 0.40 (br dd, J=8.8, 5.3 Hz, 1 H), 0.55 - 0.62 (m, 1 H), 1.47 (s, 3 H), 1.54 (s, 3 H), 1.57 (s, 1 H), 2.86 (s, 3 H), 4.06 (s, 3 H), 5.43 (s, 1 H), 5.56 (s, 1 H), 7.28 - 7.35 (m, 2 H), 7.39 (d, J=1.3 Hz, 1 H), 7.76 (d, J=1.5 Hz, 1 H), 7.90 (s, 1 H), 7.92 - 8.01 (m, 3 H), 8.63 (s, 1 H)
LC-MS (RT: 1.93, MW = 551 [M+H]⁺, METHOD S)
OR = +29.52 ° (589 nm, c 0.2405 w/v %, DMF, 20° C.)

### (-)-N-cyclopropyl-2-(5-fluoro-6-(4-fluorophenyl)-4-hydroxypropan-2-yl)pyridin-2-yl)-2-hydroxyethyl-1,1-d2)-8-methoxy-3-methylcinnoline-6-carboxamide 316

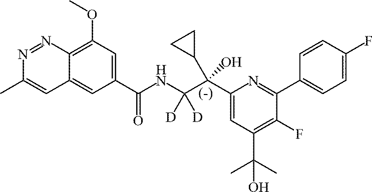

1H NMR (400 MHz, DMSO-d6) 6 ppm 0.12 - 0.21 (m, 1 H), 0.27 - 0.45 (m, 2 H), 0.54 - 0.62 (m, 1 H), 1.47 (s, 3 H), 1.54 (s, 3 H), 1.55 - 1.61 (m, 1 H), 2.86 (s, 3 H), 4.06 (s, 3 H), 5.43 (s, 1 H), 5.56 (s, 1 H), 7.28 - 7.35 (m, 2 H), 7.40 (d, J=1.3 Hz, 1 H), 7.76 (d, J=1.3 Hz, 1 H), 7.90 (s, 1 H), 7.92 - 8.01 (m, 3 H), 8.63 (s, 1 H)
LC-MS (RT: 1.93, MW = 551 [M+H]⁺, METHOD S)
OR = -30.95 ° (589 nm, c 0.252 w/v %, DMF, 20° C.)

### (-)Cyclopropoxy-N-(2-(5-Fluoro-6-(4-Fluorophenyl)-4-(2-Hydroxypropan-2-yl)Pyridin-2-yl)-2-(1-Fluorocyclopropyl)-2-Hydroxyethyl)-3-Methylcinnoline-6-Carboxamide 317

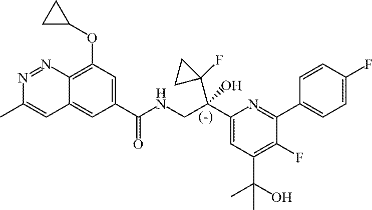

1H NMR (400 MHz, DMSO-d6) δ ppm 0.75 - 1.15 (m, 10 H) 1.54 (d, J=15.6 Hz, 6 H) 2.86 (s, 3 H) 3.99 - 4.39 (m, 2 H) 4.11 -4.12(m, 1 H) 5.61 (s, 1 H) 6.17 (s, 1 H) 7.24 - 7.41 (m, 2 H) 7.71 (d, J=1.5 Hz, 1 H) 7.80 (d, J=1.5 Hz, 1 H) 7.91 (s, 1 H) 7.94 - 8.10 (m, 3 H) 8.61 (t, J=5.7 Hz, 1 H)
LC-MS(RT: 2.04, MW = 593 [M+H]⁺, METHOD P)
OR = -32.59 ° (589 nm, c 0.2076 w/v %, DMF, 20° C.)

### (-)Cyclopropoxy-3-Methyl-N-(3.3.3-Trifluoro-2-(5-Fluoro-6-(4-Fluorophenyl)-4-(2-Hydroxypropan-2-yl)Pyridin-2-yl)-2-Hydroxypropyl)Cinnoline-6-Carboxamide 318

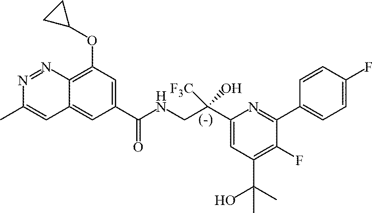

1H NMR (400 MHz, DMSO-d6) δ ppm 0.80 - 0.94 (m, 4 H) 1.52 (d, J=28.2 Hz, 5 H) 1.45 - 1.46 (m, 1 H) 2.86 (s, 3 H) 4.07 - 4.33 (m, 2 H) 4.12 - 4.13 (m, 1 H) 5.67 (s, 1 H) 7.29 (s, 1 H) 7.31 -7.41 (m, 2 H) 7.70 (d, J=1.5 Hz, 1 H) 7.79 (d, J=1.5 Hz, 1 H) 7.91 (s, 1 H) 8.00 (dd, J=7.6, 5.6 Hz, 2 H) 8.12 (d, J=5.5 Hz, 1 H) 8.16 - 8.19 (m, 1 H) 8.75 (t, J=5.9 Hz, 1 H)
LC-MS(RT: 2.07, MW = 603 [M+H]⁺, METHOD P)
OR = -89.42 ° (589 nm, c 0.3277 w/v %, DMF, 20° C.)

### N-((*R)-2,2-Difluorocyclopropyl)-2-(5-Fluoro-6-(4-Fluorophenyl)-4-Hydroxypropan-2-yl)Pyridin-2-yl)-2-Hydroxyethyl)-8-Methoxy-3-Methylcinnoline-6-Carboxamide 319

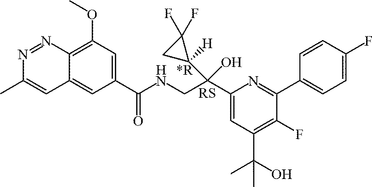

1H NMR (400 MHz, DMSO-d6) δ ppm 1.50 (d, J=31.7 Hz, 7 H) 2.58 - 2.61 (m, 1 H) 2.87 (s, 3 H) 3.84 - 4.04 (m, 2 H) 4.08 (s, 3 H) 5.59 (s, 1 H) 5.85 (s, 1 H) 7.29 - 7.40 (m, 2 H) 7.44 (d, J=1.5 Hz, 1 H) 7.81 (d, J=1.3 Hz, 1 H) 7.88 - 8.11 (m, 4 H) 8.87 (t, J=6.1 Hz, 2 H) 8.91 - 8.94 (m, 1 H)

LC-MS(RT: 2.02, MW = 585 [M+H]⁺, METHOD P)

### N-((*S)-2,2-Difluorocyclopropyl)-2-(5-Fluoro-6-(4-Fluorophenyl)-4-Hydroxypropan-2-yl)Pyridin-2-yl)-2-Hydroxyethyl)-8-MZthoxy-3-Methylcinnoline-6-Carboxamide 320

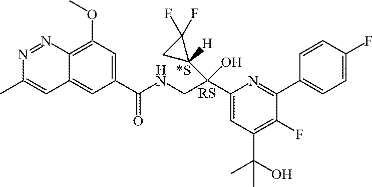

1H NMR (400 MHz, DMSO-d6) 6 ppm 1.45 - 1.83 (m, 1 H) 1.48 - 1.58 (m, 6 H) 1.70 - 1.82 (m, 1 H) 2.53 - 2.64 (m, 1 H) 2.87 (s, 3 H) 3.74 - 3.93 (m, 2 H) 3.76 - 3.82 (m, 1 H) 4.06 (s, 3 H) 5.55 - 5.64 (m, 1 H) 5.60 (s, 1 H) 5.81 - 5.94 (m, 1 H) 5.88 (s, 1 H) 7.27 - 7.36 (m, 1 H) 7.37 -7.42 (m, 1 H) 7.74 - 7.80 (m, 1 H) 7.83 - 7.87 (m, 1 H) 7.92 - 8.02 (m, 1 H) 7.98 (s, 1 H) 8.77 (t, J=6.1 Hz, 1 H)

LC-MS (RT: 1.93, MW = 585 [M+H]⁺, METHOD P)

### (-)Cyclopropyl-8-Methoxy-N-(3,3,3-Trifluoro-2-(5-Fluoro-6-(4-Fluorophenyl)-4-(2-Hydroxypropan-2-yl)Pyridin-2-yl)-2-Hydroxypropyl)Quinoline-6-Carboxamide 321

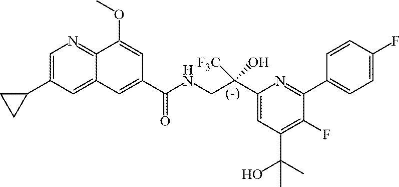

1H NMR (400 MHz, DMSO-d6) δ ppm 0.83 - 0.89 (m, 2 H) 1.06 - 1.13 (m, 2 H) 1.46 - 1.58 (m, 6 H) 2.08 - 2.21 (m, 1 H) 2.08 - 2.21 (m, 1 H) 3.95 (s, 3 H) 4.09 - 4.33 (m, 2 H) 5.67 (s, 1 H) 7.30 - 7.41 (m, 4 H) 7.78 (d, J=1.54 Hz, 1 H) 7.88 (d, J=2.20 Hz, 1 H) 8.00 (dd, J=7.59, 5.61 Hz, 2 H) 8.13 (d, J=5.50 Hz, 1 H) 8.67 (t, J=5.83 Hz, 1 H) 8.75 (d, J=2.20 Hz, 1 H)
LC-MS (RT: 1.16, MW = 602 [M+H]⁺, METHOD L)
OR = -221 ° (589 nm, c 0.281 w/v %, DMF, 20° C.)

### (-)Methoxy-3-Methyl-N-(3,3,3-Trifluoro-2-(5-Fluoro-6-(4-Fluorophenyl)-4-(2-Hydroxypropan-2-yl)Pyridm-2-yl)-2-Hydroxypropyl)Qumolme-6-Carboxamide 322

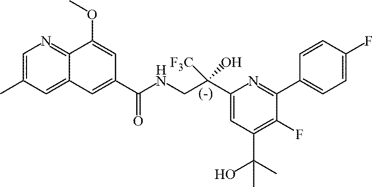

1H NMR (400 MHz, DMSO-d6) δ 8.77 (d, J=1.98 Hz, 1H), 8.72 (br t, J=5.83 Hz, 1H), 8.13 (d, J=5.50 Hz, 1H), 8.04 (s, 1H), 7.94-8.03 (m, 2H), 7.80 (s, 1H), 7.42 (s, 1H), 7.27-7.40 (m, 3H), 5.66 (s, 1H), 4.10-4.36 (m, 2H), 3.95 (s, 3H), 2.48 (s, 3H), 1.55 (s, 3H), 1.49 (s, 3H)
LCMS (RT: 1.11, MW = 576 [M+H]⁺, METHOD L)
OR = -105.71 ° (589 nm, c 0.175 w/v %, DMF, 20° C.)

### (-)Cyclopropoxy-3-Methyl-N-(3,3,3-Trifluoro-2-(5-Fluoro-6-(4-Fluorophenyl)-4-(2-Hydroxypropan-2-yl)Pyridin-2-yl)-2-Hydroxypropyl)Quinoline-6-Carboxamide 323

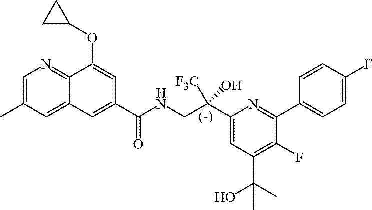

1H NMR (400 MHz, DMSO-d6) δ 8.75 (s, 1H), 8.68 (t, J=6.01 Hz, 1H), 8.14 (d, J=5.28 Hz, 1H), 7.98-8.05 (m, 3H), 7.81 (d, J=1.54 Hz, 1H), 7.67 (d, J=1.54 Hz, 1H), 7.40 (s, 1H), 7.32-7.39 (m, 2H), 5.67 (s, 1H), 4.11-4.31 (m, 2H), 4.01 (tt, J=2.92, 6.00 Hz, 1H), 2.46-2.49 (m, 3H), 1.56 (s, 3H), 1.49 (s, 3H), 0.81-0.89 (m, 2H), 0.72-0.81 (m, 2H)
LCMS (RT: 2.16, MW = 602 [M+H]⁺, METHOD Q)
OR = -167 ° (589 nm, c 0.3 w/v %, DMF, 20° C.)

### (-)(Fluoromethyl)-8-Methoxy-N-(3,3,3-Trifluoro-2-(5-Fluoro-6-(4-Fluorophenyl)-4-(2-Hydroxypropan-2-yl)Pyridin-2-yl)-2-Hydroxypropyl)Quinoline-6-Carboxamide 324

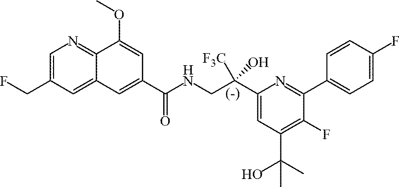

1H NMR (400 MHz, DMSO-d6) δ ppm 1.41 - 1.60 (m, 6 H) 3.98 (s, 3 H) 4.12 - 4.31 (m, 2 H) 5.68 (d, J=47.32 Hz, 2 H) 5.66 (s, 1 H) 7.30 - 7.41 (m, 3 H) 7.46 (d, J=1.32 Hz, 1 H) 7.93 (d, J=1.54 Hz, 1 H) 8.00 (dd, J=7.70, 5.50 Hz, 2 H) 8.13 (d, J=5.28 Hz, 1 H) 8.34 - 8.40 (m, 1 H) 8.74 (t, J=5.72 Hz, 1 H) 8.92 - 8.97 (m, 1 H)
LCMS (RT: 1.09, MW = 594 [M+H]⁺, METHOD L)
OR = -195.56 ° (589 nm, c 0.1125 w/v %, DMF, 20° C.)

### (-)(Difluoromethyl)-8-Methoxy-N-(3,3,3-Trifluoro-2-(5-Fluoro-6-(4-Fluorophenyl)-4-(2-Hydroxypropan-2-yl)Pyridin-2-yl)-2-Hydroxypropyl)Quinoline-6-Carboxamide 325

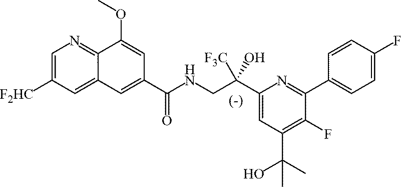

1H NMR (400 MHz, DMSO-d6) δ ppm 1.43 - 1.60 (m, 6 H) 4.00 (s, 3 H) 4.10 - 4.33 (m, 2 H) 5.66 (s, 1H) 7.18 - 7.48 (m, 1 H) 7.31 - 7.37 (m, 4 H) 7.52 (d, J=1.32 Hz, 1 H) 7.96 - 8.03 (m, 3 H) 8.13 (d, J=5.50 Hz, 1 H) 8.58 (d, J=1.76 Hz, 1 H) 8.75 (t, J=5.94 Hz, 1 H) 9.06 (d, J=2.20 Hz, 1 H)
LC-MS (RT: 1.13, MW = 612 [M+H]⁺, METHOD L)
OR = -203.66 ° (589 nm, c 0.273 w/v %, DMF, 20° C.)

### (-)(Trifluoromethyl)-8-Methoxy-N-(3,3,3-Trifluoro-2-(5-Fluoro-6-(4-Fluorophenyl)-4-(2-Hydroxypropan-2-yl)Pyridin-2-yl)-2-Hydroxypropyl)Quinoline-6-Carboxamide 326

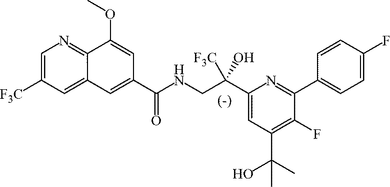

1H NMR (400 MHz, DMSO-d6) δ ppm 1.45 - 1.56 (m, 6 H) 4.01 (s, 3 H) 4.10 - 4.35 (m, 2 H) 5.67 (s, 1 H) 7.27 - 7.38 (m, 3 H) 7.58 (d, J=1.54 Hz, 1 H) 7.99 (dd, J=7.59, 5.61 Hz, 2 H) 8.07 (d, J=1.32 Hz, 1 H) 8.13 (d, J=5.28 Hz, 1 H) 8.76 (t, J=5.83 Hz, 1 H) 8.87 (d, J=1.32 Hz, 1 H) 9.19 (d, J=2.20 Hz, 1 H)
LC-MS (RT: 1.20, MW = 630 [M+H]⁺, METHOD L)
OR = -186.3 ° (589 nm, c 0.2555 w/v %, DMF, 20° C.)

### (-)(Difluoromethyl)-8-Methoxy-N-(3,3,3-Trifluoro-2-(5-Chloro-6-(4-Fluorophenyl)-4-(2-Hydroxypropan-2-yl)Pyridin-2-yl)-2-Hydroxypropyl)Quinoline-6-Carboxamide 327

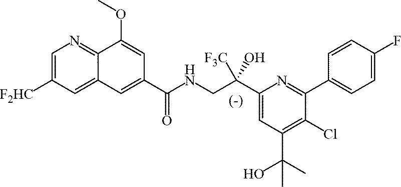

¹H NMR (400 MHz, DMSO-d6) δ 9.07 (d, J=1.98 Hz, 1H), 8.76 (t, J=5.83 Hz, 1H), 8.60 (d, J=1.54 Hz, 1H), 8.34 (s, 1H), 8.00 (d, J=1.54 Hz, 1H), 7.65-7.74 (m, 2H), 7.52 (d, J=1.32 Hz, 1H), 7.35 (d, J=9.68 Hz, 1H), 7.25-7.33 (m, 2H), 7.20 (t, J=1.00 Hz, 1H), 5.69 (s, 1H), 4.09-4.30 (m, 2H), 4.01 (s, 3H), 1.65 (s, 3H), 1.57 (s, 3H)
LC-MS (RT: 1.14, MW = 628 [M+H]⁺, METHOD L)
OR = -62.15 ° (589 nm, c 0.2655 w/v %, DMF, 20° C.)

### (-)(Trifluoromethyl)-8-Methoxy-N-(3,3,3-Trifluoro-2-(5-Chloro-6-(4-Fluorophenyl)-4-(2-Hydroxypropan-2-yl)Pyridin-2-yl)-2-Hydroxypropyl)Quinoline-6-Carboxamide 328

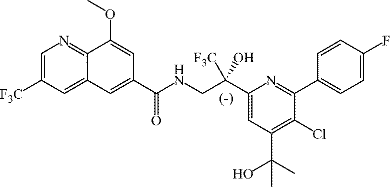

¹H NMR (400 MHz, DMSO-d6) δ 9.20 (d, J=2.20 Hz, 1H), 8.89 (d, J=1.32 Hz, 1H), 8.77 (t, J=5.94 Hz, 1H), 8.34 (s, 1H), 8.06 (d, J=1.54 Hz, 1H), 7.66-7.74 (m, 2H), 7.58 (d, J=1.32 Hz, 1H), 7.36 (s, 1H), 7.20-7.32 (m, 2H), 5.69 (s, 1H), 4.09-4.29 (m, 2H), 4.03 (s, 3H), 1.65 (s, 3H), 1.58 (s, 3H)
LC-MS (RT: 1.21, MW = 646 [M+H]⁺, METHOD L)
OR = -46.65 ° (589 nm, c 0.2615 w/v %, DMF, 20° C.)

### (-)(Trifluoromethyl)-8-Methoxy-N-(Cyclopropyl-2-(5-Fluoro-6-(4-Fluorophenyl)-4-(2-Hydroxypropan-2-yl)Pyridin-2-yl)-2-Hydroxypropyl)Quinoline-6-Carboxamide 329

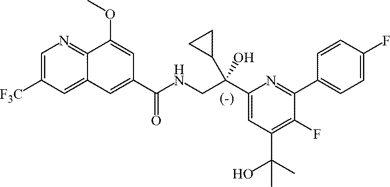

¹H NMR (400 MHz, DMSO-d6) δ 9.18 (d, J=2.20 Hz, 1H), 8.84 (d, J=1.32 Hz, 1H), 8.61 (t, J=5.83 Hz, 1H), 8.06 (d, J=1.54 Hz, 1H), 7.93-8.00 (m, 3H), 7.58 (d, J=1.54 Hz, 1H), 7.23-7.35 (m, 2H), 5.57 (s, 1H), 5.48 (s, 1H), 4.01 (s, 3H), 3.84-3.99 (m, 2H), 1.55-1.63 (m, 1H), 1.54 (s, 3H), 1.48 (s, 3H), 0.53-0.67 (m, 1H), 0.37-0.47 (m, 1H), 0.26-0.36 (m, 1H), 0.11-0.23 (m, 1H)
LC-MS (RT: 2.50, MW = 602 [M+H]⁺, METHOD U)
OR = -29.41 ° (589 nm, c 0.255 w/v %, DMF, 20° C.)

### (-)(Difluoromethyl)-8-Methoxy-N-(3,3,3-Trifluoro-2-(5-Fluoro-6-(3,4-Difluorophenyl)-4-(2-Hydroxypropan-2-yl)Pyridin-2-yl)-2-Hydroxypropyl)Quinoline-6-Carboxamide 330

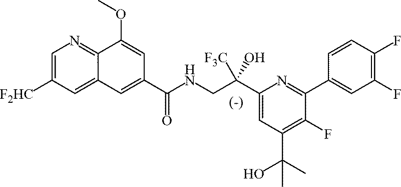

1H NMR (400 MHz, DMSO-d6) δ ppm 1.48 (s, 3 H) 1.56 (s, 3 H) 4.00 (s, 3 H) 4.23 (ddd, J=75.76, 13.81, 5.94 Hz, 2 H) 5.69 (s, 1 H) 7.34 (t, J=55.13 Hz, 1 H) 7.38 (s, 1 H) 7.50 - 7.63 (m, 2 H) 7.77 - 7.85 (m, 1 H) 7.93 - 8.04 (m, 2 H) 8.16 (d, J=5.28 Hz, 1 H) 8.59 (d, J=1.54 Hz, 1H) 8.74 (t, J=5.94 Hz, 1 H) 9.06 (d, J=1.98 Hz, 1 H)
LC-MS (RT: 1.18, MW = 630 [M+H]⁺, METHOD L)
OR = -89.87 ° (589 nm, c 0.2615 w/v %, DMF, 20° C.)

### (S)-N-(5-Chloro-6-(4-Fluorophenyl)-4-Hydroxypropan-2-yl)Pyridin-2-yl)-2-Cyclopropyl-2-Hydroxyethyl)-8-Methoxy-3-(Trifluoromethyl)Quinoline-6-Carboxamide 331

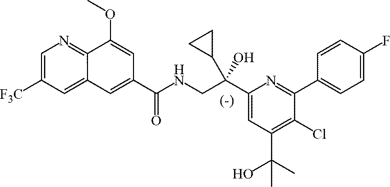

¹H NMR (400 MHz, DMSO-d6) δ ppm 0.11 - 0.21 (m, 1 H) 0.27 - 0.34 (m, 1 H) 0.35 - 0.45 (m, 1 H) 0.54 - 0.65 (m, 1 H) 1.47 - 1.54 (m, 1 H) 1.56 (s, 3 H) 1.64 (s, 3 H) 3.83 - 3.98 (m, 2 H) 4.02 (s, 3 H) 5.48 (s, 1 H) 5.58 (s, 1 H) 7.23 (m, J=8.90, 8.90 Hz, 2 H) 7.58 (d, J=1.54 Hz, 1 H) 7.68 (m, J=8.80, 5.70 Hz, 2 H) 8.04 (d, J=1.54 Hz, 1 H) 8.16 (s, 1 H) 8.64 (t, J=1.00 Hz, 1 H) 8.86 (d, J=1.32 Hz, 1 H) 9.19 (d, J=2.20 Hz, 1 H)

LC-MS (RT: 2.51, MW = 618 [M+H]⁺, METHOD U)

### (-)Cyclopropyl-N-(2-Cyclopropyl-2-(5-Fluoro-6-(4-Fluorophenyl)-4-(2-Hydroxypropan-2-yl)Pyridin-2-yl)-2-Hydroxyethyl)-8-Methoxyquinoline-6-Carboxamide 332

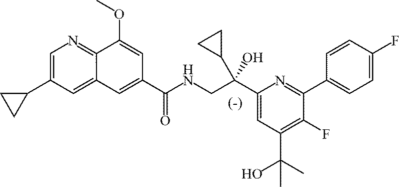

¹H NMR (400 MHz, DMSO-d6) δ 8.74 (d, J=2.42 Hz, 1H), 8.51 (t, J=5.83 Hz, 1H), 7.98 (dd, J=5.50, 7.48 Hz, 2H), 7.94 (d, J=5.72 Hz, 1H), 7.86 (d, J=2.20 Hz, 1H), 7.76 (d, J=1.54 Hz, 1H), 7.26-7.36 (m, 3H), 5.56 (d, J=2.20 Hz, 2H), 3.94 (s, 3H), 3.84-4.01 (m, 2H), 2.10-2.20 (m, 1H), 1.54 (s, 3H), 1.51-1.62 (m, 1H), 1.47 (s, 3H), 1.06-1.14 (m, 2H), 0.82-0.91 (m, 2H), 0.53-0.62 (m, 1H), 0.35-0.45 (m, 1H), 0.26-0.34 (m, 1H), 0.15 (ddt, J=3.74, 5.56, 8.78 Hz, 1H)
LCMS : (RT: 1.13, MW = 574 [M+H]⁺, METHOD L)
OR = -35.32 ° (589 nm, c 0.252 w/v %, DMF, 20° C.)

### (-)Methoxy-3-Methyl-N-(3.3.3-Trifluoro-2-(5-Fluoro-4-(2-Hydroxypropan-2-yl)-6-Phenylpyridin-2-yl)-2-Hydroxypropyl)Cinnoline-6-Carboxamide 333

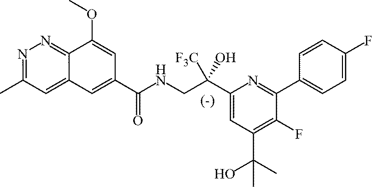

1HNMR (400 MHz, DMSO-d6) δ 8.81 (t, J=5.94 Hz, 1H), 8.13 (d, J=5.50 Hz, 1H), 7.89-7.97 (m, 3H), 7.79 (d, J=1.54 Hz, 1H), 7.45-7.56 (m, 3H), 7.40 (d, J=1.32 Hz, 1H), 7.31 (s, 1H), 5.67 (s, 1H), 4.13-4.32 (m, 2H), 4.06 (s, 3H), 2.87 (s, 3H), 1.56 (s, 3H), 1.50 (s, 3H).

LC-MS (RT: 1.00, MW = 558 [M+H]⁺, METHOD L)

### (+)Methoxy-3-Methyl-N-(3,3,3-Trifluoro-2-(5-Fluoro-6-(4-Fluorophenyl)-4-(2-Hydroxypropan-2-yl)Pyridin-2-yl)-2-Hydroxypropyl)Cinnoline-6-Carboxamide 334

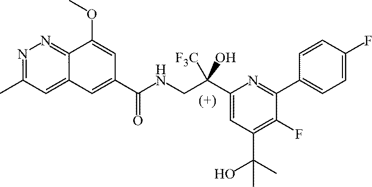

1HNMR (400 MHz, DMSO-d6) δ ppm 1.52 (d, J=27.5 Hz, 6 H) 2.87 (s, 3 H) 4.07 (s, 3 H) 4.11 - 4.21 (m, 1 H) 4.25 (br d, J=6.2 Hz, 1 H) 5.67 (s, 1 H) 7.29 (s, 1 H) 7.35 (t, J=8.8 Hz, 2 H) 7.39 (d, J=1.1 Hz, 1 H) 7.50 - 7.51 (m, 1 H) 7.78 (d, J=1.1 Hz, 1 H) 7.93 (s, 1 H) 8.00 (br dd, J=7.5, 5.7 Hz, 2 H) 8.13 (d, J=5.3 Hz, 1 H) 8.35 - 8.38 (m, 1 H) 8.79 (br t, J=5.8 Hz, 1 H)
LC-MS (RT: 1.95, MW = 577 [M+H]⁺, METHOD T)
OR = +100.46° (589 nm, c 0.218 w/v %, DMF, 20° C.)

### (-)Cyclopropoxy-N-(2-(5-Fluoro-6-(4-Fluorophenyl)-4-(2-Hydroxypropan-2-yl)Pyridin-2-yl)-2-(1-Fluorocyclopropyl)-2-Hydroxyethyl)-3-Methylcinnoline-6-Carboxamide 335

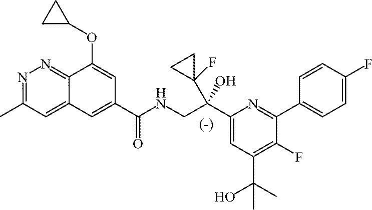

1HNMR (400 MHz, DMSO-d6) δ ppm 0.74 - 1.17 (m, 9 H) 1.54 (d, J=15.6 Hz, 6 H) 2.86 (s, 3 H) 4.05 - 4.18 (m, 2 H) 4.26 (dd, J=13.4, 5.9 Hz, 1 H) 5.61 (s, 1 H) 6.17 (s, 1 H) 7.33 (t, J=8.9 Hz, 2 H) 7.71 (d, J=1.3 Hz, 1 H) 7.80 (d, J=1.3 Hz, 1 H) 7.91 (s, 1 H) 7.95 - 8.08 (m, 2 H) 8.02 -8.05 (m, 1 H) 8.61 (t, J=5.8 Hz, 1 H)
LC-MS (RT: 2.00, MW = 593 [M+H]⁺, METHOD R)
OR = -33.25° (589 nm, c 0.412 w/v %, DMF, 20° C.)

### X-Ray Crystallography

Absolute configuration of compound 95 has been confirmed to be S by use of X-Ray crystallography.

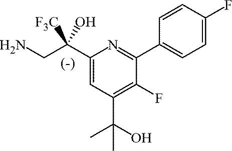

The single crystal was obtained by cooling in DMF followed by prolonged incubation at 5° C.

| | |
|---|---|
| Crystal system | Monoclinic |
| Space group | P2₁ |
| Unit cell dimensions | |
| a = 6.51860(10) Å | α= 90° |
| b = 8.9086(2) Å | β= 97.734(2)° |
| c = 15.0074(2) Å | γ= 90° |
| Volume = 863.58(3) Å³ | |
| Rfac = 2.57% | |

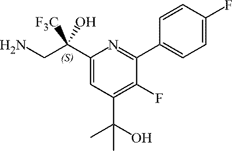 [SMILES: CC(C)(O)c1cc([C@@](O)(CN)C(F)(F)F)nc(-c2ccc(F)cc2)c1F]

For the structure 95 as presented, with the stereocentre in the S configuration at C2 The Flack parameter = -0.05(6), (Acta Cryst. B69, 2013, 249-259).

Determination of the absolute structure using Bayesian statistics on Bijvoet differences, reveals that the probability of the absolute structure as presented being correct is 1.000, while the probabilities of the absolute structure being a racemic twin or false are both 0.000. The Flack equivalent and its uncertainty are calculated through this program to be = -0.05(5). The calculation was based on 1364 Bijvoet pairs with a coverage of 96% (Hooft et al., J. Appl. Cryst., 2008, 41, 96-103).

### 5. Biological Assays

### 5.1. Antiviral Activity

Black 384-well clear-bottom microtiter plates (Corning, Amsterdam, The Netherlands) were filled via acoustic drop ejection using the echo liquid handler (Labcyte, Sunnyvale, California). 200 nL of compound stock solutions (100% DMSO) were transferred to the assay plates. 9 serial 4-fold dilutions of compound were made, creating per quadrant the same compound concentration. The assay was initiated by adding 10 µL of culture medium to each well (RPMI medium without phenol red, 10% FBS-heat inactivated, 0.04% gentamycin (50 mg/mL). All addition steps are done by using a multidrop dispenser (Thermo Scientific, Erembodegem, Belgium). Next, rgRSV224 virus (MOI = 1) diluted in culture medium was added to the plates. rgRSV224 virus is an engineered virus that includes an additional GFP gene (Hallak LK, Spillmann D, Collins PL, Peeples ME. Glycosaminoglycan sulfation requirements for respiratory syncytial virus infection; Journal of virology (2000), 74(22), 10508-13) and was in-licensed from the NIH (Bethesda, MD, USA). Finally, 20 µL of a HeLa cell suspension (3,000 cells/well) were plated. Medium, virus- and mock-infected controls were included in each test. The wells contain 0.05% DMSO per volume. Cells were incubated at 37° C. in a 5% CO₂ atmosphere. Three days post-virus exposure, viral replication was quantified by measuring GFP expression in the cells by an in house developed MSM laser microscope (Tibotec, Beerse, Belgium). The EC₅₀ was defined as the 50% inhibitory concentration for GFP expression. In parallel, compounds were incubated for three days in a set of white 384-well microtiter plates (Corning) and the cytotoxicity of compounds in HeLa cells was determined by measuring the ATP content of the cells using the ATPlite kit (Perkin Elmer, Zaventem, Belgium) according to the manufacturer’s instructions. The CC₅₀ was defined as the 50% concentration for cytotoxicity.

### 5.2. Table of Biological Activity

Table : antiviral data (averaged data of several repeat experiments)

| Co. No. | RSV HELA EC₅₀ (nM) | TOX HELA CC₅₀ (µM) | Co. No. | RSV HELA EC₅₀ (nM) | TOX HELA CC₅₀ (µM) |
|---|---|---|---|---|---|
| 200 | 1.7 | 22 | 237 | 5.1 | 51 |
| 201 | 2.6 | 43 | 238 | 2.9 | 23 |
| 202 | 4.8 | 46 | 239 | 0.5 | 19 |
| 203 | 480 | 43 | 240 | 1.2 | 46 |
| 204 | 1 | 48 | 241 | 1.5 | 50 |
| 205 | 0.81 | 32 | 242 | 170 | 48 |
| 206 | 0.46 | 15 | 243 | 3 | 45 |
| 207 | 0.77 | 13 | 244 | 58 | 43 |
| 208 | 1 | 14 | 245 | 2.9 | 48 |
| 209 | 0.41 | 25 | 246 | 200 | 46 |
| 210 | 2.9 | 23 | 247 | 1.9 | 51 |
| 211 | 2.8 | 29 | 248 | 500 | 41 |
| 212 | 7.1 | 58 | 249 | >1250 | 35 |
| 213 | 520 | 31 | 250 | 1.4 | 40 |
| 214 | 1.4 | 28 | 251 | 0.53 | 28 |
| 215 | 0.87 | 20 | 252 | 0.3 | 43 |
| 216 | 0.62 | 21 | 253 | 0.25 | 13 |
| 217 | 1.3 | 14 | 254 | 3.5 | 15 |
| 218 | 1.2 | 16 | 255 | 0.81 | 44 |
| 219 | 5 | 27 | 256 | 0.19 | 33 |
| 220 | 2.4 | 36 | 257 | 0.71 | 55 |
| 221 | 9.3 | 65 | 258 | 510 | 52 |
| 222 | 1.5 | 27 | 259 | 2.4 | 18 |
| 223 | 0.63 | 21 | 260 | 1.6 | 19 |
| 224 | 1.7 | 15 | 261 | 13 | 13 |
| 225 | 0.59 | 15 | 262 | 1.9 | 45 |
| 226 | 2.4 | 14 | 263 | 2.7 | 47 |
| 227 | 1.1 | 15 | 264 | 0.87 | 32 |
| 228 | 13 | 10 | 265 | 0.69 | 39 |
| 229 | 0.81 | 44 | 266 | 3.5 | 25 |
| 230 | 120 | 57 | 267 | 3 | 14 |
| 231 | 0.41 | 21 | 268 | 0.67 | 18 |
| 232 | 1.6 | 24 | 269 | 1.2 | 20 |
| 233 | 400 | 43 | 270 | 0.84 | 15 |
| 234 | 0.82 | 23 | 271 | 1.5 | 11 |
| 235 | 0.62 | 25 | 272 | 3.7 | 17 |
| 273 | 3.81 | 13 | 304 | 2.1 | 23 |
| 274 | 1.48 | 11 | 305 | 642 | 41 |
| 275 | 97 | 17 | 306 | 1.0 | 50 |
| 276 | 3.8 | 51 | 307 | 40 | 43 |
| 277 | 16 | 15 | 308 | 2.3 | 38 |
| 278 | 0.65 | 29 | 309 | 11 | 34 |
| 279 | 47 | 16 | 310 | >1250 | 55 |
| 280 | 449 | 9.4 | 311 | 1.3 | 42 |
| 281 | 29 | 24 | 312 | 2.8 | 13 |
| 282 | 3.2 | 13 | 313 | 1.0 | 42 |
| 283 | 8.5 | 12 | 314 | 168 | 35 |
| 284 | 4.8 | 16 | 315 | 47 | 27 |
| 285 | 6.5 | 16 | 316 | 0.34 | 33 |
| 286 | 2.6 | 8.9 | 317 | 0.87 | 29 |
| 287 | 8.9 | 37 | 318 | 1.4 | 22 |
| 288 | 4.8 | 21 | 319 | 1.2 | 6.1 |
| 289 | 1.8 | 10 | 320 | 1.0 | 24 |
| 290 | 0.21 | 28 | 321 | 54 | 13 |
| 291 | 15 | 57 | 322 | 4.5 | 15 |
| 292 | 12 | 29 | 323 | 3.8 | 17 |
| 293 | 11 | 15 | 324 | 12 | 4.0 |
| 294 | 4.9 | 13 | 325 | 3.6 | 13 |
| 295 | 180 | 12 | 326 | 26 | 13 |
| 296 | 2.0 | 56 | 327 | 9.5 | 15 |
| 297 | 58 | 45 | 328 | 40 | 14 |
| 298 | 2.6 | 52 | 329 | 13 | 14 |
| 299 | 93 | 12 | 330 | 10 | 13 |
| 300 | 0.7 | 27 | 331 | 3.8 | 14 |
| 301 | 336 | 22 | 332 | 33 | 14 |
| 302 | 1.2 | >25 | 333 | 3.1 | 41 |
| 303 | 295 | 23 | 334 | 245 | 17 |
| | | | 335 | 21 | ~14 |

### 5.3 Comparison With Compounds Exemplified in or Encompassed by WO/2015-026792

The antiviral properties of some of the compounds of the present application have been compared in the table below with one compound exemplified in WO-2015/026792 and with one compound (i.e. Compound A) that is encompassed by WO-2015/026792 but not exemplified therein.

Compound A has been specifically made to allow for this comparative testing as is structurally differs with compound (272) in the presence of R² is methyl.

Compound 321, exemplified in WO-2015/026792, on page 211 has been resynthesized and tested in the antiviral RSV assay described in Example 5.1 to allow for a direct comparison in antiviral effect.

The antiviral data (EC₅₀ values) against RSV in the table below demonstrate the unexpectedly improved antiviral properties against the respiratory syncytial virus (RSV) for the compounds that have a non-hydrogen substituent on the carbon bearing the R² substituent.

| | | | | | |
|---|---|---|---|---|---|
| Structure | EC₅₀ (nM) | CC₅₀ (µM) | Structure | EC₅₀ (nM) | CC₅₀ (µM) |
| 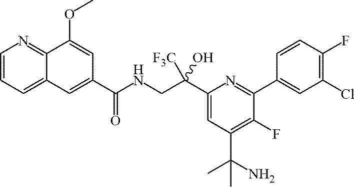 [SMILES: COc1cc(C(=O)NCC(O)(c2cc(C(C)(C)N)c(F)c(-c3ccc(F)c(Cl)c3)n2)C(F)(F)F)cc2cccnc12] | 53 | 13 | 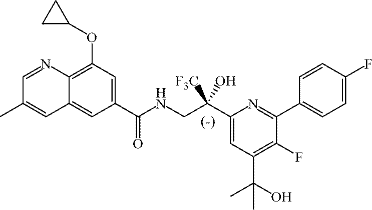 | 4 | 17 |
| Compound 321, page 211, of WO-2015/026792 | | | Co. No. (272) | | |
| 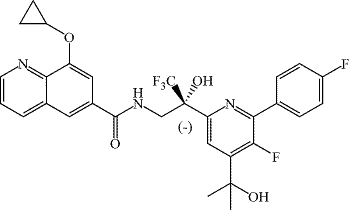 | 14 | 37 | 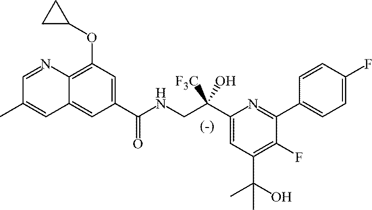 | 4 | 17 |
| Compound A covered by WO-2015/026792 | | | Co. No. (272) | | |
| | | | 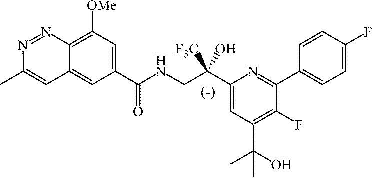 | 0.6 | 23 |
| | | | Co. No (234) | | |
| | | | 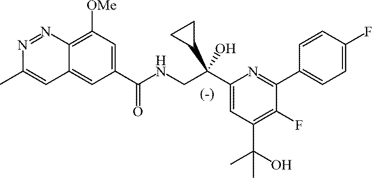 | 0.3 | 38 |
| | | | Co. No. (252) | | |
| | | | 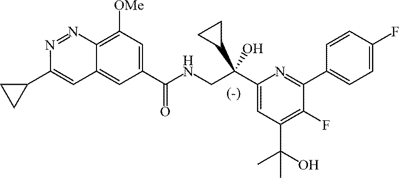 | 1.5 | 11 |
| | | | Co. No. (271) | | |

### 5.4. RSV Replicon Assay Protocol

Cell Line : APC-126
Culture Media : DMEM/Hams F-12 50/50 (Cat#10-092-CM, Mediatech)
   +10% FBS
   +1% Penicillin/Streptomycin (Cat# 30-002-CI, Mediatech)
   +1x NEAA (Cat#25-025-CI, Mediatech)
   5% Tryptose Phosphate Broth (Cat# 1682149, MP Biomedicals, lifetech silver refridger near promega freezer)
   10 µg/mL Blasticidin (Cat# Ant-BL, from Invitrogen, stored at -20C freezer door shelf in cell culture room)
Serum Shift Media: Same as above, with 40% Human Serum (BioreclamationIVT Cat# HMSRM-HI, Lot# BRH1331063, -80C in cell culture room, top shelf) replacing the 10% FBS

### Procedure :

1. 24 hours before assay, split and plate cells, total 32 white plates with clear bottom (Thursday morning). 8 plates for each condition: CC_{50_}FBS, CC_{50_}HS, EC_{50_}FBS, EC_{50_}HS
   a. Plate with Culture Media
   b. 60,000 cells/mL, 90 µL/well in 96-well Coming Cell-Culture Treated white plate with clear botttom
   c. Leave column 1 and 12 with Media only
   d. Passage generally at ~1^{∗}10^6 cells in a Coming T-175 flask (twice weekly, Mondy and Thurday)
2. Prepare dilutions (Thursday)
   a. Dilutions are made in DMSO, serially diluted at 1:5 for 9 series in singlet in v-bottom 96-well plate. (total 16 compounds, 2 plates are required.)
   b. 100 µL DMSO throughout the plate except Col#3
   c. Top Concentrations @ Col#3, 125 µl . 5-fold dilution (25µl compound to 100 µL DMSO)
      i. CC₅₀ plates: 10 mM top conc. in DMSO
      ii. EC₅₀ plates: 1 mM top conc. in DMSO (10-fold dilution from CC₅₀ plate, 10 µL compound+90 µL DMSO)
   d. Transfer 1:10 into Serum-Free Media (DMEM/Hams F-12 50/50 with no additions) (15µL compound + 135 µL medium)
3. Prepare Human Serum plates (Friday)
   a. Immediately before dosing, aspirate media from plates designated for serum shift
   b. Add 90 µL/well Serum Shift Media (SFM-APC medium 105 ml + 70 ml Human Serum)
4. Dose plates (Friday)
   a. Dose 1:10 from Serum-Free Media plates (10 µL each well)
   b. Final Top Concentrations in Cell Plates:
      i. CC₅₀: 100 µM
      ii. EC₅₀: 10 µM
5. Readout (Monday)
   a. 72 hours post-dose
   b. CC₅₀
      i. Assay: Promega Cell Titer Glo
      ii. Add 100 µL per well on top of supernatant
      iii. Measure Luminescence, 1 s per well
   c. EC₅₀
      i. Assay: Promega Renilla Glo
      ii. Add 100 µL per well on top of supernatant
      iii. Measure Luminescence, 1 s per well
Plate labeling:
CH (cytotox, 40% human Serum)
EH (Efficacy, 40% human serum)
CF (cytotox, 10% FBS)
EF (Efficacy, 10% FBS)

### 6. Prophetic Composition Examples

“Active ingredient” as used throughout these examples relates to a final compound of Formula (I), the pharmaceutically acceptable salts thereof, the solvates and the stereochemically isomeric forms and the tautomers thereof.

Typical examples of recipes for the formulation of the invention are as follows:

### 6.1. Tablets

| | |
|---|---|
| Active ingredient | 5 to 50 mg |
| Di calcium phosphate | 20 mg |
| Lactose | 30 mg |
| Talcum | 10 mg |
| Magnesium stearate | 5 mg |
| Potato starch | ad 200 mg |

In this Example, active ingredient can be replaced with the same amount of any of the compounds according to the present invention, in particular by the same amount of any of the exemplified compounds.

### 6.2. Suspension

An aqueous suspension is prepared for oral administration so that each 1 milliliter contains 1 to 5 mg of one of the active compounds, 50 mg of sodium carboxymethyl cellulose, 1 mg of sodium benzoate, 500 mg of sorbitol and water ad 1 ml.

### 6.3. Injectable

A parenteral composition is prepared by stirring 1.5 % by weight of active ingredient of the invention in 10% by volume propylene glycol in water.

### 6.4. Ointment

| | |
|---|---|
| Active ingredient | 5 to 1000 mg |
| Stearyl alcohol | 3 g |
| Lanoline | 5 g |
| White petroleum | 15 g |
| Water | ad 100 g |

In this Example, active ingredient can be replaced with the same amount of any of the compounds according to the present invention, in particular by the same amount of any of the exemplified compounds.

1. A compound of formula (I); 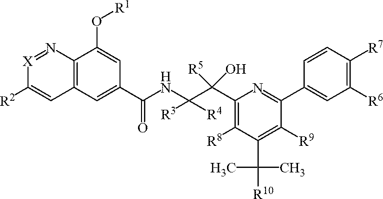
a stereochemically isomeric form thereof, or a pharmaceutically acceptable acid addition salt thereof, wherein
Xis CH, CF or N;
R¹ is C₁_₃alkyl, cyclopropyl, CHF₂ or CF₃;
R² is CH₃, CD₃, C₃_₄cycloalkyl, CH₂F, CHF₂, or CF₃;
R³ and R⁴ are each individually selected from the group consisting of hydrogen and deuterium;
R⁵ is CF₃, CHF₂, CH₃, ethyl, isopropyl or cyclopropyl, wherein the isopropyl or cyclopropyl is unsubstituted or substituted with one or two substituents each individually selected from the group consisting of halo, hydroxy, CH₃, and CH₃O;
R⁶ is hydrogen, CH₃ or halo;
R⁷ is hydrogen, halo, CF₃ or cyclopropyl;
R⁸ is hydrogen, CH₃, F, or Cl;
R⁹ is hydrogen, F, or Cl; and
R¹⁰ is hydroxy, C₁.₄alkyl—SO₂—NH— or C₁_₄alkyl-CO-NH.

2. The compound as claimed in claim 1,or a pharmaceutically acceptable acid addition salt thereof, wherein
Xis CH, CF or N;
R¹ is C₁_₃alkyl, cyclopropyl, CHF₂ or CF₃;
R² is CH₃, CD₃, C₃₋₄cycloalkyl, CH₂F, CHF₂, or CF₃;
R³ and R⁴ are each individually selected from the group consisting of hydrogen and deuterium;
R⁵ is CF₃, CHF₂, CH₃, ethyl, isopropyl or cyclopropyl, wherein the isopropyl or cyclopropyl is unsubstituted or substituted with one or two substituents each individually selected from the group consisting of halo, hydroxy, CH₃, and CH₃O;
R⁶ is hydrogen, CH₃ or halo;
R⁷ is hydrogen, halo, CF₃ or cyclopropyl;
R⁸ is hydrogen, CH₃, F, or Cl;
R⁹ is hydrogen, F, or Cl; and
with the proviso than that when R⁸ is F or Cl then R⁹ is other than hydrogen;
R¹⁰ is hydroxy, C₁₋₄alkyl—SO₂—NH— or C₁_₄alkyl-CO-NH.

3. The compound as claimed in claim 1 having the (-) specific rotation.

4. The compound as claimed in claim 1, wherein X is CH or CF.

5. The compound as claimed in claim 1, wherein X is N.

6. The compound as claimed in claim 1. wherein R¹ is CH₃ or cyclopropyl.

7. The compound as claimed in claim 1, wherein R² is CH₃, CHF₂ or cyclopropyl.

8. The compound as claimed in claim 1,wherein R¹⁰ is hydroxy.

9. The compound as claimed in claim 1, wherein X is CH; R¹ is CH₃ or cyclopropyl; R² is CH₃, CHF₂ or cyclopropyl; R³ and R⁴ are hydrogen; R⁵ is CF₃ or cyclopropyl; R⁶ is hydrogen or F; R⁷ is F; R⁸ is hydrogen or F and R⁹ is halo; and R¹⁰ is hydroxy.

10. The compound as claimed in claim 1, wherein X is N; R¹ is CH₃ or cyclopropyl; R² is CH₃, CHF₂ or cyclopropyl; R³ and R⁴ are hydrogen; R⁵ is CF₃ or cyclopropyl; R⁶ is hydrogen or F; R⁷ is F; R⁸ is hydrogen or F and R⁹ is halo; and R¹⁰ is hydroxy.

11. The compound as claimed in claim 1, wherein the compound is selected from the group consisting of 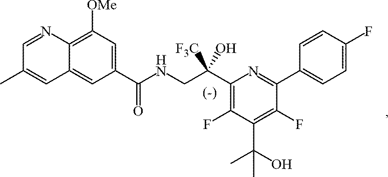 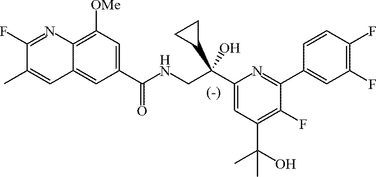 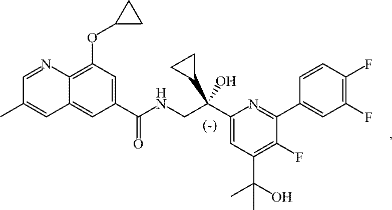 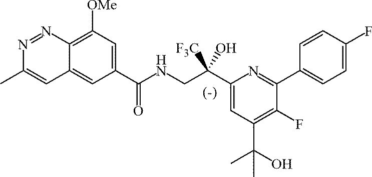 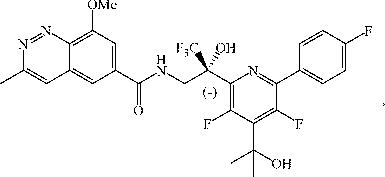 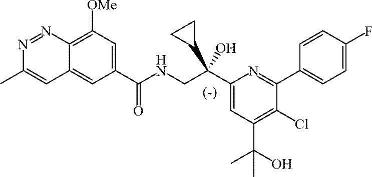 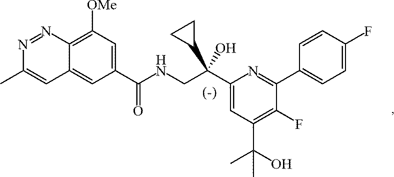 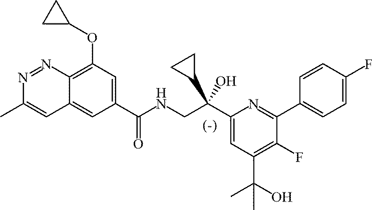 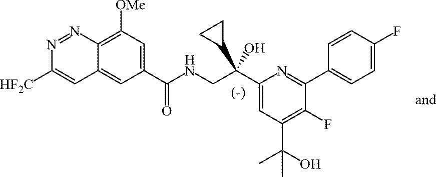 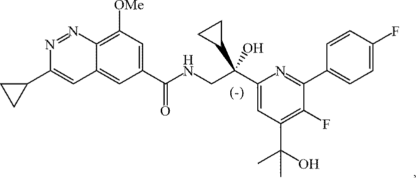
or a pharmaceutically acceptable acid addition salt thereof.

12. A pharmaceutical composition comprising a pharmaceutically acceptable carrier and a therapeutically active amount of a compound as claimed in claim 1.

13. The pharmaceutical composition according to claim 12, which further comprises another antiviral agent.

14. The pharmaceutical composition according to claim 13, wherein the other antiviral agent is a RSV inhibiting compound.

15. A process for preparing the pharmaceutical composition as claimed in claim 12, comprising mixing a therapeutically active amount of the compound of formula (I) with a pharmaceutically acceptable carrier.

16. A compound as claimed in claim 1 for use as a medicine.

17. A method of treating or preventing a respiratory syncytial virus infection in a subject in need thereof, the method comprising administering to the subject a therapeutically effective amount of the compound as claimed in claim 1.

18. A method of treating or preventing a respiratory syncytial virus infection in a subject in need thereof, the method comprising administering to the subject a therapeutically effective amount of the pharmaceutical composition as claimed in claim 12.
